(19) United States
(12) Patent Application Publication
(10) Pub. No.: US 2024/0148772 A1
(43) Pub. Date: May 9, 2024

(54) METHODS AND COMPOSITIONS FOR TREATING A PREMATURE TERMINATION CODON-MEDIATED DISORDER

(71) Applicant: Tevard Biosciences, Inc., Newton, MA (US)

(72) Inventors: Sean McFarland, Newton, MA (US); Ying-Hsin Chen, Newton, MA (US); Peter M. Eimon, Boston, MA (US)

(21) Appl. No.: 17/773,112

(22) PCT Filed: Oct. 30, 2020
(86) PCT No.: PCT/US2020/058415

§ 371 Date: Apr. 29, 2022

Related U.S. Application Data
(60) Provisional application No. 62/929,428, filed on Nov. 1, 2019.

(51) Int. Cl. A61K 31/7115; C12N 15/86

(52) U.S. Cl. CPC A61K 31/7115; C12N 15/86; C12N 2750/14143

(57) ABSTRACT

The invention relates generally to modified tRNAs and the use of modified tRNAs to express in a mammalian cell a functional gene product encoded by a gene containing a premature termination codon and/or to treat a disease mediated by a premature termination codon, e.g., Dravet syndrome.

### CROSS-REFERENCE TO RELATED APPLICATIONS

This application claims the benefit of and priority to U.S. Provisional Patent Application No. 62/929,428, filed Nov. 1, 2019, which is incorporated herein by reference in its entirety for all purposes.

### FIELD OF THE INVENTION

The invention relates generally to methods and compositions for expressing a gene product encoded by a gene containing a premature termination codon and/or treating a disorder mediated by a premature termination codon.

### BACKGROUND

Protein synthesis is directed by a genetic code that includes 61 three-base-pair codons encoding amino acids that are incorporated into the protein being synthesized and 3 three-base-pair codons (referred to as stop or termination codons) that terminate the synthesis of a protein. When a nucleic acid sequence encoding a protein is mutated to contain a premature termination codon rather than a codon for the next amino acid, the resulting protein is prematurely terminated, which is often nonfunctional or less functional than the untruncated or full length protein. Such mutations, termed nonsense mutations, are often associated with, or are a causative agent in numerous different genetic diseases.

A number of disorders are associated with, or are caused by nonsense mutations. These include epilepsies, for example, Dravet Syndrome, Genetic Epilepsy with Febrile Seizures (GEFS), Benign Familial Infantile Epilepsy (BFIE), Early Infantile Epileptic Encephalopathy (EIEE), Lennox-Gastaut Syndrome, Rett Syndrome, PPM-X Syndrome, Ohtahara Syndrome, Episodic Ataxia, Hemiplegic Migraine, Iditiopathic Generalized Epilepsy, FOXG1 Syndrome, Familial Focal Epilepsy with Variable Foci (FFEVF), Childhood-Onset Epileptic Encephalopathy, SYNGAP1-Related Intellectual Disability, Pyridoxine-Dependent Epilepsy, Familial Infantile Myoclonic Epilepsy (FIME), Myoclonic Astatic Epilepsy, X-Linked Intellectual Disability, Partial Epilepsy and Episodic Ataxia, Febrile Seizures, Autosomal Dominant Partial Epilepsy with Auditory Features (ADPEAF), PNPO-Deficiency, Progressive Myoclonus Epilepsy, Action Myoclonus—Renal Failure (AMRF), CDKL5 deficiency disorder, and Benign Familial Infantile Seizures (BFIS).

By way of example, Dravet Syndrome is a rare and catastrophic form of intractable epilepsy that begins in infancy Initially, patients experience prolonged seizures. In their second year, additional types of seizure begin to occur, which typically coincide with a developmental decline, possibly due to repeated cerebral hypoxia. This leads to poor development of language and motor skills. Mutations in SCN1A (encoding the voltage-gated sodium channel a subunit Nav1.1), SCN1B (encoding the voltage-gated sodium channel (31 subunit), SCN2A (encoding Nav1.2), SCN3A (encoding Nav1.3), SCN9A (encoding Nav1.7), GABRG2 (encoding the γ-aminobutyric acid receptor γ2 subunit), GABRD (encoding the γ-aminobutyric acid receptor A subunit) and/or PCDH19 (encoding Protocadherin-19) genes have been linked to Dravet Syndrome.

Dravet syndrome may be caused by a nonsense mutation in, for example, the SCN1A gene resulting in a premature termination codon and a lack of or reduced amount of untruncated or functional protein. The SCN1A gene normally codes for the neuronal voltage-gated sodium channel α subunit, Na(V)1.1. In mouse models, loss-of-function mutations in SCN1A have been observed to result in a decrease in sodium currents and impaired excitability of GABAergic interneurons of the hippocampus.

Despite the efforts made to date, there is a need in the art for improved compositions and methods for treating diseases mediated by premature termination codons, including Dravet syndrome.

### SUMMARY OF THE INVENTION

The invention is based, in part, upon the discovery of tRNAs (e.g., suppressor tRNAs), that permit an amino acid to be incorporated into a gene product encoded by a gene in a mammalian cell at a position that would otherwise result in a truncated gene product caused by a premature termination codon (PTC) in the gene. The invention is further based, in part, upon the discovery that a tRNA that permits an amino acid to be incorporated into a gene product encoded by a gene at a position that would otherwise result in a truncated gene product caused by a PTC in the gene, e.g., a tRNA described herein, can be used to treat a disease mediated by a PTC in a gene in a subject.

Accordingly, in one aspect, the invention provides a tRNA comprising a nucleotide sequence set forth in TABLE 2. In certain embodiments, the tRNA comprises a nucleotide sequence selected from SEQ ID NOs: 19-21, 37, 39, 40, 44, 179, 181, 182, and 186.

In certain embodiments, the tRNA comprises one or more naturally occurring nucleotide modifications, e.g., selected from 5-methyl uridine, 5-carbamoylmethyluridine, 5-carbamoyl-methyl-2-O-methyluridine, 5-methoxy-carbonylmethyluridine, 5-methoxycarbonylmethyl-2-thiouridine, pseudouridine, dihydrouridine, 1-methyladenosine, and inosine.

In another aspect, the invention provides an expression vector comprising a nucleotide sequence encoding any of the foregoing tRNAs. In certain embodiments, the expression vector comprises 1, 2, 3, 4, or more than 4 copy numbers of the nucleotide sequence encoding the tRNA. In certain embodiments, the expression vector comprises a nucleotide sequence corresponding to a genomic DNA sequence flanking a wild-type tRNA gene. For example, in certain embodiments, the expression vector comprises a nucleotide sequence set forth in TABLE 4. In certain embodiments, the nucleotide sequence set forth in TABLE 4 is selected from SEQ ID NOs: 869-888. In certain embodiments, the nucleotide sequence set forth in TABLE 4 is operably linked to the nucleotide sequence encoding the tRNA. In certain embodiments, in the expression vector, the nucleotide sequence set forth in TABLE 4 is 5′ to the nucleotide sequence encoding the tRNA. In certain embodiments, in the expression vector, the nucleotide sequence set forth in TABLE 4 is immediately 5′ to (i.e., adjacent) the nucleotide sequence encoding the tRNA.

In another aspect, the invention provides an expression vector comprising a nucleotide sequence encoding a tRNA set forth in TABLE 3, wherein the expression vector comprises 1, 2, 3, 4, or more than 4 copy numbers of the nucleotide sequence encoding the tRNA. In certain embodiments, the tRNA comprises a nucleotide sequence selected from SEQ ID NOs: 6-9, 11, 16-18, 22, 35, 36, 38, 45, 178, 180, and 187.

In another aspect, the invention provides an expression vector comprising a nucleotide sequence encoding a tRNA set forth in TABLE 3, wherein the expression vector further comprises a nucleotide sequence set forth in TABLE 4. In certain embodiments, the tRNA comprises a nucleotide sequence selected from SEQ ID NOs: 6-9, 11, 16-18, 22, 35, 36, 38, 45, 178, 180, and 187. In certain embodiments, the nucleotide sequence set forth in TABLE 4 is selected from SEQ ID NOs: 869-888. In certain embodiments, the nucleotide sequence set forth in TABLE 4 is operably linked to the nucleotide sequence encoding the tRNA. In certain embodiments, in the expression vector, the nucleotide sequence set forth in TABLE 4 is 5′ to the nucleotide sequence encoding the tRNA. In certain embodiments, in the expression vector, the nucleotide sequence set forth in TABLE 4 is immediately 5′ to (i.e., adjacent) the nucleotide sequence encoding the tRNA.

In certain embodiments of any of the foregoing expression vectors, the expression vector is a viral vector, e.g., a DNA virus vector, e.g., an adeno-associated virus (AAV) vector.

In another aspect, the invention provides a pharmaceutical composition comprising any of the foregoing tRNAs or any of the foregoing expression vectors and a pharmaceutically acceptable excipient. In certain embodiments, the tRNA or expression vector is not conjugated to, or associated with, another moiety, e.g., a carrier particle, e.g., an aminolipid particle. In certain embodiments, the composition does not comprise a nanoparticle and/or an aminolipid delivery compound.

In another aspect, the invention provides a method of expressing in a mammalian cell a functional gene product encoded by a gene containing a premature termination codon, the method comprising introducing into the cell an effective amount of any of the foregoing tRNAs or expression vectors, thereby permitting an amino acid to be incorporated into the gene product at a position that would otherwise result in a truncated gene product caused by the premature termination codon. In certain embodiments of any of the foregoing methods, the gene is selected from a gene set forth in TABLE 5 or TABLE 6. In certain embodiments, the gene is a SCN1A gene.

In another aspect, the invention provides a method of expressing in a mammalian cell a functional gene product encoded by a gene containing a premature termination codon, the method comprising introducing into the cell an effective amount of a tRNA set forth in TABLE 3 (e.g., a tRNA comprising a nucleotide sequence selected from SEQ ID NOs: 6-9, 11, 16-18, 22, 35, 36, 38, 45, 178, 180, and 187), or an expression vector comprising a nucleotide sequence encoding the tRNA, thereby permitting an amino acid to be incorporated into the gene product at a position that would otherwise result in a truncated gene product caused by the premature termination codon, wherein the gene is a gene set forth in TABLE 5. In certain embodiments, the gene is a SCN1A gene.

In certain embodiments of any of the foregoing methods, the cell contains less truncated gene product than a cell without the tRNA. In certain embodiments, the cell contains a greater amount of functional gene product than a cell without the tRNA.

In another aspect, the invention provides a method of increasing in a cell voltage-gated sodium channel activity encoded by a SCN1A gene containing a premature termination codon, the method comprising introducing into the cell an effective amount of any of the foregoing tRNAs or any of the foregoing expression vectors, thereby permitting an amino acid to be incorporated into the SCN1A gene product at a position that would otherwise result in a truncated SCN1A gene product caused by the premature termination codon.

In another aspect, the invention provides a method of increasing in a cell voltage-gated sodium channel activity encoded by a SCN1A gene containing a premature termination codon, the method comprising introducing into the cell an effective amount of a tRNA set forth in TABLE 3 (e.g., a tRNA comprising a nucleotide sequence selected from SEQ ID NOs: 6-9, 11, 16-18, 22, 35, 36, 38, 45, 178, 180, and 187), or an expression vector comprising a nucleotide sequence encoding the tRNA, thereby permitting an amino acid to be incorporated into the SCN1A gene product at a position that would otherwise result in a truncated SCN1A gene product caused by the premature termination codon.

In certain embodiments of any of the foregoing methods, wherein the gene is a SCN1A gene, the SCN1A gene product produced with the tRNA is a functional SCN1A gene product. In certain embodiments, the functional SCN1A gene product has greater activity than the truncated SCN1A gene product. In certain embodiments, the functional SCN1A gene product is the Nav1.1 protein. In certain embodiments, the functional SCN1A gene product comprises the amino acid sequence of any one of SEQ ID NOs: 863-868.

In certain embodiments of any of the foregoing methods, the cell is a human cell. In certain embodiments, the cell is a central nervous system cell, e.g., a neuron. In certain embodiments, the tRNA becomes aminoacylated in the cell.

In another aspect, the invention provides a method of treating a premature termination codon-mediated disorder in a subject in need thereof, wherein the subject has a gene with a premature termination codon, the method comprising administering to the subject an effective amount of any of the foregoing tRNAs or any of the foregoing expression vectors, thereby to treat the disorder in the subject. In certain embodiments, the disorder is selected from a disorder set forth in TABLE 5 or TABLE 6.

In another aspect, the invention provides a method of treating a premature termination codon-mediated disorder in a subject in need thereof, wherein the subject has a gene with a premature termination codon, the method comprising administering to the subject an effective amount of a tRNA set forth in TABLE 3 (e.g., a tRNA comprising a nucleotide sequence selected from SEQ ID NOs: 6-9, 11, 16-18, 22, 35, 36, 38, 45, 178, 180, and 187), or an expression vector comprising a nucleotide sequence encoding the tRNA, thereby to treat the disorder in the subject, wherein the disorder is a disorder set forth in TABLE 5.

In certain embodiments of any of the foregoing methods of treatment, the disorder is an epilepsy, e.g., Dravet syndrome. In certain embodiments, the subject is human. In certain embodiments, the method further comprises administering an effective amount of another agent, e.g., DIACOMIT® (stiripentol), EPIODOLEX® (cannabidiol), a ketogenic diet, ONFI® (clobazam), TOPAMAX® (topiramate), fenfluramine, or valproic acid, to the subject.

In certain embodiments of any of the foregoing methods, wherein the gene is a SCN1A gene, the premature termination codon in the SCN1A gene is caused by a mutation, or a combination of mutations, selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T. In certain embodiments, the premature termination codon is caused by a mutation selected from c.1738C>T and c.3985C>T.

These and other aspects and features of the invention are described in the following detailed description and claims.

### DESCRIPTION OF THE DRAWINGS

The invention can be more completely understood with reference to the following drawings.

FIG. 1 is a schematic representation of a transcript (e.g., an SCN1A transcript) containing a premature termination codon (PTC) which leads to a truncated protein product (e.g., a protein product in a subject with Dravet syndrome). Native termination codons are indicated as shaded circles, and premature termination codons are indicated as unshaded circles. Expression of a suppressor tRNA (e.g., an anticodon modified arginine tRNA) charged with its cognate amino acid (A.A.) allows read-through of the PTC and facilitates expression of the full-length protein.

FIG. 2A is a consensus tRNA secondary structure. The numbering of the residues is based on the tRNA numbering system described in Steinberg et al. (1993) NUCLEIC ACIDS RES. 21:3011-15. FIG. 2B is a table showing the modification profile for tRNA sequences from the cytosol of certain eukaryotic organisms. The ratios in the table indicate the frequency of occurrence of listed nucleotide at the numbered position shown in FIG. 2A. The abbreviations for the modified residues are defined in Motorin et al. (2005) “Transfer RNA Modification,” ENCYCLOPEDIA OF LIFE SCIENCES, John Wily & Sons, Inc.

FIG. 3 is a schematic representation of a dual fluorescent reporter construct which contains three copies of a red fluorescent protein (tdTomato), a TEV protease, a 51 bp linker region comprising a PTC+/−8 flanking codons, and three copies of a green fluorescent protein (EGFP). Expression is driven by a promoter for elongation factor EF-1a located upstream of the first copy of tdTomato. All copies of tdTomato, EGFP, and the TEV protease are separated from one another by TEV protease cleavage sites (Glu-Asn-Leu-Tyr-Phe-Gln-(Gly/Ser)).

FIG. 4 is a graph depicting readthrough activity of Arg_{TCA} suppressor tRNAs in a Flp-In-293 cell line stably expressing a dual fluorescent reporter with the subject S-PTC linker region (SEQ ID NO: 29), which is derived from a clinically relevant SCN1A nonsense mutation linked to Dravet syndrome. Cells were transfected with the indicated Arg_{TCA} suppressor tRNAs (SEQ ID NOs: 1-25 and 35) and readthrough activity was measured by flow cytometry in two independent experiments at 24 hours post-transfection. “Parental” indicates the original Flp-In-293 cell line without a fluorescent reporter, “no PTC” indicates a Flp-In-293 cell line stably expressing a dual fluorescent reporter with a version of the subject S-PTC linker region that lacks a PTC (SEQ ID NO: 194), “EV” (empty vector) indicates cells transfected with an expression construct that does not contain a tRNA, “TCG” indicates cells transfected with an expression construct that contains a wild-type Arg-tRNA with a TCG anticodon. Readthrough activity is presented as the percentage of viable cells that that express both tdTomato and EGFP above background (“Double positive %”). Error bars represent the standard deviation of the data.

FIG. 5 is a graph depicting readthrough activity of Arg_{TCA} suppressor tRNAs in a Flp-In-3T3 cell line stably expressing a dual fluorescent reporter with the R1407X-PTC linker region (SEQ ID NO: 30), which is derived from a clinically relevant SCN1A nonsense mutation linked to Dravet syndrome. Cells were transfected with the indicated Arg_{TCA} suppressor tRNAs (SEQ ID NOs: 1-25) and readthrough activity was measured by flow cytometry in three independent experiments at 24 hours post transfection. “Parental” indicates the original 3T3 cell line without a fluorescent reporter, “no PTC” indicates an 3T3 cell line stably expressing a dual fluorescent reporter with a version of the R1407X-PTC linker region that lacks a PTC (SEQ ID NO: 195), “Mock” indicates mock-transfected cells, “EV” (empty vector) indicates cells transfected with an expression construct that does not contain a tRNA or an EGFP reporter, “TCG” indicates cells transfected with an expression construct that contained a wild-type Arg-tRNA with a TCG anticodon. Readthrough activity is presented as the percentage of viable cells that that express both tdTomato and EGFP above background (“double positive %”). Error bars represent the standard deviation of the data.

FIG. 6 is a graph depicting readthrough activity of Arg_{TCA} suppressor tRNAs in Flp-In-3T3 cells transiently transfected with a dual fluorescent reporter with the subject N-PTC linker region (SEQ ID NO: 28), which is derived from a clinically relevant SCN1A nonsense mutation linked to Dravet syndrome. Cells were co-transfected with the indicated Arg_{TCA} suppressor tRNAs (SEQ ID NOs: 1-25 and 35) and readthrough activity was measured by flow cytometry. “Mock” indicates mock-transfected cells, “no PTC” indicates cells transfected with a dual fluorescent reporter with a version of the subject N-PTC linker region that lacks a PTC (SEQ ID NO: 193), “EV” (empty vector) indicates cells co-transfected with an expression construct that does not contain a tRNA, “TCG” indicates cells co-transfected with an expression construct that contains a wild-type Arg-tRNA with a TCG anticodon. Readthrough activity was measured by flow cytometry and is presented as the percentage of viable cells that that express both tdTomato and EGFP above background (“double positive %”).

FIG. 7 is a graph depicting readthrough activity of the indicated Arg_{TCA} suppressor tRNAs (as measured by percent positive GFP cells) in Neuro-2a (N2a) and Flp-In-293 (293) cells. Cells were co-transfected with an expression construct containing an EGFP-R96X-TGA reporter (SEQ ID NO: 31) and an expression construct containing the indicated ArgicA suppressor tRNA (SEQ ID NOs: 1-22). “EGFP” indicates cells transfected with a version of the EGFP reporter that lacks a PTC, “EGFP-PTC” indicates cells transfected with the EGFP-R96X-TGA reporter alone. EGFP expression was analyzed by flow cytometry at ˜24 hours post transfection in 293 cells and ˜48 hours post transfection in N2a cells. Readthrough activity is presented as the percentage of viable cells that express EGFP above background (“% GFP+”) with all values normalized to cells expressing EGFP lacking a PTC.

FIG. 8A is a depiction of an experimental approach to measure suppressor tRNA activity using a construct which contains an EGFP reporter with a PTC and a suppressor tRNA. Native termination codons are indicated as shaded circles, and premature termination codons are indicated as unshaded circles. A standard Arg-tRNA (with an anticodon that binds CGA) will result in no read-through of the PTC in EGFP, and a non-functional truncated EGFP protein. A suppressor tRNA (with an anticodon that binds UGA) allows for read-through the PTC in EGFP resulting in full-length, functional EGFP protein. FIG. 8B is a schematic representation of an exemplary reporter construct which contains EGFP with a PTC and 4 copies of a suppressor tRNA.

FIG. 9 depicts fluorescent images of Neuro-2a cells transfected with an expression construct containing an EGFP-R96X-TGA reporter (SEQ ID NO: 31) and the indicated Arg_{TCA} suppressor tRNA at the indicated copy number. Suppressor tRNAs are TCA-001 (SEQ ID NO: 11), TCA-113 (SEQ ID NO: 16), and TCA-115 (SEQ ID NO: 18). Each copy of the suppressor tRNA also contains 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32). Images were taken ˜24 hours post transfection. From left to right, the GFP controls are 1) wild-type EGFP alone, 2) wild-type EGFP on an expression construct including a single copy of the Arg_{TCA} suppressor tRNA #001 (SEQ ID NO: 11), 3) the EGFP-R96X-TGA reporter alone, and 4) the EGFP-R96X-TGA reporter on an expression construct including four copies of a wild-type Arg-tRNA with an unmodified TCG anticodon.

FIG. 10 depicts fluorescent images of Flp-In-293 cells transfected with an expression construct containing an EGFP-R96X-TGA reporter (SEQ ID NO: 31) and the indicated Arg_{TCA} suppressor tRNA at the indicated copy number. Suppressor tRNAs are TCA-001 (SEQ ID NO: 11), TCA-113 (SEQ ID NO: 16), and TCA-115 (SEQ ID NO: 18). Each copy of the suppressor tRNA also contained 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32). Images were taken ˜48 hours post transfection. From left to right, the GFP controls are 1) wild-type EGFP alone, 2) wild-type EGFP on an expression construct including a single copy of the Arg_{TCA} suppressor tRNA #001 (SEQ ID NO: 11), 3) the EGFP-R96X-TGA reporter alone, and 4) the EGFP-R96X-TGA reporter on an expression construct including four copies of a wild-type Arg-tRNA with an unmodified TCG anticodon.

FIG. 11 shows fluorescence as measured by flow cytometry for Neuro-2a and Flp-In-293 cells transfected with an expression construct containing an EGFP-R96X-TGA reporter (SEQ ID NO: 177) and the Arg_{TCA} suppressor tRNA #001 (SEQ ID NO: 11). Expression constructs included one (1×), two (2×), or four (4×) copies of the suppressor tRNA in the context of either (i) a U6 promoter including 19 bps of upstream flanking genomic DNA from tRNA-Arg-TCG-1-1 (SEQ ID NO: 33) and 46 bps of downstream flanking genomic DNA from tRNA-Arg-TCG-1-1 (SEQ ID NO: 34) (“U6”), or (ii) 200 bps upstream flanking genomic DNA from tRNA-Arg-TCG-1-1 (SEQ ID NO: 26) and 200 bps of downstream flanking genomic DNA from tRNA-Arg-TCG-1-1 (SEQ ID NO: 27) (“Flank”). “Empty vector” indicates cells transfected with an expression construct that does not contain a tRNA or an EGFP reporter, “EF1a:EGFP” indicates cells transfected with a version of the EGFP reporter that lacks a PTC. Analysis was carried out ˜48 hours post transfection. Data are presented as histograms displaying the frequency distribution of the data versus fluorescence intensity for viable cells expressing EGFP above background.

FIG. 12 depicts percentage of EGFP positive cells and mean EGFP intensity in all viable cells as measured by flow cytometry for Neuro-2a cells transfected with an expression construct containing an EGFP-R96X-TGA reporter (SEQ ID NO: 177) and the Arg_{TCA} suppressor tRNA #001 (SEQ ID NO: 11) with the indicated copy number, in the context of either (i) a U6 promoter including 19 bps of upstream flanking genomic DNA from tRNA-Arg-TCG-1-1 (SEQ ID NO: 33) and 46 bps of downstream flanking genomic DNA from tRNA-Arg-TCG-1-1 (SEQ ID NO: 34) (“U6”), or (ii) 200 bps upstream flanking genomic DNA from tRNA-Arg-TCG-1-1 (SEQ ID NO: 26) and 200 bps of downstream flanking genomic DNA from tRNA-Arg-TCG-1-1 (SEQ ID NO: 27) (“Flank”). “Empty vector” indicates cells transfected with an expression construct that does not contain a tRNA or an EGFP reporter, “EF1a:EGFP” indicates cells transfected with a version of the EGFP reporter that lacks a PTC. Analysis was carried out ˜48 hours post transfection. These plots summarize data from Neuro-2a cells in FIG. 11.

FIG. 13 shows fluorescence as measured by flow cytometry for Neuro-2a cells transfected with an expression construct containing an EGFP-R96X-TGA reporter (SEQ ID NO: 31) and the Arg_{TCA} suppressor tRNA #001 (SEQ ID NO: 11). Expression constructs included one (1×), two (2×), three (3×), or four (4×) copies of the suppressor tRNA. Each copy of the suppressor tRNA also contains 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32) (“Flank”). “CAG:EGFP” indicates cells transfected with a version of the EGFP reporter that lacks a PTC, “Mock” indicates cells transfected with the EGFP-R96X-TGA reporter alone. Analysis was carried out ˜48 hours post transfection. Data are presented as histograms displaying the frequency distribution of the data versus fluorescence intensity for all viable cells (“G1 Gate”) and for viable cells expressing EGFP above background (“GFP+Gate”).

FIG. 14 shows fluorescence as measured by flow cytometry for Neuro-2a cells transfected with an expression construct containing an EGFP-R96X-TGA reporter (SEQ ID NO: 31) and the Arg_{TCA} suppressor tRNA #113 (SEQ ID NO: 16). Expression constructs included one (1×), two (2×), three (3×), or four (4×) copies of the suppressor tRNA. Each copy of the suppressor tRNA also contained 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32) (“Flank”). “CAG:EGFP” indicates cells transfected with a version of the EGFP reporter that lacks a PTC, “Mock” indicates cells transfected with the EGFP-PTC reporter alone. Analysis was carried out ˜48 hours post transfection. Data are presented as histograms displaying the frequency distribution of the data versus fluorescence intensity for all viable cells (“G1 Gate”) and for viable cells expressing EGFP above background (“GFP+Gate”).

FIG. 15 shows fluorescence as measured by flow cytometry for Neuro-2a cells transfected with an expression construct containing an EGFP-R96X-TGA reporter (SEQ ID NO: 31) and the Arg_{TCA} suppressor tRNA #115 (SEQ ID NO: 18). Expression constructs included one (1×), two (2×), three (3×), or four (4×) copies of the suppressor tRNA. Each copy of the suppressor tRNA also contained 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32) (“Flank”). “CAG:EGFP” indicates cells transfected with a version of the EGFP reporter that lacks a PTC, “Mock” indicates cells transfected with the EGFP-R96X-TGA reporter alone. Analysis was carried out ˜48 hours post transfection. Data are presented as histograms displaying the frequency distribution of the data versus fluorescence intensity for all viable cells (“G1 Gate”) and for viable cells expressing EGFP above background (“GFP+Gate”).

FIG. 16 depicts percentage of EGFP positive cells in all viable cells (“% Cells GFP+”) and mean EGFP intensity in EGFP positive cells (“GFP+Gate”) as measured by flow cytometry for Neuro-2a cells transfected with an expression construct containing an EGFP-R96X-TGA reporter (SEQ ID NO: 31) and the indicated suppressor tRNA at the indicated copy number. Suppressor tRNAs are TCA-001 (SEQ ID NO: 11), TCA-113 (SEQ ID NO: 16), and TCA-115 (SEQ ID NO: 18). “CAG:EGFP” indicates cells transfected with a version of the EGFP reporter that lacks a PTC, “CAG:EGFP-PTC” indicates cells transfected with the EGFP-R96X-TGA reporter alone. These plots summarize data from FIGS. 13-15.

FIG. 17 depicts the percentage of EGFP positive cells as measured by flow cytometry for Neuro-2a cells transfected with an expression construct containing an EGFP-R96X-TGA reporter (SEQ ID NO: 31) and the indicated Arg_{TCA} suppressor tRNA with the indicated copy number and flanking sequence. Suppressor tRNAs are TCA-001 (SEQ ID NO: 11), TCA-113 (SEQ ID NO: 16), and TCA-115 (SEQ ID NO: 18). “U6” indicates a U6 promoter including 19 bps of upstream flanking genomic DNA from tRNA-Arg-TCG-1-1 (SEQ ID NO: 33) and 46 bps of downstream flanking genomic DNA from tRNA-Arg-TCG-1-1 (SEQ ID NO: 34), “Flank (±200 bps)” indicates 200 bps upstream flanking genomic DNA from tRNA-Arg-TCG-1-1 (SEQ ID NO: 26) and 200 bps of downstream flanking genomic DNA from tRNA-Arg-TCG-1-1 (SEQ ID NO: 27), “Flank (+200/−100 bps)” indicates 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32) (“Flank”). “Mock” indicates mock transfected cells, “Empty Vector” indicates cells transfected with an expression construct that does not contain a tRNA or an EGFP reporter, “4×TCG” indicates cells transfected with an expression construct that contains an EGFP-R96X-TGA reporter and 4 copies of a wild-type Arg-tRNA with a TCG anticodon, “CAG:EGFP” indicates cells transfected with a version of the EGFP reporter that lacks a PTC. All data are normalized to positive control (CAG:EGFP). Plots show the percentage of viable cells that express GFP above background.

FIG. 18 is a graph depicting readthrough activity of G1n_{TTA} suppressor tRNAs in two independently derived Flp-In-293 cell lines (#2 and #10) stably expressing a dual fluorescent reporter with the Dmd^{mdx}-PTC linker region (SEQ ID NO: 192), which is derived from a clinically relevant DMD nonsense mutation linked to Duchenne muscular dystrophy. Cells were transfected with the indicated G1n_{TTA} suppressor tRNAs (SEQ ID NOs: 36-48) and readthrough activity was measured by flow cytometry at 24 hours post transfection. “Parental” indicates the original Flp-In-293 cell line without a fluorescent reporter, “no PTC” indicates a Flp-In-293 cell line stably expressing a dual fluorescent reporter with a version of the Dmd^{mdx}-PTC linker region that lacks a PTC (SEQ ID NO: 191), “TTG” indicates cells transfected with an expression construct that contains a wild-type Gln-tRNA with a TTG anticodon. Readthrough activity was measured by flow cytometry and is presented as the percentage of viable cells that that express both tdTomato and EGFP above baseline (“% Double+ cells”).

FIG. 19 is a graph depicting readthrough activity of the indicated G1n_{TTA} suppressor tRNAs (SEQ ID NOs: 36-48) in Neuro-2a cells co-transfected with an expression construct containing an EGFP-Q69X-TAA reporter (SEQ ID NO: 175). “Mock” indicates mock-transfected cells, “EGFP” indicates cells transfected with a version of the EGFP reporter that lacks a PTC, “EV” (empty vector) indicates cells transfected with an expression construct that does not contain a tRNA or an EGFP reporter, “TTG” indicates cells co-transfected with the EGFP-Q69X-TAA reporter and an expression construct that contains a wild-type Gln-tRNA with a TTG anticodon. Readthrough activity was measured by flow cytometry and is presented as the percentage of viable cells that express GFP above background. Error bars represent the standard deviation of the data.

FIG. 20 is a graph depicting readthrough activity of the indicated GlncTA suppressor tRNAs (SEQ ID NOs: 78-90) in Neuro-2a cells co-transfected with an expression construct containing an EGFP-Q69X-TAG reporter (SEQ ID NO: 176). “Mock” indicates mock-transfected cells, “EGFP” indicated cells transfected with a version of the EGFP reporter that lacks a PTC, “EV” (empty vector) indicates cells transfected with an expression construct that does not contain a tRNA or an EGFP reporter, “TTG” indicates cells co-transfected with the EGFP-Q69X-TAG reporter and an expression construct that contains a wild-type Gln-tRNA with a TTG anticodon. Readthrough activity was measured by flow cytometry and is presented as the percentage of viable cells that express GFP above background. Error bars represent the standard deviation of the data.

FIG. 21 depicts fluorescent images of Neuro-2a cells ˜24 hours after transfection with an expression construct containing an EGFP-R96X-TGA reporter (“GFP-PTC”) and either (i) including the Arg_{TCA} suppressor tRNA #115 (SEQ ID NO: 18) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hours after transfection and the indicated drugs at the indicated concentrations were added at this point. Controls in the left column were transfected with an expression construct containing wild-type EGFP (“WT-GFP”) and the indicated drug or Arg_{TCA} suppressor tRNA at the indicated copy number.

FIG. 22 is a graph depicting percentage of GFP positive cells as measured by flow cytometry at ˜48 hours post transfection. Neuro-2a cells were transfected with an expression construct containing an EGFP-R96X-TGA reporter (“GFP-PTC”) and either (i) including the Arg_{TCA} suppressor tRNA #115 (SEQ ID NO: 18) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hours after transfection and the indicated drugs at the indicated concentrations were added at this point. A reporter containing wildtype EGFP without a PTC (“WT-GFP”) was used as a control. “Mock” indicates mock transfected cells. “4×TCG” indicates cells transfected with an expression construct that contains four copies of a wild-type Arg-tRNA with a TCG anticodon and the EGFP-R96X-TGA reporter. Plot shows the percentage of viable cells that express EGFP above background. The percentage of cells that expressed GFP ranged from 73.0 to 76.2% for the cells expressing the Arg_{TCA} suppressor, relative to 0.7 to 1.6% for negative controls, 0.9 to 4.9% for cells treated with ataluren, 1.2 to 6.5% for cells treated with gentamicin, and 6.7% to 26.7% for cells treated with G418.

FIG. 23 is a graph depicting cell viability from FIG. 22 as measured by flow cytometry at ˜48 hours post transfection. Neuro-2a cells were transfected with an expression construct containing the EGFP-R96X-TGA reporter (“GFP-PTC”) and either (i) transfected with an expression construct including the Arg_{TCA} suppressor tRNA #115 (SEQ ID NO: 18) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hour after transfection and the indicated drugs at the indicated concentrations were added at this point. A reporter containing wildtype GFP without a PTC (“WT-GFP”) was used as a control. “Mock” indicates mock transfected cells. “4×TCG” indicates cells transfected with an expression construct that contains four copies of a wild-type Arg-tRNA with a TCG anticodon and the EGFP-R96X-TGA reporter. Cell viability was assessed by flow cytometry using 7-Amino Actinomycin D (7-AAD), a membrane impermeant dye that is generally excluded from viable cells.

FIG. 24 is a graph depicting readthrough activity of the indicated GlncTA suppressor tRNAs (SEQ ID NOs: 178-190) in Flp-In-293 cells transiently co-transfected with the indicated dual fluorescent reporter constructs that contain linker regions (SEQ ID NOs: 889, 891, and 893) derived from three clinically relevant Gln(Q)-to-TAG PTC mutations in SCN1A (W1397*, S1505*, and Q1810*) that are linked to Dravet syndrome. “Mock” indicates mock-transfected cells, “No PTC” indicates cells transfected with versions of the dual fluorescent reporter constructs lacking a PTC (SEQ ID Nos: 890, 892, and 894), “EV” (empty vector) indicates cells transfected with an expression construct that does not contain a tRNA or a fluorescent reporter, “TTG” indicates cells co-transfected with the indicated dual fluorescent reporter construct and an expression construct that contains a wild-type Gln-tRNA with a TTG anticodon. Readthrough activity was measured by flow cytometry at ˜24 hours post-transfection and is presented as the percentage of viable cells that that express both tdTomato and EGFP above baseline (“% Double+ cells”).

FIG. 25 is a graph depicting readthrough activity of the indicated GlncTA suppressor tRNAs (SEQ ID NOs: 178-190) in a Flp-In-293 cell line containing an integrated dual fluorescent reporter construct that contains a linker region (SEQ ID NO: 889) which is derived from a clinically relevant PTC mutation in SCN1A (W1397*) that is linked to Dravet syndrome. “Mock” indicates mock-transfected cells, “RFP-EGFP” indicates a Flp-In-293 cell line containing an integrated version of the dual fluorescent reporter construct lacking a PTC (SEQ ID NO: 890), “EV” (empty vector) indicates cells transfected with an expression construct that does not contain a tRNA or an EGFP reporter, “TTG” indicates cells transfected with a wild-type Gln-tRNA with a TTG anticodon. Readthrough activity was measured by flow cytometry at ˜24 hours post transfection and is presented as the percentage of cells that that express both tdTomato and EGFP above background (“% Double+ cells”).

FIG. 26A shows fluorescence as measured by flow cytometry for Neuro-2a cells transfected with an expression construct containing an EGFP-R96X-TGA reporter (SEQ ID NO: 31) and a single copy of the Arg_{TCA} suppressor tRNA #115 (SEQ ID NO: 18), in the context of either (i) 200 bps upstream flanking genomic DNA from tRNA-Arg-TCG-1-1 (SEQ ID NO: 26) and 104 bps of downstream flanking genomic DNA from tRNA-Arg-TCG-1-1 (SEQ ID NO: 32) (“Flank300”), (ii) 20 bps upstream flanking genomic DNA from tRNA-Arg-TCG-1-1 (SEQ ID NO: 895) and 17 bps of downstream flanking genomic DNA from tRNA-Arg-TCG-1-1 (SEQ ID NO: 896) (“Flank20”), (iii) 10 bps upstream flanking genomic DNA from tRNA-Arg-TCG-1-1 (SEQ ID NO: 897) and 17 bps of downstream flanking genomic DNA from tRNA-Arg-TCG-1-1 (SEQ ID NO: 896) (“Flank10”), or (iv) 0 bps upstream flanking genomic DNA from tRNA-Arg-TCG-1-1 and 17 bps of downstream flanking genomic DNA from tRNA-Arg-TCG-1-1 (SEQ ID NO: 896) (“Flank0”). Details of the expression vectors are shown in TABLE 11. “GFP-PTC” indicates cells transfected with the EGFP-R96X-TGA reporter alone (SEQ ID NO: 31), “GFP” indicates cells transfected with a version of the EGFP reporter that lacks a PTC. Readthrough activity was measured by flow cytometry at ˜24 hours post transfection. Data are presented as histograms displaying the frequency distribution of the data versus fluorescence intensity for cells expressing EGFP above background. FIG. 26B shows the percentage of EGFP positive cells in all viable cells (“% GFP+”) and mean EGFP intensity in viable cells expressing EGFP above background (“Mean GFP Signal”) for the cells depicted in FIG. 26A.

FIG. 27 is a schematic representation of the constructs used to test the impact of a 5′ leader sequence on the read-through of a premature termination codon (PTC) by a suppressor tRNA. Constructs contain (i) a 100 bp 5′ leader sequence derived from genomic DNA located upstream of tRNA genes that are highly expressed in HEK293 cells, (ii) a single copy of either the Arg_{TCA} suppressor tRNA #115 (SEQ ID NO: 18) or the G1n_{TTA} suppressor tRNA #163 (SEQ ID NO: 45), and (iii) an RNA polymerase III termination signal (“Term”).

FIG. 28 is a graph depicting the readthrough activity of the indicated Arg_{TCA} suppressor tRNA expression constructs as measured by flow cytometry in Flp-In-293 cells at ˜24 hours post transfection. Cells were co-transfected with constructs containing (i) an EGFP-R96X-TGA reporter (SEQ ID NO: 31) and (ii) the Arg_{TCA} suppressor tRNA #115 (SEQ ID NO: 18) in the context of the indicated 100 bps upstream genomic DNA leader sequences (SEQ ID NOs: 869-888). “WT-EGFP” indicates cells transfected with a reporter containing wild-type GFP without a PTC, “EV” (empty vector) indicates cells co-transfected with an expression construct that does not contain a tRNA or an EGFP reporter, and “26/27” indicates cells co-transfected with the Arg_{TCA} suppressor tRNA #115 (SEQ ID NO: 18) in the context of 200 bps upstream flanking genomic DNA from tRNA-Arg-TCG-1-1 (SEQ ID NO: 26) and 200 bps of downstream flanking genomic DNA from tRNA-Arg-TCG-1-1 (SEQ ID NO: 27). The plot shows the percentage of viable cells that express EGFP above background. The percentage of cells that expressed EGFP ranged from 17.2 to 33.7% for the cells expressing the Arg_{TCA} suppressor, relative to 0.4% for the empty vector control.

FIG. 29 is a graph depicting the readthrough activity of the indicated Arg_{TCA} suppressor tRNA expression constructs as measured by flow cytometry in Flp-In-293 cells at ˜24 hours post transfection. Cells were co-transfected with constructs containing (i) an EGFP-R96X-TGA reporter (SEQ ID NO: 31) and (ii) the Arg_{TCA} suppressor tRNA #115 (SEQ ID NO: 18) in the context of the indicated 100 bps upstream genomic DNA leader sequences (SEQ ID NOs: 869-888). “WT-EGFP” indicates cells transfected with a reporter containing wild-type GFP without a PTC, “EV” (empty vector) indicates cells co-transfected with an expression construct that does not contain a tRNA or an EGFP reporter, and “26/27” indicates cells co-transfected with the Arg_{TCA} suppressor tRNA #115 (SEQ ID NO: 18) in the context of 200 bps upstream flanking genomic DNA from tRNA-Arg-TCG-1-1 (SEQ ID NO: 26) and 200 bps of downstream flanking genomic DNA from tRNA-Arg-TCG-1-1 (SEQ ID NO: 27). The plot shows mean EGFP intensity in cells expressing EGFP above background. The mean EGFP intensity ranged from 1990 to 4319 for the cells expressing the Arg_{TCA} suppressor, relative to 602 for the empty vector control.

FIG. 30 is a graph depicting the readthrough activity of the indicated G1n_{TTA} suppressor tRNA expression constructs as measured by flow cytometry in Flp-In-293 cells at ˜24 hours post transfection. Cells were co-transfected with constructs containing (i) an EGFP-Q69X-TAA reporter (SEQ ID NO: 175) and (ii) the G1n_{TTA} suppressor tRNA #163 (SEQ ID NO: 45) in the context of the indicated 100 bps upstream genomic DNA leader sequences (SEQ ID NOs: 869-888). “WT-EGFP” indicates cells transfected with a reporter containing wild-type GFP without a PTC, “EV” (empty vector) indicates cells co-transfected with an expression construct that does not contain a tRNA or an EGFP reporter, and “173/174” indicates cells co-transfected with the G1n_{TTA} suppressor tRNA #163 (SEQ ID NO: 45) in the context of 200 bps upstream flanking genomic DNA from tRNA-Gln-TTG-1-1 (SEQ ID NO: 173) and 200 bps of downstream flanking genomic DNA from tRNA-Gln-TTG-1-1 (SEQ ID NO: 174). The plot shows the percentage of viable cells that express EGFP above background. The percentage of cells that expressed EGFP ranged from 21.4 to 35.7% for the cells expressing the G1n_{TTA} suppressor, relative to 0.3% for the empty vector control.

FIG. 31 is a graph depicting the readthrough activity of the indicated G1n_{TTA} suppressor tRNA expression constructs as measured by flow cytometry in Flp-In-293 cells at ˜24 hours post transfection. Cells were co-transfected with constructs containing (i) an EGFP-Q69X-TAA reporter (SEQ ID NO: 175) and (ii) the G1n_{TTA} suppressor tRNA #163 (SEQ ID NO: 45) in the context of the indicated 100 bps upstream genomic DNA leader sequences (SEQ ID NOs: 869-888). “WT-EGFP” indicates cells transfected with a reporter containing wild-type GFP without a PTC, “EV” (empty vector) indicates cells co-transfected with an expression construct that does not contain a tRNA or an EGFP reporter, and “173/174” indicates cells co-transfected with the G1n_{TTA} suppressor tRNA #163(SEQ ID NO: 45) in the context of 200 bps upstream flanking genomic DNA from tRNA-Gln-TTG-1-1 (SEQ ID NO: 173) and 200 bps of downstream flanking genomic DNA from tRNA-Gln-TTG-1-1 (SEQ ID NO: 174). The plot shows mean EGFP intensity in cells expressing EGFP above background. The mean EGFP intensity ranged from 1702 to 3822 for the cells expressing the G1n_{TTA} suppressor, relative to 387 for the empty vector control.

FIG. 32 is table summarizing the results of FIGS. 28-31, where the values have been normalized to cells transfected with wild-type GFP without a PTC.

FIG. 33 is a graph depicting the percentage of EGFP positive cells as measured by flow cytometry in three independent experiments at ˜48 hours post transfection. Neuro-2a cells were transfected with an expression construct containing the EGFP-Q69X-TAA reporter (“EGFP-PTC”) (SEQ ID NO: 175) and either (i) including the G1n_{TTA} suppressor tRNA #002 (SEQ ID NO: 36) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hours after transfection and the indicated drugs at the indicated concentrations were added at this point. A reporter containing wildtype EGFP without a PTC (“EGFP”) was used as a control. “Mock” indicates mock transfected cells, “no tRNA” indicates cells transfected with the indicated EGFP expression construct alone, “4X-Gln-TTG” indicates cells transfected with an expression construct that contains four copies of a wild-type Gln-tRNA with a TTG anticodon and the EGFP-Q69X-TAA reporter. The plot shows the percentage of viable cells that express GFP above background. Error bars represent the standard deviation of the data. The percentage of cells that expressed GFP ranged from 64.7 to 67.3% for the cells expressing the G1n_{TTA} suppressor, relative to 0.2 to 0.7% for negative controls, 0.4 to 0.6% for cells treated with ataluren, 0.4% for cells treated with gentamicin, and 1.7% to 4.5% for cells treated with G418.

FIG. 34 is a graph depicting cell viability from FIG. 33 as measured by flow cytometry at ˜48 hours post transfection. Neuro-2a cells were transfected with an expression construct containing the EGFP-Q69X-TAA reporter (“EGFP-PTC”) (SEQ ID NO: 175) and either (i) including the G1n_{TTA} suppressor tRNA #002 (SEQ ID NO: 36) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hours after transfection and the indicated drugs at the indicated concentrations were added at this point. A reporter containing wildtype EGFP without a PTC (“EGFP”) was used as a control. “Mock” indicates mock transfected cells, “no tRNA” indicates cells transfected with the indicated EGFP expression construct alone, “4X-Gln-TTG” indicates cells transfected with an expression construct that contains four copies of a wild-type Gln-tRNA with a TTG anticodon and the EGFP-Q69X-TAA reporter. Cell viability was assessed by flow cytometry using 7-Amino Actinomycin D (7-AAD), a membrane impermeant dye that is generally excluded from viable cells.

FIG. 35 is a graph depicting the percentage of EGFP positive cells as measured by flow cytometry in three independent experiments at ˜48 hours post transfection. Neuro-2a cells were transfected with an expression construct containing the EGFP-Q69X-TAG reporter (“EGFP-PTC”) (SEQ ID NO: 176) and either (i) including the GlncTA suppressor tRNA #196 (SEQ ID NO: 178) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hour after transfection and the indicated drugs at the indicated concentrations were added at this point. A reporter containing wildtype EGFP without a PTC (“EGFP”) was used as a control. “Mock” indicates mock transfected cells, “no tRNA” indicates cells transfected with the indicated EGFP expression construct alone, “4X-Gln-TTG” indicates cells transfected with an expression construct that contains four copies of a wild-type Gln-tRNA with a TTG anticodon and the EGFP-Q69X-TAA reporter. Plot shows the percentage of viable cells that express GFP above background. The percentage of cells that expressed GFP ranged from 73.1 to 78.2% for the cells expressing the GlncTA suppressor, relative to 0.5 to 0.8% for negative controls, 0.5 to 0.7% for cells treated with ataluren, 0.5 to 1.1% for cells treated with gentamicin, and 13.6% to 20.6% for cells treated with G418.

FIG. 36 is a graph depicting cell viability from FIG. 35 as measured by flow cytometry at ˜48 hours post transfection. Neuro-2a cells were transfected with an expression construct containing the EGFP-Q69X-TAG reporter (“EGFP-PTC”) (SEQ ID NO: 176) and either (i) including the GlncTA suppressor tRNA #196 (SEQ ID NO: 178) at the indicated copy number on the same construct, or (ii) treated with ataluren at the indicated concentration, (iii) treated with gentamicin at the indication concentration, or (iv) treated with G418 at the indicated concentration. For all experimental conditions, cell culture medium was replaced with fresh medium ˜6 hour after transfection and the indicated drugs at the indicated concentrations were added at this point. A reporter containing wildtype EGFP without a PTC (“EGFP”) was used as a control. “Mock” indicates mock transfected cells, “no tRNA” indicates cells transfected with the indicated EGFP expression construct alone, “4X-Gln-TTG” indicates cells transfected with an expression construct that contains four copies of a wild-type Gln-tRNA with a TTG anticodon and the EGFP-Q69X-TAA reporter. Cell viability was assessed by flow cytometry using 7-Amino Actinomycin D (7-AAD), a membrane impermeant dye that is generally excluded from viable cells.

FIG. 37A is a Western blot depicting rescue of full-length SCN1A protein expression by a suppressor tRNA. Flp-In-293 cells were transfected with an expression construct containing mouse SCN1A with an Arg(R)-to-TGA PTC (R1407X) and a 3xFLAG tag peptide (DYKDHD-G-DYKDHD-I-DYKDDDDK) at the C-terminus (SEQ ID NO: 899) (“SCN1a (R1407*)”) and either (i) co-transfected with an expression construct containing the Arg_{TCA} suppressor tRNA #115 (SEQ ID NO: 18) (“Arg>TGA #115”), or (ii) treated with ataluren at the indicated concentration, (iii) treated with gentamicin at the indication concentration, or (iv) treated with G418 at the indicated concentration. “SCN1a (w.t.)” indicates cells transfected with an expression construct containing wild-type mouse SCN1A and a 3xFLAG tag peptide at the C-terminus (SEQ ID NO: 898). Protein was isolated at 24 hours post transfection and SCN1A was detected using a monoclonal anti-FLAG M2 antibody. Molecular weights based on protein molecular weight markers are indicated to the left of the gel. FIG. 37B is a quantification of the Western blot shown in FIG. 37A. The intensity of bands corresponding to the size of full-length SCN1A protein was measured using ImageJ and all intensity values were normalized to wild-type SCN1A protein (“SCN1a (w.t.)” lane). Cells co-transfected with expression vectors containing mutant SCN1A and the Arg_{TCA} suppressor tRNA expressed greater than 70% of the full-length SCN1A expressed by cells transfected with an expression construct containing wild-type SCN1A.

FIG. 38A is a Western blot depicting rescue of full-length SCN1A protein expression by suppressor tRNAs. Flp-In-293 cells were co-transfected with (i) an expression construct containing mouse SCN1A with an Arginine-to-TGA PTC (R1407X) and a 3xFLAG tag peptide (DYKDHD-G-DYKDHD-I-DYKDDDDK) at the C-terminus (SEQ ID NO: 899) (“SCN1a (R1407*)”) and (ii) either the #104 Arg_{TCA} suppressor tRNA (SEQ ID NO: 6) (“Sup #104”), the #106 Arg_{TCA} suppressor tRNA (SEQ ID NO: 8) (“Sup #106”), or the Arg_{TCA} suppressor tRNA #115 (SEQ ID NO: 18) (“Sup #115”). “SCN1a (w.t.)” indicates cells transfected with an expression construct containing wild-type mouse SCN1A and a 3xFLAG tag peptide at the C-terminus (SEQ ID NO: 898). Protein was isolated at 24 hours post transfection and SCN1A was detected using a monoclonal anti-FLAG M2 antibody. Molecular weights based on protein molecular weight markers are indicated to the left of the gel. FIG. 38B is a quantification of the Western blot shown in FIG. 38A. The intensity of bands corresponding to the size of full-length SCN1A protein was measured using ImageJ and all intensity values were normalized to wild-type SCN1A protein (“SCN1a (w.t.)” lane). Cells co-transfected with expression vectors containing mutant SCN1A and the Arg_{TCA} suppressor tRNA expressed greater than 30% (Sup #104), greater than 60% (Sup #106), or greater than 70% (Sup #115) of the full-length SCN1A expressed by cells transfected with an expression construct containing wild-type SCN1A.

FIG. 39A is a Western blot depicting rescue of full-length SCN1A protein expression by a suppressor tRNA. Flp-In-293 cells were cultured in 6-well cell culture plates and co-transfected with (i) an expression construct containing mouse SCN1A with an Arginine-to-TGA PTC (R1407X) and a 3xFLAG tag peptide (DYKDHD-G-DYKDHD-I-DYKDDDDK) at the C-terminus (SEQ ID NO: 899) (“SCN1a (R1407*)”) and (ii) the Arg_{TCA} suppressor tRNA #115 (SEQ ID NO: 18) (“Arg>TGA #115”) at the indicated concentrations. 1× indicates 400 ng of the Arg_{TCA} suppressor tRNA construct per well, 0.3× indicates 133 ng of the Arg_{TCA} suppressor tRNA construct per well, 0.1× indicates 40 ng of the Arg_{TCA} suppressor tRNA construct per well, and 0.03× indicates 13 ng of the Arg_{TCA} suppressor tRNA construct per well. “SCN1a (w.t.)” indicates cells transfected with an expression construct containing wild-type mouse SCN1A and a 3xFLAG tag peptide at the C-terminus (SEQ ID NO: 898). Protein was isolated at 24 hours post transfection and SCN1A was detected using a monoclonal anti-FLAG M2 antibody. Molecular weights based on protein molecular weight markers are indicated to the left of the gel. FIG. 39B is a quantification of the Western blot shown in FIG. 39A. The intensity of bands corresponding to the size of full-length SCN1A protein was measured using ImageJ and all intensity values were normalized to wild-type SCN1A protein (“SCN1a (w.t.)” lane).

FIG. 40 is a schematic representation of constructs that were packaged into AAV-PHP.eB capsids. Construct 262 contains wild-type EGFP driven by an EF1a promoter. Construct 269 contains EGFP-R96X-TGA driven by an EF1a promoter (SEQ ID NO: 177) and two copies of the Arg_{TCA} suppressor tRNA #115 (SEQ ID NO: 18) (“TCA-115”) in the context of 55 bps upstream flanking genomic DNA from tRNA-Tyr-GTA-5-1 (SEQ ID NO: 900). Both constructs contain 5′ and 3′ ITR sequences from AAV2, which provide cis-acting elements for AAV replication and packaging.

FIG. 41 depicts readthrough activity of suppressor tRNAs delivered by AAV. AAV-PHP.eB containing the constructs shown in FIG. 40 was produced by Vigene Biosciences. 48 hours prior to AAV transduction, 293 cells were pre-transfected with an expression construct containing the LY6A gene, which is required for robust transduction by AAV-PHP.eB. Cells were transduced at an MOI of 1E5 vg/cell. Where indicated, cells were also transfected with an expression construct containing the EGFP-R96X-TGA reporter (SEQ ID NO: 31) and an expression construct containing the Arg_{TCA} suppressor tRNA #115 (SEQ ID NO: 18). At 72 hours post transduction, the EGFP signal was quantified by immunofluorescence. Live cell images were captured on an EVOS FL Auto 2 imaging system. CellProfiler software was used to segment and extract the integrated EGFP intensity for nuclei in each image. These values were averaged across all nuclei in each condition. Background average integrated EGFP intensity from the negative control condition was subtracted from each of these averages, and all values were normalized. The plot depicts the normalized % GFP intensity with all values normalized to cells transduced with AAV-PHP.eB containing construct 262.

FIG. 42 shows fluorescence as measured by flow cytometry for Neuro-2a cells used in ribosome footprint profiling analysis. Cells were transfected with either (i) an expression construct containing an EGFP-R96X-TGA reporter (SEQ ID NO: 177) and the Arg_{TCA} suppressor tRNA #001 (SEQ ID NO: 11) on the same construct (“Arg_{TCA} EGFP-PTC”) or (ii) an expression construct containing a version of the EGFP reporter that lacks a PTC (“WT-EGFP”).

FIG. 43 depicts fold change in 3′ UTR read density distributions (as determined by ribosome profiling) between the two Neuro-2a cells populations shown in FIG. 42. Cells were transfected with either (i) an expression construct containing an EGFP-R96X-TGA reporter (SEQ ID NO: 177) and the Arg_{TCA} suppressor tRNA #001 (SEQ ID NO: 11) on the same construct (“Arg_{TCA} EGFP-PTC”) or (ii) an expression construct containing a version of the EGFP reporter that lacks a PTC (“WT-EGFP”). Cells were lysed at ˜48 hours post transfection and subjected to ribosome footprint profiling. Raw reads were trimmed of adapters using Trimmomatic then depleted for non-coding RNA by aligning against Ensembl's mouse mm10 ncRNA reference using bowtie2. Remaining reads were aligned against UCSC's mm10 mouse reference assembly, again using bowtie2. Multi-mapping reads were discarded. The resulting final set of aligned reads was quantified using the RiboProfiling package in R and custom Python scripting. Python was used to generate plots examining 3′ UTR occupancy and fold change for each gene with 20 or more uniquely mapping reads, and the distributions for genes with each native stop codon were compared using the 2 sample Kolmogorov-Smirnov test.

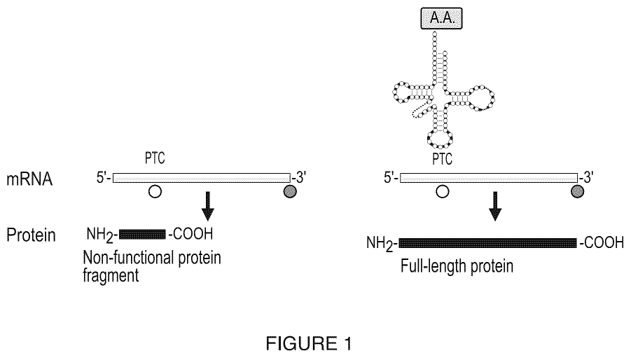

### DETAILED DESCRIPTION

The invention is based, in part, upon the discovery of tRNAs (e.g., suppressor tRNAs), that permit an amino acid to be incorporated into a gene product encoded by a gene in a mammalian cell at a position that would otherwise result in a truncated gene product caused by a premature termination codon (PTC) in the gene. The invention is further based, in part, upon the discovery that a tRNA that permits an amino acid to be incorporated into a gene product encoded by a gene at a position that would otherwise result in a truncated gene product caused by a PTC in the gene can be used to treat a disease mediated by a PTC in a gene in a subject.

Accordingly, in one aspect, the invention provides a tRNA (e.g., an isolated tRNA) comprising a nucleotide sequence set forth in TABLE 2.

In another aspect, the invention provides an expression vector comprising a nucleotide sequence encoding a tRNA, e.g., as shown in TABLEs 1-3. In certain embodiments, the expression vector comprises 1, 2, 3, 4, or more than 4 copy numbers of the nucleotide sequence encoding the tRNA. In certain embodiments, the expression vector comprises a nucleotide sequence corresponding to a genomic DNA sequence flanking a wild-type tRNA gene. For example, in certain embodiments, the expression vector comprises a nucleotide sequence set forth in TABLE 4.

In another aspect, the invention provides a pharmaceutical composition comprising any of the foregoing tRNAs or any of the foregoing expression vectors and a pharmaceutically acceptable excipient.

In another aspect, the invention provides a method of expressing in a mammalian cell a functional gene product encoded by a gene containing a premature termination codon, the method comprising introducing into the cell an effective amount of a tRNA (e.g., as shown in TABLEs 1-3 below, e.g., comprising SEQ ID NO: 6-9, 11, 16-22, 35-40, 44, 45, 178-182, 186, or 187), or an expression vector comprising a nucleotide sequence encoding the tRNA, thereby permitting an amino acid to be incorporated into the gene product at a position that would otherwise result in a truncated gene product caused by the premature termination codon.

In certain embodiments of any of the foregoing methods, the cell contains less truncated gene product than a cell without the tRNA. For example, in certain embodiments, the cell contains less than about 5%, about 10%, about 20%, about 30%, about 40%, about 50%, about 60%, about 70%, about 80%, or about 90% of the truncated gene product relative to a cell without the tRNA. In certain embodiments, the cell contains from about 5% to about 80%, about 5% to about 60%, about 5% to about 40%, about 5% to about 20%, about 5% to about 10%, about 10% to about 80%, about 10% to about 60%, about 10% to about 40%, about 10% to about 20%, about 20% to about 80%, about 20% to about 60%, about 20% to about 40%, about 40% to about 80%, about 40% to about 60%, or about 60% to about 80% of the truncated gene product relative to a cell without the tRNA. In certain embodiments, there is no detectable truncated gene product in the cell. Truncated gene product amount or expression may be measured by any method known in the art, for example, Western blot or ELISA.

In certain embodiments, the cell contains a greater amount of functional gene product than a cell without the tRNA. For example, in certain embodiments, the method increases the amount of functional gene product in a cell, tissue, or subject by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, or about 500% relative to a cell, tissue, or subject without the tRNA. In certain embodiments, the method increases the amount of functional gene product in a cell, tissue, or subject, by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject without the tRNA. Functional gene product amount or expression may be measured by any method known in the art, for example, Western blot or ELISA.

In certain embodiments, the tRNA permits an amino acid to be incorporated into the gene product at a position corresponding to a premature termination codon (i.e. the tRNA permits read-through of the premature termination codon), but the tRNA does not permit a substantial amount of amino acid to be incorporated into a gene product at a position corresponding to a native stop codon (i.e., the tRNA does not permit read-through of a native stop codon). For example, in certain embodiments, a disclosed tRNA does not increase read-through of a native stop codon (or all native stop codons) in a cell, tissue, or subject, or increases read-through by less than about 1%, about 2%, about 3%, about 4%, about 5%, about 10%, about 20%, about 30%, about 40%, or about 50%, relative to a cell, tissue, or subject that has not been contacted with the tRNA. Read-through of a native stop codon may be measured by any method known in the art, for example, ribosome profiling as described in Example 13 herein.

In certain embodiments of any of the foregoing methods, the gene is selected from a gene set forth in TABLE 5 or TABLE 6. In certain embodiments, the gene is a SCN1A gene.

In another aspect, the invention provides a method of expressing in a cell a functional SCN1A gene product encoded by a SCN1A gene containing a premature termination codon, the method comprising introducing into the cell an effective amount of a tRNA (e.g., as shown in TABLEs 1-3 below, e.g., comprising SEQ ID NO: 6-9, 11, 16-22, 35-40, 44, 45, 178-182, 186, or 187), or an expression vector comprising a nucleotide sequence encoding the tRNA, thereby permitting an amino acid to be incorporated into the SCN1A gene product at a position that would otherwise result in a truncated SCN1A gene product caused by the premature termination codon.

In another aspect, the invention provides a method of increasing in a cell voltage-gated sodium channel activity encoded by a SCN1A gene containing a premature termination codon, the method comprising introducing into the cell an effective amount of a tRNA (e.g., as shown in TABLEs 1-3 below, e.g., comprising SEQ ID NO: 6-9, 11, 16-22, 35-40, 44, 45, 178-182, 186, or 187), or an expression vector comprising a nucleotide sequence encoding the tRNA, thereby permitting an amino acid to be incorporated into the SCN1A gene product at a position that would otherwise result in a truncated SCN1A gene product caused by the premature termination codon.

In another aspect, the invention provides a method of treating a premature termination codon-mediated disorder in a subject in need thereof wherein the subject has a gene with a premature termination codon, the method comprising administering to the subject an effective amount of a tRNA (e.g., as shown in TABLEs 1-3 below, e.g., comprising SEQ ID NO: 6-9, 11, 16-22, 35-40, 44, 45, 178-182, 186, or 187), or an expression vector comprising a nucleotide sequence encoding the tRNA, thereby to treat the disorder in the subject. In certain embodiments, the disorder is selected from a disorder set forth in TABLE 5 or TABLE 6.

In another aspect, the invention provides a method of treating Dravet syndrome in a subject in need thereof wherein the subject has a SCN1A gene with a premature termination codon, the method comprising administering to the subject an effective amount of a tRNA (e.g., as shown in TABLEs 1-3 below, e.g., comprising SEQ ID NO: 6-9, 11, 16-22, 35-40, 44, 45, 178-182, 186, or 187), or an expression vector comprising a nucleotide sequence encoding the tRNA, thereby to treat Dravet syndrome in the subject.

### I. tRNAs and Suppressor tRNAs

During protein synthesis, a transfer RNA (tRNA) delivers an amino acid to a ribosome for incorporation into a growing protein (polypeptide) chain. tRNAs typically are about 70 to 100 nucleotides in length, and active tRNAs contain a 3′ CCA sequence that may be transcribed into the tRNA during its synthesis or may be added later during post-transcriptional processing. During aminoacylation, the amino acid that is attached to a given tRNA molecule is covalently attached to the 2′ or 3′ hydroxyl group of the 3′-terminal ribose to form an aminoacyl-tRNA (aa-tRNA). It is understood that an amino acid can spontaneously migrate from the 2′-hydroxyl group to the 3′-hydroxyl group and vice versa, but it is incorporated into a growing protein chain at the ribosome from the 3′-OH position. A loop at the other end of the folded aa-tRNA molecule contains a sequence of three bases known as the anticodon. When this anticodon sequence hybridizes or base-pairs with a complementary three-base codon sequence in a ribosome-bound messenger RNA (mRNA), the aa-tRNA binds to the ribosome and its amino acid is incorporated into the polypeptide chain being synthesized by the ribosome. Because all tRNAs that base-pair with a specific codon are aminoacylated with a single specific amino acid, the translation of the genetic code is effected by tRNAs. Each of the 61 non-termination codons in an mRNA directs the binding of its cognate aa-tRNA and the addition of a single specific amino acid to the growing polypeptide chain being synthesized by the ribosome.

tRNAs are generally highly conserved and are often functional across species. Accordingly, a tRNA derived from a bacterial tRNA, a non-mammalian eukaryotic tRNA, or a mammalian (e.g., human) tRNA may be useful in the practice of the invention. Nucleotide sequences encoding naturally occurring human tRNAs are known and generally available to those of skill in the art through sources such as Genbank. See also Sprinzl et al. (2005) NUCLEIC ACIDS RES. 33: D139-40; Buckland et al. (1996) GENOMICS 35(1):164-71; Schimmel et al. (Eds.) (1979) “Transfer-RNA: Structure, Properties, and Recognition,” Cold Spring Harbor Laboratory; Agris (1983) “The Modified Nucleosides of Transfer RNA, II,” Alan R. Liss Inc. tRNAs are generally highly conserved and are often functional across species.

Suppressor tRNAs are modified tRNAs that insert a suitable amino acid at a mutant site, e.g., a PTC, in protein encoding gene. The use of the word in suppressor is based on the fact, that under certain circumstance, the modified tRNA “suppresses” the phenotypic effect of the coding mutation. Suppressor tRNAs typically contain a mutation (modification) in either the anticodon, changing codon specificity, or at some position that alters the aminoacylation identity of the tRNA.

In certain embodiments, a tRNA (e.g., a suppressor tRNA) contains a modified anticodon region, such that the modified anticodon hybridizes with a different codon than the corresponding naturally occurring anticodon. In certain embodiments, the modified anticodon hybridizes with a termination codon, e.g., a PTC, and as a result, the tRNA incorporates an amino acid into a gene product rather than terminating protein synthesis. In certain embodiments, the modified anticodon hybridizes with a premature termination codon and, and as a result, the tRNA incorporates an amino acid into a gene product at a position that would otherwise result in a truncated gene product caused by the premature termination codon.

In certain embodiments, a tRNA comprises an anticodon that hybridizes to a codon selected from UAG (i.e., an “amber” termination codon), UGA (i.e., an “opal” termination codon), and UAA (i.e., an “ochre” termination codon). In certain embodiments, the anticodon hybridizes to a codon selected from UGA to UAA. In certain embodiments, the anticodon hybridizes to UGA. In certain embodiments, a tRNA comprises an anticodon that hybridizes to a non-standard termination codon, e.g., a 4-nucleotide codon (See, for example, Moore et al. (2000) J. MOL. BIOL. 298:195, and Hohsaka et al. (1999) J. AM. CHEM. SOC. 121:12194).

In certain embodiments, the tRNA is aminoacylated or is capable of being aminoacylated with any natural amino acid. For example, a tRNA may be capable of being aminoacylated with alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In certain embodiments the tRNA is capable of being aminoacylated with serine, leucine, glutamine, or arginine. In certain embodiments the tRNA is capable of being aminoacylated with glutamine or arginine. In certain embodiments the tRNA is capable of being aminoacylated with arginine.

In certain embodiments, the tRNA (i) comprises an anticodon that hybridizes to a codon as indicated in TABLE 1, and (ii) is aminoacylated or is capable of being aminoacylated with an amino acid as indicated in TABLE 1.

TABLE 1

| | | |
|---|---|---|
| codon: UAG | codon: UGA | codon: UAA |
| amino acid: alanine | amino acid: alanine | amino acid: alanine |
| codon: UAG | codon: UGA | codon: UAA |
| amino acid: arginine | amino acid: arginine | amino acid: arginine |
| codon: UAG | codon: UGA | codon: UAA |
| amino acid: asparagine | amino acid: asparagine | amino acid: asparagine |
| codon: UAG | codon: UGA | codon: UAA |
| amino acid: aspartic acid | amino acid: aspartic acid | amino acid: aspartic acid |
| codon: UAG | codon: UGA | codon: UAA |
| amino acid: cysteine | amino acid: cysteine | amino acid: cysteine |
| codon: UAG | codon: UGA | codon: UAA |
| amino acid: glutamine | amino acid: glutamine | amino acid: glutamine |
| codon: UAG | codon: UGA | codon: UAA |
| amino acid: glutamic acid | amino acid: glutamic acid | amino acid: glutamic acid |
| codon: UAG | codon: UGA | codon: UAA |
| amino acid: glycine | amino acid: glycine | amino acid: glycine |
| codon: UAG | codon: UGA | codon: UAA |
| amino acid: histidine | amino acid: histidine | amino acid: histidine |
| codon: UAG | codon: UGA | codon: UAA |
| amino acid: isoleucine | amino acid: isoleucine | amino acid: isoleucine |
| codon: UAG | codon: UGA | codon: UAA |
| amino acid: leucine | amino acid: leucine | amino acid: leucine |
| codon: UAG | codon: UGA | codon: UAA |
| amino acid: lysine | amino acid: lysine | amino acid: lysine |
| codon: UAG | codon: UGA | codon: UAA |
| amino acid: methionine | amino acid: methionine | amino acid: methionine |
| codon: UAG | codon: UGA | codon: UAA |
| amino acid: phenylalanine | amino acid: phenylalanine | amino acid: phenylalanine |
| codon: UAG | codon: UGA | codon: UAA |
| amino acid: proline | amino acid: proline | amino acid: proline |
| codon: UAG | codon: UGA | codon: UAA |
| amino acid: serine | amino acid: serine | amino acid: serine |
| codon: UAG | codon: UGA | codon: UAA |
| amino acid: threonine | amino acid: threonine | amino acid: threonine |
| codon: UAG | codon: UGA | codon: UAA |
| amino acid: tryptophan | amino acid: tryptophan | amino acid: tryptophan |
| codon: UAG | codon: UGA | codon: UAA |
| amino acid: tyrosine | amino acid: tyrosine | amino acid: tyrosine |
| codon: UAG | codon: UGA | codon: UAA |
| amino acid: valine | amino acid: valine | amino acid: valine |

In certain embodiments, the tRNA comprises, consists essentially of, or consists of a nucleotide sequence shown in TABLE 2. In certain embodiments, the tRNA comprises, consists essentially of, or consists of a nucleotide sequence having 80%, 85%, 86%, 87%, 88%, 89%, 90%, 91%, 92%, 93%, 94%, 95%, 96%, 97%, 98%, or 99% sequence identity to a nucleotide sequence shown in TABLE 2. In certain embodiments, the tRNA comprises, consists essentially of, or consists of a nucleotide sequence selected from SEQ ID NOs: 19-21, 37, 39, 40, 44, 179, 181, 182, and 186, or a nucleotide sequence having 80%, 85%, 86%, 87%, 88%, 89%, 90%, 91%, 92%, 93%, 94%, 95%, 96%, 97%, 98%, or 99% sequence identity to a nucleotide sequence selected from SEQ ID NOs: 19-21, 37, 39, 40, 44, 179, 181, 182, and 186. It is understood that, throughout the description, in each instance where a tRNA comprises, consists essentially of, or consists of a nucleotide sequence including one or more thymines (T), a tRNA is also contemplated that comprises, consists essentially of, or consists of the same nucleotide sequence including a uracil (U) in place of one or more of the thymines (T), or a uracil (U) in place of all the thymines (T). Similarly, in each instance where a tRNA comprises, consists essentially of, or consists of a nucleotide sequence including one or more uracils (U), a tRNA is also contemplated that comprises, consists essentially of, or consists of a nucleotide sequence including a thymine (T) in place of the one or more of the uracils (U), or a thymine (T) in place of all the uracils (U).

TABLE 2

| SEQ ID NO | Suppressor tRNA Sequence (anticodon lowercase) |
|---|---|
| 2 | GGGCCAGTGGCGCAATGGATAACGCGTCTGACTtcaGATCAGAAGATTGTAG |
| | GTTCGACTCCTACCTGGCTCG |
| 5 | GGCCGCGTGGCCTAATGGATAAGGCGTCTGATTtcaGATCAGAAGATTGGGG |
| | GTTCGAGTCCCTTCGTGGTCG |
| 14 | GACCACGTGGCCTAACGGATAAGGCGTCTGACTtcaGATCAGAAGATTGAGG |
| | GTTCGAATCCCTTCGTGGTTA |
| 15 | GGCTCTGTGGCGCAATGGATAGCGCATTGGACTtcaAGTGACGAGAAAGCGA |
| | TTCAAAGGTTGTGGGTTCGAATCCCACCAGAGTCG |
| 19 | GGCTCTGTGGCGCAATGGATAGCGCATTGGACTtcaAGCATGATTGAGAGAT |
| | TCAAAGGTTGCGGGTTCGAGTCCCGCCAGAGTCG |
| 20 | GGCTCTGTGGCGCAATGGATAGCGCATTGGACTtcaAATTCAAAGGTTGCGG |
| | GTTCGAGTCCCGCCAGAGTCG |
| 21 | GGCTCTGTGGCGCAATGGATAGCGCATTGGACTtcaAGACAAATGGAGGCAT |
| | TCAAAGGTTGTGGGTTCGAGTCCCACCAGAGTCG |
| 23 | GTCTCTGTGGCGCAATGGACGAGCGCGCTGGACTtcaAATCCAGAGGTTCTG |
| | GGTTCGAGTCCCGGCAGAGATG |
| 24 | GGCTCTGTGGAGCAATGGATAGCACATTGGACTtcaAGCATGACCGAGAGAT |
| | TCAAAGGTTGCGGGTTCGAGTCCCACCAGAGTTG |
| 25 | GGCTCTGTGGAGCAATGGATAGCACATTGGACTtcaAATTCAAAGGTTGCGG |
| | GTTCGAGTCCCACCAGAGTTG |
| 37 | GGTCCCATGGTGTAATGGTTAGCACTCTGGACTttaAATCCAGCGATCCGAG |
| | TTCAAATCTCGGTGGGACCT |
| 39 | GGTTCCATGGTGTAATGGTTAGCACTCTGGACTttaAATCCAGCGATCCGAG |
| | TTCAAATCTCGGTGGAACCT |
| 40 | GGTTCCATGGTGTAATGGTTAGCACTCTGGACTttaAATCCAGCGATCCGAG |
| | TTCAAATCTCGGTGGGACCT |
| 41 | GGTTCCATGGTGTAATGGTGAGCACTCTGGACTttaAATCCAGCGATCCGAG |
| | TTCAAATCTCGGTGGGACCT |
| 42 | GGTTCCATGGTGTAATGGCTAGCACTCTGGACTttaAATCCAGCGATCCGAG |
| | TTCAAATCTCGGTGGGATTT |
| 43 | GGTTCCATGGTGTAATGGTTAGCACTCTGGACTttaAATCCAGCCATACAAG |
| | TTCAAATCTCAGTGGAACCT |
| 44 | GGTTCCTTGGTGTAAGATGAGCACTCTGGATTttaAATCCAGCGATCAGAGT |
| | TCAAATCTCGGTGGGACCT |
| 46 | GGCCCCATGGTGTAATGGTTAGCACTCTGGACTttaAATCCAGCGATCCGAG |
| | TTCAAATCTCGGTGGGACCT |
| 47 | GGTCTCATGGTGTAATGGTTAGCACACTGGACTttaAGTCCAGCAATCCGAG |
| | TTCGAGTCTTGGTGAGACCA |
| 48 | GGACCCATGGTGTAATGGTTAGCACTCTGGACTttaAATCCAGCAATCCAAG |
| | TTCAAATCTCGGTGGGACCT |
| 49 | GTCAGGATGGCCGAGTGGTCTAAGGCGCCAGACTctaGCTATGGCTTCCTCG |
| | CTCTGAGGGTTCTGGTCTCCCCTGGAGGCGTGGGTTCGAATCCCACTTCTGA |
| | CA |
| 50 | GTCAGGATGGCCGAGTGGTCTAAGGCGCCAGACTctaGCTTAGCTTCCCTGT |
| | CTGGGGATTCTGGTCTCCGTATGGAGGCGTGGGTTCGAATCCCACTTCTGAC |
| | A |
| 51 | GTCAGGATGGCCGAGTGGTCTAAGGCGCCAGACTctaGGTGACAAGCCTTAC |
| | CTACGGGTGTTCTGGTCTCCGAATGGAGGCGTGGGTTCGAATCCCACTTCTG |
| | ACA |
| 52 | GTCAGGATGGCCGAGTGGTCTAAGGCGCCAGACTctaGCGTTCGCTTCCTCT |
| | ACTGAGGGTTCTGGTCTCCGTGTGGAGGCGTGGGTTCGAATCCCACTTCTGA |
| | CA |
| 53 | GTCAGGATGGCCGAGTGGTCTAAGGCGCCAGACTtcaGCTATGGCTTCCTCG |
| | CTCTGAGGGTTCTGGTCTCCCCTGGAGGCGTGGGTTCGAATCCCACTTCTGA |
| | CA |
| 54 | GTCAGGATGGCCGAGTGGTCTAAGGCGCCAGACTtcaGCTTAGCTTCCCTGT |
| | CTGGGGATTCTGGTCTCCGTATGGAGGCGTGGGTTCGAATCCCACTTCTGAC |
| | A |
| 55 | GTCAGGATGGCCGAGTGGTCTAAGGCGCCAGACTtcaGGTGACAAGCCTTAC |
| | CTACGGGTGTTCTGGTCTCCGAATGGAGGCGTGGGTTCGAATCCCACTTCTG |
| | ACA |
| 56 | GTCAGGATGGCCGAGTGGTCTAAGGCGCCAGACTtcaGCGTTCGCTTCCTCT |
| | ACTGAGGGTTCTGGTCTCCGTGTGGAGGCGTGGGTTCGAATCCCACTTCTGA |
| | CA |
| 57 | GTCAGGATGGCCGAGTGGTCTAAGGCGCCAGACTttaGCTATGGCTTCCTCG |
| | CTCTGAGGGTTCTGGTCTCCCCTGGAGGCGTGGGTTCGAATCCCACTTCTGA |
| | CA |
| 58 | GTCAGGATGGCCGAGTGGTCTAAGGCGCCAGACTttaGCTTAGCTTCCCTGT |
| | CTGGGGATTCTGGTCTCCGTATGGAGGCGTGGGTTCGAATCCCACTTCTGAC |
| | A |
| 59 | GTCAGGATGGCCGAGTGGTCTAAGGCGCCAGACTttaGGTGACAAGCCTTAC |
| | CTACGGGTGTTCTGGTCTCCGAATGGAGGCGTGGGTTCGAATCCCACTTCTG |
| | ACA |
| 60 | GTCAGGATGGCCGAGTGGTCTAAGGCGCCAGACTttaGCGTTCGCTTCCTCT |
| | ACTGAGGGTTCTGGTCTCCGTGTGGAGGCGTGGGTTCGAATCCCACTTCTGA |
| | CA |
| 61 | CCTTCGATAGCTCAGTTGGTAGAGCGGAGGACTctaGAGTTACTAGAATAGT |
| | GATCCTTAGGTCGCTGGTTCGAATCCGGCTCGAAGGA |
| 62 | CCTTCGATAGCTCAGTTGGTAGAGCGGAGGACTctaGTCAGTACAATATGGT |
| | AATCCTTAGGTCGCTGGTTCGATTCCGGCTCGAAGGA |
| 63 | CCTTCGATAGCTCAGCTGGTAGAGCGGAGGACTctaGGCTTGTGGCTGTGGA |
| | CATCCTTAGGTCGCTGGTTCGATTCCGGCTCGAAGGA |
| 64 | CCTTCGATAGCTCAGCTGGTAGAGCGGAGGACTctaGCTAACTCCCCGTTAG |
| | AAGACATCCTTAGGTCGCTGGTTCGACTCCGGCTCGAAGGA |
| 65 | CTTTCGATAGTTCAGTTGGTAGAGCGGAGGACTctaGAGTATTAACGTTGGT |
| | GATCCTTAGGTCGCTGGTTCGAGTCCGGCTCGAAGGA |
| 66 | CCTTCGATAGCTCAGTTGGTAGAGCGGAGGACTttaGAGTTACTAGAATAGT |
| | GATCCTTAGGTCGCTGGTTCGAATCCGGCTCGAAGGA |
| 67 | CCTTCGATAGCTCAGTTGGTAGAGCGGAGGACTttaGTCAGTACAATATGGT |
| | AATCCTTAGGTCGCTGGTTCGATTCCGGCTCGAAGGA |
| 68 | CCTTCGATAGCTCAGCTGGTAGAGCGGAGGACTttaGGCTTGTGGCTGTGGA |
| | CATCCTTAGGTCGCTGGTTCGATTCCGGCTCGAAGGA |
| 69 | CCTTCGATAGCTCAGCTGGTAGAGCGGAGGACTttaGCTAACTCCCCGTTAG |
| | AAGACATCCTTAGGTCGCTGGTTCGACTCCGGCTCGAAGGA |
| 70 | CTTTCGATAGTTCAGTTGGTAGAGCGGAGGACTttaGAGTATTAACGTTGGT |
| | GATCCTTAGGTCGCTGGTTCGAGTCCGGCTCGAAGGA |
| 71 | GGGGGTATAGCTCAGTGGTAGAGCATTTGACTtcaGATCAAGAGGTCCCTGG |
| | TTCAAATCCAGGTGCCCCCT |
| 72 | GGGGGTATAGCTCAGGGGTAGAGCATTTGACTtcaGATCAAGAGGTCCCTGG |
| | TTCAAATCCAGATGCCCCCT |
| 73 | GGGGGTATAGCTCAGGGGTAGAGTATTTGGCTtcaGATCAAGAGGTCCCTGG |
| | TTCAAATCCAGGTGCCCCCT |
| 74 | GGGGGTATAGCTCAGGGGTAGAGCATTTGACTtcaGATCAAGAGGTCCTTGG |
| | TTCAAATCCAGGTGTCCCCT |
| 75 | GGGGGTATAGCTCAGAGGTAGAGCATTTGACTtcaGATCAAGAGATCTCTGG |
| | TTCAAATCCAGGTGCCCCCT |
| 76 | GGGGGTATAGCTCAGGGGTAGAGCATTTGACTtcaGATCAAGAGGTCCCTAG |
| | TTCAAATCCAGGTGCCCCCT |
| 77 | GGTGGTATAGCTCAGGGGTAGAGCATTTGACTtcaGATCAAGAGATCCCTGG |
| | TTCGAATCCAGGTGCCCCCT |
| 78 | GGGGGTATAACTCAGGGGTAGAGCATTTGACTtcaGATCAAGAGGTCCCTGG |
| | TTCAAATCCAGGTGCCCCCT |
| 79 | TGGGGTATAGCTCAGGGGTAGAGCATTTGACTtcaGATCAAGAGGTCCCTGG |
| | TTCAAATCCAGGTGCCCCCT |
| 80 | GGGGGTATAGCTCAGAGGAAGAGCATTTGACTtcaGATCAAGAGGTCCCTGA |
| | TTCAAATCCAGGTGCCCCCT |
| 81 | GGGGGTAAAGCTCAGGGGTAGAGCATTTGACTtcaGATTAAGAGGTCCCTGG |
| | TTCAAATCCAGGTACCCCCT |
| 83 | GGGGTTATAGCTCAGGTGTAGAGCATTTGACTtcaGATCAAGAGGTCCCTGG |
| | TTCAAATCCAGGTGCCCCCT |
| 84 | GGGGGTATAGCTCAGGGGTAGAGCATTTGACTtcaGATCACGAGGTCCCTGG |
| | TTCAAATCGAGGTGCCCCCT |
| 85 | GGGGGTATAGCTCAGGGGTGGAGCATTTGACTtcaGATCAAGGGGTCCCTGT |
| | TTCAAATCCAGGTGCCCCCT |
| 86 | GGGGGTATAGCTCAGTGGTAGAGCATTTGACTtcaGATCAAGAGGTCCCCGG |
| | TTCAAATCCGGGTGCCCCCT |
| 88 | GGGGGTATAGCTCAGGGGTAGAGCATTTGACTtcaGATCAAGAGGTCCCTGG |
| | TTCAAATCCGGGTGCCCCCT |
| 89 | GGGGGTATAGCTCAGGGGTAGAGCATTTGACTtcaGATCAAGAGGTCCCTGG |
| | TTCAAATCCAGGTACCCCCT |
| 91 | GGGGGCATAGCTCAGGGGTAGAGCATTTGACTtcaGATCAAGAGGTCCCCGG |
| | TTCAAATCCGGGTGCTCCCT |
| 92 | GGGGGTATAGCTCAGGGGTAGAGCATTTGACTtcaGATTAAGAGGTCCCTGG |
| | TTCAAATCCAGGTGCCCCCT |
| 94 | TCCCTGGTGGTCTAGTGGTTAGGATTTGGCGCTctaACCGCCGCGGCCTGGG |
| | TTCGATTCCCGGTCAGGGAA |
| 95 | TCCCTGGTGGTCTAGTGGTTAGGCTTTGGTGCTctaACCTCCATGGCCCAGG |
| | TTTGATTCCTGGTCAGGGAA |
| 97 | TCCCTGGTGGTCTAGTGGTTAGGATTTGGCGCTttaACCGCCGCGGCCTGGG |
| | TTCGATTCCCGGTCAGGGAA |
| 98 | TCCCTGGTGGTCTAGTGGTTAGGCTTTGGTGCTttaACCTCCATGGCCCAGG |
| | TTTGATTCCTGGTCAGGGAA |
| 100 | TCCCATATGGTCTAGCGGTTAGGATTCCTGGTTctaACCCAGGCGGCCCGGG |
| | TTCGACTCCCGGTATGGGAA |
| 103 | TCCCATATGGTCTAGCGGTTAGGATTCCTGGTTttaACCCAGGCGGCCCGGG |
| | TTCGACTCCCGGTATGGGAA |
| 105 | GTTTCCGTAGTGTAGTGGTTAGCGCGTTCGCCTtcaAAAGCGAAAGGTCCCC |
| | GGTTCGAAACCGGGCGGAAACA |
| 107 | GCATTGGTAGTTCAATGGTAGAATTCTCGCCTtcaACGCGGGTGACCCGGGT |
| | TCGATTCCCGGCCAATGCA |
| 108 | GCATTGGTGGTTCAATGGTAGAATTCTCGCCTtcaACGCGGGTGACCCGGGT |
| | TCGATTCCCGGCCAATGCA |
| 109 | GCATTGGTGGTTCAATGGTAGAATTCTCGCCTtcaACTCGGGTGACCCGGGT |
| | TCGATTCCCGGCCAATGCA |
| 112 | GCATTGGTGGTTCAGTGGTAGAATTCTCGCCTtcaACGCGGGAGGCCCGGGT |
| | TTGATTCCCGGCCAATGCA |
| 113 | GCATTGGTGGTTCAGTGGTAGAATTCTCGCCTtcaACGCGGGAGGCCCGGGT |
| | TCGGTTCCCGGCCAATGCA |
| 115 | GGTAGCGTGGCCGAGCGGTCTAAGGCGCTGGATTctaGCTCCAGTCTCTTCG |
| | GGGGCGTGGGTTCGAATCCCACCGCTGCCA |
| 116 | GGTAGTGTGGCCGAGCGGTCTAAGGCGCTGGATTctaGCTCCAGTCTCTTCG |
| | GGGGCGTGGGTTCGAATCCCACCGCTGCCA |
| 118 | GGTAGCGTGGCCGAGCGGTCTAAGGCGCTGGATTtcaGCTCCAGTCTCTTCG |
| | GGGGCGTGGGTTCGAATCCCACCGCTGCCA |
| 119 | GGTAGTGTGGCCGAGCGGTCTAAGGCGCTGGATTtcaGCTCCAGTCTCTTCG |
| | GGGGCGTGGGTTCGAATCCCACCGCTGCCA |
| 121 | GGTAGCGTGGCCGAGCGGTCTAAGGCGCTGGATTttaGCTCCAGTCTCTTCG |
| | GGGGCGTGGGTTCGAATCCCACCGCTGCCA |
| 122 | GGTAGTGTGGCCGAGCGGTCTAAGGCGCTGGATTttaGCTCCAGTCTCTTCG |
| | GGGGCGTGGGTTCGAATCCCACCGCTGCCA |
| 124 | GTCAGGATGGCCGAGTGGTCTAAGGCGCCAGACTctaGTTCTGGTCTCCCCT |
| | GGAGGCGTGGGTTCGAATCCCACTTCTGACA |
| 126 | GTCAGGATGGCCGAGTGGTCTAAGGCGCCAGACTctaGTTCTGGTCTCCGAA |
| | TGGAGGCGTGGGTTCGAATCCCACTTCTGACA |
| 127 | GTCAGGATGGCCGAGTGGTCTAAGGCGCCAGACTctaGTTCTGGTCTCCGTG |
| | TGGAGGCGTGGGTTCGAATCCCACTTCTGACA |
| 128 | GTCAGGATGGCCGAGTGGTCTAAGGCGCCAGACTtcaGTTCTGGTCTCCCCT |
| | GGAGGCGTGGGTTCGAATCCCACTTCTGACA |
| 130 | GTCAGGATGGCCGAGTGGTCTAAGGCGCCAGACTtcaGTTCTGGTCTCCGAA |
| | TGGAGGCGTGGGTTCGAATCCCACTTCTGACA |
| 131 | GTCAGGATGGCCGAGTGGTCTAAGGCGCCAGACTtcaGTTCTGGTCTCCGTG |
| | TGGAGGCGTGGGTTCGAATCCCACTTCTGACA |
| 132 | GTCAGGATGGCCGAGTGGTCTAAGGCGCCAGACTttaGTTCTGGTCTCCCCT |
| | GGAGGCGTGGGTTCGAATCCCACTTCTGACA |
| 134 | GTCAGGATGGCCGAGTGGTCTAAGGCGCCAGACTttaGTTCTGGTCTCCGAA |
| | TGGAGGCGTGGGTTCGAATCCCACTTCTGACA |
| 135 | GTCAGGATGGCCGAGTGGTCTAAGGCGCCAGACTttaGTTCTGGTCTCCGTG |
| | TGGAGGCGTGGGTTCGAATCCCACTTCTGACA |
| 136 | GTCAGGATGGCCGAGCGGTCTAAGGCGCTGCGTTctaGTCGCAGTCTCCCCT |
| | GGAGGCGTGGGTTCGAATCCCACTCCTGACA |
| 137 | GTCAGGATGGCCGAGCGGTCTAAGGCGCTGCGTTctaGTCGCAGTCTCCCCT |
| | GGAGGCGTGGGTTCGAATCCCACTTCTGACA |
| 138 | GTCAGGATGGCCGAGTGGTCTAAGGAGCTGTGTTctaGTCGCAGTCTCCCCT |
| | GGAGGCGTGGGTTCGAATCCCACTCCTGACA |
| 139 | GTCAGGATGGCCGAGCAGTCTAAGGCACTGCGTTctaGTCGCAGTCTCCCCT |
| | GGAGGCGTGGATTCGAATCCCACTCCTGACA |
| 140 | GTCAGGATGGCCGAGCGGTCTAAGGCGCTGCGTTtcaGTCGCAGTCTCCCCT |
| | GGAGGCGTGGGTTCGAATCCCACTCCTGACA |
| 141 | GTCAGGATGGCCGAGCGGTCTAAGGCGCTGCGTTtcaGTCGCAGTCTCCCCT |
| | GGAGGCGTGGGTTCGAATCCCACTTCTGACA |
| 142 | GTCAGGATGGCCGAGTGGTCTAAGGAGCTGTGTTtcaGTCGCAGTCTCCCCT |
| | GGAGGCGTGGGTTCGAATCCCACTCCTGACA |
| 143 | GTCAGGATGGCCGAGCAGTCTAAGGCACTGCGTTtcaGTCGCAGTCTCCCCT |
| | GGAGGCGTGGATTCGAATCCCACTCCTGACA |
| 144 | GTCAGGATGGCCGAGCGGTCTAAGGCGCTGCGTTttaGTCGCAGTCTCCCCT |
| | GGAGGCGTGGGTTCGAATCCCACTCCTGACA |
| 145 | GTCAGGATGGCCGAGCGGTCTAAGGCGCTGCGTTttaGTCGCAGTCTCCCCT |
| | GGAGGCGTGGGTTCGAATCCCACTTCTGACA |
| 146 | GTCAGGATGGCCGAGTGGTCTAAGGAGCTGTGTTttaGTCGCAGTCTCCCCT |
| | GGAGGCGTGGGTTCGAATCCCACTCCTGACA |
| 147 | GTCAGGATGGCCGAGCAGTCTAAGGCACTGCGTTttaGTCGCAGTCTCCCCT |
| | GGAGGCGTGGATTCGAATCCCACTCCTGACA |
| 148 | ACCAGAATGGCCGAGTGGTTAAGGCGTTGGACTctaGATCCAATGGATTTAT |
| | ATCCGCGTGGGTTCGAACCCCACTTCTGGTA |
| 149 | ACCAGGATGGCCGAGTGGTTAAGGCGTTGGACTctaGATCCAATGGACATAT |
| | GTCTGCGTGGGTTCGAACCCCACTCCTGGTA |
| 150 | ACTGGGATGGCTGAGTGGTTAAGGCGTTGGACTctaGATCCAATGGGCGGTT |
| | GCCTGCGTGGGTTCGAACCCCACTCCCAGTA |
| 151 | GATGGGATGGCTGAGAGGTTAAGGCTTTGGACTctaGATCCAATGGGCAGAT |
| | GCCTGCGTGGGTTTGAACCCCACTCCCAATA |
| 152 | ACCAGAATGGCCGAGTGGTTAAGGCGTTGGACTtcaGATCCAATGGATTTAT |
| | ATCCGCGTGGGTTCGAACCCCACTTCTGGTA |
| 153 | ACCAGGATGGCCGAGTGGTTAAGGCGTTGGACTtcaGATCCAATGGACATAT |
| | GTCTGCGTGGGTTCGAACCCCACTCCTGGTA |
| 154 | ACTGGGATGGCTGAGTGGTTAAGGCGTTGGACTtcaGATCCAATGGGCGGTT |
| | GCCTGCGTGGGTTCGAACCCCACTCCCAGTA |
| 155 | GATGGGATGGCTGAGAGGTTAAGGCTTTGGACTtcaGATCCAATGGGCAGAT |
| | GCCTGCGTGGGTTTGAACCCCACTCCCAATA |
| 156 | ACCAGAATGGCCGAGTGGTTAAGGCGTTGGACTttaGATCCAATGGATTTAT |
| | ATCCGCGTGGGTTCGAACCCCACTTCTGGTA |
| 157 | ACCAGGATGGCCGAGTGGTTAAGGCGTTGGACTttaGATCCAATGGACATAT |
| | GTCTGCGTGGGTTCGAACCCCACTCCTGGTA |
| 158 | ACTGGGATGGCTGAGTGGTTAAGGCGTTGGACTttaGATCCAATGGGCGGTT |
| | GCCTGCGTGGGTTCGAACCCCACTCCCAGTA |
| 159 | GATGGGATGGCTGAGAGGTTAAGGCTTTGGACTttaGATCCAATGGGCAGAT |
| | GCCTGCGTGGGTTTGAACCCCACTCCCAATA |
| 161 | GGTAGTGTGGCCGAGCGGTCTAAGGCGCTGGATTctaGCTCCAGTCTCTTCG |
| | GAGGCGTGGGTTCGAATCCCACCACTGCCA |
| 164 | GGTAGTGTGGCCGAGCGGTCTAAGGCGCTGGATTtcaGCTCCAGTCTCTTCG |
| | GAGGCGTGGGTTCGAATCCCACCACTGCCA |
| 167 | GGTAGTGTGGCCGAGCGGTCTAAGGCGCTGGATTttaGCTCCAGTCTCTTCG |
| | GAGGCGTGGGTTCGAATCCCACCACTGCCA |
| 169 | GCCCAGCTAGCTCAGTTGGTAGAGCGTGGGACTctaAATCCTAGGGTCGTGG |
| | GTTCGAACCCCACGTTGGGCG |
| 170 | GCCCAGCTAGCTCAGTCTGTAGAGCATGAGACTctaAGTCTCAGGGTCATGG |
| | GTTGGAGCCCCATGTTGTGCA |
| 171 | GCCTAGCTAGTTCAGTCGGTAGAGCATGAGACTctaAATCTCAGGTTCATGA |
| | GTTTGAGCCCCATGTTGGTTTGGCA |
| 172 | CCCCGGCTAGCTCAGTCAGTAGAGCTTGAGAATctaAATCTCAGGGTCGTGG |
| | GTTGGAGCCCCACGTTGGGCG |
| 179 | GGTTCCATGGTGTAATGGTTAGCACTCTGGACTctaAATCCAGCGACCCGAG |
| | TTCAAATCTCGGTGGGACCT |
| 181 | GGTTCCATGGTGTAATGGTTAGCACTCTGGACTctaAATCCAGCGATCCGAG |
| | TTCAAATCTCGGTGGGACCT |
| 182 | GGTTCCATGGTGTAATGGTGAGCACTCTGGACTctaAATCCAGCGATCCGAG |
| | TTCAAATCTCGGTGGGACCT |
| 183 | GGTTCCATGGTGTAATGGCTAGCACTCTGGACTctaAATCCAGCGATCCGAG |
| | TTCAAATCTCGGTGGGATTT |
| 184 | GGTTCCATGGTGTAATGGTTAGCACTCTGGACTctaAATCCAGCCATACAAG |
| | TTCAAATCTCAGTGGAACCT |
| 185 | GGTTCCTTGGTGTAAGATGAGCACTCTGGATTctaAATCCAGCGATCAGAGT |
| | TCAAATCTCGGTGGGACCT |
| 186 | GGTCCCATGGTGTAATGGTTAGCACTCTGGACTctaAATCCAGCAATCTGAG |
| | TTCAAATCTCGGTGGGACCT |
| 188 | GGTCTCATGGTGTAATGGTTAGCACACTGGACTctaAGTCCAGCAATCCGAG |
| | TTCGAGTCTTGGTGAGACCA |
| 189 | GGACCCATGGTGTAATGGTTAGCACTCTGGACTctaAATCCAGCAATCCAAG |
| | TTCAAATCTCGGTGGGACCT |
| 190 | GTTTCCATGGTGTAATGGTTGGCACTCTGGACTctaAATCCAGCAATCCAAG |
| | TTCAAGTCTCTGTGGGACCT |
| 196 | GCCCGGCTAGCTCAGTCGGTAGAGCATGGGACTctaAATCCCAGGGTCGTGG |
| | GTTCGAGCCCCACGTTGGGCG |
| 197 | GCCCGGCTAGCTCAGTCGGTAGAGCATGAGACTctaAATCTCAGGGTCGTGG |
| | GTTCGAGCCCCACGTTGGGCG |
| 198 | GCCCAGCTAGCTCAGTCTGTAGAGCATGAGACTctaAATCTCAGGGTCGTGA |
| | GTTCGAGCCCCACGTTGGGTG |
| 199 | GCCCAGATAGCTCAGTGGGTAGAGCATGAGACTctaAATCTCAGGGTCATGG |
| | GTTCATGCCCCATGTTGGGTA |
| 200 | GTCCTGCTGGCTCAGTCGGTACAGCATGGGACTctaAATCCCAGGGTCGTGG |
| | GTTCGAGCTCCACGTTGGGTA |
| 201 | GCCTGGCTAGCTCAGTCCATAGAGCATGGGACTctaAATCCCAGGGTCATGG |
| | GTTCGAGCCCCATATTAGGCA |
| 202 | GCCCAGCTAGCTTAGTTGGTAGAGCATGAGACTctaAATCTCAGAGTCATGG |
| | GTTCAGGCCTCATGTTTGGCA |
| 203 | AACCTGGCTAGGTCAGTTGGTAGATCATGAGACTctaAATCTCAGGGTCATG |
| | GGTTCAAGCCCCATGTTGGTTT |
| 204 | GCCCAGCTAGCTCAGTTGGTAGAGCGTGGGACTttaAATCCTAGGGTCGTGG |
| | GTTCGAACCCCACGTTGGGCG |
| 205 | GCCCAGCTAGCTCAGTCTGTAGAGCATGAGACTttaAGTCTCAGGGTCATGG |
| | GTTGGAGCCCCATGTTGTGCA |
| 206 | GCCTAGCTAGTTCAGTCGGTAGAGCATGAGACTttaAATCTCAGGTTCATGA |
| | GTTTGAGCCCCATGTTGGTTTGGCA |
| 207 | CCCCGGCTAGCTCAGTCAGTAGAGCTTGAGAATttaAATCTCAGGGTCGTGG |
| | GTTGGAGCCCCACGTTGGGCG |
| 208 | GCCCGGCTAGCTCAGTCGGTAGAGCATGGGACTttaAATCCCAGGGTCGTGG |
| | GTTCGAGCCCCACGTTGGGCG |
| 209 | GCCCGGCTAGCTCAGTCGGTAGAGCATGAGACTttaAATCTCAGGGTCGTGG |
| | GTTCGAGCCCCACGTTGGGCG |
| 210 | GCCCAGCTAGCTCAGTCTGTAGAGCATGAGACTttaAATCTCAGGGTCGTGA |
| | GTTCGAGCCCCACGTTGGGTG |
| 211 | GCCCAGATAGCTCAGTGGGTAGAGCATGAGACTttaAATCTCAGGGTCATGG |
| | GTTCATGCCCCATGTTGGGTA |
| 212 | GTCCTGCTGGCTCAGTCGGTACAGCATGGGACTttaAATCCCAGGGTCGTGG |
| | GTTCGAGCTCCACGTTGGGTA |
| 213 | GCCTGGCTAGCTCAGTCCATAGAGCATGGGACTttaAATCCCAGGGTCATGG |
| | GTTCGAGCCCCATATTAGGCA |
| 214 | GCCCAGCTAGCTTAGTTGGTAGAGCATGAGACTttaAATCTCAGAGTCATGG |
| | GTTCAGGCCTCATGTTTGGCA |
| 215 | AACCTGGCTAGGTCAGTTGGTAGATCATGAGACTttaAATCTCAGGGTCATG |
| | GGTTCAAGCCCCATGTTGGTTT |
| 216 | GCCCGGATAGCTCAGTCGGTAGAGCATCAGACTctaAATCTGAGGGTCCAGG |
| | GTTCAAGTCCCTGTTCGGGCG |
| 218 | GCCTGGATAGCTCAATTGGTAGAGCATCAGACTctaAATCTGAGGGTTCAGG |
| | GTTCAAGTCCCTGTTCAGGCG |
| 219 | GCCCAGCCAGCTCAGTAGGTAGAGTATGAGACTctaAATCTCAGGGTGGTGG |
| | GTTCGAGCCCCATGTTGGGGG |
| 220 | TGTGGTGTAGCTCAGTCGGTAGAGCATCAGACTctaAATCTGAGGGTCCAGG |
| | GTTCAGGTCCCTGTTCGGGTGCCAAAA |
| 221 | GCCCGGATAGCTCAGTCGGTAGAGCATCAGACTttaAATCTGAGGGTCCAGG |
| | GTTCAAGTCCCTGTTCGGGCG |
| 223 | GCCTGGATAGCTCAATTGGTAGAGCATCAGACTttaAATCTGAGGGTTCAGG |
| | GTTCAAGTCCCTGTTCAGGCG |
| 224 | GCCCAGCCAGCTCAGTAGGTAGAGTATGAGACTttaAATCTCAGGGTGGTGG |
| | GTTCGAGCCCCATGTTGGGGG |
| 225 | TGTGGTGTAGCTCAGTCGGTAGAGCATCAGACTttaAATCTGAGGGTCCAGG |
| | GTTCAGGTCCCTGTTCGGGTGCCAAAA |
| 228 | GTAGTCGTGGCCAAGTGAGTAAGGCAATGGACTctaAATCCATTGGGGTCTC |
| | CCAGCACAGGTTCAAATCCTGCTGACTATG |
| 231 | GTAGTCGTGGCCAAGTGAGTAAGGCAATGGACTtcaAATCCATTGGGGTCTC |
| | CCAGCACAGGTTCAAATCCTGCTGACTATG |
| 234 | GTAGTCGTGGCCAAGTGAGTAAGGCAATGGACTttaAATCCATTGGGGTCTC |
| | CCAGCACAGGTTCAAATCCTGCTGACTATG |
| 237 | GCTGTGATGGCCGAGTGGTTAAGGCGTTGGACTctaAATCCAATGGGTTCTT |
| | CCCGCGCAGGTTCAAATCCTGCTCACAGCG |
| 240 | GCTGTGATGGCCGAGTGGTTAAGGCGTTGGACTtcaAATCCAATGGGTTCTT |
| | CCCGCGCAGGTTCAAATCCTGCTCACAGCG |
| 243 | GCTGTGATGGCCGAGTGGTTAAGGCGTTGGACTttaAATCCAATGGGTTCTT |
| | CCCGCGCAGGTTCAAATCCTGCTCACAGCG |
| 245 | GACGAGGTGGCCGAGTGGTTAAGGCGATGGACTctaAATCCATTGTGCTCTG |
| | CACGCATGGGTTCGAATCCCATCCTCGTCG |
| 246 | GACGAGGTGGCCGAGTGGTTAAGGCGATGGACTctaAATCCATTGTGCTTTG |
| | CACGCGTGGGTTCGAATCCCATCCTCGTCG |
| 247 | GACGAGGTGGCCGAGTGGTTAAGGCGATGGACTctaAATCCATTGTGCTCTG |
| | CACGCGTGGGTTCGAATCCCATCCTCGTCG |
| 248 | GATGAGGTGGCCGAGTGGTTAAGGCGATGGACTctaAATCCATTGTGCTCTG |
| | CACGCATGGGTTCGAATCCCATCCTCATCG |
| 250 | GACGAGGTGGCCGAGTGGTTAAGGCGATGGACTtcaAATCCATTGTGCTCTG |
| | CACGCATGGGTTCGAATCCCATCCTCGTCG |
| 251 | GACGAGGTGGCCGAGTGGTTAAGGCGATGGACTtcaAATCCATTGTGCTTTG |
| | CACGCGTGGGTTCGAATCCCATCCTCGTCG |
| 252 | GACGAGGTGGCCGAGTGGTTAAGGCGATGGACTtcaAATCCATTGTGCTCTG |
| | CACGCGTGGGTTCGAATCCCATCCTCGTCG |
| 253 | GATGAGGTGGCCGAGTGGTTAAGGCGATGGACTtcaAATCCATTGTGCTCTG |
| | CACGCATGGGTTCGAATCCCATCCTCATCG |
| 255 | GACGAGGTGGCCGAGTGGTTAAGGCGATGGACTttaAATCCATTGTGCTCTG |
| | CACGCATGGGTTCGAATCCCATCCTCGTCG |
| 256 | GACGAGGTGGCCGAGTGGTTAAGGCGATGGACTttaAATCCATTGTGCTTTG |
| | CACGCGTGGGTTCGAATCCCATCCTCGTCG |
| 257 | GACGAGGTGGCCGAGTGGTTAAGGCGATGGACTttaAATCCATTGTGCTCTG |
| | CACGCGTGGGTTCGAATCCCATCCTCGTCG |
| 258 | GATGAGGTGGCCGAGTGGTTAAGGCGATGGACTttaAATCCATTGTGCTCTG |
| | CACGCATGGGTTCGAATCCCATCCTCATCG |
| 259 | GCTGAAATAGCTCAGTTGGGAGAGCATTAGACTctaGATCTAAAGGTCCCTG |
| | GTTTGATCCCGGGTTTCGGCA |
| 260 | GCTGAAATAGCTCAGTTGGGAGAGCATTAGACTtcaGATCTAAAGGTCCCTG |
| | GTTTGATCCCGGGTTTCGGCA |
| 261 | GCTGAAATAGCTCAGTTGGGAGAGCATTAGACTttaGATCTAAAGGTCCCTG |
| | GTTTGATCCCGGGTTTCGGCA |
| 265 | GACCTCGTGGCGCAATGGTAGCGCGTCTGACTctaGATCAGAAGGTTGCGTG |
| | TTCAAATCACGTCGGGGTCA |
| 270 | GACCTCGTGGCACAATGGTAGCACGTCTGACTctaGATCAGAAGGTTGCGTG |
| | TTCAAATCACGTCGGGGTCA |
| 280 | CCTTCGATAGCTCAGCTGGTAGAGCGGAGGACTctaGATCCTTAGGTCGCTG |
| | GTTCGACTCCGGCTCGAAGGA |
| 281 | CTTTCGATAGTTCAGTTGGTAGAGCGGAGGACTctaGATCCTTAGGTCGCTG |
| | GTTCGAGTCCGGCTCGAAGGA |
| 285 | CCTTCGATAGCTCAGCTGGTAGAGCGGAGGACTttaGATCCTTAGGTCGCTG |
| | GTTCGACTCCGGCTCGAAGGA |
| 286 | CTTTCGATAGTTCAGTTGGTAGAGCGGAGGACTttaGATCCTTAGGTCGCTG |
| | GTTCGAGTCCGGCTCGAAGGA |

In certain embodiments, the tRNA comprises, consists essentially of, or consists of a nucleotide sequence shown in TABLE 3. In certain embodiments, the tRNA comprises, consists essentially of, or consists of a nucleotide sequence having 80%, 85%, 86%, 87%, 88%, 89%, 90%, 91%, 92%, 93%, 94%, 95%, 96%, 97%, 98%, or 99% sequence identity to a nucleotide sequence shown in TABLE 3. In certain embodiments, the tRNA comprises, consists essentially of, or consists of a nucleotide sequence selected from SEQ ID NOs: 6-9, 11, 16-18, 22, 35, 36, 38, 45, 178, 180, and 187, or a nucleotide sequence having 80%, 85%, 86%, 87%, 88%, 89%, 90%, 91%, 92%, 93%, 94%, 95%, 96%, 97%, 98%, or 99% sequence identity to a nucleotide sequence selected from SEQ ID NOs: 6-9, 11, 16-18, 22, 35, 36, 38, 45, 178, 180, and 187.

TABLE 3

| SEQ ID NO | Suppressor tRNA Sequence |
|---|---|
| 1 | GGGCCAGTGGCGCAATGGATAACGCGTCTGACTtcaGATCAGAAGATTCCAG |
| | GTTCGACTCCTGGCTGGCTCG |
| 3 | GGGCCAGTGGCGCAATGGATAACGCGTCTGACTtcaGATCAGAAGATTCTAG |
| | GTTCGACTCCTGGCTGGCTCG |
| 4 | GGCCGCGTGGCCTAATGGATAAGGCGTCTGATTtcaGATCAGAAGATTGAGG |
| | GTTCGAGTCCCTTCGTGGTCG |
| 6 | GACCCAGTGGCCTAATGGATAAGGCATCAGCCTtcaGAGCTGGGGATTGTGG |
| | GTTCGAGTCCCATCTGGGTCG |
| 7 | GCCCCAGTGGCCTAATGGATAAGGCACTGGCCTtcaAAGCCAGGGATTGTGG |
| | GTTCGAGTCCCACCTGGGGTA |
| 8 | GCCCCAGTGGCCTAATGGATAAGGCACTGGCCTtcaAAGCCAGGGATTGTGG |
| | GTTCGAGTCCCACCTGGGGTG |
| 9 | GCCCCGGTGGCCTAATGGATAAGGCATTGGCCTtcaAAGCCAGGGATTGTGG |
| | GTTCGAGTCCCACCCGGGGTA |
| 10 | GCCCCAGTGGCCTAATGGATAAGGCATTGGCCTtcaAAGCCAGGGATTGTGG |
| | GTTCGAGTCCCATCTGGGGTG |
| 11 | GGCCGCGTGGCCTAATGGATAAGGCGTCTGACTtcaGATCAGAAGATTGCAG |
| | GTTCGAGTCCTGCCGCGGTCG |
| 12 | GACCGCGTGGCCTAATGGATAAGGCGTCTGACTtcaGATCAGAAGATTGAGG |
| | GTTCGAGTCCCTTCGTGGTCG |
| 13 | GACCACGTGGCCTAATGGATAAGGCGTCTGACTtcaGATCAGAAGATTGAGG |
| | GTTCGAATCCCTTCGTGGTTG |
| 16 | GGCTCTGTGGCGCAATGGATAGCGCATTGGACTtcaAATTCAAAGGTTGTGG |
| | GTTCGAATCCCACCAGAGTCG |
| 17 | GGCTCCGTGGCGCAATGGATAGCGCATTGGACTtcaAGAGGCTGAAGGCATT |
| | CAAAGGTTCCGGGTTCGAGTCCCGGCGGAGTCG |
| 18 | GGCTCCGTGGCGCAATGGATAGCGCATTGGACTtcaAATTCAAAGGTTCCGG |
| | GTTCGAGTCCCGGCGGAGTCG |
| 22 | GGCTCTGTGGCGCAATGGATAGCGCATTGGACTtcaAATTCAAAGGTTGTGG |
| | GTTCGAGTCCCACCAGAGTCG |
| 35 | GTCTCTGTGGCGCAATGGACGAGCGCGCTGGACTtcaAATCCAGAGGTTCCG |
| | GGTTCGAGTCCCGGCAGAGATG |
| 36 | GGTCCCATGGTGTAATGGTTAGCACTCTGGACTttaAATCCAGCGATCCGAG |
| | TTCAAATCTCGGTGGGACCT |
| 38 | GGTTCCATGGTGTAATGGTTAGCACTCTGGACTttaAATCCAGCGATCCGAG |
| | TTCAAATCTCGGTGGAACCT |
| 45 | GGCCCCATGGTGTAATGGTTAGCACTCTGGACTttaAATCCAGCGATCCGAG |
| | TTCAAATCTCGGTGGGACCT |
| 178 | GGTCCCATGGTGTAATGGTTAGCACTCTGGACTctaAATCCAGCGATCCGAG |
| | TTCAAATCTCGGTGGGACCT |
| 180 | GGTTCCATGGTGTAATGGTTAGCACTCTGGACTctaAATCCAGCGATCCGAG |
| | TTCAAATCTCGGTGGAACCT |
| 187 | GGCCCCATGGTGTAATGGTTAGCACTCTGGACTctaAATCCAGCGATCCGAG |
| | TTCAAATCTCGGTGGGACCT |
| 287 | GGCCTCGTGGCGCAACGGTAGCGCGTCTGACTTCAGATCAGAAGGTTGCGGG |
| | TTCAAATCCCGTCGGGGTCA |
| 288 | GGCCTCGTGGCGCAACGGTAGCGCGTCTGACTTCAGATCAGAAGGTTACGGG |
| | TTCAAATCCCGTCGGGGTCA |
| 289 | GGCCTCGTGGCGCAACGGTAGCGCGTCTGACTTCAGATCAGAAGGTTCCGGG |
| | TTCAAATCCCGGCGGGGTCA |
| 290 | CGTCGGCTCTGTGGCGCAATGGATAGCGCATTGGACTTCAAATTCAAAGGTT |
| | GTGGGTTCGAGTCCCAGAGTCG |
| 291 | CGTCGCCCCAGTGGCCTAATGGATAAGGCACTGGCCTTCAAAGCCAGGGATT |
| | GTGGGTTCGAGTCCCACCTGGGGTG |
| 292 | CGTCGGCTCCGTGGCGCAATGGATAGCGCATTGGACTTCAAATTCAAAGGTT |
| | CCGGGTTCGAGTCCCGGCGGAGTCG |
| 293 | CGTCGCCCCAGTGGCCTAATGGATAAGGCATTGGCCTTCAAAGCCAGGGATT |
| | GTGGGTTCGAGTCCCATCTGGGGTG |
| 294 | CGTCGGCTCTGTGGCGCAATGGATAGCGCATTGGACTTCAAATTCAAAGGTT |
| | GTGGGTTCGAATCCCACCAGAGTCG |
| 295 | CGTCGGCTCTGTGGCGCAATGGATAGCGCATTGGACTTCAAGCTGAGCCTAG |
| | TGTGGTCATTCAAAGGTTGTGGGTTCGAGTCCCACCAGAGTCG |
| 296 | CGTCGCCCCGGTGGCCTAATGGATAAGGCATTGGCCTTCAAAGCCAGGGATT |
| | GTGGGTTCGAGTCCCACCCGGGGTA |
| 297 | CGTCGGCTCCGTGGCGCAATGGATAGCGCATTGGACTTCAAGAGGCTGAAGG |
| | CATTCAAAGGTTCCGGGTTCGAGTCCCGGCGGAGTCG |
| 298 | CGTCGGCTCTGTGGCGCAATGGATAGCGCATTGGACTTCAAGTGACGAATAG |
| | AGCAATTCAAAGGTTGTGGGTTCGAATCCCACCAGAGTCG |
| 299 | CGTCGGCCGCGTGGCCTAATGGATAAGGCGTCTGACTTCAGATCAGAAGATT |
| | GCAGGTTCGAGTCCTGCCGCGGTCG |
| 300 | CGTCGACCGCGTGGCCTAATGGATAAGGCGTCTGACTTCAGATCAGAAGATT |
| | GAGGGTTCGAGTCCCTTCGTGGTCG |
| 301 | CGTCGGCTCTGTGGCGCAATGGATAGCGCATTGGACTTCAAGATAGTTAGAG |
| | AAATTCAAAGGTTGTGGGTTCGAGTCCCACCAGAGTCG |
| 302 | CGTCGGTTCCATGGTGTAATGGTGAGCACTCTGGACTCTAAATCCAGCGATC |
| | CGAGTTCGAGTCTCGGTGGAACCT |
| 303 | CGTCGGCCCCATGGTGTAATGGTTAGCACTCTGGACTCTAAATCCAGCGATC |
| | CGAGTTCAAATCTCGGTGGGACCT |
| 304 | CGTCGGTCCCATGGTGTAATGGTTAGCACTCTGGACTCTAAATCCAGCAATC |
| | CGAGTTCGAATCTCGGTGGGACCT |
| 305 | CGTCGGTCCCATGGTGTAATGGTTAGCACTCTGGACTCTAAATCCAGCGATC |
| | CGAGTTCAAATCTCGGTGGGACCT |
| 306 | CGTCGGCCCCATGGTGTAATGGTCAGCACTCTGGACTCTAAATCCAGCGATC |
| | CGAGTTCAAATCTCGGTGGGACCC |
| 307 | CGTCGGTTCCATGGTGTAATGGTAAGCACTCTGGACTCTAAATCCAGCGATC |
| | CGAGTTCGAGTCTCGGTGGAACCT |
| 308 | CGTCGGTTCCATGGTGTAATGGTTAGCACTCTGGACTCTAAATCCGGTAATC |
| | CGAGTTCAAATCTCGGTGGAACCT |
| 309 | CGTCGGTTCCATGGTGTAATGGTTAGCACTCTGGACTCTAAATCCAGCGATC |
| | CGAGTTCAAGTCTCGGTGGAACCT |
| 310 | CGTCGGTTCCATGGTGTAATGGTAAGCACTCTGGACTTTAAATCCAGCGATC |
| | CGAGTTCGAGTCTCGGTGGAACCT |
| 311 | CGTCGGCCCCATGGTGTAATGGTTAGCACTCTGGACTTTAAATCCAGCGATC |
| | CGAGTTCAAATCTCGGTGGGACCT |
| 312 | CGTCGGTTCCATGGTGTAATGGTGAGCACTCTGGACTTTAAATCCAGCGATC |
| | CGAGTTCGAGTCTCGGTGGAACCT |
| 313 | CGTCGGTTCCATGGTGTAATGGTTAGCACTCTGGACTTTAAATCCAGCGATC |
| | CGAGTTCAAATCTCGGTGGAACCT |
| 314 | CGTCGGTCCCATGGTGTAATGGTTAGCACTCTGGACTTTAAATCCAGCGATC |
| | CGAGTTCAAATCTCGGTGGGACCT |
| 315 | CGTCGGTCCCATGGTGTAATGGTTAGCACTCTGGACTTTAAATCCAGCAATC |
| | CGAGTTCGAATCTCGGTGGGACCT |
| 316 | CGTCGGTTCCATGGTGTAATGGTTAGCACTCTGGACTTTAAATCCGGTAATC |
| | CGAGTTCAAATCTCGGTGGAACCT |
| 317 | CGTCGGCCCCATGGTGTAATGGTCAGCACTCTGGACTTTAAATCCAGCGATC |
| | CGAGTTCAAATCTCGGTGGGACCC |
| 318 | CGTCGGTTCCATGGTGTAATGGTTAGCACTCTGGACTTTAAATCCAGCGATC |
| | CGAGTTCAAGTCTCGGTGGAACCT |
| 319 | CGTCGACCTCGTGGCGCAATGGTAGCGCGTCTGACTCTAGATCAGAAGGTTG |
| | CGTGTTCAAGTCACGTCGGGGTCA |
| 320 | CGTCGACCTCGTGGCGCAACGGTAGCGCGTCTGACTCTAGATCAGAAGGTTG |
| | CGTGTTCAAATCACGTCGGGGTCA |
| 321 | CGTCGGCCTCGTGGCGCAACGGTAGCGCGTCTGACTCTAGATCAGAAGGTTG |
| | CGTGTTCAAATCACGTCGGGGTCA |
| 322 | CGTCGACCTCGTGGCGCAACGGTAGCGCGTCTGACTCTAGATCAGAAGGCTG |
| | CGTGTTCGAATCACGTCGGGGTCA |
| 323 | CGTCGACCTCGTGGCGCAACGGCAGCGCGTCTGACTCTAGATCAGAAGGTTG |
| | CGTGTTCAAATCACGTCGGGGTCA |
| 324 | CGTCTCCCACATGGTCTAGCGGTTAGGATTCCTGGTTCTAACCCAGGCGGCC |
| | CGGGTTCGACTCCCGGTGTGGGAA |
| 325 | CGTCTCCCATATGGTCTAGCGGTTAGGATTCCTGGTTCTAACCCAGGTGGCC |
| | CGGGTTCGACTCCCGGTATGGGAA |
| 326 | CGTCTCCCTGGTGGTCTAGTGGCTAGGATTCGGCGCTCTAACCGCCGCGGCC |
| | CGGGTTCGATTCCCGGTCAGGGAA |
| 327 | CGTCTCCCTGGTGGTCTAGTGGTTAGGATTCGGCGCTCTAACCGCCGCGGCC |
| | CGGGTTCGATTCCCGGTCAGGGAA |
| 328 | CGTCTCCCTGGTCTAGTGGCTAGGATTCGGCGCTCTAACCGCCGCGGCCCGG |
| | GTTCGATTCCCGGCCAGGGAA |
| 329 | CGTCTCCCACATGGTCTAGCGGTTAGGATTCCTGGTTCTAACCCAGGCGGCC |
| | CGGGTTCGACTCCCGGTGTGGGAA |
| 330 | CGTCTCCCATATGGTCTAGCGGTTAGGATTCCTGGTTCTAACCCAGGTGGCC |
| | CGGGTTCGACTCCCGGTATGGGAA |
| 331 | CGTCTCCCTGGTGGTCTAGTGGTTAGGATTCGGCGCTCTAACCGCCGCGGCC |
| | CGGGTTCGATTCCCGGTCAGGGAA |
| 332 | CGTCTCCCTGGTGGTCTAGTGGTTAGGATTCGGCGCTCTAACCGCCGCGGCC |
| | CGGGTTCGATTCCCGGTCAGGAAA |
| 333 | CGTCTCCCTGGTGGTCTAGTGGCTAGGATTCGGCGCTCTAACCGCCGCGGCC |
| | CGGGTTCGATTCCCGGCCAGGGAA |
| 334 | GGCCTCGTGGCGCAACGGTAGCGCGTCTGACTTCAGATCAGAAGGTTGCGTG |
| | TTCAAATCACGTCGGGGTCA |
| 335 | GACCTCGTGGCGCAATGGTAGCGCGTCTGACTTCAGATCAGAAGGTTGCGTG |
| | TTCAAGTCACGTCGGGGTCA |
| 336 | GACCTCGTGGCGCAACGGTAGCGCGTCTGACTTCAGATCAGAAGGTTGCGTG |
| | TTCAAATCACGTCGGGGTCA |
| 337 | GACCTCGTGGCGCAACGGTAGCGCGTCTGACTTCAGATCAGAAGGCTGCGTG |
| | TTCGAATCACGTCGGGGTCA |
| 338 | GACCTCGTGGCGCAACGGCAGCGCGTCTGACTTCAGATCAGAAGGTTGCGTG |
| | TTCAAATCACGTCGGGGTCA |
| 339 | GCGTTGGTGGTATAGTGGTTAGCATAGCTGCCTTCAAAGCAGTTGACCCGGG |
| | TTCGATTCCCGGCCAACGCA |
| 340 | GCGTTGGTGGTATAGTGGTGAGCATAGCTGCCTTCAAAGCAGTTGACCCGGG |
| | TTCGATTCCCGGCCAACGCA |
| 341 | GCGTTGGTGGTATAGTGGTAAGCATAGCTGCCTTCAAAGCAGTTGACCCGGG |
| | TTCGATTCCCGGCCAACGCA |
| 342 | GGCCTCGTGGCGCAACGGTAGCGCGTCTGACTTCAGATCAGAAGGTTGCGTG |
| | TTCAAATCACGTCGGGGTCA |
| 343 | GACCTCGTGGCGCAATGGTAGCGCGTCTGACTTCAGATCAGAAGGTTGCGTG |
| | TTCAAGTCACGTCGGGGTCA |
| 344 | GACCTCGTGGCGCAACGGTAGCGCGTCTGACTTCAGATCAGAAGGTTGCGTG |
| | TTCAAATCACGTCGGGGTCA |
| 345 | GACCTCGTGGCGCAACGGTAGCGCGTCTGACTTCAGATCAGAAGGCTGCGTG |
| | TTCGAATCACGTCGGGGTCA |
| 346 | GACCTCGTGGCGCAACGGCAGCGCGTCTGACTTCAGATCAGAAGGTTGCGTG |
| | TTCAAATCACGTCGGGGTCA |
| 347 | GGCCTCATGGTGCAACAGTAGTGTGTCTGACTTCAGATCAGAAGGTTGTATG |
| | TTCAAATCACGTAGGGGTCA |
| 348 | GGCCTCGTGGCGCAACGGTAGCGCGTCTGACTCTAGATCAGAAGGTTGCGTG |
| | TTCAAATCACGTCGGGGTCA |
| 349 | GACCTCGTGGCGCAATGGTAGCGCGTCTGACTCTAGATCAGAAGGTTGCGTG |
| | TTCAAGTCACGTCGGGGTCA |
| 350 | GACCTCGTGGCGCAACGGTAGCGCGTCTGACTCTAGATCAGAAGGTTGCGTG |
| | TTCAAATCACGTCGGGGTCA |
| 351 | GACCTCGTGGCGCAACGGTAGCGCGTCTGACTCTAGATCAGAAGGCTGCGTG |
| | TTCGAATCACGTCGGGGTCA |
| 352 | GACCTCGTGGCGCAACGGCAGCGCGTCTGACTCTAGATCAGAAGGTTGCGTG |
| | TTCAAATCACGTCGGGGTCA |
| 353 | GGCCTCATGGTGCAACAGTAGTGTGTCTGACTCTAGATCAGAAGGTTGTATG |
| | TTCAAATCACGTAGGGGTCA |
| 354 | GCATTGGTGGTTCAGTGGTAGAATTCTCGCCTTCAACGCGGGAGACCCGGGT |
| | TCAATTCCCGGCCAATGCA |
| 355 | GCGCCGCTGGTGTAGTGGTATCATGCAAGATTTCAATTCTTGCGACCCGGGT |
| | TCGATTCCCGGGCGGCGCA |
| 356 | GCATTGGTGGTTCAATGGTAGAATTCTCGCCTTCAACGCAGGAGACCCAGGT |
| | TCGATTCCTGGCCAATGCA |
| 357 | GCGTTGGTGGTTTAGTGGTAGAATTCTCGCCTTCAATGCGGGAGACCCGGGT |
| | TCAATTCCCGGCCACTGCA |
| 358 | GCCTTGGTGGTGCAGTGGTAGAATTCTCGCCTTCAACGTGGGAGACCCGGGT |
| | TCAATTCCCGGCCAATGCA |
| 359 | GGTGGTTCAGTGGTAGAATTCTCGCCTTCAACGCGGGAGACCCGGGTTTAAT |
| | TCCCGGTCA |
| 360 | GTGGTCTAGTGGTTAGGATTCAGCGCTTCAACCGCCGCAGCCCGGGTTCGAT |
| | TCCCGGTCA |
| 361 | GCGTCAGTGGTTTAGTGGTGGAATTCCTGCCTTCAATGCACGAGATCCGTGT |
| | TCAACTCCTGGTTGGTGCA |
| 362 | GCGTCAGTGGTTTTAGTGGTGGAATTCCTGCCTTCAATGCACGAGATCCGTG |
| | TTCAACTCCTGGTTGGTGCA |
| 363 | GCGTTGGCAGTTCAGTGGTAGAATTCTCGCCTTCAACCCGGGAGACCTGGAT |
| | TCCATTTCCGGCAAATGCA |
| 364 | GCATGGGTGGTTCAGTGGTAGAATTCTCGCCTTCAACGCGGGAGGCCCGGGT |
| | TCGATTCCCGGCCCATGCA |
| 365 | GCATTGGTGGTTCAGTGGTAGAATTCTCGCCTTCAACGCGGGAGGCCCGGGT |
| | TCGATTCCCGGCCAATGCA |
| 366 | GCATTGGTGGTTCAGTGGTAGAATTCTCGCCTTCAACGCGGGAGGCCCGGGT |
| | TTGATTCCCGGCCAGTGCA |
| 367 | GCATAGGTGGTTCAGTGGTAGAATTCTTGCCTTCAACGCAGGAGGCCCAGGT |
| | TTGATTCCTGGCCCATGCA |
| 368 | GCATTGGTGGTTCAGTGGTAGAATTCTCGCCTTCAATGCGGGCGGCCGGGCT |
| | TCGATTCCTGGCCAATGCA |
| 369 | GCATGGGTGATTCAGTGGTAGAATTTTCACCTTCAATGCAGGAGGTCCAGGT |
| | TCATTTCCTGGCCTATGCA |
| 370 | GCGTTGGTGGTATAGTGGTTAGCATAGCTGCCTTCAAAGCAGTTGACCCGGG |
| | TTCGATTCCCGGCCAACGCA |
| 371 | GCGTTGGTGGTATAGTGGTGAGCATAGCTGCCTTCAAAGCAGTTGACCCGGG |
| | TTCGATTCCCGGCCAACGCA |
| 372 | GCGTTGGTGGTATAGTGGTAAGCATAGCTGCCTTCAAAGCAGTTGACCCGGG |
| | TTCGATTCCCGGCCAACGCA |
| 373 | GCGTTGGTGGTATAGTGGTGAGCATAGTTGCCTTCAAAGCAGTTGACCCGGG |
| | CTCGATTCCCGCCCAACGCA |
| 374 | GCGTTGGTGGTATAGTGGTGAGCATAGTTGCCTTCAAAGCAGTTGACCCGGG |
| | CTCGATTCCCGGCCAACGCA |
| 375 | GGGCCAGTGGCGCAATGGATAACGCGTCTGACTTCAGATCAGAAGATTCCAG |
| | GTTCGACTCCTGGCTGGCTCG |
| 376 | GGGCCAGTGGCGCAATGGATAACGCGTCTGACTTCAGATCAGAAGATTCTAG |
| | GTTCGACTCCTGGCTGGCTCG |
| 377 | GGCCGCGTGGCCTAATGGATAAGGCGTCTGATTTCAGATCAGAAGATTGAGG |
| | GTTCGAGTCCCTTCGTGGTCG |
| 378 | GACCCAGTGGCCTAATGGATAAGGCATCAGCCTTCAGAGCTGGGGATTGTGG |
| | GTTCGAGTCCCATCTGGGTCG |
| 379 | GCCCCAGTGGCCTAATGGATAAGGCACTGGCCTTCAAAGCCAGGGATTGTGG |
| | GTTCGAGTCCCACCTGGGGTA |
| 380 | GCCCCAGTGGCCTAATGGATAAGGCACTGGCCTTCAAAGCCAGGGATTGTGG |
| | GTTCGAGTCCCACCTGGGGTG |
| 381 | GCCCCGGTGGCCTAATGGATAAGGCATTGGCCTTCAAAGCCAGGGATTGTGG |
| | GTTCGAGTCCCACCCGGGGTA |
| 382 | GCCCCAGTGGCCTAATGGATAAGGCATTGGCCTTCAAAGCCAGGGATTGTGG |
| | GTTCGAGTCCCATCTGGGGTG |
| 383 | GCCCCAGTGGCCTGATGGATAAGGTACTGGCCTTCAAAGCCAGGGATTGTGG |
| | GTTCGAGTTCCACCTGGGGTA |
| 384 | GGCCGCGTGGCCTAATGGATAAGGCGTCTGACTTCAGATCAGAAGATTGCAG |
| | GTTCGAGTCCTGCCGCGGTCG |
| 385 | GACCACGTGGCCTAATGGATAAGGCGTCTGACTTCAGATCAGAAGATTGAGG |
| | GTTCGAATCCCTCCGTGGTTA |
| 386 | GACCGCGTGGCCTAATGGATAAGGCGTCTGACTTCAGATCAGAAGATTGAGG |
| | GTTCGAGTCCCTTCGTGGTCG |
| 387 | GACCACGTGGCCTAATGGATAAGGCGTCTGACTTCAGATCAGAAGATTGAGG |
| | GTTCGAATCCCTTCGTGGTTA |
| 388 | GACCACGTGGCCTAATGGATAAGGCGTCTGACTTCAGATCAGAAGATTGAGG |
| | GTTCGAATCCCTTCGTGGTTG |
| 389 | GGCCGTGTGGCCTAATGGATAAGGCGTCTGACTTCAGATCAAAAGATTGCAG |
| | GTTTGAGTTCTGCCACGGTCG |
| 390 | GGCTCCGTGGCGCAATGGATAGCGCATTGGACTTCAAGAGGCTGAAGGCATT |
| | CAAAGGTTCCGGGTTCGAGTCCCGGCGGAGTCG |
| 391 | GGCTCCGTGGCGCAATGGATAGCGCATTGGACTTCAAATTCAAAGGTTCCGG |
| | GTTCGAGTCCCGGCGGAGTCG |
| 392 | GGCTCTGTGGCGCAATGGATAGCGCATTGGACTTCAAGTGACGAATAGAGCA |
| | ATTCAAAGGTTGTGGGTTCGAATCCCACCAGAGTCG |
| 393 | GGCTCTGTGGCGCAATGGATAGCGCATTGGACTTCAAATTCAAAGGTTGTGG |
| | GTTCGAATCCCACCAGAGTCG |
| 394 | GGCTCTGTGGCGCAATGGATAGCGCATTGGACTTCAAGCTGAGCCTAGTGTG |
| | GTCATTCAAAGGTTGTGGGTTCGAGTCCCACCAGAGTCG |
| 395 | GGCTCTGTGGCGCAATGGATAGCGCATTGGACTTCAAATTCAAAGGTTGTGG |
| | GTTCGAGTCCCACCAGAGTCG |
| 396 | GGCTCTGTGGCGCAATGGATAGCGCATTGGACTTCAAGATAGTTAGAGAAAT |
| | TCAAAGGTTGTGGGTTCGAGTCCCACCAGAGTCG |
| 397 | GTCTCTGTGGCGCAATGGACGAGCGCGCTGGACTTCAAATCCAGAGGTTCCG |
| | GGTTCGAGTCCCGGCAGAGATG |
| 398 | GGCTCTGTGGCGCAATGGATAGCGCATTGGACTTCAAGCCTAAATCAAGAGA |
| | TTCAAAGGTTGCGGGTTCGAGTCCCTCCAGAGTCG |
| 399 | GGCTCTGTGGCGCAATGGATAGCGCATTGGACTTCAAATTCAAAGGTTGCGG |
| | GTTCGAGTCCCTCCAGAGTCG |
| 400 | GGCAGCATAGCAGAGTGGTTCAGGTTACAGGTTCAAGATGTAAACTGAGTTC |
| | AAATCCCAGTTCTGCCA |
| 401 | TGGTGTAATAGGTAGCACAGAGAATTCTAGATTCTCAGGGGTAGGTTCAATT |
| | CCTAT |
| 402 | TAGGACATGGTGTGATAGGTAGCATGGAGAATTCTAGATTCTCAGGGGTAGG |
| | TTCAATTCCTACAGTTCTAG |
| 403 | TAGGACGTGGTGTGATAGGTAGCATGGGGAATTCTAGATTCTCAGGGGTGGG |
| | TTCAATTCCTATAGTTCTAG |
| 404 | TAGGACGTGGTGTAGTAGGTAGCATGGAGAATGCTAAATTCTCAGGGGTAGG |
| | TTCAATTCCTATAGTTCTAG |
| 405 | TAGGACATGGTGTAATAGGTAGAATGGAGAATTCTAAATTCTCAGGGGTAGG |
| | TTCAATTCCTATAGTTCTAG |
| 406 | TAGGATGTGGTGTATTAGGTAGCACAGAGAATTCTAGATTCTCAGGGGTAGG |
| | TTCGATTCCTATAATTCTAC |
| 407 | TAGGACTTGGTGTAATGGGTAGCACAGAGAATTCTAGATTCTCAGGGGTGGG |
| | TTCAATTCCTTTCGTCCTAG |
| 408 | TCTAGGATGTGGTGTGATAGGTAGCATGGAGAATTCTAGATTCTCAGGGGTA |
| | GGTTCAATTCCTATATTCTAGAA |
| 409 | TAGGACGTGGTGTGATAGGTAGCATGGAGAATTCTAGATTCTCAGGGATGGG |
| | TTCAATTCCTATAGTCCTAG |
| 410 | TAGGACGTGGTGTGATAGGTAGCACGGAGAATTCTAGATTCTCAGGGATGGG |
| | TTCAATTCCTGTAGTTCTAG |
| 411 | GGTTCCATGGTGTAATGGTTAGCACTCTGGACTCTAAATCCAGCGATCCGAG |
| | TTCAAATCTCGGTGGAACCT |
| 412 | GGTTCCATGGTGTAATGGTGACCACTTTGGACTCTAAATACAGTGATCAGAG |
| | TTCAAGTCTCACTGGAACCT |
| 413 | GGTTCCATGGTGTAATGGTGAGGGCTTTGGACTCTAACTACAGTGATCAGAG |
| | TTCAAGTCTCAGTGGGACCT |
| 414 | GGTTCCATGGTGTAATGGTAAGCACCCTGGACTCTAAATCCAGCAACCAGAG |
| | TTCCAGTCTCAGCGTGGACCT |
| 415 | GGTAGTGTAGTCTACTGGTTAAACGCTTGGGCTCTAACATTAACGTCCTGGG |
| | TTCAAATCCCAGCTTTGTCA |
| 416 | GGTTCCATGGTGTAATGGTTAGCACTCTGGACTCTAAATCCAGCGATCCGAG |
| | TTCAAGTCTCGGTGGAACCT |
| 417 | GGTTCCATGGTGTAATGGTGAGCACTCTGGACTCTAAATCCAGCGATCCGAG |
| | TTCGAGTCTCGGTGGAACCT |
| 418 | GGTTCCATGGTGTAATGGTAAGCACTCTGGACTCTAAATCCAGCGATCCGAG |
| | TTCGAGTCTCGGTGGAACCT |
| 419 | GGTTCCATGGTGTAATGGTTAGCACTCTGGACTCTAAATCCGGTAATCCGAG |
| | TTCAAATCTCGGTGGAACCT |
| 420 | GGCCCCATGGTGTAATGGTCAGCACTCTGGACTCTAAATCCAGCGATCCGAG |
| | TTCAAATCTCGGTGGGACCC |
| 421 | GGTTCCATGGTGTAATGGTAAGCACTCTGGACTCTAAATCCAGCCATCTGAG |
| | TTCGAGTCTCTGTGGAACCT |
| 422 | GGTTCCATGGTGTAATGGTGAGCACTTTGGACTCTAAATACAGTGATCAGAG |
| | TTCAAGTCTCACTGGGACCT |
| 423 | GGTTCCATGGGTTAATGGTGAGCACCCTGGACTCTAAATCAAGCGATCCGAG |
| | TTCAAATCTCGGTGGTACCT |
| 424 | GTTTCCATGGTGTAATGGTGAGCACTCTGGACTCTAAATCCAGAAATACATT |
| | CAAAGAATTAAGAACA |
| 425 | GGTCCCATGGTGTAATGGTTAGCACTCTGGACTCTAAATCCAGCGATCCGAG |
| | TTCAAATCTCGGTGGGACCT |
| 426 | GGTCCCATGGTGTAATGGTTAGCACTCTGGACTCTAAATCCAGCAATCCGAG |
| | TTCGAATCTCGGTGGGACCT |
| 427 | GGCCCCATGGTGTAATGGTTAGCACTCTGGACTCTAAATCCAGCGATCCGAG |
| | TTCAAATCTCGGTGGGACCT |
| 428 | GGTCCCATGGTGTAATGGTTAGCACTCTGGGCTCTAAATCCAGCAATCCGAG |
| | TTCGAATCTTGGTGGGACCT |
| 429 | GGCTGTGTACCTCAGTGGGCAAGGGTATGGACTCTAAAGCCAGACTATTTGG |
| | GTTCAAATCCCAGCTTGGCCT |
| 430 | GACCATGTGGCCTAAGGGAAAAGACATCTCACTCTAGGTCAGAAGATTGAGG |
| | GTTCAAGTCCTTTCATGGTCA |
| 431 | GGTACAGTGTTAAAGGGGAGAAAAATTGCTGACTCTAAATACAGTAGACCTA |
| | GGTTTGAATCCTGGCTTTACCA |
| 432 | TGGTGTAATAGGTAGCACAGAGAATTTTAGATTCTCAGGGGTAGGTTCAATT |
| | CCTAT |
| 433 | TAGGACATGGTGTGATAGGTAGCATGGAGAATTTTAGATTCTCAGGGGTAGG |
| | TTCAATTCCTACAGTTCTAG |
| 434 | TAGGACGTGGTGTGATAGGTAGCATGGGGAATTTTAGATTCTCAGGGGTGGG |
| | TTCAATTCCTATAGTTCTAG |
| 435 | TAGGACGTGGTGTAGTAGGTAGCATGGAGAATGTTAAATTCTCAGGGGTAGG |
| | TTCAATTCCTATAGTTCTAG |
| 436 | TAGGACATGGTGTAATAGGTAGAATGGAGAATTTTAAATTCTCAGGGGTAGG |
| | TTCAATTCCTATAGTTCTAG |
| 437 | TAGGATGTGGTGTATTAGGTAGCACAGAGAATTTTAGATTCTCAGGGGTAGG |
| | TTCGATTCCTATAATTCTAC |
| 438 | TAGGACTTGGTGTAATGGGTAGCACAGAGAATTTTAGATTCTCAGGGGTGGG |
| | TTCAATTCCTTTCGTCCTAG |
| 439 | TCTAGGATGTGGTGTGATAGGTAGCATGGAGAATTTTAGATTCTCAGGGGTA |
| | GGTTCAATTCCTATATTCTAGAA |
| 440 | TAGGACGTGGTGTGATAGGTAGCATGGAGAATTTTAGATTCTCAGGGATGGG |
| | TTCAATTCCTATAGTCCTAG |
| 441 | TAGGACGTGGTGTGATAGGTAGCACGGAGAATTTTAGATTCTCAGGGATGGG |
| | TTCAATTCCTGTAGTTCTAG |
| 442 | GGTTCCATGGTGTAATGGTTAGCACTCTGGACTTTAAATCCAGCGATCCGAG |
| | TTCAAATCTCGGTGGAACCT |
| 443 | GGTTCCATGGTGTAATGGTGACCACTTTGGACTTTAAATACAGTGATCAGAG |
| | TTCAAGTCTCACTGGAACCT |
| 444 | GGTTCCATGGTGTAATGGTGAGGGCTTTGGACTTTAACTACAGTGATCAGAG |
| | TTCAAGTCTCAGTGGGACCT |
| 445 | GGTTCCATGGTGTAATGGTAAGCACCCTGGACTTTAAATCCAGCAACCAGAG |
| | TTCCAGTCTCAGCGTGGACCT |
| 446 | GGTAGTGTAGTCTACTGGTTAAACGCTTGGGCTTTAACATTAACGTCCTGGG |
| | TTCAAATCCCAGCTTTGTCA |
| 447 | GGTTCCATGGTGTAATGGTTAGCACTCTGGACTTTAAATCCAGCGATCCGAG |
| | TTCAAGTCTCGGTGGAACCT |
| 448 | GGTTCCATGGTGTAATGGTGAGCACTCTGGACTTTAAATCCAGCGATCCGAG |
| | TTCGAGTCTCGGTGGAACCT |
| 449 | GGTTCCATGGTGTAATGGTAAGCACTCTGGACTTTAAATCCAGCGATCCGAG |
| | TTCGAGTCTCGGTGGAACCT |
| 450 | GGTTCCATGGTGTAATGGTTAGCACTCTGGACTTTAAATCCGGTAATCCGAG |
| | TTCAAATCTCGGTGGAACCT |
| 451 | GGCCCCATGGTGTAATGGTCAGCACTCTGGACTTTAAATCCAGCGATCCGAG |
| | TTCAAATCTCGGTGGGACCC |
| 452 | GGTTCCATGGTGTAATGGTAAGCACTCTGGACTTTAAATCCAGCCATCTGAG |
| | TTCGAGTCTCTGTGGAACCT |
| 453 | GGTTCCATGGTGTAATGGTGAGCACTTTGGACTTTAAATACAGTGATCAGAG |
| | TTCAAGTCTCACTGGGACCT |
| 454 | GGTTCCATGGGTTAATGGTGAGCACCCTGGACTTTAAATCAAGCGATCCGAG |
| | TTCAAATCTCGGTGGTACCT |
| 455 | GTTTCCATGGTGTAATGGTGAGCACTCTGGACTTTAAATCCAGAAATACATT |
| | CAAAGAATTAAGAACA |
| 456 | GGTCCCATGGTGTAATGGTTAGCACTCTGGACTTTAAATCCAGCGATCCGAG |
| | TTCAAATCTCGGTGGGACCT |
| 457 | GGTCCCATGGTGTAATGGTTAGCACTCTGGACTTTAAATCCAGCAATCCGAG |
| | TTCGAATCTCGGTGGGACCT |
| 458 | GGCCCCATGGTGTAATGGTTAGCACTCTGGACTTTAAATCCAGCGATCCGAG |
| | TTCAAATCTCGGTGGGACCT |
| 459 | GGTCCCATGGTGTAATGGTTAGCACTCTGGGCTTTAAATCCAGCAATCCGAG |
| | TTCGAATCTTGGTGGGACCT |
| 460 | GGCTGTGTACCTCAGTGGGCAAGGGTATGGACTTTAAAGCCAGACTATTTGG |
| | GTTCAAATCCCAGCTTGGCCT |
| 461 | GACCATGTGGCCTAAGGGAAAAGACATCTCACTTTAGGTCAGAAGATTGAGG |
| | GTTCAAGTCCTTTCATGGTCA |
| 462 | GGTACAGTGTTAAAGGGGAGAAAAATTGCTGACTTTAAATACAGTAGACCTA |
| | GGTTTGAATCCTGGCTTTACCA |
| 463 | TCCCTGGTGGTCTAGTGGTTAGGATTCGGCGCTTTAACCGCCGCGGCCCGGG |
| | TTCGATTCCCGGTCAGGGAA |
| 464 | TCCCTGGTGGTCTAGTGGTTAGGATTCGGCGCTTTAACCGCCGCGGCCCGGG |
| | TTCGATTCCCGGTCAGGAAA |
| 465 | CCCCTGGTGGTCTAGTGCTTAGGATTCGGTGCTTTAACCGCTGCTGCCTGCG |
| | TTCGATTCCCGGTCAGGGAA |
| 466 | TCCTTGATGTCTAGTGGTTAGGATTTGGTGCTTTAACTGCAGCAGCCTGGGT |
| | TCATTTCTCAGTCAGGGAA |
| 467 | TCCCATATGGTCTAGCGGTTAGGATTCCTGGTTTTAACCCAGGTGGCCCGGG |
| | TTCGACTCCCGGTATGGGAA |
| 468 | TCCGTGGTGGTCTAGTGGCTAGGATTCGGCGCTTTAACCGCCTGCAGCTCGA |
| | GTTCGATTCCTGGTCAGGGAA |
| 469 | CCCTGTGGTCTAGTGGCTAAGACTTTGTGCTTTAATTGCTGCATCCTAGGTT |
| | CAATTCCCAGTCAGGGA |
| 470 | TCCCACATGGTCTAGCGGTTAGGATTCCTGGTTTTAACCCAGGCGGCCCGGG |
| | TTCGACTCCCGGTGTGGGAA |
| 471 | TCCCTGGTGGTCTAGTGGCTAGGATTCGGCGCTTTAACCGCCGCGGCCCGGG |
| | TTCGATTCCCGGCCAGGGAA |
| 472 | TCCCTGGTGGTCTAGTGGCTAGGATTCGGCGCTTTAACCGCCGCGGCCCGGG |
| | TTCGATTCCCGGTCAGGGAA |
| 473 | GCGTTGGTGGTGTAGTGGTGAGCACAGCTGCCTTTAAAGCAGTTAACGCGGG |
| | TTCGATTCCCGGGTAACGAA |
| 474 | TCCTTGGTGGTCTAGTGGCTAGGATTCGGTGCTTTAACCTGTGCGGCCCGGG |
| | TTCAATTCCCGATGAAGGAA |
| 475 | TGTCTGGTGGTCAAGTGGCTAGGATTTGGCGCTTTAACTGCCGCGGCCCGCG |
| | TTCGATTCCCGGTCAGGGAA |
| 476 | TCCCTGGTGGTCTAGTGGCTAGGATTCGGCGCTTTAACCGCCTGCAGCTCGA |
| | GTTCGATTCCTGGTCAGGGAA |
| 477 | GCAATGGTGGTTCAGTGGTAGAATTCTCGCCTTTAACACAGGAGACCCGGGT |
| | TCAATTCCTGACCCATGTA |
| 478 | TCCCTGGTGGTCTAGTGGTTAGGATTCGGCGCTCTAACCGCCGCGGCCCGGG |
| | TTCGATTCCCGGTCAGGGAA |
| 479 | TCCCTGGTGGTCTAGTGGTTAGGATTCGGCGCTCTAACCGCCGCGGCCCGGG |
| | TTCGATTCCCGGTCAGGAAA |
| 480 | CCCCTGGTGGTCTAGTGCTTAGGATTCGGTGCTCTAACCGCTGCTGCCTGCG |
| | TTCGATTCCCGGTCAGGGAA |
| 481 | TCCTTGATGTCTAGTGGTTAGGATTTGGTGCTCTAACTGCAGCAGCCTGGGT |
| | TCATTTCTCAGTCAGGGAA |
| 482 | TCCCATATGGTCTAGCGGTTAGGATTCCTGGTTCTAACCCAGGTGGCCCGGG |
| | TTCGACTCCCGGTATGGGAA |
| 483 | TCCGTGGTGGTCTAGTGGCTAGGATTCGGCGCTCTAACCGCCTGCAGCTCGA |
| | GTTCGATTCCTGGTCAGGGAA |
| 484 | CCCTGTGGTCTAGTGGCTAAGACTTTGTGCTCTAATTGCTGCATCCTAGGTT |
| | CAATTCCCAGTCAGGGA |
| 485 | TCCCACATGGTCTAGCGGTTAGGATTCCTGGTTCTAACCCAGGCGGCCCGGG |
| | TTCGACTCCCGGTGTGGGAA |
| 486 | TCCCTGGTGGTCTAGTGGCTAGGATTCGGCGCTCTAACCGCCGCGGCCCGGG |
| | TTCGATTCCCGGCCAGGGAA |
| 487 | TCCCTGGTGGTCTAGTGGCTAGGATTCGGCGCTCTAACCGCCGCGGCCCGGG |
| | TTCGATTCCCGGTCAGGGAA |
| 488 | GCGTTGGTGGTGTAGTGGTGAGCACAGCTGCCTCTAAAGCAGTTAACGCGGG |
| | TTCGATTCCCGGGTAACGAA |
| 489 | TCCTTGGTGGTCTAGTGGCTAGGATTCGGTGCTCTAACCTGTGCGGCCCGGG |
| | TTCAATTCCCGATGAAGGAA |
| 490 | TGTCTGGTGGTCAAGTGGCTAGGATTTGGCGCTCTAACTGCCGCGGCCCGCG |
| | TTCGATTCCCGGTCAGGGAA |
| 491 | TCCCTGGTGGTCTAGTGGCTAGGATTCGGCGCTCTAACCGCCTGCAGCTCGA |
| | GTTCGATTCCTGGTCAGGGAA |
| 492 | GCAATGGTGGTTCAGTGGTAGAATTCTCGCCTCTAACACAGGAGACCCGGGT |
| | TCAATTCCTGACCCATGTA |
| 493 | CCTTCAATAGTTCAGCTGGTAGAGCAGAGGACTTTAGCTACTTCCTCAGTAG |
| | GAGACGTCCTTAGGTTGCTGGTTCGATTCCAGCTTGAAGGA |
| 494 | CCTTCAATAGTTCAGCTGGTAGAGCAGAGGACTTTAGGTCCTTAGGTTGCTG |
| | GTTCGATTCCAGCTTGAAGGA |
| 495 | GGTAAAATGGCTGAGTAAGCTTTAGACTTTAAATCTAAAGAGAGATTGAGCT |
| | CTCTTTTTACCA |
| 496 | GGTAAAATGACTGAGTAAGCATTAGACTTTAAATCTAAAGACAGAGGTCAAG |
| | ACCTCTTTTTACCA |
| 497 | GGTAAAATGGCTGAGTAAGCATTAGACTTTAAATCTAAAGACAGAGGTCAAG |
| | GCCTCTTTTTACCA |
| 498 | GGTAAAATGGCTGAGTAAGCATTAGACTTTAAATCTAAAGACAGAGGTCAAG |
| | GCCTTTTTACCA |
| 499 | CCTTCGATAGCTCAGTTGGTAGAGCGGAGGACTTTAGTTGGCTGTGTCCTTA |
| | GACATCCTTAGGTCGCTGGTTCGAATCCGGCTCGAAGGA |
| 500 | CCTTCGATAGCTCAGTTGGTAGAGCGGAGGACTTTAGATCCTTAGGTCGCTG |
| | GTTCGAATCCGGCTCGAAGGA |
| 501 | GGGGGTATAGCTCAGGGCTAGAGCTTTTTGACTTTAGAGCAAGAGGTCCCTG |
| | GTTCAAATCCAGGTTCTCCCT |
| 502 | TATAGCTCAGTGGTAGAGCATTTAACTTTAGATCAAGAGGTCCCTGGATCAA |
| | CTCTGGGTG |
| 503 | GTCAGTGTTGCACAACGGTTAAGTGAAGAGGCTTTAAACCCAGACTGGATGG |
| | GTTCAATTCCCATCTCTGCCG |
| 504 | CCTTCGATAGCTCAGTTGGTAGAGCGGAGGACTTTAGTGGATAGGGCGTGGC |
| | AATCCTTAGGTCGCTGGTTCGATTCCGGCTCGAAGGA |
| 505 | CCTTCGATAGCTCAGTTGGTAGAGCGGAGGACTTTAGATCCTTAGGTCGCTG |
| | GTTCGATTCCGGCTCGAAGGA |
| 506 | CCTTCGATAGCTCAGTTGGTAGAGCGGAGGACTTTAGGCTCATTAAGCAAGG |
| | TATCCTTAGGTCGCTGGTTCGAATCCGGCTCGGAGGA |
| 507 | CCTTCGATAGCTCAGTTGGTAGAGCGGAGGACTTTAGATCCTTAGGTCGCTG |
| | GTTCGAATCCGGCTCGGAGGA |
| 508 | CCTTCGATAGCTCAGCTGGTAGAGCGGAGGACTTTAGATTGTATAGACATTT |
| | GCGGACATCCTTAGGTCGCTGGTTCGATTCCAGCTCGAAGGA |
| 509 | CCTTCGATAGCTCAGCTGGTAGAGCGGAGGACTTTAGATCCTTAGGTCGCTG |
| | GTTCGATTCCAGCTCGAAGGA |
| 510 | CCTTCGATAGCTCAGCTGGTAGAGCGGAGGACTTTAGCTACTTCCTCAGCAG |
| | GAGACATCCTTAGGTCGCTGGTTCGATTCCGGCTCGAAGGA |
| 511 | CCTTCGATAGCTCAGCTGGTAGAGCGGAGGACTTTAGATCCTTAGGTCGCTG |
| | GTTCGATTCCGGCTCGAAGGA |
| 512 | CCTTCGATAGCTCAGCTGGTAGAGCGGAGGACTTTAGGCGCGCGCCCGTGGC |
| | CATCCTTAGGTCGCTGGTTCGATTCCGGCTCGAAGGA |
| 513 | CCTTCGATAGCTCAGCTGGTAGAGCGGAGGACTTTAGATCCTTAGGTCGCTG |
| | GTTCGATTCCGGCTCGAAGGA |
| 514 | CCTTCGATAGCTCAGCTGGTAGAGCGGAGGACTTTAGCCTGTAGAAACATTT |
| | GTGGACATCCTTAGGTCGCTGGTTCGATTCCGGCTCGAAGGA |
| 515 | CCTTCGATAGCTCAGCTGGTAGAGCGGAGGACTTTAGATCCTTAGGTCGCTG |
| | GTTCGATTCCGGCTCGAAGGA |
| 516 | CCTTCGATAGCTCAGCTGGTAGAGCGGAGGACTTTAGATTGTACAGACATTT |
| | GCGGACATCCTTAGGTCGCTGGTTCGATTCCGGCTCGAAGGA |
| 517 | CCTTCGATAGCTCAGCTGGTAGAGCGGAGGACTTTAGATCCTTAGGTCGCTG |
| | GTTCGATTCCGGCTCGAAGGA |
| 518 | CCTTCGATAGCTCAGCTGGTAGAGCGGAGGACTTTAGTACTTAATGTGTGGT |
| | CATCCTTAGGTCGCTGGTTCGATTCCGGCTCGAAGGA |
| 519 | CCTTCGATAGCTCAGCTGGTAGAGCGGAGGACTTTAGATCCTTAGGTCGCTG |
| | GTTCGATTCCGGCTCGAAGGA |
| 520 | CCTTCGATAGCTCAGCTGGTAGAGCGGAGGACTTTAGGGGTTTGAATGTGGT |
| | CATCCTTAGGTCGCTGGTTCGAATCCGGCTCGGAGGA |
| 521 | CCTTCGATAGCTCAGCTGGTAGAGCGGAGGACTTTAGATCCTTAGGTCGCTG |
| | GTTCGAATCCGGCTCGGAGGA |
| 522 | CCTTCGATAGCTCAGCTGGTAGAGCGGAGGACTTTAGACTGCGGAAACGTTT |
| | GTGGACATCCTTAGGTCGCTGGTTCAATTCCGGCTCGAAGGA |
| 523 | CCTTCGATAGCTCAGCTGGTAGAGCGGAGGACTTTAGATCCTTAGGTCGCTG |
| | GTTCAATTCCGGCTCGAAGGA |
| 524 | CTTTCGATAGCTCAGTTGGTAGAGCGGAGGACTTTAGGTTCATTAAACTAAG |
| | GCATCCTTAGGTCGCTGGTTCGAATCCGGCTCGAAGGA |
| 525 | CTTTCGATAGCTCAGTTGGTAGAGCGGAGGACTTTAGATCCTTAGGTCGCTG |
| | GTTCGAATCCGGCTCGAAGGA |
| 526 | TCTTCAATAGCTCAGCTGGTAGAGCGGAGGACTTTAGGTGCACGCCCGTGGC |
| | CATTCTTAGGTGCTGGTTTGATTCCGACTTGGAGAG |
| 527 | TCTTCAATAGCTCAGCTGGTAGAGCGGAGGACTTTAGATTCTTAGGTGCTGG |
| | TTTGATTCCGACTTGGAGAG |
| 528 | GGTAAAATGGCTGAGTGAAGCATTGGACTTTAAATCTAAAGACAGGGGTTAA |
| | GCCTCTTTTTACCA |
| 529 | GGTAAAATGGCTGAGCAAGCATTGGACTTTAAATCTAAAGACAGATGTTGAG |
| | CCATCTTTTTAGCA |
| 530 | GGTAAAATGGCTGAGTGAAGCATTGGACTTTAAATCTAAAGACAGGGGCTAA |
| | GCCTCTTTTTACCA |
| 531 | GGTAAAATGGCTGAGCAAGCATTAGACTTTAAATCTAAAGACAGAGGTTAAG |
| | GCCTCTTTTTACCA |
| 532 | GGTAAAATGGCTGAGTAAGCATTAGACTTTAAATCTAAAGACAGAGGTCAAG |
| | GCCTCTTTTTTCCT |
| 533 | GGTAAAATGGCTGAGCAAGCATTAGACTTTAAATCTGAAAACAGAGGTCAAA |
| | GGTCTCTTTTTACCA |
| 534 | GGTAAAATGGCTGAGTAAGCATTAGACTTTAAATCTAAAGACAGAGGTCAAG |
| | GCCTCTTTTTACCA |
| 535 | GGTAAAATGACTGAATAAGCCTTAGACTTTAAATCTGAAGACAGAGGTCAAG |
| | GCCTCTTTTTACCA |
| 536 | GGTAAAATGGCTGAGTAAGCATTGGACTTTAAATCTAAAGACAGAGGTCAAG |
| | ACCTCTTTTTACCA |
| 537 | GGTAAAATGGCTGAGTAAAGCATTAGACTTTAAATCTAAGGACAGAGGCTAA |
| | ACCTCTTTTTACCA |
| 538 | CCTTCAATAGTTCAGCTGGTAGAGCAGAGGACTCTAGCTACTTCCTCAGTAG |
| | GAGACGTCCTTAGGTTGCTGGTTCGATTCCAGCTTGAAGGA |
| 539 | CCTTCAATAGTTCAGCTGGTAGAGCAGAGGACTCTAGGTCCTTAGGTTGCTG |
| | GTTCGATTCCAGCTTGAAGGA |
| 540 | GGTAAAATGGCTGAGTAAGCTTTAGACTCTAAATCTAAAGAGAGATTGAGCT |
| | CTCTTTTTACCA |
| 541 | GGTAAAATGACTGAGTAAGCATTAGACTCTAAATCTAAAGACAGAGGTCAAG |
| | ACCTCTTTTTACCA |
| 542 | GGTAAAATGGCTGAGTAAGCATTAGACTCTAAATCTAAAGACAGAGGTCAAG |
| | GCCTCTTTTTACCA |
| 543 | GGTAAAATGGCTGAGTAAGCATTAGACTCTAAATCTAAAGACAGAGGTCAAG |
| | GCCTTTTTACCA |
| 544 | CCTTCGATAGCTCAGTTGGTAGAGCGGAGGACTCTAGTTGGCTGTGTCCTTA |
| | GACATCCTTAGGTCGCTGGTTCGAATCCGGCTCGAAGGA |
| 545 | CCTTCGATAGCTCAGTTGGTAGAGCGGAGGACTCTAGATCCTTAGGTCGCTG |
| | GTTCGAATCCGGCTCGAAGGA |
| 546 | GGGGGTATAGCTCAGGGCTAGAGCTTTTTGACTCTAGAGCAAGAGGTCCCTG |
| | GTTCAAATCCAGGTTCTCCCT |
| 547 | TATAGCTCAGTGGTAGAGCATTTAACTCTAGATCAAGAGGTCCCTGGATCAA |
| | CTCTGGGTG |
| 548 | GTCAGTGTTGCACAACGGTTAAGTGAAGAGGCTCTAAACCCAGACTGGATGG |
| | GTTCAATTCCCATCTCTGCCG |
| 549 | CCTTCGATAGCTCAGTTGGTAGAGCGGAGGACTCTAGTGGATAGGGCGTGGC |
| | AATCCTTAGGTCGCTGGTTCGATTCCGGCTCGAAGGA |
| 550 | CCTTCGATAGCTCAGTTGGTAGAGCGGAGGACTCTAGATCCTTAGGTCGCTG |
| | GTTCGATTCCGGCTCGAAGGA |
| 551 | CCTTCGATAGCTCAGTTGGTAGAGCGGAGGACTCTAGGCTCATTAAGCAAGG |
| | TATCCTTAGGTCGCTGGTTCGAATCCGGCTCGGAGGA |
| 552 | CCTTCGATAGCTCAGTTGGTAGAGCGGAGGACTCTAGATCCTTAGGTCGCTG |
| | GTTCGAATCCGGCTCGGAGGA |
| 553 | CCTTCGATAGCTCAGCTGGTAGAGCGGAGGACTCTAGATTGTATAGACATTT |
| | GCGGACATCCTTAGGTCGCTGGTTCGATTCCAGCTCGAAGGA |
| 554 | CCTTCGATAGCTCAGCTGGTAGAGCGGAGGACTCTAGATCCTTAGGTCGCTG |
| | GTTCGATTCCAGCTCGAAGGA |
| 555 | CCTTCGATAGCTCAGCTGGTAGAGCGGAGGACTCTAGCTACTTCCTCAGCAG |
| | GAGACATCCTTAGGTCGCTGGTTCGATTCCGGCTCGAAGGA |
| 556 | CCTTCGATAGCTCAGCTGGTAGAGCGGAGGACTCTAGATCCTTAGGTCGCTG |
| | GTTCGATTCCGGCTCGAAGGA |
| 557 | CCTTCGATAGCTCAGCTGGTAGAGCGGAGGACTCTAGGCGCGCGCCCGTGGC |
| | CATCCTTAGGTCGCTGGTTCGATTCCGGCTCGAAGGA |
| 558 | CCTTCGATAGCTCAGCTGGTAGAGCGGAGGACTCTAGATCCTTAGGTCGCTG |
| | GTTCGATTCCGGCTCGAAGGA |
| 559 | CCTTCGATAGCTCAGCTGGTAGAGCGGAGGACTCTAGCCTGTAGAAACATTT |
| | GTGGACATCCTTAGGTCGCTGGTTCGATTCCGGCTCGAAGGA |
| 560 | CCTTCGATAGCTCAGCTGGTAGAGCGGAGGACTCTAGATCCTTAGGTCGCTG |
| | GTTCGATTCCGGCTCGAAGGA |
| 561 | CCTTCGATAGCTCAGCTGGTAGAGCGGAGGACTCTAGATTGTACAGACATTT |
| | GCGGACATCCTTAGGTCGCTGGTTCGATTCCGGCTCGAAGGA |
| 562 | CCTTCGATAGCTCAGCTGGTAGAGCGGAGGACTCTAGATCCTTAGGTCGCTG |
| | GTTCGATTCCGGCTCGAAGGA |
| 563 | CCTTCGATAGCTCAGCTGGTAGAGCGGAGGACTCTAGTACTTAATGTGTGGT |
| | CATCCTTAGGTCGCTGGTTCGATTCCGGCTCGAAGGA |
| 564 | CCTTCGATAGCTCAGCTGGTAGAGCGGAGGACTCTAGATCCTTAGGTCGCTG |
| | GTTCGATTCCGGCTCGAAGGA |
| 565 | CCTTCGATAGCTCAGCTGGTAGAGCGGAGGACTCTAGGGGTTTGAATGTGGT |
| | CATCCTTAGGTCGCTGGTTCGAATCCGGCTCGGAGGA |
| 566 | CCTTCGATAGCTCAGCTGGTAGAGCGGAGGACTCTAGATCCTTAGGTCGCTG |
| | GTTCGAATCCGGCTCGGAGGA |
| 567 | CCTTCGATAGCTCAGCTGGTAGAGCGGAGGACTCTAGACTGCGGAAACGTTT |
| | GTGGACATCCTTAGGTCGCTGGTTCAATTCCGGCTCGAAGGA |
| 568 | CCTTCGATAGCTCAGCTGGTAGAGCGGAGGACTCTAGATCCTTAGGTCGCTG |
| | GTTCAATTCCGGCTCGAAGGA |
| 569 | CTTTCGATAGCTCAGTTGGTAGAGCGGAGGACTCTAGGTTCATTAAACTAAG |
| | GCATCCTTAGGTCGCTGGTTCGAATCCGGCTCGAAGGA |
| 570 | CTTTCGATAGCTCAGTTGGTAGAGCGGAGGACTCTAGATCCTTAGGTCGCTG |
| | GTTCGAATCCGGCTCGAAGGA |
| 571 | TCTTCAATAGCTCAGCTGGTAGAGCGGAGGACTCTAGGTGCACGCCCGTGGC |
| | CATTCTTAGGTGCTGGTTTGATTCCGACTTGGAGAG |
| 572 | TCTTCAATAGCTCAGCTGGTAGAGCGGAGGACTCTAGATTCTTAGGTGCTGG |
| | TTTGATTCCGACTTGGAGAG |
| 573 | GGTAAAATGGCTGAGTGAAGCATTGGACTCTAAATCTAAAGACAGGGGTTAA |
| | GCCTCTTTTTACCA |
| 574 | GGTAAAATGGCTGAGCAAGCATTGGACTCTAAATCTAAAGACAGATGTTGAG |
| | CCATCTTTTTAGCA |
| 575 | GGTAAAATGGCTGAGTGAAGCATTGGACTCTAAATCTAAAGACAGGGGCTAA |
| | GCCTCTTTTTACCA |
| 576 | GGTAAAATGGCTGAGCAAGCATTAGACTCTAAATCTAAAGACAGAGGTTAAG |
| | GCCTCTTTTTACCA |
| 577 | GGTAAAATGGCTGAGTAAGCATTAGACTCTAAATCTAAAGACAGAGGTCAAG |
| | GCCTCTTTTTTCCT |
| 578 | GGTAAAATGGCTGAGCAAGCATTAGACTCTAAATCTGAAAACAGAGGTCAAA |
| | GGTCTCTTTTTACCA |
| 579 | GGTAAAATGGCTGAGTAAGCATTAGACTCTAAATCTAAAGACAGAGGTCAAG |
| | GCCTCTTTTTACCA |
| 580 | GGTAAAATGACTGAATAAGCCTTAGACTCTAAATCTGAAGACAGAGGTCAAG |
| | GCCTCTTTTTACCA |
| 581 | GGTAAAATGGCTGAGTAAGCATTGGACTCTAAATCTAAAGACAGAGGTCAAG |
| | ACCTCTTTTTACCA |
| 582 | GGTAAAATGGCTGAGTAAAGCATTAGACTCTAAATCTAAGGACAGAGGCTAA |
| | ACCTCTTTTTACCA |
| 583 | GTTAAGATGGCAGAGCCTGGTAATTGCATTAAACTTAAAATTTTATAATCAG |
| | AGGTTCAACTCCTCTTCTTAACA |
| 584 | GTTAAGATGGCAGAGCCCGGCAATTGCATTAGACTTAAAACTTTATAATCAG |
| | AGGTTCAACTCCTCTCATTAACA |
| 585 | GGTAGCGTGGCCGAGCGGTCTAAGGCGCTGGATTTTAGCTCCAGTCTCTTCG |
| | GGGGCGTGGGTTCAAATCCCACCGCTGCCA |
| 586 | GGTAGCGTGGCCGAGTGGTCTAAGACGCTGGATTTTAGCTCCAGTCTCTTCG |
| | GGGGCGTGGGTTTGAATCCCACCGCTGCCA |
| 587 | GGGCCAGTGGCTCAATGGATAATGCGTCTGACTTTAAATCAGAAGATTCCAG |
| | CCTTGACTCCTGGCTGGCTCA |
| 588 | GGTAGGGTGGCCGAGCGGTCTAAGGCACTGTATTTTAACTCCAGTCTCTTCA |
| | GAGGCATGGGTTTGAATCCCACTGCTGCCA |
| 589 | GCCGAGCGGTCTAAGGCTCCGGATTTTAGCGCCGGTGTCTTCGGAGGCATGG |
| | GTTCGAATTCCAC |
| 590 | GTCAGGATGGCCGAGTGGTCTAAGGCGCCAGACTTTAGCTAAGCTTCCTCCG |
| | CGGTGGGGATTCTGGTCTCCAATGGAGGCGTGGGTTCGAATCCCACTTCTGA |
| | CA |
| 591 | GTCAGGATGGCCGAGTGGTCTAAGGCGCCAGACTTTAGTTCTGGTCTCCAAT |
| | GGAGGCGTGGGTTCGAATCCCACTTCTGACA |
| 592 | GTCAGGATGGCCGAGTGGTCTAAGGCGCCAGACTTTAGCTTGGCTTCCTCGT |
| | GTTGAGGATTCTGGTCTCCAATGGAGGCGTGGGTTCGAATCCCACTTCTGAC |
| | A |
| 593 | GTCAGGATGGCCGAGTGGTCTAAGGCGCCAGACTTTAGTTCTGGTCTCCAAT |
| | GGAGGCGTGGGTTCGAATCCCACTTCTGACA |
| 594 | GTCAGGATGGCCGAGTGGTCTAAGGCGCCAGACTTTAGCTTACTGCTTCCTG |
| | TGTTCGGGTCTTCTGGTCTCCGTATGGAGGCGTGGGTTCGAATCCCACTTCT |
| | GACA |
| 595 | GTCAGGATGGCCGAGTGGTCTAAGGCGCCAGACTTTAGTTCTGGTCTCCGTA |
| | TGGAGGCGTGGGTTCGAATCCCACTTCTGACA |
| 596 | GTCAGGATGGCCGAGTGGTCTAAGGCGCCAGACTTTAGTTGCTACTTCCCAG |
| | GTTTGGGGCTTCTGGTCTCCGCATGGAGGCGTGGGTTCGAATCCCACTTCTG |
| | ACA |
| 597 | GTCAGGATGGCCGAGTGGTCTAAGGCGCCAGACTTTAGTTCTGGTCTCCGCA |
| | TGGAGGCGTGGGTTCGAATCCCACTTCTGACA |
| 598 | GTCAGGATGGCCGAGTGGTCTAAGGCGCCAGACTTTAGGTAAGCACCTTGCC |
| | TGCGGGCTTTCTGGTCTCCGGATGGAGGCGTGGGTTCGAATCCCACTTCTGA |
| | CA |
| 599 | GTCAGGATGGCCGAGTGGTCTAAGGCGCCAGACTTTAGTTTCTGGTCTCCGG |
| | ATGGAGGCGTGGGTTCGAATCCCACTTCTGACA |
| 600 | GCCTCCTTAGTGCAGTAGGTAGCGCATCAGTCTTTAAATCTGAATGGTCCTG |
| | AGTTCAAGCCTCAGAGGGGGCA |
| 601 | GTCAGGATGGCCGAGCAGTCTTAAGGCGCTGCGTTTTAATCGCACCCTCCGC |
| | TGGAGGCGTGGGTTCGAATCCCACTTTTGACA |
| 602 | GGTTCCATGGTGTAATGGTGAGCACTCTGGACTTTAAATCCAGAAGTAGTGC |
| | TGGAACAA |
| 603 | GTCAGGGTGGCTGAGCAGTCTGAGGGGCTGCGTTTTAGTCGCAGTCTGCCCT |
| | GGAGGCGTGGGTTCGAATCCCACTCCTGAAA |
| 604 | ACCAGGATGGCCGAGTGGTTAAGGCGTTGGACTTTAGATCCAATGGACATAT |
| | GTCCGCGTGGGTTCGAACCCCACTCCTGGTA |
| 605 | ACCGGGATGGCCGAGTGGTTAAGGCGTTGGACTTTAGATCCAATGGGCTGGT |
| | GCCCGCGTGGGTTCGAACCCCACTCTCGGTA |
| 606 | ACCAGAATGGCCGAGTGGTTAAGGCGTTGGACTTTAGATCCAATGGATTCAT |
| | ATCCGCGTGGGTTCGAACCCCACTTCTGGTA |
| 607 | ACCGGGATGGCTGAGTGGTTAAGGCGTTGGACTTTAGATCCAATGGACAGGT |
| | GTCCGCGTGGGTTCGAGCCCCACTCCCGGTA |
| 608 | ACTCATTTGGCTGAGTGGTTAAGGCATTGGACTTTAGATCCAATGGAGTAGT |
| | GGCTGTGTGGGTTTAAACCCCACTACTGGTA |
| 609 | GAGAAAGTCATCGTAGTTACGAAGTTGGCTTTAACCCAGTTTTGGGAGGTTC |
| | AATTCCTTCCTTTCTCT |
| 610 | ACCAGGATGGCCAAGTAGTTAAAGGCACTGGACTTTAGAGCCAATGGACATA |
| | TGTCTGTGTGGGTTTGAACCCCACTCCTGGTG |
| 611 | GGTAGCGTGGCCGAGCGGTCTAAGGCGCTGGATTTTAGCTCCAGTCTCTTCG |
| | GAGGCGTGGGTTCGAATCCCACCGCTGCCA |
| 612 | GGTAGTGTGGCCGAGCGGTCTAAGGCGCTGGATTTTAGCTCCAGTCTCTTCG |
| | GGGGCGTGGGTTCGAATCCCACCACTGCCA |
| 613 | GGTAGCGTGGCCGAGTGGTCTAAGGCGCTGGATTTTAGCTCCAGTCATTTCG |
| | ATGGCGTGGGTTCGAATCCCACCGCTGCCA |
| 614 | GGTAGTGTGGTTGAATGGTCTAAGGCACTGAATTTTAGCTCCAGTCTCTTTG |
| | GGGACGTGGGTTTAAATCCCACTGCTGCAA |
| 615 | GTTAAGATGGCAGAGCCTGGTAATTGCACTAAACTTAAAATTTTATAATCAG |
| | AGGTTCAACTCCTCTTCTTAACA |
| 616 | GTTAAGATGGCAGAGCCCGGCAATTGCACTAGACTTAAAACTTTATAATCAG |
| | AGGTTCAACTCCTCTCATTAACA |
| 617 | GGTAGCGTGGCCGAGCGGTCTAAGGCGCTGGATTCTAGCTCCAGTCTCTTCG |
| | GGGGCGTGGGTTCAAATCCCACCGCTGCCA |
| 618 | GGTAGCGTGGCCGAGTGGTCTAAGACGCTGGATTCTAGCTCCAGTCTCTTCG |
| | GGGGCGTGGGTTTGAATCCCACCGCTGCCA |
| 619 | GGGCCAGTGGCTCAATGGATAATGCGTCTGACTCTAAATCAGAAGATTCCAG |
| | CCTTGACTCCTGGCTGGCTCA |
| 620 | GGTAGGGTGGCCGAGCGGTCTAAGGCACTGTATTCTAACTCCAGTCTCTTCA |
| | GAGGCATGGGTTTGAATCCCACTGCTGCCA |
| 621 | GCCGAGCGGTCTAAGGCTCCGGATTCTAGCGCCGGTGTCTTCGGAGGCATGG |
| | GTTCGAATTCCAC |
| 622 | GTCAGGATGGCCGAGTGGTCTAAGGCGCCAGACTCTAGCTAAGCTTCCTCCG |
| | CGGTGGGGATTCTGGTCTCCAATGGAGGCGTGGGTTCGAATCCCACTTCTGA |
| | CA |
| 623 | GTCAGGATGGCCGAGTGGTCTAAGGCGCCAGACTCTAGTTCTGGTCTCCAAT |
| | GGAGGCGTGGGTTCGAATCCCACTTCTGACA |
| 624 | GTCAGGATGGCCGAGTGGTCTAAGGCGCCAGACTCTAGCTTGGCTTCCTCGT |
| | GTTGAGGATTCTGGTCTCCAATGGAGGCGTGGGTTCGAATCCCACTTCTGAC |
| | A |
| 625 | GTCAGGATGGCCGAGTGGTCTAAGGCGCCAGACTCTAGTTCTGGTCTCCAAT |
| | GGAGGCGTGGGTTCGAATCCCACTTCTGACA |
| 626 | GTCAGGATGGCCGAGTGGTCTAAGGCGCCAGACTCTAGCTTACTGCTTCCTG |
| | TGTTCGGGTCTTCTGGTCTCCGTATGGAGGCGTGGGTTCGAATCCCACTTCT |
| | GACA |
| 627 | GTCAGGATGGCCGAGTGGTCTAAGGCGCCAGACTCTAGTTCTGGTCTCCGTA |
| | TGGAGGCGTGGGTTCGAATCCCACTTCTGACA |
| 628 | GTCAGGATGGCCGAGTGGTCTAAGGCGCCAGACTCTAGTTGCTACTTCCCAG |
| | GTTTGGGGCTTCTGGTCTCCGCATGGAGGCGTGGGTTCGAATCCCACTTCTG |
| | ACA |
| 629 | GTCAGGATGGCCGAGTGGTCTAAGGCGCCAGACTCTAGTTCTGGTCTCCGCA |
| | TGGAGGCGTGGGTTCGAATCCCACTTCTGACA |
| 630 | GTCAGGATGGCCGAGTGGTCTAAGGCGCCAGACTCTAGGTAAGCACCTTGCC |
| | TGCGGGCTTTCTGGTCTCCGGATGGAGGCGTGGGTTCGAATCCCACTTCTGA |
| | CA |
| 631 | GTCAGGATGGCCGAGTGGTCTAAGGCGCCAGACTCTAGTTTCTGGTCTCCGG |
| | ATGGAGGCGTGGGTTCGAATCCCACTTCTGACA |
| 632 | GCCTCCTTAGTGCAGTAGGTAGCGCATCAGTCTCTAAATCTGAATGGTCCTG |
| | AGTTCAAGCCTCAGAGGGGGCA |
| 633 | GTCAGGATGGCCGAGCAGTCTTAAGGCGCTGCGTTCTAATCGCACCCTCCGC |
| | TGGAGGCGTGGGTTCGAATCCCACTTTTGACA |
| 634 | GGTTCCATGGTGTAATGGTGAGCACTCTGGACTCTAAATCCAGAAGTAGTGC |
| | TGGAACAA |
| 635 | GTCAGGGTGGCTGAGCAGTCTGAGGGGCTGCGTTCTAGTCGCAGTCTGCCCT |
| | GGAGGCGTGGGTTCGAATCCCACTCCTGAAA |
| 636 | ACCAGGATGGCCGAGTGGTTAAGGCGTTGGACTCTAGATCCAATGGACATAT |
| | GTCCGCGTGGGTTCGAACCCCACTCCTGGTA |
| 637 | ACCGGGATGGCCGAGTGGTTAAGGCGTTGGACTCTAGATCCAATGGGCTGGT |
| | GCCCGCGTGGGTTCGAACCCCACTCTCGGTA |
| 638 | ACCAGAATGGCCGAGTGGTTAAGGCGTTGGACTCTAGATCCAATGGATTCAT |
| | ATCCGCGTGGGTTCGAACCCCACTTCTGGTA |
| 639 | ACCGGGATGGCTGAGTGGTTAAGGCGTTGGACTCTAGATCCAATGGACAGGT |
| | GTCCGCGTGGGTTCGAGCCCCACTCCCGGTA |
| 640 | ACTCATTTGGCTGAGTGGTTAAGGCATTGGACTCTAGATCCAATGGAGTAGT |
| | GGCTGTGTGGGTTTAAACCCCACTACTGGTA |
| 641 | GAGAAAGTCATCGTAGTTACGAAGTTGGCTCTAACCCAGTTTTGGGAGGTTC |
| | AATTCCTTCCTTTCTCT |
| 642 | ACCAGGATGGCCAAGTAGTTAAAGGCACTGGACTCTAGAGCCAATGGACATA |
| | TGTCTGTGTGGGTTTGAACCCCACTCCTGGTG |
| 643 | GGTAGCGTGGCCGAGCGGTCTAAGGCGCTGGATTCTAGCTCCAGTCTCTTCG |
| | GAGGCGTGGGTTCGAATCCCACCGCTGCCA |
| 644 | GGTAGTGTGGCCGAGCGGTCTAAGGCGCTGGATTCTAGCTCCAGTCTCTTCG |
| | GGGGCGTGGGTTCGAATCCCACCACTGCCA |
| 645 | GGTAGCGTGGCCGAGTGGTCTAAGGCGCTGGATTCTAGCTCCAGTCATTTCG |
| | ATGGCGTGGGTTCGAATCCCACCGCTGCCA |
| 646 | GGTAGTGTGGTTGAATGGTCTAAGGCACTGAATTCTAGCTCCAGTCTCTTTG |
| | GGGACGTGGGTTTAAATCCCACTGCTGCAA |
| 647 | GTTAAGATGGCAGAGCCTGGTAATTGCATCAAACTTAAAATTTTATAATCAG |
| | AGGTTCAACTCCTCTTCTTAACA |
| 648 | GTTAAGATGGCAGAGCCCGGCAATTGCATCAGACTTAAAACTTTATAATCAG |
| | AGGTTCAACTCCTCTCATTAACA |
| 649 | GGTAGCGTGGCCGAGCGGTCTAAGGCGCTGGATTTCAGCTCCAGTCTCTTCG |
| | GGGGCGTGGGTTCAAATCCCACCGCTGCCA |
| 650 | GGTAGCGTGGCCGAGTGGTCTAAGACGCTGGATTTCAGCTCCAGTCTCTTCG |
| | GGGGCGTGGGTTTGAATCCCACCGCTGCCA |
| 651 | GGGCCAGTGGCTCAATGGATAATGCGTCTGACTTCAAATCAGAAGATTCCAG |
| | CCTTGACTCCTGGCTGGCTCA |
| 652 | GGTAGGGTGGCCGAGCGGTCTAAGGCACTGTATTTCAACTCCAGTCTCTTCA |
| | GAGGCATGGGTTTGAATCCCACTGCTGCCA |
| 653 | GCCGAGCGGTCTAAGGCTCCGGATTTCAGCGCCGGTGTCTTCGGAGGCATGG |
| | GTTCGAATTCCAC |
| 654 | GTCAGGATGGCCGAGTGGTCTAAGGCGCCAGACTTCAGCTAAGCTTCCTCCG |
| | CGGTGGGGATTCTGGTCTCCAATGGAGGCGTGGGTTCGAATCCCACTTCTGA |
| | CA |
| 655 | GTCAGGATGGCCGAGTGGTCTAAGGCGCCAGACTTCAGTTCTGGTCTCCAAT |
| | GGAGGCGTGGGTTCGAATCCCACTTCTGACA |
| 656 | GTCAGGATGGCCGAGTGGTCTAAGGCGCCAGACTTCAGCTTGGCTTCCTCGT |
| | GTTGAGGATTCTGGTCTCCAATGGAGGCGTGGGTTCGAATCCCACTTCTGAC |
| | A |
| 657 | GTCAGGATGGCCGAGTGGTCTAAGGCGCCAGACTTCAGTTCTGGTCTCCAAT |
| | GGAGGCGTGGGTTCGAATCCCACTTCTGACA |
| 658 | GTCAGGATGGCCGAGTGGTCTAAGGCGCCAGACTTCAGCTTACTGCTTCCTG |
| | TGTTCGGGTCTTCTGGTCTCCGTATGGAGGCGTGGGTTCGAATCCCACTTCT |
| | GACA |
| 659 | GTCAGGATGGCCGAGTGGTCTAAGGCGCCAGACTTCAGTTCTGGTCTCCGTA |
| | TGGAGGCGTGGGTTCGAATCCCACTTCTGACA |
| 660 | GTCAGGATGGCCGAGTGGTCTAAGGCGCCAGACTTCAGTTGCTACTTCCCAG |
| | GTTTGGGGCTTCTGGTCTCCGCATGGAGGCGTGGGTTCGAATCCCACTTCTG |
| | ACA |
| 661 | GTCAGGATGGCCGAGTGGTCTAAGGCGCCAGACTTCAGTTCTGGTCTCCGCA |
| | TGGAGGCGTGGGTTCGAATCCCACTTCTGACA |
| 662 | GTCAGGATGGCCGAGTGGTCTAAGGCGCCAGACTTCAGGTAAGCACCTTGCC |
| | TGCGGGCTTTCTGGTCTCCGGATGGAGGCGTGGGTTCGAATCCCACTTCTGA |
| | CA |
| 663 | GTCAGGATGGCCGAGTGGTCTAAGGCGCCAGACTTCAGTTTCTGGTCTCCGG |
| | ATGGAGGCGTGGGTTCGAATCCCACTTCTGACA |
| 664 | GCCTCCTTAGTGCAGTAGGTAGCGCATCAGTCTTCAAATCTGAATGGTCCTG |
| | AGTTCAAGCCTCAGAGGGGGCA |
| 665 | GTCAGGATGGCCGAGCAGTCTTAAGGCGCTGCGTTTCAATCGCACCCTCCGC |
| | TGGAGGCGTGGGTTCGAATCCCACTTTTGACA |
| 666 | GGTTCCATGGTGTAATGGTGAGCACTCTGGACTTCAAATCCAGAAGTAGTGC |
| | TGGAACAA |
| 667 | GTCAGGGTGGCTGAGCAGTCTGAGGGGCTGCGTTTCAGTCGCAGTCTGCCCT |
| | GGAGGCGTGGGTTCGAATCCCACTCCTGAAA |
| 668 | ACCAGGATGGCCGAGTGGTTAAGGCGTTGGACTTCAGATCCAATGGACATAT |
| | GTCCGCGTGGGTTCGAACCCCACTCCTGGTA |
| 669 | ACCGGGATGGCCGAGTGGTTAAGGCGTTGGACTTCAGATCCAATGGGCTGGT |
| | GCCCGCGTGGGTTCGAACCCCACTCTCGGTA |
| 670 | ACCAGAATGGCCGAGTGGTTAAGGCGTTGGACTTCAGATCCAATGGATTCAT |
| | ATCCGCGTGGGTTCGAACCCCACTTCTGGTA |
| 671 | ACCGGGATGGCTGAGTGGTTAAGGCGTTGGACTTCAGATCCAATGGACAGGT |
| | GTCCGCGTGGGTTCGAGCCCCACTCCCGGTA |
| 672 | ACTCATTTGGCTGAGTGGTTAAGGCATTGGACTTCAGATCCAATGGAGTAGT |
| | GGCTGTGTGGGTTTAAACCCCACTACTGGTA |
| 673 | GAGAAAGTCATCGTAGTTACGAAGTTGGCTTCAACCCAGTTTTGGGAGGTTC |
| | AATTCCTTCCTTTCTCT |
| 674 | ACCAGGATGGCCAAGTAGTTAAAGGCACTGGACTTCAGAGCCAATGGACATA |
| | TGTCTGTGTGGGTTTGAACCCCACTCCTGGTG |
| 675 | GGTAGCGTGGCCGAGCGGTCTAAGGCGCTGGATTTCAGCTCCAGTCTCTTCG |
| | GAGGCGTGGGTTCGAATCCCACCGCTGCCA |
| 676 | GGTAGTGTGGCCGAGCGGTCTAAGGCGCTGGATTTCAGCTCCAGTCTCTTCG |
| | GGGGCGTGGGTTCGAATCCCACCACTGCCA |
| 677 | GGTAGCGTGGCCGAGTGGTCTAAGGCGCTGGATTTCAGCTCCAGTCATTTCG |
| | ATGGCGTGGGTTCGAATCCCACCGCTGCCA |
| 678 | GGTAGTGTGGTTGAATGGTCTAAGGCACTGAATTTCAGCTCCAGTCTCTTTG |
| | GGGACGTGGGTTTAAATCCCACTGCTGCAA |
| 679 | GAGAAGGTCACAGAGGTTATGGGATTGGCTTTAAACCAGTCTGTGGGGGGTT |
| | CGATTCCCTCCTTTTTCA |
| 680 | GAGAAGGTCATAGAGGTTATGGGATTGGCTTTAAACCAGTCTCTGGGGGGTT |
| | CGATTCCCTCCTTTTTCA |
| 681 | GAAAAAGTCATAGGGGTTATGAGGCTGGCTTTAAACCAGCCTTAGGAGGTTC |
| | AATTCCTTCCTTTTTTG |
| 682 | GGCCGGTTAGCTCAGTTGGTTAGAGCGTGCTGCTTTAAATGCCAGGGTCGAG |
| | GTTTCGATCCCCGTACGGGCCT |
| 683 | GTAGTCGTGGCCGAGTGGTTAAGGCGATGGACTTTAAATCCATTGGGGTTTC |
| | CCCGCGCAGGTTCGAATCCTGCCGACTACG |
| 684 | GTAGTCGTGGCCGAGTGGTTAAGGCGATGGACTTTAAATCCATTGGGGTTTC |
| | CCCACGCAGGTTCGAATCCTGCCGACTACG |
| 685 | GTAGTCGTGGCCGAGTGGTTAAGGTGATGGACTTTAAACCCATTGGGGTCTC |
| | CCCGCGCAGGTTCGAATCCTGCCGACTACG |
| 686 | GGGTGTATGGCTCAGGGGTAGAGAATTTGACTTTAGATCAAGAGGTCCCTGG |
| | TTCAAATCCAGGTGCCCCCT |
| 687 | AGTTGTAGCTGAGTGGTTAAGGCAACGAGCTTTAAATTCGTTGGTTTCTCTC |
| | TGTGCAGGTTTGAATCCTGCTAATTA |
| 688 | CAAGAAATTCATAGAGGTTATGGGATTGGCTTTAAACCAGTTTCAGGAGGTT |
| | CGATTCCTTCCTTTTTGG |
| 689 | GCTGTGATGGCCGAGTGGTTAAGGCGTTGGACTTTAAATCCAATGGGGTCTC |
| | CCCGCGCAGGTTCGAATCCTGCTCACAGCG |
| 690 | GCTGTGATGGCCGAGTGGTTAAGGCGTTGGACTTTAAATCCAATGGGGTCTC |
| | CCCGCGCAGGTTCAAATCCTGCTCACAGCG |
| 691 | GCTGTGATGGCCGAGTGGTTAAGGTGTTGGACTTTAAATCCAATGGGGGTTC |
| | CCCGCGCAGGTTCAAATCCTGCTCACAGCG |
| 692 | GTCACGGTGGCCGAGTGGTTAAGGCGTTGGACTTTAAATCCAATGGGGTTTC |
| | CCCGCACAGGTTCGAATCCTGTTCGTGACG |
| 693 | GACGAGGTGGCCGAGTGGTTAAGGCGATGGACTTTAAATCCATTGTGCTCTG |
| | CACGCGTGGGTTCGAATCCCACCCTCGTCG |
| 694 | GACGAGGTGGCCGAGTGGTTAAGGCGATGGACTTTAAATCCATTGTGCTCTG |
| | CACGCGTGGGTTCGAATCCCACCTTCGTCG |
| 695 | GGCCGGTTAGCTCAGTTGGTTAGAGCGTGCTTTAACTAATGCCAGGGTCGAG |
| | GTTTCGATCCCCGTACGGGCCT |
| 696 | GACGAGGTGGCCGAGTGGTTAAGGCGATGGACTTTAAATCCATTGTGCTCTG |
| | CACACGTGGGTTCGAATCCCATCCTCGTCG |
| 697 | GAGGCCTGGCCGAGTGGTTAAGGCGATGGACTTTAAATCCATTGTGCTCTGC |
| | ACGCGTGGGTTCGAATCCCATCCTCG |
| 698 | GCAGCGATGGCCGAGTGGTTAAGGCGTTGGACTTTAAATCCAATGGGGTCTC |
| | CCCGCGCAGGTTCGAACCCTGCTCGCTGCG |
| 699 | GTAGTCGTGGCCGAGTGGTTAAGGCGATGGACTTTAAATCCATTGGGGTTTC |
| | CCCGCGCAGGTTCGAATCCTGCCGACTACG |
| 700 | GTAGTCGTGGCCGAGTGGTTAAGGCGATGGACTTTAAATCCATTGGGGTCTC |
| | CCCGCGCAGGTTCGAATCCTGCCGACTACG |
| 701 | GTAGTCGTGGCCGAGTGGTTAAGGCGATGGACTTTAAATCCATTGGGGTTTC |
| | CCCGCGCAGGTTCGAATCCTGTCGGCTACG |
| 702 | GAGAAGGTCACAGAGGTTATGGGATTGGCTCTAAACCAGTCTGTGGGGGGTT |
| | CGATTCCCTCCTTTTTCA |
| 703 | GAGAAGGTCATAGAGGTTATGGGATTGGCTCTAAACCAGTCTCTGGGGGGTT |
| | CGATTCCCTCCTTTTTCA |
| 704 | GAAAAAGTCATAGGGGTTATGAGGCTGGCTCTAAACCAGCCTTAGGAGGTTC |
| | AATTCCTTCCTTTTTTG |
| 705 | GGCCGGTTAGCTCAGTTGGTTAGAGCGTGCTGCTCTAAATGCCAGGGTCGAG |
| | GTTTCGATCCCCGTACGGGCCT |
| 706 | GTAGTCGTGGCCGAGTGGTTAAGGCGATGGACTCTAAATCCATTGGGGTTTC |
| | CCCGCGCAGGTTCGAATCCTGCCGACTACG |
| 707 | GTAGTCGTGGCCGAGTGGTTAAGGCGATGGACTCTAAATCCATTGGGGTTTC |
| | CCCACGCAGGTTCGAATCCTGCCGACTACG |
| 708 | GTAGTCGTGGCCGAGTGGTTAAGGTGATGGACTCTAAACCCATTGGGGTCTC |
| | CCCGCGCAGGTTCGAATCCTGCCGACTACG |
| 709 | GGGTGTATGGCTCAGGGGTAGAGAATTTGACTCTAGATCAAGAGGTCCCTGG |
| | TTCAAATCCAGGTGCCCCCT |
| 710 | AGTTGTAGCTGAGTGGTTAAGGCAACGAGCTCTAAATTCGTTGGTTTCTCTC |
| | TGTGCAGGTTTGAATCCTGCTAATTA |
| 711 | CAAGAAATTCATAGAGGTTATGGGATTGGCTCTAAACCAGTTTCAGGAGGTT |
| | CGATTCCTTCCTTTTTGG |
| 712 | GCTGTGATGGCCGAGTGGTTAAGGCGTTGGACTCTAAATCCAATGGGGTCTC |
| | CCCGCGCAGGTTCGAATCCTGCTCACAGCG |
| 713 | GCTGTGATGGCCGAGTGGTTAAGGCGTTGGACTCTAAATCCAATGGGGTCTC |
| | CCCGCGCAGGTTCAAATCCTGCTCACAGCG |
| 714 | GCTGTGATGGCCGAGTGGTTAAGGTGTTGGACTCTAAATCCAATGGGGGTTC |
| | CCCGCGCAGGTTCAAATCCTGCTCACAGCG |
| 715 | GTCACGGTGGCCGAGTGGTTAAGGCGTTGGACTCTAAATCCAATGGGGTTTC |
| | CCCGCACAGGTTCGAATCCTGTTCGTGACG |
| 716 | GACGAGGTGGCCGAGTGGTTAAGGCGATGGACTCTAAATCCATTGTGCTCTG |
| | CACGCGTGGGTTCGAATCCCACCCTCGTCG |
| 717 | GACGAGGTGGCCGAGTGGTTAAGGCGATGGACTCTAAATCCATTGTGCTCTG |
| | CACGCGTGGGTTCGAATCCCACCTTCGTCG |
| 718 | GGCCGGTTAGCTCAGTTGGTTAGAGCGTGCTCTAACTAATGCCAGGGTCGAG |
| | GTTTCGATCCCCGTACGGGCCT |
| 719 | GACGAGGTGGCCGAGTGGTTAAGGCGATGGACTCTAAATCCATTGTGCTCTG |
| | CACACGTGGGTTCGAATCCCATCCTCGTCG |
| 720 | GAGGCCTGGCCGAGTGGTTAAGGCGATGGACTCTAAATCCATTGTGCTCTGC |
| | ACGCGTGGGTTCGAATCCCATCCTCG |
| 721 | GCAGCGATGGCCGAGTGGTTAAGGCGTTGGACTCTAAATCCAATGGGGTCTC |
| | CCCGCGCAGGTTCGAACCCTGCTCGCTGCG |
| 722 | GTAGTCGTGGCCGAGTGGTTAAGGCGATGGACTCTAAATCCATTGGGGTTTC |
| | CCCGCGCAGGTTCGAATCCTGCCGACTACG |
| 723 | GTAGTCGTGGCCGAGTGGTTAAGGCGATGGACTCTAAATCCATTGGGGTCTC |
| | CCCGCGCAGGTTCGAATCCTGCCGACTACG |
| 724 | GTAGTCGTGGCCGAGTGGTTAAGGCGATGGACTCTAAATCCATTGGGGTTTC |
| | CCCGCGCAGGTTCGAATCCTGTCGGCTACG |
| 725 | GAGAAGGTCACAGAGGTTATGGGATTGGCTTCAAACCAGTCTGTGGGGGGTT |
| | CGATTCCCTCCTTTTTCA |
| 726 | GAGAAGGTCATAGAGGTTATGGGATTGGCTTCAAACCAGTCTCTGGGGGGTT |
| | CGATTCCCTCCTTTTTCA |
| 727 | GAAAAAGTCATAGGGGTTATGAGGCTGGCTTCAAACCAGCCTTAGGAGGTTC |
| | AATTCCTTCCTTTTTTG |
| 728 | GGCCGGTTAGCTCAGTTGGTTAGAGCGTGCTGCTTCAAATGCCAGGGTCGAG |
| | GTTTCGATCCCCGTACGGGCCT |
| 729 | GTAGTCGTGGCCGAGTGGTTAAGGCGATGGACTTCAAATCCATTGGGGTTTC |
| | CCCGCGCAGGTTCGAATCCTGCCGACTACG |
| 730 | GTAGTCGTGGCCGAGTGGTTAAGGCGATGGACTTCAAATCCATTGGGGTTTC |
| | CCCACGCAGGTTCGAATCCTGCCGACTACG |
| 731 | GTAGTCGTGGCCGAGTGGTTAAGGTGATGGACTTCAAACCCATTGGGGTCTC |
| | CCCGCGCAGGTTCGAATCCTGCCGACTACG |
| 732 | GGGTGTATGGCTCAGGGGTAGAGAATTTGACTTCAGATCAAGAGGTCCCTGG |
| | TTCAAATCCAGGTGCCCCCT |
| 733 | AGTTGTAGCTGAGTGGTTAAGGCAACGAGCTTCAAATTCGTTGGTTTCTCTC |
| | TGTGCAGGTTTGAATCCTGCTAATTA |
| 734 | CAAGAAATTCATAGAGGTTATGGGATTGGCTTCAAACCAGTTTCAGGAGGTT |
| | CGATTCCTTCCTTTTTGG |
| 735 | GCTGTGATGGCCGAGTGGTTAAGGCGTTGGACTTCAAATCCAATGGGGTCTC |
| | CCCGCGCAGGTTCGAATCCTGCTCACAGCG |
| 736 | GCTGTGATGGCCGAGTGGTTAAGGCGTTGGACTTCAAATCCAATGGGGTCTC |
| | CCCGCGCAGGTTCAAATCCTGCTCACAGCG |
| 737 | GCTGTGATGGCCGAGTGGTTAAGGTGTTGGACTTCAAATCCAATGGGGGTTC |
| | CCCGCGCAGGTTCAAATCCTGCTCACAGCG |
| 738 | GTCACGGTGGCCGAGTGGTTAAGGCGTTGGACTTCAAATCCAATGGGGTTTC |
| | CCCGCACAGGTTCGAATCCTGTTCGTGACG |
| 739 | GACGAGGTGGCCGAGTGGTTAAGGCGATGGACTTCAAATCCATTGTGCTCTG |
| | CACGCGTGGGTTCGAATCCCACCCTCGTCG |
| 740 | GACGAGGTGGCCGAGTGGTTAAGGCGATGGACTTCAAATCCATTGTGCTCTG |
| | CACGCGTGGGTTCGAATCCCACCTTCGTCG |
| 741 | GGCCGGTTAGCTCAGTTGGTTAGAGCGTGCTTCAACTAATGCCAGGGTCGAG |
| | GTTTCGATCCCCGTACGGGCCT |
| 742 | GACGAGGTGGCCGAGTGGTTAAGGCGATGGACTTCAAATCCATTGTGCTCTG |
| | CACACGTGGGTTCGAATCCCATCCTCGTCG |
| 743 | GAGGCCTGGCCGAGTGGTTAAGGCGATGGACTTCAAATCCATTGTGCTCTGC |
| | ACGCGTGGGTTCGAATCCCATCCTCG |
| 744 | GCAGCGATGGCCGAGTGGTTAAGGCGTTGGACTTCAAATCCAATGGGGTCTC |
| | CCCGCGCAGGTTCGAACCCTGCTCGCTGCG |
| 745 | GTAGTCGTGGCCGAGTGGTTAAGGCGATGGACTTCAAATCCATTGGGGTTTC |
| | CCCGCGCAGGTTCGAATCCTGCCGACTACG |
| 746 | GTAGTCGTGGCCGAGTGGTTAAGGCGATGGACTTCAAATCCATTGGGGTCTC |
| | CCCGCGCAGGTTCGAATCCTGCCGACTACG |
| 747 | GTAGTCGTGGCCGAGTGGTTAAGGCGATGGACTTCAAATCCATTGGGGTTTC |
| | CCCGCGCAGGTTCGAATCCTGTCGGCTACG |
| 748 | GCCCAGCTAGCTCAGTCGGTAGAGCATAAGACTTTAAATCTCAGGGTTGTGG |
| | ATTCGTGCCCCATGCTGGGTG |
| 749 | CTGCAGCTAGCTCAGTCGGTAGAGCATGAGACTTTAAATCTCAGGGTCATGG |
| | GTTCGTGCCCCATGTTGGG |
| 750 | CCAGCATGTCTCAGTCGGTATAGTGTGAGACTTTAAATCTCAGGGTCGTGGG |
| | TTCAAGCCCCACATTGGG |
| 751 | GTCTAGCTAGATCAGTTGGTAGAGCATAAGACTTTAAATCTCAGGGTCATGG |
| | GTTTGAGCCCTACGTTGGGCG |
| 752 | GCCCAGCTAGCTCAGCCGGTAGAGCACAAGACTTTAAATCTCAGGGTCGTGG |
| | GTTTGAGCCCTGTGTTGAGCA |
| 753 | CCGAATAGCTTAGTTGATGAAGCGTGAGACTTTAAATCTCAGGGTAGTGGGT |
| | TCAAGCCCCACATTGGA |
| 754 | GCCTGGCTACCTCAGTTGGTAGAGCATGGGACTTTAAATCCCAGAGTCAGTG |
| | GGTTCAAGCCTCACATTGAGTG |
| 755 | GCCCGGCTAGCTCAGTCGGTAGAGCATGAGACCTTAAATCTCAGGGTCGTGG |
| | GTTCGAGCCCCACGTTGGGCG |
| 756 | GCCCGGCTAGCTCAGTCGGTAGAGCATGGGACTTTAAATCTCAGGGTCGTGG |
| | GTTCGAGCCCCACGTTGGGCG |
| 757 | GCCCGGCTAGCTCAGTCGATAGAGCATGAGACTTTAAATCTCAGGGTCGTGG |
| | GTTCGAGCCGCACGTTGGGCG |
| 758 | GCCCAGCTAGCTCAGTCGGTAGAGCATGAGACTTTAAATCTCAGGGTCATGG |
| | GTTTGAGCCCCACGTTTGGTG |
| 759 | GCCTGGCTAGCTCAGTCGGCAAAGCATGAGACTTTAAATCTCAGGGTCGTGG |
| | GCTCGAGCTCCATGTTGGGCG |
| 760 | GCCCGACTACCTCAGTCGGTGGAGCATGGGACTTTACATCCCAGGGTTGTGG |
| | GTTCGAGCCCCACATTGGGCA |
| 761 | CCCCGGCTGGCTCAGTCAGTAGATCATGAGACTTTAAATCTCAGGGTCGTGG |
| | GTTCACGCCCCACACTGGGCG |
| 762 | GCGCTAGTCAGTAGAGCATGAGACTTTAAATCTCAGGGTCGTGGGTTCGAGC |
| | CCCACATCGGGCG |
| 763 | GCCTGGATAGCTCAGTTGGTAGAGCATCAGACTTTAAATCTGAGGGTCCAGG |
| | GTTCAAGTCCCTGTTCAGGCA |
| 764 | GCCAGGATAGTTCAGGTGGTAGAGCATCAGACTTTAAACCTGAGGGTTCAGG |
| | GTTCAAGTCTCTGTTTGGGCG |
| 765 | ACCCAGATAGCTCAGTCAGTAGAGCATCAGACTTTAAATCTGAGGGTCCAAG |
| | GTTCATGTCCCTTTTTGGGTG |
| 766 | ACCTGGGTAGCTTAGTTGGTAGAGCATTGGACTTTAAATTTGAGGGCCCAGG |
| | TTTCAAGTCCCTGTTTGGGTG |
| 767 | GCCTGGGTAGCTCAGTCGGTAGAGCTATCAGACTTTAAGCCTGAGGATTCAG |
| | GGTTCAATCCCTTGCTGGGGCG |
| 768 | GATAGCTCAGTTGATAGAGCATCAGACTTTAAATCTGAGGGTCCAGGGTTCA |
| | TGTCCCTGTT |
| 769 | GTTGGGGTAACTCAGTTGGTAGAGTAGCAGACTTTACATCTGAGGGTCCAGG |
| | GTTTAAGTCCATGTCCAGGCA |
| 770 | GCCTGGATAGCTCAGTTGGTAGAGCATCAGACTTTAAATCTGAGGGTCCAGG |
| | GTTCAAGTCCCTGTTCAGGCG |
| 771 | GCCTGGATAGCTCAGTCGGTAGAGCATCAGACTTTAAATCTGAGGGTCCAGG |
| | GTTCAAGTCCCTGTTCAGGCG |
| 772 | GCCCGGATAGCTCAGTCGGTAGAGCATCAGACTTTAAATCTGAGGGTCCGGG |
| | GTTCAAGTCCCTGTTCGGGCG |
| 773 | GCCTGGGTAGCTCAGTCGGTAGAGCATCAGACTTTAAATCTGAGGGTCCAGG |
| | GTTCAAGTCCCTGTCCAGGCG |
| 774 | GCCTGGATAGCTCAGTTGGTAGAACATCAGACTTTAAATCTGACGGTGCAGG |
| | GTTCAAGTCCCTGTTCAGGCG |
| 775 | GCCCGGAGAGCTCAGTGGGTAGAGCATCAGACTTTAAATCTGAGGGTCCAGG |
| | GTTCAAGTCCTCGTTCGGGCA |
| 776 | ACCTGGGTAGCTCAGTAGGTAGAACATCAGACTTTAAATCTGAGGGTCTAGG |
| | GTTCAAGTCCCTGTCCAGGCG |
| 777 | GCCTGGATAGCTCCTTCGGTAGAGCATCATCAGACTTTAAATGTGAGGGTCC |
| | AGGGTTCAAGTTCCTGTTTGGGCG |
| 778 | GCCCAGCTAGCTCAGTCGGTAGAGCATAAGACTCTAAATCTCAGGGTTGTGG |
| | ATTCGTGCCCCATGCTGGGTG |
| 779 | CTGCAGCTAGCTCAGTCGGTAGAGCATGAGACTCTAAATCTCAGGGTCATGG |
| | GTTCGTGCCCCATGTTGGG |
| 780 | CCAGCATGTCTCAGTCGGTATAGTGTGAGACTCTAAATCTCAGGGTCGTGGG |
| | TTCAAGCCCCACATTGGG |
| 781 | GTCTAGCTAGATCAGTTGGTAGAGCATAAGACTCTAAATCTCAGGGTCATGG |
| | GTTTGAGCCCTACGTTGGGCG |
| 782 | GCCCAGCTAGCTCAGCCGGTAGAGCACAAGACTCTAAATCTCAGGGTCGTGG |
| | GTTTGAGCCCTGTGTTGAGCA |
| 783 | CCGAATAGCTTAGTTGATGAAGCGTGAGACTCTAAATCTCAGGGTAGTGGGT |
| | TCAAGCCCCACATTGGA |
| 784 | GCCTGGCTACCTCAGTTGGTAGAGCATGGGACTCTAAATCCCAGAGTCAGTG |
| | GGTTCAAGCCTCACATTGAGTG |
| 785 | GCCCGGCTAGCTCAGTCGGTAGAGCATGAGACCCTAAATCTCAGGGTCGTGG |
| | GTTCGAGCCCCACGTTGGGCG |
| 786 | GCCCGGCTAGCTCAGTCGGTAGAGCATGGGACTCTAAATCTCAGGGTCGTGG |
| | GTTCGAGCCCCACGTTGGGCG |
| 787 | GCCCGGCTAGCTCAGTCGATAGAGCATGAGACTCTAAATCTCAGGGTCGTGG |
| | GTTCGAGCCGCACGTTGGGCG |
| 788 | GCCCAGCTAGCTCAGTCGGTAGAGCATGAGACTCTAAATCTCAGGGTCATGG |
| | GTTTGAGCCCCACGTTTGGTG |
| 789 | GCCTGGCTAGCTCAGTCGGCAAAGCATGAGACTCTAAATCTCAGGGTCGTGG |
| | GCTCGAGCTCCATGTTGGGCG |
| 790 | GCCCGACTACCTCAGTCGGTGGAGCATGGGACTCTACATCCCAGGGTTGTGG |
| | GTTCGAGCCCCACATTGGGCA |
| 791 | CCCCGGCTGGCTCAGTCAGTAGATCATGAGACTCTAAATCTCAGGGTCGTGG |
| | GTTCACGCCCCACACTGGGCG |
| 792 | GCGCTAGTCAGTAGAGCATGAGACTCTAAATCTCAGGGTCGTGGGTTCGAGC |
| | CCCACATCGGGCG |
| 793 | GCCTGGATAGCTCAGTTGGTAGAGCATCAGACTCTAAATCTGAGGGTCCAGG |
| | GTTCAAGTCCCTGTTCAGGCA |
| 794 | GCCAGGATAGTTCAGGTGGTAGAGCATCAGACTCTAAACCTGAGGGTTCAGG |
| | GTTCAAGTCTCTGTTTGGGCG |
| 795 | ACCCAGATAGCTCAGTCAGTAGAGCATCAGACTCTAAATCTGAGGGTCCAAG |
| | GTTCATGTCCCTTTTTGGGTG |
| 796 | ACCTGGGTAGCTTAGTTGGTAGAGCATTGGACTCTAAATTTGAGGGCCCAGG |
| | TTTCAAGTCCCTGTTTGGGTG |
| 797 | GCCTGGGTAGCTCAGTCGGTAGAGCTATCAGACTCTAAGCCTGAGGATTCAG |
| | GGTTCAATCCCTTGCTGGGGCG |
| 798 | GATAGCTCAGTTGATAGAGCATCAGACTCTAAATCTGAGGGTCCAGGGTTCA |
| | TGTCCCTGTT |
| 799 | GTTGGGGTAACTCAGTTGGTAGAGTAGCAGACTCTACATCTGAGGGTCCAGG |
| | GTTTAAGTCCATGTCCAGGCA |
| 800 | GCCTGGATAGCTCAGTTGGTAGAGCATCAGACTCTAAATCTGAGGGTCCAGG |
| | GTTCAAGTCCCTGTTCAGGCG |
| 801 | GCCTGGATAGCTCAGTCGGTAGAGCATCAGACTCTAAATCTGAGGGTCCAGG |
| | GTTCAAGTCCCTGTTCAGGCG |
| 802 | GCCCGGATAGCTCAGTCGGTAGAGCATCAGACTCTAAATCTGAGGGTCCGGG |
| | GTTCAAGTCCCTGTTCGGGCG |
| 803 | GCCTGGGTAGCTCAGTCGGTAGAGCATCAGACTCTAAATCTGAGGGTCCAGG |
| | GTTCAAGTCCCTGTCCAGGCG |
| 804 | GCCTGGATAGCTCAGTTGGTAGAACATCAGACTCTAAATCTGACGGTGCAGG |
| | GTTCAAGTCCCTGTTCAGGCG |
| 805 | GCCCGGAGAGCTCAGTGGGTAGAGCATCAGACTCTAAATCTGAGGGTCCAGG |
| | GTTCAAGTCCTCGTTCGGGCA |
| 806 | ACCTGGGTAGCTCAGTAGGTAGAACATCAGACTCTAAATCTGAGGGTCTAGG |
| | GTTCAAGTCCCTGTCCAGGCG |
| 807 | GCCTGGATAGCTCCTTCGGTAGAGCATCATCAGACTCTAAATGTGAGGGTCC |
| | AGGGTTCAAGTTCCTGTTTGGGCG |
| 808 | GGCAGAATGGTGCAGCGGTTCAGCACCCAGGCTCTTCAGCCAGCTGTTGCCT |
| | GGGCTCAAATCCCAGCTCTGCCA |
| 809 | GGCTGTATAGCTCAGTGGTAGAGCATTTGACTTCAGAATCCTATACTCAGGG |
| | GAAGGAGAACTGGGGGTTTCTCAGTGGGTCAAAGGACTTGTAGTGGTAAATC |
| | AAAAGCAACTCTATAAGCTATGTAACAAACTTTAAAGTCATATGTAGCTGGG |
| | TTCAAATCCTGTTTCTGCCA |
| 810 | GGCTGTATAGCTCAGTGGTAGAGCATTTGACTTCAGCTTTAAAGTCATATGT |
| | AGCTGGGTTCAAATCCTGTTTCTGCCA |
| 811 | GGGGGCATAGCTCAGTGGTAGAGCATTTGACTTCAGATCAAGAGGTCCCTGG |
| | TTCAAATCCAGGTGCCCCCT |
| 812 | GGGGGTATAGCTCAGGGGTAGAGCATTTGACTTCAGATCAAGAGGTCCCTGG |
| | TTCAAATCCAGGTGCCCCCC |
| 813 | GGGGGTATAGCTTAGCGGTAGAGCATTTGACTTCAGATCAAGAGGTCCCCGG |
| | TTCAAATCCGGGTGCCCCCT |
| 814 | GGGGGTATAGCTTAGGGGTAGAGCATTTGACTTCAGATCAAAAGGTCCCTGG |
| | TTCAAATCCAGGTGCCCCTT |
| 815 | GGGGGTATAGCTCAGGGGTAGAGCATTTGACTTCAGATCAAGAGGTCCCCAG |
| | TTCAAATCTGGGTGCCCCCT |
| 816 | GGGGGTATAGCTCAGGGGTAGAGCATTTGACTTCAGATCAAGAAGTCCCCGG |
| | TTCAAATCCGGGTGCCCCCT |
| 817 | GGGGGTATAGCTCAGGGGTAGAGCATTTGACTTCAGATCAAGAGGTCTCTGG |
| | TTCAAATCCAGGTGCCCCCT |
| 818 | GGGGGTATAGCTCAGGGGTAGAGCACTTGACTTCAGATCAAGAAGTCCTTGG |
| | TTCAAATCCAGGTGCCCCCT |
| 819 | GGGGATATAGCTCAGGGGTAGAGCATTTGACTTCAGATCAAGAGGTCCCCGG |
| | TTCAAATCCGGGTGCCCCCC |
| 820 | GGGGGTATAGTTCAGGGGTAGAGCATTTGACTTCAGATCAAGAGGTCCCTGG |
| | TTCAAATCCAGGTGCCCCCT |
| 821 | GGGGGTATAGCTCAGGGGTAGAGCATTTGACTTCAAATCAAGAGGTCCCTGA |
| | TTCAAATCCAGGTGCCCCCT |
| 822 | GGGCGTATAGCTCAGGGGTAGAGCATTTGACTTCAGATCAAGAGGTCCCCAG |
| | TTCAAATCTGGGTGCCCCCT |
| 823 | GGGGGTATAGCTCACAGGTAGAGCATTTGACTTCAGATCAAGAGGTCCCCGG |
| | TTCAAATCTGGGTGCCCCCT |
| 824 | GGGCGTATAGCTCAGGGGTAGAGCATTTGACTTCAGATCAAGAGGTCCCCAG |
| | TTCAAATCTGGGTGCCCA |
| 825 | GGGGGTATAGCTCACAGGTAGAGCATTTGACTTCAGATCAAGAGGTCCCCGG |
| | TTCAAATCCGGTTACTCCCT |
| 826 | GGGGGTAGGGCTCAGGGATAGAGCATTTGACTTCAGATCAAGAGGTCCCCGG |
| | TTCGAATCTAGGTGCCCCCT |
| 827 | GGTATATCTCAGGGGGCAGAGCATTTGACTTCAGATCAAGAGGTCCCCGGTT |
| | GAAATCCGGGTGCT |
| 828 | GGGGGTATAGCTCAGGGGTAGAGCACTTGACTTCAGATCAAGAGGTCCCTGG |
| | TTCAAATCCAGGTGCCCCCT |
| 829 | GGGGGTATAGCTCAGTGGTAGAGCATTTGACTTCAGATCAAGAGGTCCCTGG |
| | TTCAAATCCGGGTGCCCCCT |
| 830 | GGGGGTATAGCTCAGTGGGTAGAGCATTTGACTTCAGATCAAGAGGTCCCCG |
| | GTTCAAATCCGGGTGCCCCCT |
| 831 | GGGGGTGTAGCTCAGTGGTAGAGCATTTGACTTCAGATCAAGAGGTCCCTGG |
| | TTCAAATCCAGGTGCCCCCT |
| 832 | GGGGGTATAGCTCAGGGGTAGAGCATTTGACTTCAGATCAAGAGGTCCCCGG |
| | TTCAAATCCGGGTGCCCCCT |
| 833 | GGGGGTATAGCTCAGGGGTAGAGCATTTGACTTCAGATCAAGAGGTCCCTGG |
| | TTCAAATCCAGGTGCCCCCT |
| 834 | GACCTCGTGGCGCAATGGTAGCGCGTCTGACTTCAGATCAGAAGGTTGCGTG |
| | TTCAAATCACGTCGGGGTCA |
| 835 | GACCTCGTGGCACAATGGTAGCACGTCTGACTTCAGATCAGAAGGTTGCGTG |
| | TTCAAATCACGTCGGGGTCA |
| 836 | GAAGCGGTGGCTCAATGGTAGAGCTTTCGACTTCAATTAAATCTTGGAAATT |
| | CCACGGAATAAGATTGCAATCGAAGGGTTGCAGGTTCAATTCCTGTCCGTTT |
| | CA |
| 837 | GAAGCGGTGGCTCAATGGTAGAGCTTTCGACTTCAAATCGAAGGGTTGCAGG |
| | TTCAATTCCTGTCCGTTTCA |
| 838 | GGCCTCATGGTGCAACAGTAGTGTGTCTGACTTCAGATCAGAAGGTTGTATG |
| | TTCAAATCACATAGGGGTCA |
| 839 | GACCTCGTGGTGAAATGGTAGCATGTTTGACTTCAAATCAGGAGGTTGTGTG |
| | TTCAAGTCACATCAGGGTCA |
| 840 | GACCTTGTGGCGCAATGGTAGCATGTTTGACTTCAAATCAGGAGGTTGTGTG |
| | TTCAAGTCACATCAGGGTCA |
| 841 | GACCTCGTGGCGCAACGGTAGCGCGTCTGACTTCAGATCAGAAGGCTGCGTG |
| | TTCGAATCACGCCGGGGTCA |
| 842 | GACCTTGTGGCTCAATGGTAGCGCATCTGACTTCAGATCAGGAGGTTGCACG |
| | TTCAAATCATGCCGGGGTCA |
| 843 | GACCTTGTGGCGCAACGGTAGCGCGTCTGACTTCAGATCAGAAGGTTGCGTG |
| | TTCAAATCACGTCGGGGTCA |
| 844 | GACCTCGTGGCGCAACGGTAGCGCGTCTGACTTCAGATCAGAAGGTTGCGTA |
| | TTCAAATCACGTCGGGGTCA |
| 845 | GACCTCGTGGCGCAACGGCAGCGCGTCTGACTTCACATTAGAAGGTTGCGTG |
| | TTCAAATCACGTCGGGGTCA |
| 846 | GACCTCATGGCGCAACGGTAGCGCGTCTGACTTCAGATCAGAAGGTTGCGTG |
| | TTCAAATCACATCGGGGTCA |
| 847 | GACCTCGTGGTGCAACGGTAGCGCGTATGATTTCAGATCAGAAGGTTGCGTG |
| | TTCAAATCACGTCGGGGTCA |
| 848 | GACCTCGTAGCGCAACGGTAGCGCGTCTGACTTCAGATCAGAAGGTTGCGTG |
| | TTCAAATCACGTCGGGGTCA |
| 849 | AGGGGTATAGCTCAATTGGCAGAGCGTCGGTCTTCAAAACCGAAGGTTGTAG |
| | GTTCGATTCCTACTGCCCCTGCCA |
| 850 | GACCTCATGGCGCAACGGTAGCGCGTCTGACTTCAGATCAGAAGGTTGCGTG |
| | TTCAAATCACGTCGGGGTCA |
| 851 | GACCTCGTGGCGCAACGGTAGCGCGTCTAACTTCAGATCAGAAGGTTGCGTG |
| | TTCAAATCACGTCGGGGTCA |
| 852 | ACGGGAGTAGCTCAGTTGGTAGAGCACCGGTCTTCAAAACCGGGTGTCGGGA |
| | GTTCGAGCCTCTCCTCCCGTG |
| 853 | GACCTCGTGGCGCAACGGTAGCGCGTCTGACTTCAGATCAGAAGGTTGCATG |
| | TTCAAATCACGTCGGGGTCA |
| 854 | GACTCCGTGGCGCAACGGTAGCGCGTCCGACTTCAGATCGGAAGGTTGCGTG |
| | TTCAAATCACGTCGGGGTCA |
| 855 | GACTCCGTGGCGCAACGGTAGCGCGTCTGACTTCAGATCAGAAGGTTGCGTG |
| | TTCAAATCACGTCGGGGTCA |
| 856 | GGCCTCGTGGCGCAACGGTAGCGCGTCTGACTCCAGATCAGAAGGTTGCGTG |
| | TTCAAATCACGTCGGGGTCA |
| 857 | GGCCTCGTGGCGCAACGGTAGCACGTCTGACTCCAGATCAGAAGGTTGCGTG |
| | TTCAAATCACGTCGGGGTCA |
| 858 | CGGCCTCGTGGCGCAACGGTAGCACGTCTGACTTCAGATCAGAAGGTTGCGT |
| | GTTCAAATCACGTCGGGGTCA |
| 859 | GGCCTCGTCGCGCAACGGTAGCGCGTCTGACTCCAGATCAGAAGGTTGCGTG |
| | TTCAAATCACGTCGGGGTCA |
| 860 | GGCCTCGTCGCGCAACGGTAGCGCGTCTGACTTCAGATCAGAAGGTTGCGTG |
| | TTCAAATCACGTCGGGGTCA |
| 861 | GGCCTCGTCGCGCAACGGTAGCACGTCTGACTCCAGATCAGAAGGTTGCGTG |
| | TTCAAATCACGTCGGGGTCA |
| 862 | GGCCTCGTCGCGCAACGGTAGCACGTCTGACTTCAGATCAGAAGGTTGCGTG |
| | TTCAAATCACGTCGGGGTCA |

In certain embodiments, the tRNA comprises, consists essentially of, or consists of a nucleotide sequence shown in any one of TABLEs 8-10. In certain embodiments, the tRNA comprises, consists essentially of, or consists of a nucleotide sequence having 80%, 85%, 86%, 87%, 88%, 89%, 90%, 91%, 92%, 93%, 94%, 95%, 96%, 97%, 98%, or 99% sequence identity to a nucleotide sequence in any one of TABLEs 8-10.

In certain embodiments, the tRNA comprises, consists essentially of, or consists of the nucleotide sequence of SEQ ID NO: 6. In certain embodiments, the tRNA comprises, consists essentially of, or consists of the nucleotide sequence of SEQ ID NO: 7. In certain embodiments, the tRNA comprises, consists essentially of, or consists of the nucleotide sequence of SEQ ID NO: 8. In certain embodiments, the tRNA comprises, consists essentially of, or consists of the nucleotide sequence of SEQ ID NO: 9. In certain embodiments, the tRNA comprises, consists essentially of, or consists of the nucleotide sequence of SEQ ID NO: 11. In certain embodiments, the tRNA comprises, consists essentially of, or consists of the nucleotide sequence of SEQ ID NO: 16. In certain embodiments, the tRNA comprises, consists essentially of, or consists of the nucleotide sequence of SEQ ID NO: 17. In certain embodiments, the tRNA comprises, consists essentially of, or consists of the nucleotide sequence of SEQ ID NO: 18. In certain embodiments, the tRNA comprises, consists essentially of, or consists of the nucleotide sequence of SEQ ID NO: 19. In certain embodiments, the tRNA comprises, consists essentially of, or consists of the nucleotide sequence of SEQ ID NO: 20. In certain embodiments, the tRNA comprises, consists essentially of, or consists of the nucleotide sequence of SEQ ID NO: 21. In certain embodiments, the tRNA comprises, consists essentially of, or consists of the nucleotide sequence of SEQ ID NO: 22. In certain embodiments, the tRNA comprises, consists essentially of, or consists of the nucleotide sequence of SEQ ID NO: 35. In certain embodiments, the tRNA comprises, consists essentially of, or consists of the nucleotide sequence of SEQ ID NO: 36. In certain embodiments, the tRNA comprises, consists essentially of, or consists of the nucleotide sequence of SEQ ID NO: 37. In certain embodiments, the tRNA comprises, consists essentially of, or consists of the nucleotide sequence of SEQ ID NO: 38. In certain embodiments, the tRNA comprises, consists essentially of, or consists of the nucleotide sequence of SEQ ID NO: 39. In certain embodiments, the tRNA comprises, consists essentially of, or consists of the nucleotide sequence of SEQ ID NO: 40. In certain embodiments, the tRNA comprises, consists essentially of, or consists of the nucleotide sequence of SEQ ID NO: 44. In certain embodiments, the tRNA comprises, consists essentially of, or consists of the nucleotide sequence of SEQ ID NO: 45. In certain embodiments, the tRNA comprises, consists essentially of, or consists of the nucleotide sequence of SEQ ID NO: 178. In certain embodiments, the tRNA comprises, consists essentially of, or consists of the nucleotide sequence of SEQ ID NO: 179. In certain embodiments, the tRNA comprises, consists essentially of, or consists of the nucleotide sequence of SEQ ID NO: 180. In certain embodiments, the tRNA comprises, consists essentially of, or consists of the nucleotide sequence of SEQ ID NO: 181. In certain embodiments, the tRNA comprises, consists essentially of, or consists of the nucleotide sequence of SEQ ID NO: 182. In certain embodiments, the tRNA comprises, consists essentially of, or consists of the nucleotide sequence of SEQ ID NO: 186. In certain embodiments, the tRNA comprises, consists essentially of, or consists of the nucleotide sequence of SEQ ID NO: 187.

In certain embodiments, the tRNA may comprise one or more mutations (e.g., nucleotide substitutions, deletions, or insertions) relative to a reference tRNA sequence (e.g., a tRNA disclosed herein). In certain embodiments, the tRNA may comprise, consist, or consist essentially of, a single mutation, or a combination of 2, 3, 4, 5, 6, 7, 8, 9, 10, 11, 12, 13, 14, 15 or more than 15 mutations. It is contemplated that the tRNA may comprise, consist, or consist essentially 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 mutations.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul (1993) J. MOL. EVOL. 36, 290-300; Altschul et al., (1997) NUCLEIC ACIDS RES. 25:3389-3402) are tailored for sequence similarity searching. For a discussion of basic issues in searching sequence databases see Altschul et al. (1994) NATURE GENETICS 6:119-129. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; —X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

It is contemplated that a tRNA may comprise on or more modifications. Exemplary modified tRNAs include: acylated tRNA; alkylated tRNA; a tRNA containing one or more bases other than adenine, cytosine, guanine, or uracil; a tRNA covalently modified by the attachment of a specific ligand or antigenic, fluorescent, affinity, reactive, spectral, or other probe moiety; a tRNA containing one or more ribose moieties that are methylated or otherwise modified; aa-tRNAs that are aminoacylated with an amino acid other than the 20 natural amino acids, including non-natural amino acids that function as a carrier for reagents, specific ligands, or as an antigenic, fluorescent, reactive, affinity, spectral, or other probe; or any combination of these compositions. Exemplary modified tRNA molecules are described in Soll et al. (1995) “tRNA: Structure, Biosynthesis, and Function,” ASM Press; El Yacoubi et al. (2012) ANNU. REV. GENET. 46:69-95; Grosjean et al. (1998) “Modification and Editing of RNA.” ASM Press; Hendrickson et al. (2004) ANNU. REV. BIOCHEM. 73:147-176, 2004; Ibba et al. (2000) ANNU. REV. BIOCHEM. 69:617-650; Johnson et al. (1995) COLD SPRING HARBOR SYMP. QUANT. BIOL. 60:71-82; Johnson et al. (1982) J. MOL. BIOL. 156:113-140; Crowley et al. (1994) CELL 78:61-71; Beier et al. (2001) NUCLEIC ACIDS RES. 29:4767-4782; Tones et al. (2014) TRENDS MOL. MED. 20:306-314; Bjork et al. (1987) ANNU. REV. BIOCHEM. 56:263-287; Schaffrath et al. (2017) RNA BIOL. 14(9):1209-1222; and Johansson et al. (2008) MOL. CELL. BIOL. 28(10):3301-12.

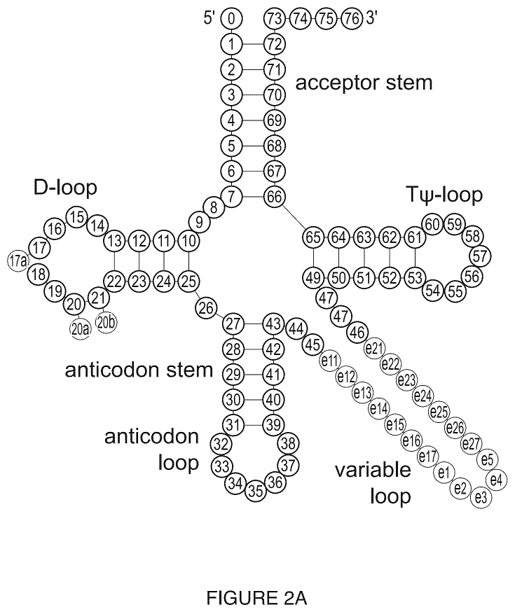

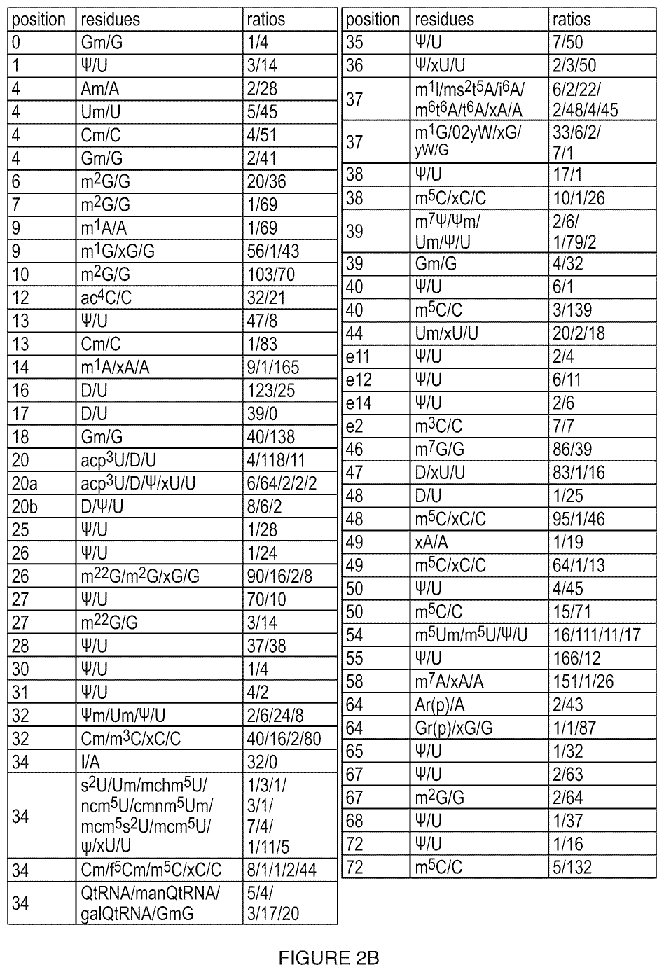

In certain embodiments, a tRNA comprises a naturally occurring nucleotide modification. Naturally occurring tRNAs contain a wide variety of post-transcriptionally modified nucleotides, which are described, for example, in Machnicka et al. (2014) RNA BIOLOGY 11(12): 1619-1629, and include one or more of the residues as shown in FIG. 2B. In certain embodiments, the tRNA comprises one or more of the residues selected from the group consisting of: 2′-O-methylguanosine or G at position 0; pseudouridine or U at position 1; 2′-O-methyladenosine, A, 2′-O-methyluridine, U, 2′-O-methylcytidine, C, 2′-O-methylguanosine, or G at position 4; N2-methylguanosine or G at position 6; N2-methylguanosine or G at position 7; 1-methyladenosine, A, 1-methylguanosine, G, or a modified G at position 9; N2-methylguanosine or G at position 10; N4-acetylcytidine or C at position 12; pseudouridine, U, 2′-O-methylcytidine, or C at position 13; 1-methyladenosine, A, or a modified A at position 14; dihydrouridine (D) or U at position 16; D or U at position 17; 2′-O-methylguanosine or G at position 18; 3-(3-amino-3-carboxypropyl)uridine, D, or U at position 20; 3-(3-amino-3-carboxypropyl)uridine, D, pseudouridine, U, or a modified U at position 20a; D, pseudouridine, or U at position 20b; pseudouridine or U at position 25; pseudouridine, U, N2,N2-dimethylguanosine, N2-methylguanosine, G, or a modified G at position 26; pseudouridine, U, N2,N2-dimethylguanosine, or G at position 27; pseudouridine or U at position 28; pseudouridine or U at position 30; pseudouridine or U at position 31; 2′-O-methylpseudouridine, 2′-O-methyluridine, pseudouridine, U, 2′-O-methylcytidine, 3-methylcytidine, C, or a modified C at position 32; inosine, A, 2-thiouridine, 2′-O-methyluridine, 5-(carboxyhydroxymethyl)uridine methyl ester, 5-carbamoylmethyluridine, 5-carboxymethylaminomethyl-2′-O-methyluridine, 5-methoxycarbonylmethyl-2-thiouridine, 5-methoxycarbonylmethyluridine, pseudouridine, U, a modified U, 2′-O-methylcytidine, 5-formyl-2′-O-methylcytidine, 5-methylcytidine, C, a modified C, queuosine, mannosyl-queuosine, galactosyl-queuosine, 2′-O-methylguanosine, or G at position 34; pseudouridine or U at position 35; pseudouridine, U, or a modified U at position 36; 1-methylinosine, 2-methylthio-N6-threonylcarbamoyladenosine, N6-isopentenyladenosine, N6-methyl-N6-threonylcarbamoyladenosine, N6-threonylcarbamoyladenosine, A, a modified A, 1-methylguanosine, peroxywybutosine, wybutosine, G, or a modified G at position 37; pseudouridine, U, 5-methylcytidine, C, or a modified C at position 38; 1-methylpseudouridine, 2′-O-methylpseudouridine, 2′-O-methyluridine, pseudouridine, U, 2′-O-methylguanosine, or G at position 39; pseudouridine, U, 5-methylcytidine, or C at position 40; 2′-O-methyluridine, U, or a modified U at position 44; pseudouridine or U at position e 11; pseudouridine or U at position el2; pseudouridine or U at position el4; 3-methylcytidine or C at position e2; 7-methylguanosine or G at position 46; D, U, or a modified U at position 47; D, U, 5-methylcytidine, C, or a modified C at position 48; A, a modified A, 5-methylcytidine, C, or a modified C at position 49; pseudouridine, U, 5-methylcytidine, or C at position 50; 5,2′-O-dimethyluridine, 5-methyluridine, pseudouridine, or U at position 54; pseudouridine or U at position 55; 1-methyladenosine, A, or a modified A at position 58; 2′-O-ribosyladenosine (phosphate), A, 2′-O-ribosylguanosine (phosphate), G, or a modified G at position 64; pseudouridine or U at position 65; pseudouridine, U, N2-methylguanosine, or G at position 67; pseudouridine or U at position 68; and, pseudouridine, U, 5-methylcytidine, or C at position 72. A, C, G, and U, refer to unmodified adenine, cytosine, guanine, and uracil, respectively. The numbering of the residues is based on the tRNA numbering system described in Steinberg et al., (1993) NUCLEIC ACIDS RES. 21:3011-15.

In certain embodiments, the tRNA comprises one or more nucleotide modifications selected from 5-methyl uridine, 5-carbamoylmethyluridine, 5-carbamoyl-methyl-2-O-methyluridine, 5-methoxy-carbonylmethyluridine, 5-methoxycarbonylmethyl-2-thiouridine, pseudouridine, dihydrouridine, 1-methyladenosine, and inosine.

### II. Methods of Making tRNAs

It is contemplated the tRNA molecules (e.g., suppressor tRNAs) useful in the practice of the invention can be produced by methods known in the art, including extracellular production by synthetic chemical methods, intracellular production by recombinant DNA methods, or purification from natural sources.

For example, DNA molecules encoding tRNAs can be synthesized chemically or by recombinant DNA methodologies. For example, the sequences of the tRNAs can be synthesized or cloned from libraries by conventional hybridization techniques or polymerase chain reaction (PCR) techniques, using the appropriate synthetic nucleic acid primers. The resulting DNA molecules encoding the tRNAs can be ligated to other appropriate nucleotide sequences, including, for example, expression control sequences to produce conventional gene expression constructs (i.e., expression vectors) encoding the tRNAs. Production of defined gene constructs is within routine skill in the art. Nucleic acids encoding desired tRNAs can be incorporated (ligated) into expression vectors, such as the expression vectors described in the following section, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the tRNAs. Specific expression and purification conditions will vary depending upon the expression system employed.

Alternatively, tRNAs can be chemically synthesized or purified from natural sources by methods known in art. When a tRNA is aminoacylated prior to introduction into the cell or administration to the subject, the tRNA may be aminoacylated with a desired amino acid by any method known in the art, including chemical or enzymatic aminoacylation.

### III. Expression Vectors

The tRNAs of interest may be expressed in a cell of interest by incorporating a gene encoding a tRNA of interest into an appropriate expression vector. As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain embodiments, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is a RNA virus vector. Exemplary RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the tRNA. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

tRNA genes preferably have strong promoters that are active in a variety of cell types. The promoters for eukaryotic tRNA genes typically are present within the structural sequences encoding the tRNA molecule itself. Although there are elements which regulate transcriptional activity within the 5′ upstream region, the length of an active transcriptional unit may be considerably less than 500 base pairs.

Additional exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein.

In certain embodiments, an expression vector comprises a tRNA coding sequence that encodes a tRNA that comprises, consists essentially of, or consists of a nucleotide sequence shown in TABLE 2 or TABLE 3. In certain embodiments, an expression vector comprises a tRNA coding sequence that encodes a tRNA that comprises, consists essentially of, or consists of a nucleotide sequence having 80%, 85%, 86%, 87%, 88%, 89%, 90%, 91%, 92%, 93%, 94%, 95%, 96%, 97%, 98%, or 99% to a nucleotide sequence shown in TABLE 2 or TABLE 3.

In certain embodiments, in addition to a tRNA coding sequence, the expression vector comprises a nucleotide sequence corresponding to a genomic DNA sequence flanking a wild-type tRNA gene (i.e., a DNA sequence from the same genome as a wild-type tRNA gene and which is 5′ or 3′ to the wild-type tRNA gene in the genome, e.g., immediately 5′ or 3′ to the wild-type tRNA gene in the genome). In certain embodiments, in addition to a tRNA coding sequence, the expression vector comprises a nucleotide sequence corresponding to an exogenous promoter.

In certain embodiments, the expression vector comprises a nucleotide sequence shown in TABLE 4. In certain embodiments, the expression vector comprises a nucleotide sequence having 80%, 85%, 86%, 87%, 88%, 89%, 90%, 91%, 92%, 93%, 94%, 95%, 96%, 97%, 98%, or 99% sequence identity to a nucleotide sequence shown in TABLE 4. In certain embodiments, in the expression vector, the nucleotide sequence set forth in TABLE 4 is operably linked to the nucleotide sequence encoding the tRNA. In certain embodiments, in the expression vector, the nucleotide sequence set forth in TABLE 4 is 5′ or 3′ (e.g., immediately 5′ or immediately 3) to the nucleotide sequence encoding the tRNA. In certain embodiments, the expression vector comprises a nucleotide sequence selected from SEQ ID NOs: 869-888, or a nucleotide sequence having 80%, 85%, 86%, 87%, 88%, 89%, 90%, 91%, 92%, 93%, 94%, 95%, 96%, 97%, 98%, or 99% sequence identity to a sequence selected from SEQ ID NOs: 869-888.

In certain embodiments, the expression vector is an expression vector described in Example 8 or Example 9 herein.

TABLE 4

| | Location | |
|---|---|---|
| | (Relative | |
| SEQ ID NO | to tRNA) | Nucleotide Sequence |
| 26 | 5′ | CTACCCAGAGGCAGGCGGGAGACTCCCCCGAGCGTCCAATAAGAGC |
| | | GCCGCCAATGGAGCCGCCCGCCCGCGGGGGTGCAGAGGGACTTCCG |
| | | GGTGAGGTCCTCCGCTACTTCCCTCCCCACGGAAAAGATAGACCAG |
| | | TCTGACGCGAGCCTGAAGGCGGCTACACGCTTTAAGCTAAGTAAAG |
| | | GCACCTTCTCGCTGGC |
| 27 | 3′ | ACTTGTATGTTGTTTTTATCTGTCAGTTTGTTAATCCCAAGATTCC |
| | | CTTTGGAAATAAAGCGAAATTGACCGTAGTGGTTATGACCAACTTC |
| | | TAGTCTAAACTTAATTCTTGGAACTCAAGGATCTGAGCAAACAACT |
| | | GTCAGGGTGACACATTGCTTAAACGGTGACAGCGGTCGAGAGCCTT |
| | | GTCCCGGATGGAGAGT |
| 32 | 3′ | ACTTGTATGTTGTTTTTATCTGTCAGTTTGTTAATCCCAAGATTCC |
| | | CTTTGGAAATAAAGCGAAATTGACCGTAGTGGTTATGACCAACTTC |
| | | TAGTCTAAACTT |
| 33 | 5′ | GAGGGCCTATTTCCCATGATTCCTTCATATTTGCATATACGATACA |
| | | AGGCTGTTAGAGAGATAATTAGAATTAATTTGACTGTAAACACAAA |
| | | GATATTAGTACAAAATACGTGACGTAGAAAGTAATAATTTCTTGGG |
| | | TAGTTTGCAGTTTTAAAATTATGTTTTAAAATGGACTATCATATGC |
| | | TTACCGTAACTTGAAAGTATTTCGATTTCTTGGCTTTATATATCTT |
| | | GTGGAAAGGACGGGCGGAGGAAGGCACCTTCTCGCTGGC |
| 34 | 3′ | ACTTGTATGTTGTTTTTATCTGTCAGTTTGTTAATCCCAAGATTCC |
| 173 | 5′ | GATCACCGGAAGAGGTGACAGACACCTCGGGGCCCATGAACGTTTG |
| | | GAATTCGTAAGGACATGAGAATCTCGGTGGTTCCGTGTCTGCCCGC |
| | | CATCGCGGCCACCGGCCACGGGCCCAAGCCAAGTGTAGCGAAGCTT |
| | | AGAAAAGGTTGCCCAACGTCATGTGGCTTGAGAAGGCTGCCGGGCG |
| | | CCTTAAGCCGCCAGCA |
| 174 | 3′ | CACTGAACCTTTTTTTGGCCTTAGAATCCCTGTTTTGGGGCCTGCA |
| | | GGAAGTAGCAACCAACCCGAGCCTCCGCAGGGAATGCACTGACCTG |
| | | TAGAATGGACGTTCAGCTTCCCTCCCTGTGTCTCAACACGATTACA |
| | | TTTCAGGAACAGCCTGGGCTGGGAGGCACTGCGCACGCGCGCCGAG |
| | | TCGGGCGGAAAAATAA |
| 869 | 5′ | CCAAAACATCTTTTACTGTAGTATCTACTTACCATACTACCCAAGA |
| | | ATGGCACACTGCTCACATCTTCAAAAGCTTAAACCAAGAGCACTAC |
| | | ACAGGTGC |
| 870 | 5′ | TGTGTGTCGGGGCCGGTACCCTGCTTCCGGTTCCCGCACGCATTCC |
| | | CGGATTGCAGTGCGGACCCCTTCTGTAAGCGCGCGATAAAGCGCGG |
| | | TTTTGGAA |
| 871 | 5′ | TCATGTCATATAAGTAGAACCATACAATATATATATAAAATCCAGG |
| | | TTAATAGCCAATCTTACAACATTTCTCATATTTTTTGCAGTTGCTA |
| | | AGCCATGG |
| 872 | 5′ | ACATTACAATACATATCAACATATCACCATAATTAAATTGCAAGTC |
| | | TTCGTCAAAAGCAAGCCTTAAAGGAGTATCCCAAAAACACATTTTC |
| | | CCCAGAAG |
| 873 | 5′ | AGACCTTTAGAGCGTGGTTAAACCCATATGTTGGGATTTATGCTGC |
| | | TTTTATGGTAGCAATACCCTATATTAAGATTTGAAGTAGACCCGGA |
| | | AAGTTAGT |
| 874 | 5′ | GTTCATGAAAGAATAAATAAATGTTTAAAAAAAAAAAAAACTGAGG |
| | | TAAATTTCTATATTCTTTCATAAAAGCAGTTTAAAGACGAACGTTT |
| | | TTCGAGGT |
| 875 | 5′ | GCTGGGTCTCGGTGACACTGACGACGGGAGGCGCGGTCGGAAGAGC |
| | | GCGGGGCCGTCGCCTCTGGCTTAACATAGCAGATGCGCTGAGACTC |
| | | CAACAGGT |
| 876 | 5′ | CAGTGGCGGCGAAAACTCTCTGCGTTCTGGAGGGAGGGTGCGGGCA |
| | | GGAGGAGGTAGAGGATGCCTTGTAAGCGGAGCAAAAACAAGGTTCA |
| | | ACGTCTGC |
| 877 | 5′ | CAAATCACTTGCCTCTCGGCGCGAGACCGCGATGCGCGGGGGCGGG |
| | | AGCGTGATGATGGCATCGCGTAAGGAGAGGGTGTGAGAAGCCGGAT |
| | | CCTGTGGT |
| 878 | 5′ | CCCTGTGTCCGAAGAGGTCTGCGTTGCGACTTACGTGGTAGTGCTT |
| | | GGAAGGTGCGGAGTAGATGAGAGATAAGTGAATGTGGACAAACCTG |
| | | TCACGTAG |
| 879 | 5′ | GAGCGGAGCTCAGAGGGTGCGCGCTCCGCCCTTTCGCGGGCCTGGC |
| | | ATGAGCGCAGTGGTTGTTACACTAAAGTGTCTCCGCCTGTCGAATA |
| | | TTCTCGTG |
| 880 | 5′ | GTGTCACTGGTTTCAAATCAACCTCAATTTTTTTGGAGACGTGAGT |
| | | GCTGAGCATTTTTTCTTCAGTGAAGTGACTTGGCAGCCAAAATCGC |
| | | CAACGCCC |
| 881 | 5′ | TCCTGGCATGTCCCGCCCAAGTCCCTTAGCCCCGCTCCCCAACCCT |
| | | GCCCCATTCCCACTCTAGTACCCGTAAGCTACAAGACGCCGCCGTT |
| | | CGTCGGGT |
| 882 | 5′ | TGCTCAGTCGTCCTGCCGGGCGGGCCCTGAGGTTGCAAGGGACGGA |
| | | GGAAGTTTCGTGCGTGCGCCCTTCCTATAGCGCCCAGTAGAACTGA |
| | | CAGTACCT |
| 883 | 5′ | TCCTCGGATTACGCATGCTCAGTGCAATCTTCGGTTGCCTGGACTA |
| | | GCGCTCCGGTTTTTCTGTGCTGAACCTCAGGGGACGCCGACACACG |
| | | TACACGTC |
| 884 | 5′ | GATAATTTCCTGAAAGAAAAGATCAATTCGATGTTACCAAATCTGG |
| | | GATATCCAGAAAAATTTTCTTCTTCTCCTAGGAGAAAAACTATCAA |
| | | ATGTCAGG |
| 885 | 5′ | TCTCTCACGGCAAACTGTTGCAGACTGTAGAGACGCTATGCCAAGA |
| | | ATCTTTTACTTAAAAGCAGGAATAGATTCAATAGGCAACTTCACTG |
| | | CACATGTA |
| 886 | 5′ | CAACCTCCCCTTCTCAAGGAGCAGGTGGATTGGTCCCGAGCTAGCT |
| | | GGTGGGCGGAGGTGACGTTTTTATAAGTTGCTCAAGAGACGGTAAC |
| | | AACCGACG |
| 887 | 5′ | GTGGAACTTCCACTGAATTACTCTTTTCGCATGTAAGATCACTGAA |
| | | CCGTGATAATCATTGATCCTATTTGTAGAACTGTATGAAACAGTTC |
| | | CCTAAGGA |
| 888 | 5′ | TCGCTCAACAGGCGGCCAGGGTGCGAGCAGTGAAGCTGCGGCACGC |
| | | CGGAGCGTTTAATGGCCATCAAATTGGCCTCTCTAGGAGGTAGCTG |
| | | CAGCCGGA |
| 895 | 5′ | AAAGGCACCTTCTCGCTGGC |
| 896 | 3′ | ACTTGTATGTTGTTTTT |
| 897 | 5′ | TCTCGCTGGC |
| 900 | 5′ | AGCGCTCCGGTTTTTCTGTGCTGAACCTCAGGGGACGCCGACACAC |
| | | GTACACGTC |

### Adeno-Associated Virus (AAV) Vectors

In certain embodiments, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the present invention can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain embodiments, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Pat. No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh.8, rh.10, rh.39, rh.43, and rh.74, are also contemplated in the instant invention. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes Ela, Elb55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any adenoviral type may be used, and a person of skill in the art will be able to identify AAV and adenoviral types suitable for the production of their desired recombinant AAV vector (rAAV). AAV particles may be purified, for example by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are suitable for use in the method of the present invention.

Non-limiting examples of AAV vectors include pAAV-MCS (Agilent Technologies), pAAVK-EF1α-MCS (System Bio Catalog #AAV502A-1), pAAVK-EF1α-MCS1-CMV-MCS2 (System Bio Catalog #AAV503A-1), pAAV-ZsGreenl (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVSl_Puro_PGK1_3 xFLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified to be suitable for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

In certain embodiments, the expression vector is an AAV vector capable of targeting the nervous system, e.g., the central nervous system, in a subject, e.g., a human subject. Exemplary AAV vectors that can target the nervous system include the AAV9 variants AAV-PHP.B (See, e.g., Deverman et al. (2016) NAT. BIOTECHNOL. 34(2):204-209), AAV-AS (See, e.g., Choudhury et al. (2016) MOL. THER. 24:726-35), and AAV-PHP.eB (See, e.g., Chan et al. (2017) NAT. NEUROSCI. 20:1172-79). Additional exemplary AAV-based strategies for targeting the nervous system are described in Bedrook et al. (2018) ANNU REV NEUROSCI. 41:323-348. In certain embodiments, the AAV vector is an AAV-PHP.eB vector.

### Lentivirus Vectors

In certain embodiments, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also contemplated to be useful in the practice of the invention.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69(4):2101-09). The packaging signal may be a minimal packaging signal (also referred to as the psi [Ψ] sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector of the invention comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65: 1053; and Cullen et al., (1991) CELL 58: 423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors of the invention lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors of the invention lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the invention, includes an ideal polyadenylation sequence (e.g., AATAAA, ATTAAA AGTAAA), a bovine growth hormone polyadenylation sequence (BGHpA), a rabbit 0-globin polyadenylation sequence (rOgpA), or another suitable heterologous or endogenous polyadenylation sequence known in the art.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. SCI., USA, 99:16433; and Zhan et al., 2001, HUM. GENET., 109:471). In certain embodiments, the retroviral vector comprises an insulator element in one or both LTRs or elsewhere in the region of the vector that integrates into the cellular genome. Suitable insulators for use in the invention include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a β-globin locus, such as chicken HS4.

Non-limiting examples of lentiviral vectors include pLVX-EFlalpha-AcGFP1-C1 (Clontech Catalog #631984), pLVX-EFlalpha-IRES-mCherry (Clontech Catalog #631987), pLVX-Puro (Clontech Catalog #632159), pLVX-IRES-Puro (Clontech Catalog #632186), pLenti6IV5-DEST™ (Thermo Fisher), pLenti6.21V5-DEST™ (Thermo Fisher), pLKO.1 (Plasmid #10878 at Addgene), pLKO.3G (Plasmid #14748 at Addgene), pSico (Plasmid #11578 at Addgene), pUM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionll (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

### Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 1 1, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serogroup or serotype. Adenoviral serotypes 1 through 51 are available from the American Type Culture Collection (ATCC, Manassas, Virginia). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLoS PATHOG. 5(7): e1000503. A gorilla adenovirus can be used as the source of the adenoviral genome for the adenoviral vector. Gorilla adenoviruses and adenoviral vectors are described in, e.g., PCT Publication Nos. WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

Preferably, the adenoviral vector is replication-deficient, such that the replication-deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95: 965-976, Chen et al. (1997) PROC. NATL. ACAD. SCI. USA 94: 1645-1650, and Kochanek et al. (1999) Hum. GENE THER. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. VIROL. 71: 9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Patent Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### Viral Vector Production

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or tRNAs can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

In certain embodiments, producer cells may be directly administered to a subject, however, in other embodiments, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### IV. Pharmaceutical Compositions

For therapeutic use, a tRNA and/or expression vector preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29). In certain embodiments, the composition does not comprise (or is substantially free of, for example, the composition comprises less than 5%, 4%, 3%, 2%, 1%, 0.5% or 0.1% of) a nanoparticle or an aminolipid delivery compound, e.g., as described in U.S. Patent Publication No. 2017/0354672. In certain embodiments, the tRNA or expression vector introduced into the cell or administered to the subject is not conjugated to or associated with another moiety, e.g., a carrier particle, e.g., an aminolipid particle. As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing a tRNA and/or expression vector disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a tRNA and/or expression vector is administered intrathecally. In certain embodiments, a tRNA and/or expression vector is administered by injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

In general, any method of delivering a nucleic acid molecule can be adapted for use with a tRNA (see e.g., Akhtar et al. (1992) TRENDS CELL. BIOL. 2(5):139-144 and PCT Publication No. WO94/02595). The tRNA can be modified or alternatively delivered using a drug delivery system to prevent the rapid degradation of the tRNA by endo- and exo-nucleases in vivo. tRNA molecules can be modified by chemical conjugation to lipophilic groups such as cholesterol to enhance cellular uptake and prevent degradation. tRNA molecules can also be conjugated to or otherwise associated with an aptamer. A tRNA can also be delivered using drug delivery systems such as a nanoparticle, a dendrimer, a polymer, liposomes, or a cationic delivery system. Positively charged cationic delivery systems facilitate binding of a tRNA molecule (negatively charged) and also enhance interactions at the negatively charged cell membrane to permit efficient uptake of a tRNA by the cell. Cationic lipids, dendrimers, or polymers can either be bound to the RNA, e.g., tRNA, or induced to form a vesicle or micelle (see e.g., Kim et al. (2008) JOURNAL OF CONTROLLED RELEASE 129(2):107-116) that encases the RNA. Methods for making and administering cationic-RNA complexes are well within the abilities of one skilled in the art (see, e.g., Sorensen et al. (2003) J. MOL. BIOL 327:761-766; Verma et al. (2003) CLIN. CANCER RES. 9:1291-1300; Arnold et al. (2007) J. HYPERTENS. 25:197-205). Some non-limiting examples of drug delivery systems useful for systemic delivery of RNAs, e.g., tRNAs include DOTAP (Sorensen et al. (2003) supra; Verma et al. (2003), supra), Oligofectamine, solid nucleic acid lipid particles (Zimmermann et al. (2006) NATURE 441:111-114), cardiolipin (Chien et al. (2005) CANCER GENE ThER. 12:321-328; Pal et al. (2005) INT J. ONCOL. 26:1087-1091), polyethyleneimine (Bonnet et al. (2008) PHARM. RES. 25(12):2972-82; Aigner (2006) J. BIOMED. BIOTECHNOL. 71659), Arg-Gly-Asp (RGD) peptides (Liu (2006) MOL. PHARM. 3:472-487), and polyamidoamines (Tomalia et al. (2007) BIOCHEM. SOC. TRANS. 35:61-67; Yoo et al. (1999) PHARM. RES. 16:1799-1804). In certain embodiments, a tRNA forms a complex with cyclodextrin for systemic administration. Methods for administration and pharmaceutical compositions of RNAs and cyclodextrins can be found in U.S. Pat. No. 7,427,605.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, a tRNA and/or expression vector, is in the range of 0.1 mg/kg to 100 mg/kg, e.g., 1 mg/kg to 100 mg/kg, 1 mg/kg to 10 mg/kg. In certain embodiments, a therapeutically effective amount of a viral expression vector is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life, and the disease being treated. Exemplary dosing frequencies are once per day, once per week and once every two weeks. A preferred route of administration is parenteral, e.g., intravenous infusion. In certain embodiments, a polypeptide and/or multimeric protein is lyophilized, and then reconstituted in buffered saline, at the time of administration.

In certain embodiments, the tRNA or expression vector is not conjugated to or associated with another moiety, e.g., a carrier particle, e.g., an aminolipid particle. In certain embodiments, the tRNA or expression vector is introduced into the cell or administered to subject in a dosage form lacking a nanoparticle. In certain embodiments, the tRNA or expression vector is introduced into the cell or administered to subject in a dosage form lacking an aminolipid delivery compound, e.g., as described in U.S. Patent Publication No. 2017/0354672.

### V. Therapeutic Uses

The compositions and methods disclosed herein can be used to treat a premature termination codon (PTC)-mediated disorder in a subject. As used herein, the term “PTC-mediated disorder” refers to a disorder that is mediated, enhanced, exacerbated, or otherwise facilitated by or associated with a PTC in a gene.

The invention provides a method of treating a PTC-mediated disorder in a subject in need thereof. The method comprises administering to the subject an effective amount of a tRNA and/or expression vector, e.g., a tRNA and/or expression vector disclosed herein, either alone or in a combination with another therapeutic agent to treat the PTC-mediated disorder in the subject.

In certain embodiments, the premature termination codon-mediated disorder is a disorder listed in TABLE 5 below, and/or the gene with a premature termination codon is a gene listed in the corresponding row of TABLE 5 below.

TABLE 5

| Gene | Disorder |
|---|---|
| SCN1A | Dravet Syndrome; Genetic Epilepsy with Febrile Seizures |
| | (GEFS) |
| KCNQ2 | Benign Familial Infantile Epilepsy (BFIE); Early Infantile |
| | Epileptic Encephalopathy (EIEE) |
| SCN2A | Benign Familial Infantile Epilepsy (BFIE); Early Infantile |
| | Epileptic Encephalopathy (EIEE) |
| CDKL5 | Early Infantile Epileptic Encephalopathy (EIEE); Lennox- |
| | Gastaut Syndrome; CDKL5 deficiency disorder |
| MECP2 | Rett Syndrome; PPM-X Syndrome |
| STXBP1 | Early Infantile Epileptic Encephalopathy (EIEE); Ohtahara |
| | Syndrome; Dravet Syndrome |
| SCN8A | Benign Familial Infantile Epilepsy (BFIE); Early Infantile |
| | Epileptic Encephalopathy (EIEE) |
| CACNA1A | Episodic Ataxia; Hemiplegic Migraine |
| SLC2A1 | Iditiopathic Generalized Epilepsy |
| FOXG1 | FOXG1 Syndrome |
| PCDH19 | Early Infantile Epileptic Encephalopathy (EIEE) |
| GRIN2B | Early Infantile Epileptic Encephalopathy (EIEE) |
| DEPDC5 | Familial Focal Epilepsy with Variable Foci (FFEVF) |
| GRIN2A | Early Infantile Epileptic Encephalopathy (EIEE); Lennox- |
| | Gastaut Syndrome |
| CHD2 | Childhood-onset epileptic encephalopathy |
| SCN9A | Congenital insensitivity to pain, etc |
| SYNGAP1 | SYNGAP1-related intellectual disability |
| ALDH7A1 | Pyridoxine-dependent epilepsy |
| GRIN1 | Early Infantile Epileptic Encephalopathy (EIEE); Lennox- |
| | Gastaut Syndrome |
| TBC1D24 | Early Infantile Epileptic Encephalopathy (EIEE); Familial |
| | Infantile Myoclonic Epilepsy (FIME) |
| SLC6A1 | Myoclonic Astatic Epilepsy |
| DNM1 | Early Infantile Epileptic Encephalopathy (EIEE) |
| ARX | Early Infantile Epileptic Encephalopathy (EIEE); X-linked |
| | Intellectual Disability |
| KCNB1 | Early Infantile Epileptic Encephalopathy (EIEE) |
| KCNA1 | Partial Epilepsy and Episodic Ataxia |
| GABRG2 | Genetic Epilepsy with Febrile Seizures (GEFS); Early |
| | Infantile Epileptic Encephalopathy (EIEE); Febrile seizures |
| WWOX | Early Infantile Epileptic Encephalopathy (EIEE) |
| GABRB3 | Early Infantile Epileptic Encephalopathy (EIEE); Lennox- |
| | Gastaut Syndrome |
| SZT2 | Early Infantile Epileptic Encephalopathy (EIEE) |
| LGI1 | Autosomal Dominant Partial Epilepsy with Auditory |
| | Features (ADPEAF) |
| PNPO | PNPO-Deficiency |
| SCN1B | Genetic Epilepsy with Febrile Seizures (GEFS); Early |
| | Infantile Epileptic Encephalopathy (EIEE) |
| UBA5 | Early Infantile Epileptic Encephalopathy (EIEE) |
| KCTD7 | Progressive Myoclonus Epilepsy |
| SCARB2 | Action Myoclonus - Renal Failure (AMRF); Progressive |
| | Myoclonic Epilepsy |
| SLC13A5 | Early Infantile Epileptic Encephalopathy (EIEE) |
| CSTB | Progressive Myoclonic Epilepsy |
| EPM2A | Progressive Myoclonic Epilepsy |
| PRRT2 | Benign Familial Infantile Seizures (BFIS) |
| NHLRC1 | Progressive Myoclonic Epilepsy |
| SLC25A22 | Early Infantile Epileptic Encephalopathy (EIEE) |
| PRRT2 | Benign Familial Infantile Seizures (BFIS) |
| ALG13 | Early Infantile Epileptic Encephalopathy (EIEE) |

In certain embodiments, the premature termination codon-mediated disorder is a disorder listed in TABLE 6 below, and/or the gene with a premature termination codon is a gene listed in the corresponding row of TABLE 6 below.

TABLE 6

| Gene | Disorder |
|---|---|
| β-globin | β-thalassemia |
| CHM | Choroideremia |
| CFTR | Cystic Fibrosis |
| dystrophin | Duchenne Muscular Dystrophy |
| α-L-iduronidase | Hurler Syndrome |
| KIF1A | KIF1A |
| FBN1 | Marfan Syndrome |
| ARSB | Maroteaux-Lamy Syndrome |
| SMPD1 | Niemann Pick Disease |
| NAGLU | Sanfilippo Syndrome |
| DHCR7 | Smith-Lemli-Opitz Syndrome |
| SCN5A | Brugada Syndrome |
| KCNH2 (hERG) | Long QT Syndrome type 2 |
| KCNQ1 | Long QT Syndrome type 1 |
| TTN | Dilated Cardiomyopathy |
| MYBPC3 | Familial Hypertrophic |
| | Cardiomyopathy |
| LMNA | Dilated Cardiomyopathy |
| | (sometimes Emery-Dreifuss |
| | Muscular Dystrophy) |
| PKP2 | Familial Arrythmogenic Right |
| | Ventricular Dysplasia |
| PLN | Familial Isolated Dilated |
| | Cardiomyopathy |
| TSC1/2 | Tuberous Sclerosis |
| LDLR | Familial Hypercholesterolemia |
| SMN1 | Spinal Muscular Atrophy |

In certain embodiments, the PTC-mediated disorder is an epilepsy (e.g., Dravet syndrome), wherein the method reduces seizure frequency, seizure severity, and/or cognitive impairment in the subject. For example, in certain embodiments, the method reduces seizure frequency in the subject by at least 10%, 20%, 30%, 40%, 50%, 60%, 70%, 80%, 90% or 100% over the period of, e.g., a day, a week, or a month. In certain embodiments, the method reduces seizure frequency by 50% over the period of, e.g., a day, a week, or a month.

In certain embodiments, the PTC-mediated disorder is Dravet and/or the gene with a premature termination codon is SCN1A. In certain embodiments, a premature termination codon in the SCN1A gene is caused by a mutation, or a combination of mutations, selected from c.5745C>G, c.5713G>T, c.5701C>T, c.5677C>T, c.5641C>T, c.5629C>T, c.5623C>T, c.5503A>T, c.5473G>T, c.5437G>T, c.5428C>T, c.5403G>A, c.5402G>A, c.5383G>T, c.5371G>T, c.5049T>G, c.4921G>T, c.4900C>T, c.4873C>T, c.4779de1, c.4778G>A, c.4774G>T, c.4761T>G, c.4648G>T, c.4540C>T, c.4516A>T, c.4514C>A, c.4508T>G, c.4488C>G, c.4471G>T, c.4300A>T, c.4269G>A, c.4268G>A, c.4233T>A, c.4222G>T, c.4191G>A, c.4190G>A, c.4186C>T, c.4159A>T, c.4155C>A, c.3964de1, c.3952C>T, c.3825G>A, c.3824G>A, c.3819G>A, c.3818G>A, c.3795T>A, c.3789T>G, c.3779G>A, c.3750C>G, c.3724G>T, c.3700C>T, c.3697C>T, c.3657dup, c.3624G>A, c.3604C>T, c.3582G>A, c.3578G>A, c.3574C>T, c.3463C>T, c.3454de1, c.3424G>T, c.3422C>A, c.3406G>T, c.3328G>T, c.3273C>A, c.3262G>T, c.3073C>T, c.3060T>A, c.2844T>A, c.2749C>T, c.2695C>T, c.2645T>A, c.2560C>T, c.2551C>T, c.2546C>A, c.2462G>A, c.2298de1, c.2228G>A, c.2181G>A, c.2180G>A, c.2101C>T, c.2038A>T, c.1958T>A, c.1837C>T, c.1834C>T, c.1804G>T, c.1795G>T, c.1738C>T, c.1702C>T, c.1660C>T, c.1624C>T, c.1516C>T, c.1378C>T, c.1363C>T, c.1354A>T, c.1348C>T, c.1345G>T, c.1344dup, c.1306G>T, c.1278C>A, c.1278C>G, c.1151G>A, c.1129C>T, c.1118T>A, c.942de1, c.75 ldel, c.644T>A, c.327C>G, c.249C>A, c.121A>T, c.4846_4850 dup, c.4787_4788de1, c.4578_4612dup, c.4211_4212de1, c.4125_4130delinsATAATCATACTGATTGCCTAAAACTAAT, c.3690_3693de1, c.3338_3339de1, c.1247_1248insGTAGA, c.825_826 insGTATA, and c.278_279dup. In certain embodiments, a premature termination codon in the SCN1A gene is caused by a mutation, or a combination of mutations, selected from c.58G>T, c.575G>A, c.664C>T, c.962C>G, c.1095dupT, c.1129C>T, c.1315C>T, c.1348C>T, c.1366G>T, c.1492A>T, c.1537G>T, c.1624C>T, c.1738C>T, c.1804G>T, c.1837C>T, c.2134C>T, c.2370T>A, c.2495G>A, c.2593C>T, c.2635delC, c.2904C>A, c.3295G>T, c.3311C>A, c.3452C>G, c.3637C>T, c.3656G>A, c.3733C>T, c.3783C>A, c.3829C>T, c.3985C>T, c.4359T>G, c.4547C>A, c.4573C>T, c.4721C>G, c.4954G>T, c.5641G>T, c.5656C>T, and c.5734C>T. In certain embodiments, a premature termination codon in the SCN1A gene is caused by a mutation selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T. In certain embodiments, a premature termination codon in the SCN1A gene is caused by a mutation selected from c.1738C>T and c.3985C>T.

In certain embodiments, a premature termination codon in the SCN1A gene is caused by a mutation set forth in TABLE 7, or a combination of mutations set forth in TABLE 7.

TABLE 7

| Mutation | Mutation | Suppressor |
|---|---|---|
| (coding DNA) | (Protein) | Class |
| c.664C > T | Arg222Ter | Arg > TGA |
| c.3637C > T | Arg1213Ter | Arg > TGA |
| c.3733C > T | Arg1245Ter | Arg > TGA |
| c.2134C > T | Arg712Ter | Arg > TGA |
| c.1837C > T | Arg613Ter | Arg > TGA |
| c.4188C > A | Cys1396Ter | Cys > TGA |
| c.2877T > A | Cys959Ter | Cys > TGA |
| c.3183T > A | Cys1061Ter | Cys > TGA |
| c.3607C > T | Gln1203Ter | Gln > TAA |
| c.2782C > T | Gln928Ter | Gln > TAA |
| c.3829C > T | Gln1277Ter | Gln > TAA |
| c.2893C > T | Gln965Ter | Gln > TAA |
| c.3106C > T | Gln1036Ter | Gln > TAG |
| c.3496C > T | Gln1166Ter | Gln > TAG |
| c.5662C > T | Gln1888Ter | Gln > TAG |
| c.5461C > T | Gln1821Ter | Gln > TAG |
| c.3730C > T | Gln1244Ter | Gln > TAG |
| c.5506G > T | Glu1836Ter | Glu > TAA |
| c.5470G > T | Glu1824Ter | Glu > TAA |
| c.3757G > T | Glu1253Ter | Glu > TAA |
| c.3439G > T | Glu1147Ter | Glu > TAA |
| c.1345G > T | Glu449Ter | Glu > TAA |
| c.5404G > T | Glu1802Ter | Glu > TAG |
| c.1804G > T | Glu602Ter | Glu > TAG |
| c.5416G > T | Glu1806Ter | Glu > TAG |
| c.1795G > T | Glu599Ter | Glu > TAG |
| c.1549G > T | Glu517Ter | Glu > TAG |
| c.4255G > T | Gly1419Ter | Gly > TGA |
| c.4954G > T | Gly1652Ter | Gly > TGA |
| c.4807G > T | Gly1603Ter | Gly > TGA |
| c.487G > T | Gly163Ter | Gly > TGA |
| c.1843G > T | Gly615Ter | Gly > TGA |
| c.539T > A | Leu180Ter | Leu > TAA |
| c.2678T > A | Leu893Ter | Leu > TAA |
| c.644T > A | Leu215Ter | Leu > TAG |
| c.1958T > A | Leu653Ter | Leu > TAG |
| c.1118T > A | Leu373Ter | Leu > TAG |
| c.4541T > G | Leu1514Ter | Leu > TGA |
| c.2627T > G | Leu876Ter | Leu > TGA |
| c.4549A > T | Lys1517Ter | Lys > TAA |
| c.5536A > T | Lys1846Ter | Lys > TAA |
| c.121A > T | Lys41Ter | Lys > TAA |
| c.4192A > T | Lys1398Ter | Lys > TAA |
| c.1354A > T | Lys452Ter | Lys > TAA |
| c.2071A > T | Lys691Ter | Lys > TAG |
| c.3455C > A | Ser1152Ter | Ser > TAA |
| c.2579C > A | Ser860Ter | Ser > TAA |
| c.1883C > A | Ser628Ter | Ser > TAA |
| c.4547C > A | Ser1516Ter | Ser > TAG |
| c.2213G > A | Trp738Ter | Trp > TAG |
| c.3611G > A | Trp1204Ter | Trp > TAG |
| c.4811G > A | Trp1604Ter | Trp > TAG |
| c.4223G > A | Trp1408Ter | Trp > TAG |
| c.5435G > A | Trp1812Ter | Trp > TAG |
| c.3615G > A | Trp1205Ter | Trp > TGA |
| c.4224G > A | Trp1408Ter | Trp > TGA |
| c.4302G > A | Trp1434Ter | Trp > TGA |
| c.3858G > A | Trp1286Ter | Trp > TGA |
| c.5436G > A | Trp1812Ter | Trp > TGA |
| c.3762T > A | Tyr1254Ter | Tyr > TAA |
| c.3828T > A | Tyr1276Ter | Tyr > TAA |
| c.4266T > A | Tyr1422Ter | Tyr > TAA |
| c.3306C > A | Tyr1102Ter | Tyr > TAA |
| c.249C > A | Tyr83Ter | Tyr > TAA |
| c.5082T > G | Tyr1694Ter | Tyr > TAG |
| c.4794T > G | Tyr1598Ter | Tyr > TAG |
| c.4521C > G | Tyr1507Ter | Tyr > TAG |
| c.3822T > G | Tyr1274Ter | Tyr > TAG |
| c.5778C > G | Tyr1926Ter | Tyr > TAG |

Additional exemplary mutations, including exemplary mutations causing a premature termination codon in a gene, e.g., the SCN1A gene, can be found in ClinVar (available on the world wide web at ncbi.nlm.nih.gov/clinvar/), “A catalog of SCN1A variants” Lossin et al. (2009) BRAIN DEV. 2009 31(2):114-30, the SCN1A Registry (available on the world wide web at scnla.net/scnla-registry/), the SCN1A Mutation Database (available on the world wide web at gzneurosci.com/scnladatabase), and the Leiden Open Variation Database (LOVD v. 3.0; available on the world wide web at databases.lovd.nl/shared/genes/SCN1A). Unless indicated otherwise, any SCN1A mutations described herein are relative to SCN1a isoform 1 (NCBI reference sequence NM_001165963, SEQ ID NO: 863).

In another aspect, the invention provides a method of treating Dravet syndrome in a subject in need thereof wherein the subject has a SCN1A gene with a mutation set forth in a row TABLE 7, the method comprising administering to the subject an effective amount of a suppressor tRNA of the suppressor class indicated in the same row of TABLE 7 as the mutation, or an expression vector comprising a nucleotide sequence encoding the tRNA. “Suppressor Class” as used in TABLE 7 (e.g., Arg>TGA) refers to the endogenous tRNA type from which the suppressor tRNA is derived (e.g., an arginine tRNA) and the termination codon recognized by the suppressor tRNA (e.g., TGA). Exemplary Arg>TGA suppressor tRNAs include tRNAs comprising a nucleotide sequence selected from SEQ ID NOs: 6-9, 11, 16-18, 19-22, and 35. Exemplary Gln>TAA suppressor tRNAs include tRNAs comprising a nucleotide sequence selected from SEQ ID NOs: 36-40, 44, and 45. Exemplary Gln>TAG suppressor tRNAs include tRNAs comprising a nucleotide sequence selected from SEQ ID NOs: 178-182, 186, and 187.

For example, in certain embodiments, the subject has a SCN1A gene with a premature termination codon selected from c.664C>T, c.3637C>T, c.3733C>T, c.2134C>T, and c.1837C>T, and the method comprises administering to the subject an effective amount of a suppressor tRNA comprising a nucleotide sequence selected from SEQ ID NOs: 6-9, 11, 16-18, 19-22, and 35. In certain embodiments, the subject has a SCN1A gene with a premature termination codon selected from c.3607C>T, c.2782C>T, c.3829C>T, and c.2893C>T, and the method comprises administering to the subject an effective amount of a suppressor tRNA comprising a nucleotide sequence selected from SEQ ID NOs: 36-40, 44, and 45. In certain embodiments, the subject has a SCN1A gene with a premature termination codon selected from c.3106C>T, c.3496C>T, c.5662C>T, c.5461C>T, and c.3730C>T, and the method comprises administering to the subject an effective amount of a suppressor tRNA comprising a nucleotide sequence selected from SEQ ID NOs: 178-182, 186, and 187.

In certain embodiments, wherein the gene is a SCN1A gene, the SCN1A gene product produced with the tRNA is a functional SCN1A gene product. In certain embodiments, the functional SCN1A gene product has greater activity than the truncated SCN1A gene product, e.g., greater voltage-gated sodium channel activity. In certain embodiments, the method increases voltage-gated sodium channel activity in a cell, tissue, or subject by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a cell, tissue, or subject without the tRNA. In certain embodiments, the method increases voltage-gated sodium channel activity in a cell, tissue, or subject by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject without the tRNA. Voltage-gated sodium channel activity may be measured by any method known in the art, for example, as described in Kalume et al. (2007) J. NEUROSCI. 27(41):11065-74, Yu et al. (2007) NAT. NEUROSCI. 9(9): 1142-9, and Han et al. (2012) NATURE 489(7416): 385-390.

In certain embodiments, the functional SCN1A gene product is the Naᵥ1.1 protein. In certain embodiments, the functional SCN1A gene product comprises, consists essentially of, or consists of the amino acid sequence of any one of the following amino acid sequences, or an amino acid sequence having 80%, 85%, 86%, 87%, 88%, 89%, 90%, 91%, 92%, 93%, 94%, 95%, 96%, 97%, 98%, or 99% sequence identity to any one of the following amino acid sequences (each corresponding to different isoforms of SCN1A):

(SEQ ID NO: 863)
MEQTVLVPPGPDSENFFTRESLAAIERRIAEEKAKNPKPDKKDDDENGPKPNSDLEAGKNLPFI
YGDIPPEMVSEPLEDLDPYYINKKTFIVLNKGKAIFRESATSALYILTPENPLRKIAIKILVHS
LFSMLIMCTILTNCVFMTMSNPPDWTKNVEYTFTGIYTFESLIKIIARGFCLEDFTELRDPWNW
LDFTVITFAYVTEFVDLGNVSALRTFRVLRALKTISVIPGLKTIVGALIQSVKKLSDVMILTVE
CLSVFALIGLQLEMGNLRNKCIQWPPTNASLEEHSIEKNITVNYNGTLINETVFEFDWKSYIQD
SRYHYFLEGELDALLCGNSSDAGQCPEGYMCVKAGRNPNYGYTSEDTESWAFLSLERLMTQDEW
ENLYQLTLRAAGKTYMIFFVLVIFLGSFYLINLILAVVAMAYEEQNQATLEEAEQKEAEFQQMI
EQLKKQQEAAQQAATATASEHSREPSAAGRLSDSSSEASKLSSKSAKERRNRRKKRKQKEQSGG
EEKDEDEFQKSESEDSIRRKGFRESIEGNRLTYEKRYSSPHQSLLSIRGSLESPRRNSRTSLES
FRGRAKDVGSENDFADDEHSTFEDNESRRDSLFVPRRHGERRNSNLSQTSRSSRMLAVEPANGK
MHSTVDCNGVVSLVGGPSVPTSPVGQLLPEVIIDKPATDDNGTTTETEMRKRRSSSFHVSMDEL
EDPSQRQRAMSIASILTNTVEELEESRQKCPPCWYKESNIFLIWDCSPYWLKVKHVVNLVVMDP
FVDLAITICIVLNTLFMAMEHYPMTDHENNVLTVGNLVFTGIFTAEMELKIIAMDPYYYFQEGW
NIFDGFIVTLSLVELGLANVEGLSVLRSFRLLRVFKLAKSWPTLNMLIKIIGNSVGALGNLTLV
LAIIVFIFAVVGMQLFGKSYKDCVCKIASDCQLPRWHMNDFFHSFLIVERVLCGEWIETMWDCM
EVAGQAMCLTVFMMVMVIGNLVVLNLFLALLLSSFSADNLAATDDDNEMNNLQIAVDRMHKGVA
YVKRKIYEFIQQSFIRKQKILDEIKPLDDLNNKKDSCMSNHTAEIGKDLDYLKDVNGTTSGIGT
GSSVEKYIIDESDYMSFINNPSLTVTVPIAVGESDFENLNTEDESSESDLEESKEKLNESSSSS
EGSTVDIGAPVEEQPVVEPEETLEPEACFTEGCVQRFKCCQINVEEGRGKQWWNLRRTCFRIVE
HNWFETFIVEMILLSSGALAFEDIYIDQRKTIKTMLEYADKVETYIFILEMLLKWVAYGYQTYE
TNAWCWLDFLIVDVSLVSLTANALGYSELGAIKSLRTLRALRPLRALSRFEGMRVVVNALLGAI
PSIMNVLLVCLIFWLIFSIMGVNLFAGKFYHCINTTTGDREDIEDVNNHTDCLKLIERNETARW
KNVKVNFDNVGFGYLSLLQVATEKGWMDIMYAAVDSRNVELQPKYEESLYMYLYFVIFIIFGSF
FTLNLFIGVIIDNFNQQKKKFGGQDIFMTEEQKKYYNAMKKLGSKKPQKPIPRPGNKFQGMVED
FVTRQVEDISIMILICLNMVTMMVETDDQSEYVTTILSRINLVFIVLFTGECVLKLISLRHYYE
TIGWNIFDFVVVILSIVGMFLAELIEKYFVSPTLERVIRLARIGRILRLIKGAKGIRTLLFALM
MSLPALFNIGLLLFLVMFIYAIFGMSNFAYVKREVGIDDMENFETEGNSMICLFQITTSAGWDG
LLAPILNSKPPDCDPNKVNPGSSVKGDCGNPSVGIFFFVSYIIISELVVVNMYIAVILENESVA
TEESAEPLSEDDFEMFYEVWEKFDPDATQFMEFEKLSQFAAALEPPLNLPQPNKLQLIAMDLPM
VSGDRIHCLDILFAFTKRVLGESGEMDALRIQMEERFMASNPSKVSYQPITTTLKRKQEEVSAV
IIQRAYRRHLLKRTVKQASFTYNKNKIKGGANLLIKEDMIIDRINENSITEKTDLTMSTAACPP
SYDRVTKPIVEKHEQEGKDEKAKGK;
(SEQ ID NO: 864)
MEQTVLVPPGPDSENFFTRESLAAIERRIAEEKAKNPKPDKKDDDENGPKPNSDLEAGKNLPFI
YGDIPPEMVSEPLEDLDPYYINKKTFIVLNKGKAIFRESATSALYILTPENPLRKIAIKILVHS
LFSMLIMCTILTNCVFMTMSNPPDWTKNVEYTFTGIYTFESLIKIIARGFCLEDFTELRDPWNW
LDFTVITFAYVTEFVDLGNVSALRTFRVLRALKTISVIPGLKTIVGALIQSVKKLSDVMILTVE
CLSVFALIGLQLFMGNLRNKCIQWPPTNASLEEHSIEKNITVNYNGTLINETVFEFDWKSYIQD
SRYHYFLEGELDALLCGNSSDAGQCPEGYMCVKAGRNPNYGYTSFDTFSWAFLSLERLMTQDEW
ENLYQLTLRAAGKTYMIFFVLVIFLGSFYLINLILAVVAMAYEEQNQATLEEAEQKEAEFQQMI
EQLKKQQEAAQQAATATASEHSREPSAAGRLSDSSSEASKLSSKSAKERRNRRKKRKQKEQSGG
EEKDEDEFQKSESEDSIRRKGFRESIEGNRLTYEKRYSSPHQSLLSIRGSLFSPRRNSRTSLES
FRGRAKDVGSENDFADDEHSTFEDNESRRDSLFVPRRHGERRNSNLSQTSRSSRMLAVEPANGK
MHSTVDCNGVVSLVGGPSVPTSPVGQLLPEGTTTETEMRKRRSSSFHVSMDELEDPSQRQRAMS
IASILTNTVEELEESRQKCPPCWYKESNIFLIWDCSPYWLKVKHVVNLVVMDPFVDLAITICIV
LNTLFMAMEHYPMTDHFNNVLTVGNLVFTGIFTAEMELKIIAMDPYYYFQEGWNIEDGFIVTLS
LVELGLANVEGLSVLRSFRLLRVFKLAKSWPTLNMLIKIIGNSVGALGNLTLVLAIIVFIFAVV
GMQLFGKSYKDCVCKIASDCQLPRWHMNDFFHSFLIVERVLCGEWIETMWDCMEVAGQAMCLTV
FMMVMVIGNLVVLNLFLALLLSSESADNLAATDDDNEMNNLQIAVDRMHKGVAYVKRKIYEFIQ
QSFIRKQKILDEIKPLDDLNNKKDSCMSNHTAEIGKDLDYLKDVNGTTSGIGTGSSVEKYIIDE
SDYMSFINNPSLTVTVPIAVGESDFENLNTEDESSESDLEESKEKLNESSSSSEGSTVDIGAPV
EEQPVVEPEETLEPEACFTEGCVQRFKCCQINVEEGRGKQWWNLRRTCFRIVEHNWFETFIVEM
ILLSSGALAFEDIYIDQRKTIKTMLEYADKVETYIFILEMLLKWVAYGYQTYFTNAWCWLDFLI
VDVSLVSLTANALGYSELGAIKSLRTLRALRPLRALSRFEGMRVVVNALLGAIPSIMNVLLVCL
IFWLIFSIMGVNLFAGKFYHCINTTTGDRFDIEDVNNHTDCLKLIERNETARWKNVKVNEDNVG
FGYLSLLQVATFKGWMDIMYAAVDSRNVELQPKYEESLYMYLYFVIFIIFGSFFTLNLFIGVII
DNFNQQKKKFGGQDIFMTEEQKKYYNAMKKLGSKKPQKPIPRPGNKFQGMVEDEVTRQVEDISI
MILICLNMVTMMVETDDQSEYVTTILSRINLVFIVLFTGECVLKLISLRHYYFTIGWNIFDFVV
VILSIVGMFLAELIEKYFVSPTLERVIRLARIGRILRLIKGAKGIRTLLFALMMSLPALENIGL
LLFLVMFIYAIFGMSNFAYVKREVGIDDMENFETEGNSMICLFQITTSAGWDGLLAPILNSKPP
DCDPNKVNPGSSVKGDCGNPSVGIFFFVSYIIISELVVVNMYIAVILENESVATEESAEPLSED
DFEMFYEVWEKFDPDATQFMEFEKLSQFAAALEPPLNLPQPNKLQLIAMDLPMVSGDRIHCLDI
LFAFTKRVLGESGEMDALRIQMEERFMASNPSKVSYQPITTTLKRKQEEVSAVIIQRAYRRHLL
KRTVKQASFTYNKNKIKGGANLLIKEDMIIDRINENSITEKTDLTMSTAACPPSYDRVTKPIVE
KHEQEGKDEKAKGK;
(SEQ ID NO: 865)
MEQTVLVPPGPDSENFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPENPLRKIAIKILVHS
LFSMLIMCTILTNCVFMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVE
CLSVFALIGLQLFMGNLRNKCIQWPPTNASLEEHSIEKNITVNYNGTLINETVFEFDWKSYIQD
SRYHYFLEGELDALLCGNSSDAGQCPEGYMCVKAGRNPNYGYTSFDTFSWAFLSLERLMTQDEW
ENLYQLTLRAAGKTYMIFFVLVIFLGSFYLINLILAVVAMAYEEQNQATLEEAEQKEAEFQQMI
EQLKKQQEAAQQAATATASEHSREPSAAGRLSDSSSEASKLSSKSAKERRNRRKKRKQKEQSGG
EEKDEDEFQKSESEDSIRRKGFRESIEGNRLTYEKRYSSPHQSLLSIRGSLFSPRRNSRTSLES
FRGRAKDVGSENDFADDEHSTFEDNESRRDSLFVPRRHGERRNSNLSQTSRSSRMLAVEPANGK
MHSTVDCNGVVSLGTTTETEMRKRRSSSFHVSMDFLEDPSQRQRAMSIASILTNTVEELEESRQ
KCPPCWYKFSNIFLIWDCSPYWLKVKHVVNLVVMDPFVDLAITICIVLNTLFMAMEHYPMTDHF
NNVLTVGNLVFTGIFTAEMELKIIAMDPYYYFQEGWNIFDGFIVTLSLVELGLANVEGLSVLRS
FRLLRVEKLAKSWPTLNMLIKIIGNSVGALGNLTLVLAIIVFIFAVVGMQLFGKSYKDCVCKIA
SDCQLPRWHMNDFFHSFLIVERVLCGEWIETMWDCMEVAGQAMCLTVFMMVMVIGNLVVLNLEL
ALLLSSFSADNLAATDDDNEMNNLQIAVDRMHKGVAYVKRKIYEFIQQSFIRKQKILDEIKPLD
DLNNKKDSCMSNHTAEIGKDLDYLKDVNGTTSGIGTGSSVEKYIIDESDYMSFINNPSLTVTVP
IAVGESDFENLNTEDESSESDLEESKEKLNESSSSSEGSTVDIGAPVEEQPVVEPEETLEPEAC
FTEGCVQRFKCCQINVEEGRGKQWWNLRRTCFRIVEHNWFETFIVEMILLSSGALAFEDIYIDQ
RKTIKTMLEYADKVETYIFILEMLLKWVAYGYQTYFTNAWCWLDFLIVDVSLVSLTANALGYSE
LGAIKSLRTLRALRPLRALSRFEGMRVVVNALLGAIPSIMNVLLVCLIFWLIFSIMGVNLFAGK
FYHCINTTTGDRFDIEDVNNHTDCLKLIERNETARWKNVKVNEDNVGEGYLSLLQVATFKGWMD
IMYAAVDSRNVELQPKYEESLYMYLYFVIFIIFGSFFTLNLFIGVIIDNENQQKKKFGGQDIEM
TEEQKKYYNAMKKLGSKKPQKPIPRPGNKFQGMVFDFVTRQVEDISIMILICLNMVTMMVETDD
QSEYVTTILSRINLVFIVLFTGECVLKLISLRHYYFTIGWNIFDFVVVILSIVGMFLAELIEKY
FVSPTLERVIRLARIGRILRLIKGAKGIRTLLFALMMSLPALFNIGLLLFLVMFIYAIFGMSNF
AYVKREVGIDDMENFETEGNSMICLFQITTSAGWDGLLAPILNSKPPDCDPNKVNPGSSVKGDC
GNPSVGIFFFVSYIIISELVVVNMYIAVILENFSVATEESAEPLSEDDFEMFYEVWEKEDPDAT
QFMEFEKLSQFAAALEPPLNLPQPNKLQLIAMDLPMVSGDRIHCLDILFAFTKRVLGESGEMDA
LRIQMEERFMASNPSKVSYQPITTTLKRKQEEVSAVIIQRAYRRHLLKRTVKQASFTYNKNKIK
GGANLLIKEDMIIDRINENSITEKTDLTMSTAACPPSYDRVTKPIVEKHEQEGKDEKAKGK;
(SEQ ID NO: 866)
MEQTVLVPPGPDSENFFTRESLAAIERRIAEEKAKNPKPDKKDDDENGPKPNSDLEAGKNLPFI
YGDIPPEMVSEPLEDLDPYYINKKTFIVLNKGKAIFRFSATSALYILTPENPLRKIAIKILVHS
LFSMLIMCTILTNCVFMTMSNPPDWTKNVEYTFTGIYTFESLIKIIARGFCLEDFTELRDPWNW
LDFTVITFAYVTEFVDLGNVSALRTFRVLRALKTISVIPGLKTIVGALIQSVKKLSDVMILTVE
CLSVFALIGLQLEMGNLRNKCIQWPPTNASLEEHSIEKNITVNYNGTLINETVFEFDWKSYIQD
SRYHYFLEGELDALLCGNSSDAGQCPEGYMCVKAGRNPNYGYTSEDTESWAFLSLERLMTQDEW
ENLYQLTLRAAGKTYMIFFVLVIFLGSFYLINLILAVVAMAYEEQNQATLEEAEQKEAEFQQMI
EQLKKQQEAAQAATATASEHSREPSAAGRLSDSSSEASKLSSKSAKERRNRRKKRKQKEQSGGE
EKDEDEFQKSESEDSIRRKGFRESIEGNRLTYEKRYSSPHQSLLSIRGSLESPRRNSRTSLESE
RGRAKDVGSENDFADDEHSTFEDNESRRDSLFVPRRHGERRNSNLSQTSRSSRMLAVEPANGKM
HSTVDCNGVVSLVGGPSVPTSPVGQLLPEGTTTETEMRKRRSSSFHVSMDFLEDPSQRQRAMSI
ASILTNTVEELEESRQKCPPCWYKESNIFLIWDCSPYWLKVKHVVNLVVMDPFVDLAITICIVL
NTLFMAMEHYPMTDHENNVLTVGNLVFTGIFTAEMELKIIAMDPYYYFQEGWNIFDGFIVTLSL
VELGLANVEGLSVLRSFRLLRVEKLAKSWPTLNMLIKIIGNSVGALGNLTLVLAIIVFIFAVVG
MQLFGKSYKDCVCKIASDCQLPRWHMNDFFHSFLIVERVLCGEWIETMWDCMEVAGQAMCLTVE
MMVMVIGNLVVLNLFLALLLSSFSADNLAATDDDNEMNNLQIAVDRMHKGVAYVKRKIYEFIQQ
SFIRKQKILDEIKPLDDLNNKKDSCMSNHTAEIGKDLDYLKDVNGTTSGIGTGSSVEKYIIDES
DYMSFINNPSLTVTVPIAVGESDFENLNTEDESSESDLEESKEKLNESSSSSEGSTVDIGAPVE
EQPVVEPEETLEPEACFTEGCVQRFKCCQINVEEGRGKQWWNLRRTCFRIVEHNWFETFIVEMI
LLSSGALAFEDIYIDQRKTIKTMLEYADKVETYIFILEMLLKWVAYGYQTYFTNAWCWLDFLIV
DVSLVSLTANALGYSELGAIKSLRTLRALRPLRALSRFEGMRVVVNALLGAIPSIMNVLLVCLI
FWLIFSIMGVNLFAGKFYHCINTTTGDRFDIEDVNNHTDCLKLIERNETARWKNVKVNEDNVGF
GYLSLLQVATFKGWMDIMYAAVDSRNVELQPKYEESLYMYLYFVIFIIFGSFFTLNLFIGVIID
NFNQQKKKFGGQDIFMTEEQKKYYNAMKKLGSKKPQKPIPRPGNKFQGMVFDFVTRQVEDISIM
ILICLNMVTMMVETDDQSEYVTTILSRINLVFIVLFTGECVLKLISLRHYYFTIGWNIFDFVVV
ILSIVGMFLAELIEKYFVSPTLERVIRLARIGRILRLIKGAKGIRTLLFALMMSLPALENIGLL
LFLVMFIYAIFGMSNFAYVKREVGIDDMENFETEGNSMICLFQITTSAGWDGLLAPILNSKPPD
CDPNKVNPGSSVKGDCGNPSVGIFFFVSYIIISELVVVNMYIAVILENFSVATEESAEPLSEDD
FEMFYEVWEKFDPDATQFMEFEKLSQFAAALEPPLNLPQPNKLQLIAMDLPMVSGDRIHCLDIL
FAFTKRVLGESGEMDALRIQMEERFMASNPSKVSYQPITTTLKRKQEEVSAVIIQRAYRRHLLK
RTVKQASFTYNKNKIKGGANLLIKEDMIIDRINENSITEKTDLTMSTAACPPSYDRVTKPIVEK
HEQEGKDEKAKGK;
(SEQ ID NO: 867)
MEQTVLVPPGPDSENFFTRESLAAIERRIAEEKAKNPKPDKKDDDENGPKPNSDLEAGKNLPFI
YGDIPPEMVSEPLEDLDPYYINKKTFIVLNKGKAIFRESATSALYILTPENPLRKIAIKILVHS
LFSMLIMCTILTNCVFMTMSNPPDWTKNVEYTFTGIYTFESLIKIIARGFCLEDFTFLRDPWNW
LDFTVITFAYVTEFVDLGNVSALRTFRVLRALKTISVIPGLKTIVGALIQSVKKLSDVMILTVE
CLSVFALIGLQLFMGNLRNKCIQWPPTNASLEEHSIEKNITVNYNGTLINETVFEEDWKSYIQD
SRYHYFLEGELDALLCGNSSDAGQCPEGYMCVKAGRNPNYGYTSEDTESWAFLSLERLMTQDEW
ENLYQLTLRAAGKTYMIFFVLVIFLGSFYLINLILAVVAMAYEEQNQATLEEAEQKEAEFQQMI
EQLKKQQEAAQAATATASEHSREPSAAGRLSDSSSEASKLSSKSAKERRNRRKKRKQKEQSGGE
EKDEDEFQKSESEDSIRRKGFRESIEGNRLTYEKRYSSPHQSLLSIRGSLFSPRRNSRTSLESF
RGRAKDVGSENDFADDEHSTFEDNESRRDSLFVPRRHGERRNSNLSQTSRSSRMLAVEPANGKM
HSTVDCNGVVSLGTTTETEMRKRRSSSFHVSMDFLEDPSQRQRAMSIASILTNTVEELEESRQK
CPPCWYKESNIFLIWDCSPYWLKVKHVVNLVVMDPFVDLAITICIVLNTLFMAMEHYPMTDHEN
NVLTVGNLVFTGIFTAEMELKIIAMDPYYYFQEGWNIFDGFIVTLSLVELGLANVEGLSVLRSE
RLLRVFKLAKSWPTLNMLIKIIGNSVGALGNLTLVLAIIVFIFAVVGMQLFGKSYKDCVCKIAS
DCQLPRWHMNDFFHSFLIVERVLCGEWIETMWDCMEVAGQAMCLTVEMMVMVIGNLVVLNLFLA
LLLSSESADNLAATDDDNEMNNLQIAVDRMHKGVAYVKRKIYEFIQQSFIRKQKILDEIKPLDD
LNNKKDSCMSNHTAEIGKDLDYLKDVNGTTSGIGTGSSVEKYIIDESDYMSFINNPSLTVTVPI
AVGESDFENLNTEDESSESDLEESKEKLNESSSSSEGSTVDIGAPVEEQPVVEPEETLEPEACE
TEGCVQRFKCCQINVEEGRGKQWWNLRRTCFRIVEHNWFETFIVEMILLSSGALAFEDIYIDQR
KTIKTMLEYADKVETYIFILEMLLKWVAYGYQTYFTNAWCWLDELIVDVSLVSLTANALGYSEL
GAIKSLRTLRALRPLRALSRFEGMRVVVNALLGAIPSIMNVLLVCLIFWLIFSIMGVNLFAGKF
YHCINTTTGDREDIEDVNNHTDCLKLIERNETARWKNVKVNEDNVGFGYLSLLQVATFKGWMDI
MYAAVDSRNVELQPKYEESLYMYLYFVIFIIFGSFFTLNLFIGVIIDNENQQKKKEGGQDIFMT
EEQKKYYNAMKKLGSKKPQKPIPRPGNKFQGMVFDFVTRQVEDISIMILICLNMVTMMVETDDQ
SEYVTTILSRINLVFIVLFTGECVLKLISLRHYYFTIGWNIFDFVVVILSIVGMFLAELIEKYE
VSPTLERVIRLARIGRILRLIKGAKGIRTLLFALMMSLPALFNIGLLLFLVMFIYAIFGMSNFA
YVKREVGIDDMENFETEGNSMICLFQITTSAGWDGLLAPILNSKPPDCDPNKVNPGSSVKGDCG
NPSVGIFFFVSYIIISELVVVNMYIAVILENESVATEESAEPLSEDDFEMFYEVWEKFDPDATQ
FMEFEKLSQFAAALEPPLNLPQPNKLQLIAMDLPMVSGDRIHCLDILFAFTKRVLGESGEMDAL
RIQMEERFMASNPSKVSYQPITTTLKRKQEEVSAVIIQRAYRRHLLKRTVKQASFTYNKNKIKG
GANLLIKEDMIIDRINENSITEKTDLTMSTAACPPSYDRVTKPIVEKHEQEGKDEKAKGK;
or
(SEQ ID NO: 868)
MFLKIIAMDPYYYFQEGWNIFDGFIVTLSLVELGLANVEGLSVLRSERLLRVEKLAKSWPTLNM
LIKIIGNSVGALGNLTLVLAIIVFIFAVVGMQLFGKSYKDCVCKIASDCQLPRWHMNDFFHSEL
IVFRVLCGEWIETMWDCMEVAGQAMCLTVFMMVMVIGNLVVLNLFLALLLSSFSADNLAATDDD
NEMNNLQIAVDRMHKGVAYVKRKIYEFIQQSFIRKQKILDEIKPLDDLNNKKDSCMSNHTAEIG
KDLDYLKDVNGTTSGIGTGSSVEKYIIDESDYMSFINNPSLTVTVPIAVGESDFENLNTEDESS
ESDLEESKEKLNESSSSSEGSTVDIGAPVEEQPVVEPEETLEPEACFTEGCVQRFKCCQINVEE
GRGKQWWNLRRTCFRIVEHNWFETFIVEMILLSSGALAFEDIYIDQRKTIKTMLEYADKVETYI
FILEMLLKWVAYGYQTYFTNAWCWLDFLIVDVSLVSLTANALGYSELGAIKSLRTLRALRPLRA
LSRFEGMRVVVNALLGAIPSIMNVLLVCLIFWLIFSIMGVNLFAGKFYHCINTTTGDREDIEDV
NNHTDCLKLIERNETARWKNVKVNFDNVGFGYLSLLQVATEKGWMDIMYAAVDSRNVELQPKYE
ESLYMYLYFVIFIIFGSFFTLNLFIGVIIDNFNQQKKKFGGQDIEMTEEQKKYYNAMKKLGSKK
PQKPIPRPGNKFQGMVFDFVTRQVEDISIMILICLNMVTMMVETDDQSEYVTTILSRINLVFIV
LFTGECVLKLISLRHYYFTIGWNIFDFVVVILSIVGMFLAELIEKYFVSPTLERVIRLARIGRI
LRLIKGAKGIRTLLFALMMSLPALFNIGLLLFLVMFIYAIFGMSNFAYVKREVGIDDMENFETE
GNSMICLFQITTSAGWDGLLAPILNSKPPDCDPNKVNPGSSVKGDCGNPSVGIFFFVSYIIISF
LVVVNMYIAVILENFSVATEESAEPLSEDDFEMFYEVWEKFDPDATQFMEFEKLSQFAAALEPP
LNLPQPNKLQLIAMDLPMVSGDRIHCLDILFAFTKRVLGESGEMDALRIQMEERFMASNPSKVS
YQPITTTLKRKQEEVSAVIIQRAYRRHLLKRTVKQASFTYNKNKIKGGANLLIKEDMIIDRINE
NSITEKTDLTMSTAACPPSYDRVTKPIVEKHEQEGKDEKAKGK.

The term “effective amount” as used herein refers to the amount of an active agent (e.g., tRNA or expression vector according to the present invention or a secondary active agent in a combination therapy) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein is administered in combination with one or more additional therapeutic agents, e.g., DIACOMIT® (stiripentol), EPIODOLEX® (cannabidiol), a ketogenic diet, ONFI® (clobazam), TOPAMAX® (topiramate), fenfluramine, or valproic acid. For example, during the treatment of Dravet Syndrome, a method or composition described herein is administered in combination with one or more additional therapeutic agents, e.g., DIACOMIT® (stiripentol), EPIODOLEX® (cannabidiol), a ketogenic diet, ONFI® (clobazam), TOPAMAX® (topiramate), fenfluramine, or valproic acid.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes arginine aminoacylated suppressor tRNAs that facilitate read-through of a premature termination codon (PTC).

Suppressor tRNAs were generated from endogenous mouse arginine-tRNAs by converting their normal anticodons to TCA anticodons that recognize TGA termination codons (referred to as Arg_{TCA} suppressor tRNAs). Five of the endogenous arginine-tRNAs contained introns that must be spliced out in order to produce mature tRNAs; corresponding Arg_{TCA} suppressor tRNAs with and without these intronic sequences were generated. Suppressor tRNA sequences are shown in TABLE 8.

TABLE 8

| SEQ | Suppressor | | |
|---|---|---|---|
| ID | tRNA ID | | Suppressor tRNA Sequence |
| NO | Number | Suppressor tRNA Name | (anticodon lowercase) |
| 1 | 089 | tRNA-Arg-ACG-1-1-TCA-SUP | GGGCCAGTGGCGCAATGGATAACG |
| | | | CGTCTGACTtcaGATCAGAAGATTC |
| | | | CAGGTTCGACTCCTGGCTGGCTCG |
| 2 | 090 | tRNA-Arg-ACG-2-1-TCA-SUP | GGGCCAGTGGCGCAATGGATAACG |
| | | | CGTCTGACTtcaGATCAGAAGATTG |
| | | | TAGGTTCGACTCCTACCTGGCTCG |
| 3 | 101 | tRNA-Arg-ACG-3-1-TCA-SUP | GGGCCAGTGGCGCAATGGATAACG |
| | | | CGTCTGACTtcaGATCAGAAGATTC |
| | | | TAGGTTCGACTCCTGGCTGGCTCG |
| 4 | 102 | tRNA-Arg-CCG-1-1-TCA-SUP | GGCCGCGTGGCCTAATGGATAAGG |
| | | | CGTCTGATTtcaGATCAGAAGATTG |
| | | | AGGGTTCGAGTCCCTTCGTGGTCG |
| 5 | 103 | tRNA-Arg-CCG-2-1-TCA-SUP | GGCCGCGTGGCCTAATGGATAAGG |
| | | | CGTCTGATTtcaGATCAGAAGATTG |
| | | | GGGGTTCGAGTCCCTTCGTGGTCG |
| 6 | 104 | tRNA-Arg-CCG-3-1-TCA-SUP | GACCCAGTGGCCTAATGGATAAGG |
| | | | CATCAGCCTtcaGAGCTGGGGATTG |
| | | | TGGGTTCGAGTCCCATCTGGGTCG |
| 7 | 105 | tRNA-Arg-CCT-1-1-TCA-SUP | GCCCCAGTGGCCTAATGGATAAGG |
| | | | CACTGGCCTtcaAAGCCAGGGATTG |
| | | | TGGGTTCGAGTCCCACCTGGGGTA |
| 8 | 106 | tRNA-Arg-CCT-2-1-TCA-SUP | GCCCCAGTGGCCTAATGGATAAGG |
| | | | CACTGGCCTtcaAAGCCAGGGATTG |
| | | | TGGGTTCGAGTCCCACCTGGGGTG |
| 9 | 107 | tRNA-Arg-CCT-3-1-TCA-SUP | GCCCCGGTGGCCTAATGGATAAGGC |
| | | | ATTGGCCTtcaAAGCCAGGGATTGTG |
| | | | GGTTCGAGTCCCACCCGGGGTA |
| 10 | 108 | tRNA-Arg-CCT-4-1-TCA-SUP | GCCCCAGTGGCCTAATGGATAAGGC |
| | | | ATTGGCCTtcaAAGCCAGGGATTGTG |
| | | | GGTTCGAGTCCCATCTGGGGTG |
| 11 | 001 | tRNA-Arg-TCG-1-1-TCA-SUP | GGCCGCGTGGCCTAATGGATAAGGC |
| | | | GTCTGACTtcaGATCAGAAGATTGCA |
| | | | GGTTCGAGTCCTGCCGCGGTCG |
| 12 | 109 | tRNA-Arg-TCG-2-1-TCA-SUP | GACCGCGTGGCCTAATGGATAAGGC |
| | | | GTCTGACTtcaGATCAGAAGATTGAG |
| | | | GGTTCGAGTCCCTTCGTGGTCG |
| 13 | 110 | tRNA-Arg-TCG-3-1-TCA-SUP | GACCACGTGGCCTAATGGATAAGGC |
| | | | GTCTGACTtcaGATCAGAAGATTGAG |
| | | | GGTTCGAATCCCTTCGTGGTTG |
| 14 | 111 | tRNA-Arg-TCG-4-1-TCA-SUP | GACCACGTGGCCTAACGGATAAGGC |
| | | | GTCTGACTtcaGATCAGAAGATTGAG |
| | | | GGTTCGAATCCCTTCGTGGTTA |
| 15 | 112 | tRNA-Arg-TCT-1-1-TCA- | GGCTCTGTGGCGCAATGGATAGCGC |
| | | SUP_contains intron | ATTGGACTtcaAGTGACGAGAAAGCG |
| | | | ATTCAAAGGTTGTGGGTTCGAATCCC |
| | | | ACCAGAGTCG |
| 16 | 113 | tRNA-Arg-TCT-1-1-TCA- | GGCTCTGTGGCGCAATGGATAGCGC |
| | | SUP_no intron | ATTGGACTtcaAATTCAAAGGTTGTG |
| | | | GGTTCGAATCCCACCAGAGTCG |
| 17 | 114 | tRNA-Arg-TCT-2-1-TCA- | GGCTCCGTGGCGCAATGGATAGCGC |
| | | SUP_contains intron | ATTGGACTtcaAGAGGCTGAAGGCAT |
| | | | TCAAAGGTTCCGGGTTCGAGTCCCGG |
| | | | CGGAGTCG |
| 18 | 115 | tRNA-Arg-TCT-2-1-TCA- | GGCTCCGTGGCGCAATGGATAGCGC |
| | | SUP_no intron | ATTGGACTtcaAATTCAAAGGTTCCG |
| | | | GGTTCGAGTCCCGGCGGAGTCG |
| 19 | 116 | tRNA-Arg-TCT-3-1-TCA- | GGCTCTGTGGCGCAATGGATAGCGC |
| | | SUP_contains intron | ATTGGACTtcaAGCATGATTGAGAGA |
| | | | TTCAAAGGTTGCGGGTTCGAGTCCCG |
| | | | CCAGAGTCG |
| 20 | 117 | tRNA-Arg-TCT-3-1-TCA- | GGCTCTGTGGCGCAATGGATAGCGC |
| | | SUP_no intron | ATTGGACTtcaAATTCAAAGGTTGCG |
| | | | GGTTCGAGTCCCGCCAGAGTCG |
| 21 | 118 | tRNA-Arg-TCT-4-1-TCA- | GGCTCTGTGGCGCAATGGATAGCGC |
| | | SUP_contains intron | ATTGGACTtcaAGACAAATGGAGGCA |
| | | | TTCAAAGGTTGTGGGTTCGAGTCCCA |
| | | | CCAGAGTCG |
| 22 | 119 | tRNA-Arg-TCT-4-1-TCA- | GGCTCTGTGGCGCAATGGATAGCGC |
| | | SUP_no intron | ATTGGACTtcaAATTCAAAGGTTGTG |
| | | | GGTTCGAGTCCCACCAGAGTCG |
| 23 | 120 | tRNA-Arg-TCT-5-1-TCA-SUP | GTCTCTGTGGCGCAATGGACGAGCG |
| | | | CGCTGGACTtcaAATCCAGAGGTTCT |
| | | | GGGTTCGAGTCCCGGCAGAGATG |
| 24 | 121 | tRNA-Arg-TCT-6-1-TCA- | GGCTCTGTGGAGCAATGGATAGCAC |
| | | SUP_contains intron | ATTGGACTtcaAGCATGACCGAGAGA |
| | | | TTCAAAGGTTGCGGGTTCGAGTCCCA |
| | | | CCAGAGTTG |
| 25 | 122 | tRNA-Arg-TCT-6-1-TCA- | GGCTCTGTGGAGCAATGGATAGCAC |
| | | SUP_no intron | ATTGGACTtcaAATTCAAAGGTTGCG |
| | | | GGTTCGAGTCCCACCAGAGTTG |
| 35 | 179 | tRNA-Arg-TCT-5-1-TCA- | GTCTCTGTGGCGCAATGGACGAGCG |
| | | SUP_T51C | CGCTGGACTtcaAATCCAGAGGTTCC |
| | | | GGGTTCGAGTCCCGGCAGAGATG |

In this Example, all mature tRNA sequences (as predicted by GtRNAdb; http://gtrnadb.ucsc.edu) were expressed in the context of upstream and downstream genomic flanking sequences (±200 bps) from tRNA-Arg-TCG-1-1—a highly expressed arginine-tRNA, i.e., the tRNA sequences were expressed with a 5′ flanking sequence of SEQ ID NO: 26 and a 3′ flanking sequence of SEQ ID NO: 27. All mature tRNA sequences including upstream and downstream genomic flanking sequences were generated in a pGL4 vector backbone.

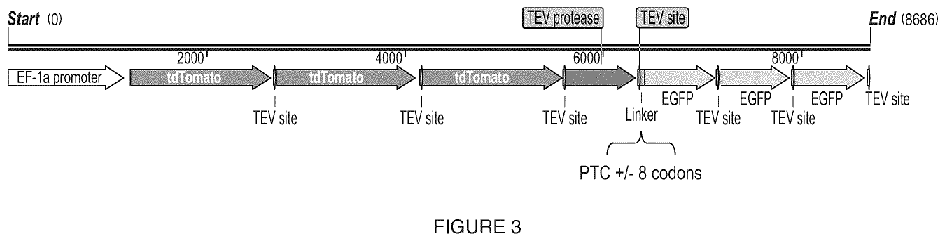

The Arg_{TCA} suppressors were tested for PTC readthrough activity by flow cytometry in cell lines containing dual fluorescent readthrough reporters. These reporters include three copies of a red fluorescent protein (tdTomato), TEV protease, a linker region containing a PTC, and three copies of a green fluorescent protein (EGFP). A schematic of a reporter construct is shown in FIG. 3. In the absence of any PTC readthrough as a result of a suppressor tRNA, translation will be terminated by the PTC within the linker region, and only tdTomato will be expressed (and therefore only red fluorescence detected). PTC readthrough activity as a result of a suppressor tRNA will allow translation to proceed through the PTC in the linker region, and for both tdTomato and EGFP to be expressed (and therefore both red and green fluorescence detected). Accordingly, readthrough can be assessed by quantifying the percentage of viable cells expressing both the red and green fluorescent reporters above background (double positive %).

To screen for suppressor tRNAs with readthrough activity at PTCs relevant to Dravet syndrome, linker regions were generated containing the PTC and eight flanking codons on either side of the PTC from the SCN1A transcript of two patients with nonsense mutations in the SCN1A gene: subject N and subject S.

The linker region derived from the subject N SCN1A transcript is as follows, and the reporter including this linker region is referred to as the subject N-PTC reporter:

(SEQ ID NO: 28)
CTGAGACCTCTAAGAGCCTTATCTtgaTTTGAAGGGATGAGGGTGGTTGT
G.

A corresponding linker with a wild-type Arg codon in place of the PTC was used as a control, and had the following sequence:

(SEQ ID NO: 193)
CTGAGACCTCTAAGAGCCTTATCTcgaTTTGAAGGGATGAGGGTGGTTGT
G.

The linker region derived from the subject S SCN1A transcript is as follows, and the reporter including this linker region is referred to as the subject S-PTC reporter:

(SEQ ID NO: 29)
ACAAGCCTTTTCAGCTTTAGAGGGtgaGCAAAGGATGTGGGATCTGAGAA
C.

A corresponding linker with a wild-type Arg codon in place of the PTC was used as a control, and had the following sequence:

(SEQ ID NO: 194)
ACAAGCCTTTTCAGCTTTAGAGGGcgaGCAAAGGATGTGGGATCTGAGAA
C.

An additional 51 base pair linker region was derived from a mouse model of Dravet syndrome caused by an R1407X nonsense mutation in SCN1A (Ogiwara et al., 2007, Neurobiology of Disease). The linker region derived from the SCN1A R1407X transcript is as follows, and the reporter including this linker region is referred to as R1407X-PTC reporter:

(SEQ ID NO: 30)
CTAATAGAAAGAAATGAGACCGCCtgaTGGAAAAATGTGAAAGTAAACTT
T.

A corresponding linker with a wild-type Arg codon in place of the PTC was used as a control, and had the following sequence:

(SEQ ID NO: 195)
CTAATAGAAAGAAATGAGACCGCCcggTGGAAAAATGTGAAAGTAAACTT
T.

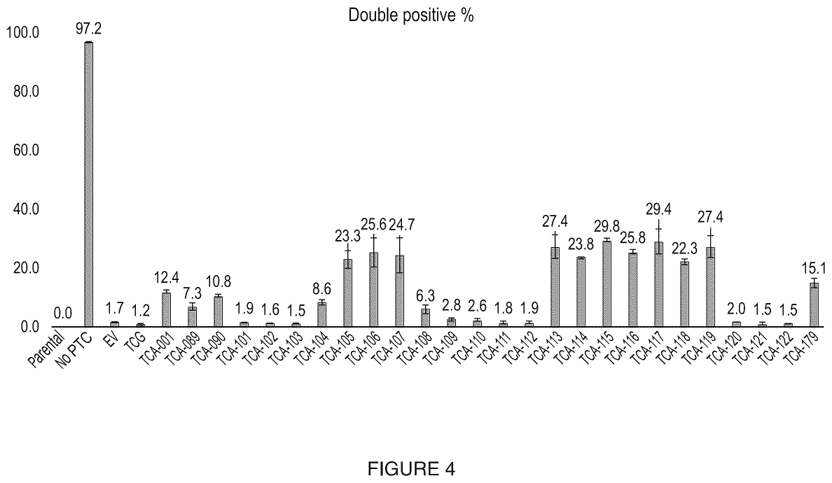

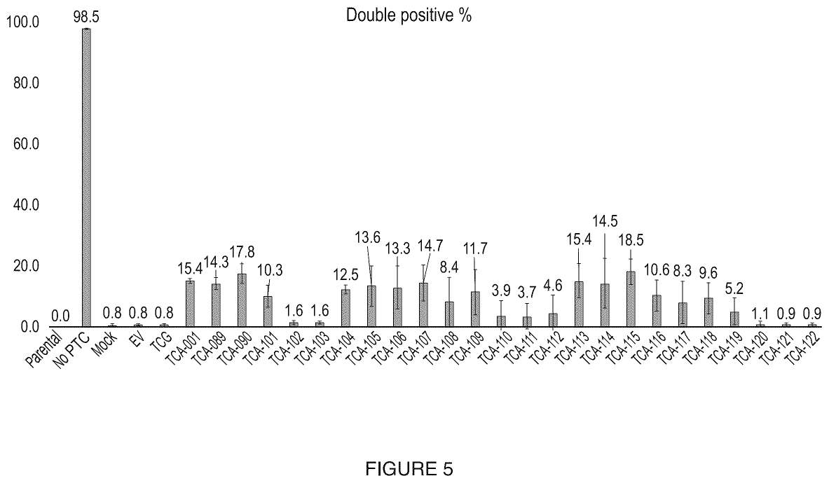

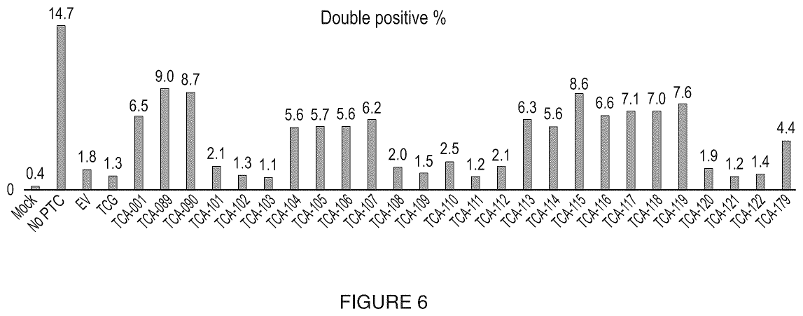

Arg_{TCA} suppressors were tested in multiple assay contexts, including (i) a human Flp-In-293 cell line stably expressing the subject S-PTC reporter and transiently transfected with a plasmid encoding an Arg_{TCA} suppressor (results shown in FIG. 4), (ii) a murine Flp-In-3T3 cell line stably expressing the R1407X-PTC reporter and transiently transfected with a plasmid encoding an Arg_{TCA} suppressor (results shown in FIG. 5), and (iii) Flp-In-293 cells transiently co-transfected with plasmids encoding the subject N-PTC reporter and an Arg_{TCA} suppressor tRNA (results shown in FIG. 6). Transfections were done using the Lipofectamine 3000 Transfection Reagent according to the manufacturer's protocol.

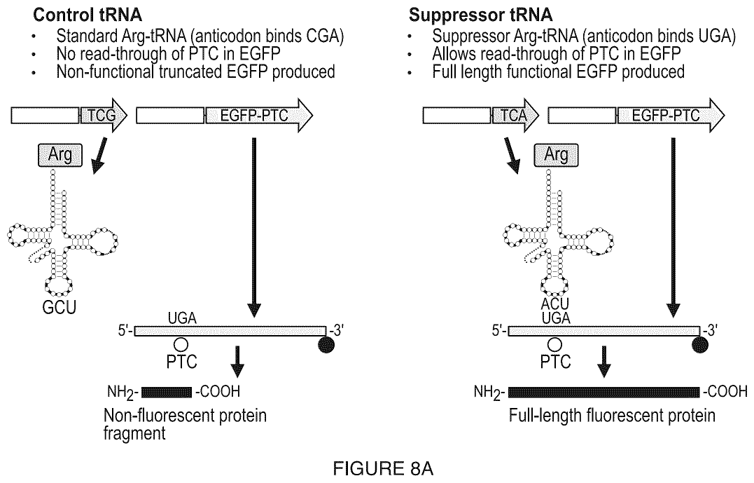

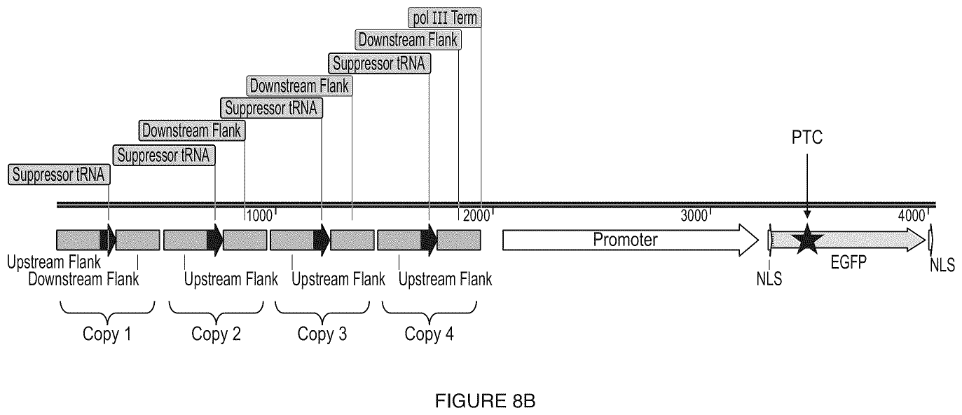

An additional reporter construct was generated including EGFP with a nuclear localization signal (NLS) and containing an arginine-to-TGA mutation (R96X) within the EGFP open reading frame that abolishes fluorescence in the absence of PTC readthrough. The general experimental approach is shown in FIG. 8A. EGFP expression was driven by the CMV early enhancer/chicken 13 actin (CAG) promoter. The reporter construct is referred to as CAG:NLS-EGFP (R96X-TGA) and its sequence is as follows:

(SEQ ID NO: 31)
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTACGCCCCCTATTGACG
TCAATGACGGTAAATGGCCCGCCTGGCATTATGCCCAGTACATGACCTTA
TGGGACTTTCCTACTTGGCAGTACATCTACGTATTAGTCATCGCTATTAC
CATGGTCGAGGTGAGCCCCACGTTCTGCTTCACTCTCCCCATCTCCCCCC
CCTCCCCACCCCCAATTTTGTATTTATTTATTTTTTAATTATTTTGTGCA
GCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATC
AGAGCGGCGCGCTCCGAAAGTTTCCTTTTATGGCGAGGCGGCGGCGGCGG
CGGCCCTATAAAAAGCGAAGCGCGCGGCGGGCGGGAGTCGCTGCGCGCTG
CCTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTCT
CCTCCGGGCTGTAATTAGCGCTTGGTTTAATGACGGCTTGTTTCTTTTCT
GTGGCTGCGTGAAAGCCTTGAGGGGCTCCGGGAGCGCCGGCAGGAAGGAA
ATGGGCGGGGAGGGCCTTCGTGCGTCGCCGCGCCGCCGTCCCCTTCTCCC
TCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCC
TCTGCTAACCATGTTCATGCCTTCTTCTTTTTCCTACAGCTCCTGGGCAA
CGTGCTGGTTATTGTGCTGTCTCATCATTTTGGCAAAGAATTGCGGCCCA
ACGGTACCGGATCCACCGGCCGCCACCATGGGAAGCCCAAAGAAGAAGCG
TAAGGTAATGGTGAGCAAGGGCGAGGAGCTGTTCACCGGGGTGGTGCCCA
TCCTGGTCGAGCTGGACGGCGACGTAAACGGCCACAAGTTCAGCGTGTCC
GGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCC
TGACCTACGGCGTGCAGTGCTTCAGCCGCTACCCCGACCACATGAAGCAG
CACGACTTCTTCAAGTCCGCCATGCCCGAAGGCTACGTCCAGGAGTGAAC
CATCTTCTTCAAGGACGACGGCAACTACAAGACCCGCGCCGAGGTGAAGT
TCGAGGGCGACACCCTGGTGAACCGCATCGAGCTGAAGGGCATCGACTTC
AAGGAGGACGGCAACATCCTGGGGCACAAGCTGGAGTACAACTACAACAG
CCACAACGTCTATATCATGGCCGACAAGCAGAAGAACGGCATCAAGGTGA
ACTTCAAGATCCGCCACAACATCGAGGACGGCAGCGTGCAGCTCGCCGAC
CACTACCAGCAGAACACCCCCATCGGCGACGGCCCCGTGCTGCTGCCCGA
CAACCACTACCTGAGCACCCAGTCCGCCCTGAGCAAAGACCCCAACGAGA
AGCGCGATCACATGGTCCTGCTGGAGTTCGTGACCGCCGCCGGGATCACT
CTCGGCATGGACGAGCTGTACAAGGGAAGCCCCAAGAAAAAGCGGAAGGT
GTAA.

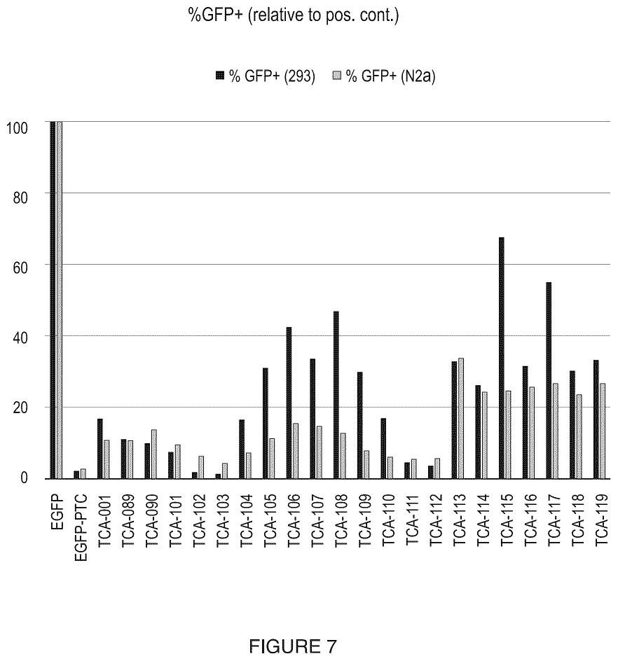

The activity of the Arg_{TCA} suppressors was assessed in HEK293 cells and murine Neuro-2a cells (a neural crest-derived cell line extensively used to study neuronal differentiation) transiently co-transfected with a plasmid encoding the Arg_{TCA} suppressor tRNA and a plasmid encoding the CAG:NLS-EGFP (R96X-TGA) reporter. Transfections were done using the Lipofectamine 3000 Transfection Reagent according to the manufacturer's protocol. Co-transfections were done using equal amounts of the indicated suppressor plasmid and the CAG:NLS-EGFP (R96X-TGA) reporter plasmid. EGFP expression was analyzed by flow cytometry at ˜24 hours post transfection in 293 cells and ˜48 hours post transfection in Neuro-2a cells. The results are shown in FIG. 7.

In general, the relative readthrough activity of Arg_{TCA} suppressor tRNAs remained consistent across multiple assay formats. The following suppressors reliably showed readthrough activity above baseline: TCA-001 (SEQ ID NO: 11), TCA-89 (SEQ ID NO: 1), TCA-90 (SEQ ID NO: 2), TCA-105 (SEQ ID NO: 7), TCA-106 (SEQ ID NO: 8), TCA-107 (SEQ ID NO: 9), TCA-113 (SEQ ID NO: 16), TCA-114 (SEQ ID NO: 17), TCA-115 (SEQ ID NO: 18), TCA-116 (SEQ ID NO: 19), TCA-117 (SEQ ID NO: 20), TCA-118 (SEQ ID NO: 21), and TCA-119 (SEQ ID NO: 22).

Together, these results demonstrate that the described suppressor tRNAs can facilitate expression of transcripts, e.g., SCN1A transcripts, containing premature termination codons associated with disorders, e.g., Dravet syndrome.

### Example 2

This Example describes the impact of expression vector features on the read-through of a premature termination codon (PTC) by arginine aminoacylated suppressor tRNAs.

Expression constructs containing both Arg_{TCA} suppressor tRNAs and EGFP (R96X-TGA) reporters on the same plasmid were generated in a pGL4 vector backbone. These constructs included 1, 2, 3 or 4 copies of Arg_{TCA} suppressor tRNAs 113 (SEQ ID NO: 16), 115 (SEQ ID NO: 18), and 001 (SEQ ID NO: 11) (as described in Example 1 and shown in TABLE 8). Each copy of the tRNA sequences was expressed in the context of either (i) 200 bps upstream genomic flanking sequences from tRNA-Arg-TCG-1-1 (SEQ ID NO: 26) and 200 bps of downstream genomic flanking sequence from tRNA-Arg-TCG-1-1 (SEQ ID NO: 27), (ii) 200 bps upstream genomic flanking sequences from tRNA-Arg-TCG-1-1 (SEQ ID NO: 26) and 104 bps of downstream genomic flanking sequence from tRNA-Arg-TCG-1-1 (SEQ ID NO: 32), or (iii) an upstream U6 promoter including 19 bps of upstream flanking genomic sequence from tRNA-Arg-TCG-1-1 (SEQ ID NO: 33) and 46 bps of downstream flanking genomic sequence from tRNA-Arg-TCG-1-1 (SEQ ID NO: 34). The general experimental approach is shown in FIG. 8A and a schematic of an exemplary reporter construct containing 4 copies of the suppressor tRNA is shown in FIG. 8B. Reporter constructs were either a CAG:NLS-EGFP (R96X-TGA) reporter construct (as described in Example 1) or an EF1a:NLS-EGFP (R96X-TGA) reporter construct in which the CAG promoter was replaced with an elongation factor-1 alpha (EF1a) promoter, whose sequence is as follows:

(SEQ ID NO: 177)
GGCTCCGGTGCCCGTCAGTGGGCAGAGCGCACATCGCCCACAGTCCCCGA
GAAGTTGGGGGGAGGGGTCGGCAATTGAACCGGTGCCTAGAGAAGGTGGC
GCGGGGTAAACTGGGAAAGTGATGTCGTGTACTGGCTCCGCCTTTTTCCC
GAGGGTGGGGGAGAACCGTATATAAGTGCACTAGTCGCCGTGAACGTTCT
TTTTCGCAACGGGTTTGCCGCCAGAACACAGGTAAGTGCCGTGTGTGGTT
CCCGCGGGCCTGGCCTCTTTACGGGTTATGGCCCTTGCGTGCCTTGAATT
ACTTCCACCTGGCTGCAGTACGTGATTCTTGATCCCGAGCTTCGGGTTGG
AAGTGGGTGGGAGAGTTCGTGGCCTTGCGCTTAAGGAGCCCCTTCGCCTC
GTGCTTGAGTTGTGGCCTGGCCTGGGCGCTGGGGCCGCCGCGTGCGAATC
TGGTGGCACCTTCGCGCCTGTCTCGCTGCTTTCGATAAGTCTCTAGCCAT
TTAAAATTTTTGATGACCTGCTGCGACGCTTTTTTTCTGGCAAGATAGTC
TTGTAAATGCGGGCCAAGATCAGCACACTGGTATTTCGGTTTTTGGGGCC
GCGGGCGGCGACGGGGCCCGTGCGTCCCAGCGCACATGTTCGGCGAGGCG
GGGCCTGCGAGCGCGGCCACCGAGAATCGGACGGGGGTAGTCTCAAGCTG
CCCGGCCTGCTCTGGTGCCTGGCCTCGCGCCGCCGTGTATCGCCCCGCCC
TGGGCGGCAAGGCTGGCCCGGTCGGCACCAGTTGCGTGAGCGGAAAGATG
GCCGCTTCCCGGCCCTGCTGCAGGGAGCACAAAATGGAGGACGCGGCGCT
CGGGAGAGCGGGCGGGTGAGTCACCCACACAAAGGAAAAGGGCCTTTCCG
TCCTCAGCCGTCGCTTCATGTGACTCCACGGAGTACCGGGCGCCGTCCAG
GCACCTCGATTAGTTCTCCAGCTTTTGGAGTACGTCGTCTTTAGGTTGGG
GGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCT
TTTTGAGTTTGGATCTTGGTTCATTCTCAAGCCTCAGACAGTGGTTCAAA
GTTTTTTTCTTCCATTTCAGGTGTCGTGAGGTACCGGATCCACCGGCCGC
CACCATGGGAAGCCCAAAGAAGAAGCGTAAGGTAATGGTGAGCAAGGGCG
AGGAGCTGTTCACCGGGGTGGTGCCCATCCTGGTCGAGCTGGACGGCGAC
GTAAACGGCCACAAGTTCAGCGTGTCCGGCGAGGGCGAGGGCGATGCCAC
CTACGGCAAGCTGACCCTGAAGTTCATCTGCACCACCGGCAAGCTGCCCG
TGCCCTGGCCCACCCTCGTGACCACCCTGACCTACGGCGTGCAGTGCTTC
AGCCGCTACCCCGACCACATGAAGCAGCACGACTTCTTCAAGTCCGCCAT
GCCCGAAGGCTACGTCCAGGAGTGAACCATCTTCTTCAAGGACGACGGCA
ACTACAAGACCCGCGCCGAGGTGAAGTTCGAGGGCGACACCCTGGTGAAC
CGCATCGAGCTGAAGGGCATCGACTTCAAGGAGGACGGCAACATCCTGGG
GCACAAGCTGGAGTACAACTACAACAGCCACAACGTCTATATCATGGCCG
ACAAGCAGAAGAACGGCATCAAGGTGAACTTCAAGATCCGCCACAACATC
GAGGACGGCAGCGTGCAGCTCGCCGACCACTACCAGCAGAACACCCCCAT
CGGCGACGGCCCCGTGCTGCTGCCCGACAACCACTACCTGAGCACCCAGT
CCGCCCTGAGCAAAGACCCCAACGAGAAGCGCGATCACATGGTCCTGCTG
GAGTTCGTGACCGCCGCCGGGATCACTCTCGGCATGGACGAGCTGTACAA
GGGAAGCCCCAAGAAAAAGCGGAAGGTGTAA.

Neuro-2a cells or HEK293 (Flpin-293) cells were transfected with these constructs and readthrough was assayed by fluorescent imaging or flow cytometry ˜24 hours or 48 hours post transfection. Transfections were done using the Lipofectamine 3000 Transfection Reagent according to the manufacturer's protocol.

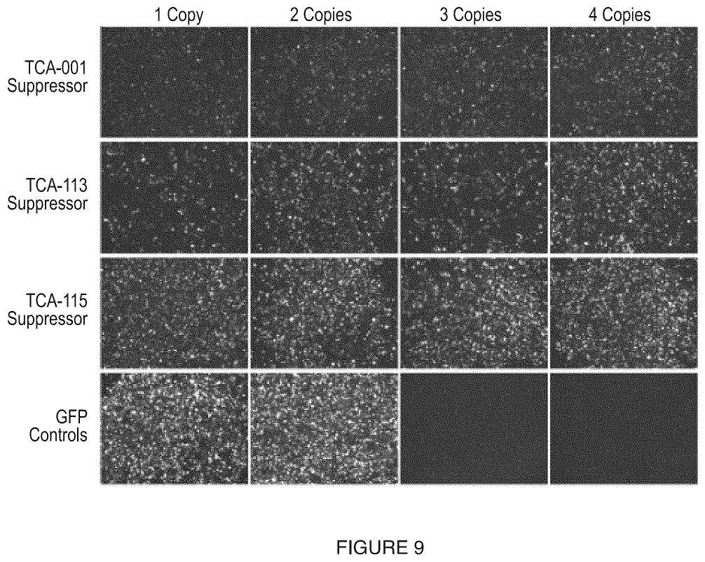

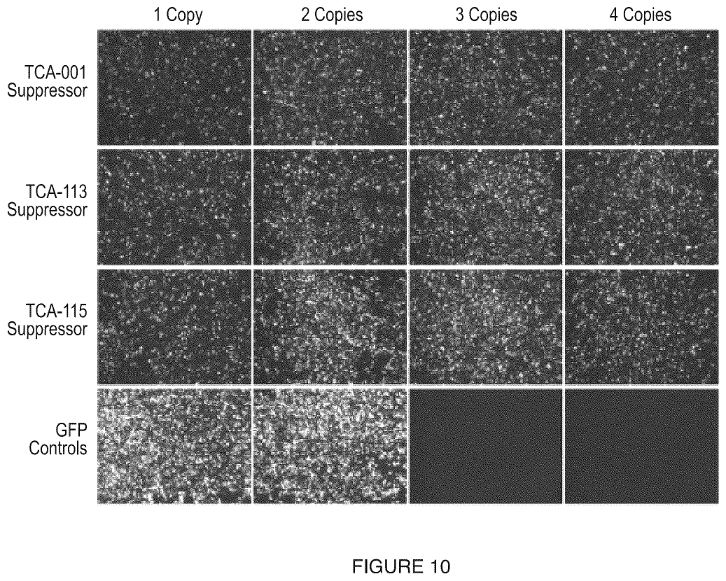

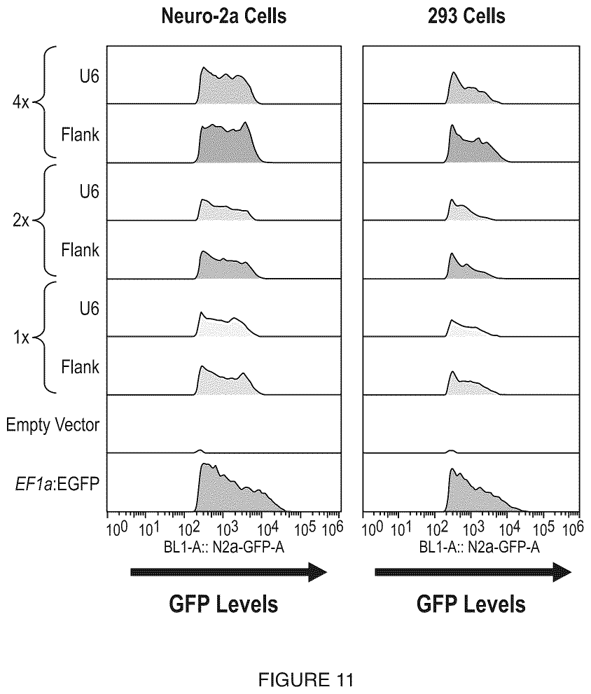

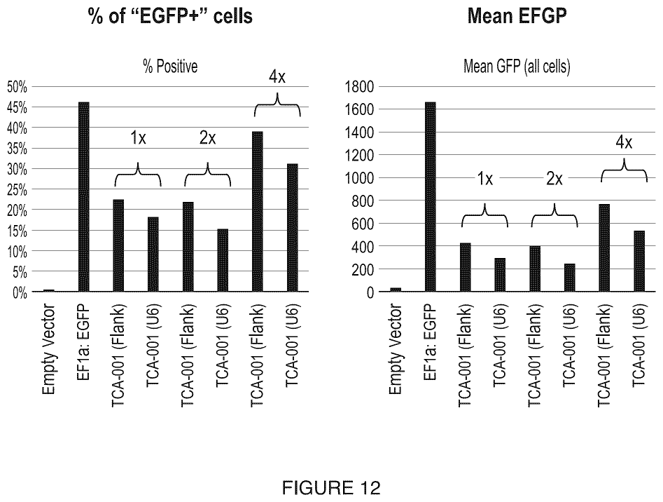

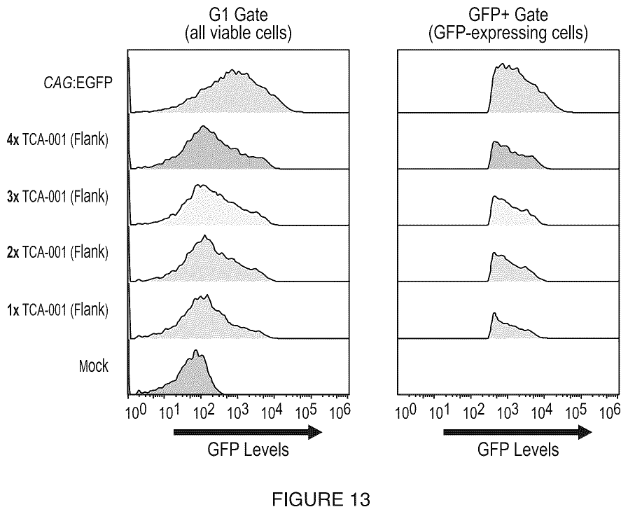

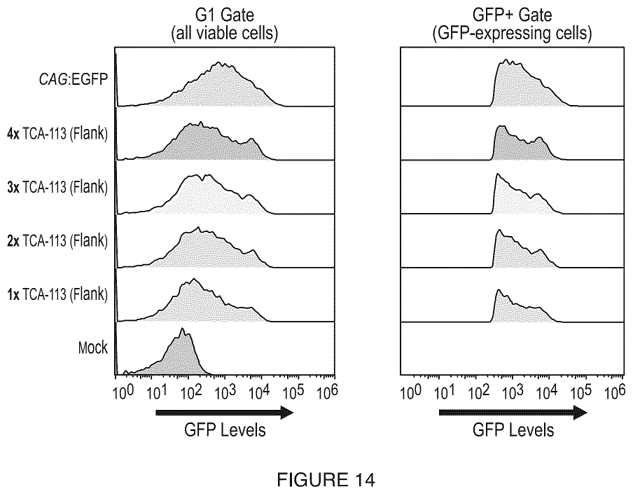

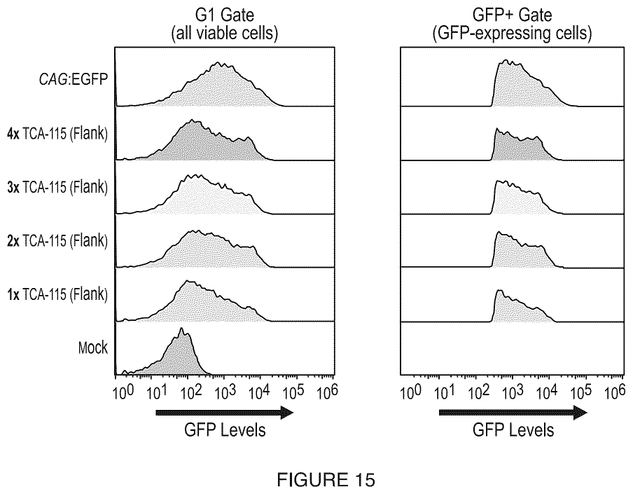

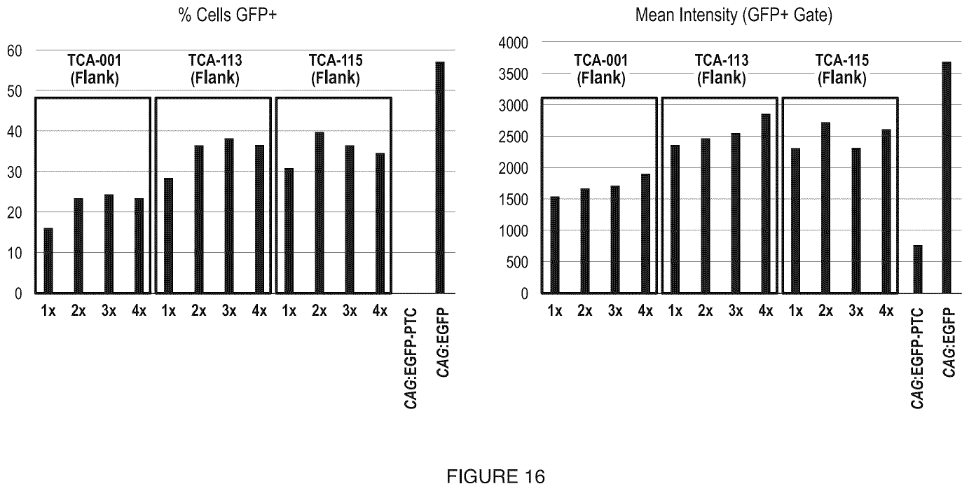

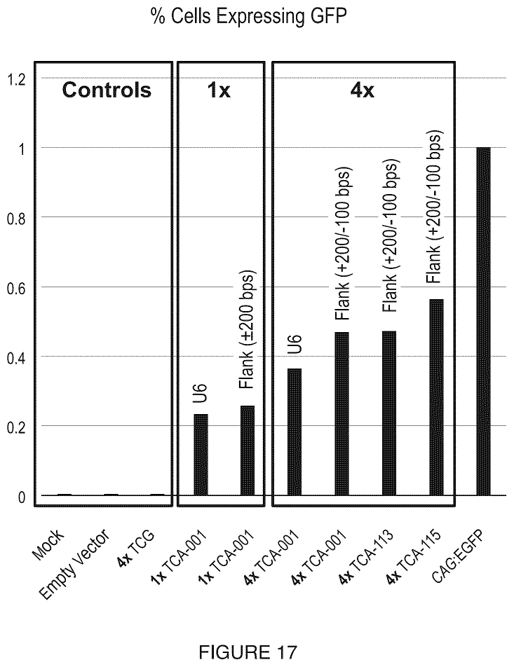

The results are shown FIGS. 9-10 (fluorescent images) and FIGS. 11-17 (quantification of fluorescent signal as measured by flow cytometry). Improved readthrough upon increased copy number was seen for the 001 and 113 suppressors. Together, the results show that increasing the copy number of the suppressor tRNA modules in reporter constructs often results in enhanced readthrough activity. Additionally, U6-containing constructs exhibited PTC readthrough activity, although generally not as much as was seen for equivalent suppressor tRNAs expressed in the flanking genomic context.

### Example 3

This example describes the design of a functional arginine aminoacylated suppressor tRNA that facilitates read-through of a premature termination codon (PTC) in a transcript.

C57BL/6J mice have a naturally occurring C-to-T mutation in the T-loop (position 51) of Arg-TCT-5-1 that has been shown to affect pre-tRNA processing and function (Ryuta Ishimura et al., Science, 2014). Arg_{TCA} suppressor 120 (as described in Example 1 and shown in TABLE 8) contains a T at position 51. A modified suppressor tRNA was generated that included a substitution of C for Tat position 51 of Arg_{TCA} suppressor 120 (referred to as Arg_{TCA} suppressor 179 and having a nucleotide sequence of SEQ ID NO: 35). Arg_{TCA} suppressors 120 and 179 were tested by transfection into a Flp-In-293 cell line stably expressing the subject S-PTC Reporter and by co-transfection with a plasmid encoding the subject N-PTC reporter into Flp-In-3T3 cells. Transfections were done using the Lipofectamine 3000 Transfection Reagent according to the manufacturer's protocol. Results are shown in FIG. 4 and FIG. 6. While Arg_{TCA} suppressor 120 was non-functional, Arg_{TCA} suppressor 179, containing only a single substitution, showed PTC readthrough activity.

### Example 4

This Example describes glutamine aminoacylated suppressor tRNAs that facilitate read-through of a premature termination codon (PTC).

Suppressor tRNAs were generated from endogenous mouse glutamine-tRNAs by converting their normal anticodons to TTA or CTA anticodons (referred to as G1n_{TTA} or G1n_{CTA} suppressor tRNAs, respectively). Suppressor tRNA sequences are shown in TABLE 9.

TABLE 9

| SEQ | Suppressor | | |
|---|---|---|---|
| ID | tRNA ID | | Suppressor tRNA Sequence |
| NO | Number | Suppressor tRNA Name | (anticodon lowercase) |
| 36 | 002 | tRNA-Gln-TTG-1-1-TTA-SUP | GGTCCCATGGTGTAATGGTTAGCAC |
| | | | TCTGGACTttaAATCCAGCGATCCG |
| | | | AGTTCAAATCTCGGTGGGACCT |
| 37 | 155 | tRNA-Gln-CTG-1-1-TTA-SUP | GGTTCCATGGTGTAATGGTTAGCAC |
| | | | TCTGGACTttaAATCCAGCGACCCG |
| | | | AGTTCAAATCTCGGTGGGACCT |
| 38 | 156 | tRNA-Gln-CTG-2-1-TTA-SUP | GGTTCCATGGTGTAATGGTTAGCAC |
| | | | TCTGGACTttaAATCCAGCGATCCG |
| | | | AGTTCAAATCTCGGTGGAACCT |
| 39 | 157 | tRNA-Gln-CTG-3-1-TTA-SUP | GGTTCCATGGTGTAATGGTTAGCAC |
| | | | TCTGGACTttaAATCCAGCGATCCG |
| | | | AGTTCAAATCTCGGTGGGACCT |
| 40 | 158 | tRNA-Gln-CTG-4-1-TTA-SUP | GGTTCCATGGTGTAATGGTGAGCAC |
| | | | TCTGGACTttaAATCCAGCGATCCG |
| | | | AGTTCAAATCTCGGTGGGACCT |
| 41 | 159 | tRNA-Gln-CTG-5-1-TTA-SUP | GGTTCCATGGTGTAATGGCTAGCAC |
| | | | TCTGGACTttaAATCCAGCGATCCG |
| | | | AGTTCAAATCTCGGTGGGATTT |
| 42 | 160 | tRNA-Gln-CTG-6-1-TTA-SUP | GGTTCCATGGTGTAATGGTTAGCAC |
| | | | TCTGGACTttaAATCCAGCCATACA |
| | | | AGTTCAAATCTCAGTGGAACCT |
| 43 | 161 | tRNA-Gln-CTG-7-1-TTA-SUP | GGTTCCTTGGTGTAAGATGAGC |
| | | | ACTCTGGATTttaAATCCAGCG |
| | | | ATCAGAGTTCAAATCTCGGTGG |
| | | | GACCT |
| 44 | 162 | tRNA-Gln-TTG-2-1-TTA-SUP | GGTCCCATGGTGTAATGGTTAG |
| | | | CACTCTGGACTttaAATCCAGC |
| | | | AATCTGAGTTCAAATCTCGGTG |
| | | | GGACCT |
| 45 | 163 | tRNA-Gln-TTG-3-1-TTA-SUP | GGCCCCATGGTGTAATGGTTAG |
| | | | CACTCTGGACTttaAATCCAGC |
| | | | GATCCGAGTTCAAATCTCGGTG |
| | | | GGACCT |
| 46 | 164 | tRNA-Gln-TTG-4-1-TTA-SUP | GGTCTCATGGTGTAATGGTTAG |
| | | | CACACTGGACTttaAGTCCAGC |
| | | | AATCCGAGTTCGAGTCTTGGTG |
| | | | AGACCA |
| 47 | 165 | tRNA-Gln-TTG-5-1-TTA-SUP | GGACCCATGGTGTAATGGTTAG |
| | | | CACTCTGGACTttaAATCCAGC |
| | | | AATCCAAGTTCAAATCTCGGTG |
| | | | GGACCT |
| 48 | 166 | tRNA-Gln-TTG-6-1-TTA-SUP | GTTTCCATGGTGTAATGGTTGG |
| | | | CACTCTGGACTttaAATCCAGC |
| | | | AATCCAAGTTCAAGTCTCTGTG |
| | | | GGACCT |
| 178 | 196 | tRNA-Gln-CTG-1-1-CTA- | GGTCCCATGGTGTAATGGTTAG |
| | | SUP | CACTCTGGACTctaAATCCAGC |
| | | | GATCCGAGTTCAAATCTCGGTG |
| | | | GGACCT |
| 179 | 189 | tRNA-Gln-CTG-2-1-CTA- | GGTTCCATGGTGTAATGGTTAG |
| | | SUP | CACTCTGGACTctaAATCCAGC |
| | | | GACCCGAGTTCAAATCTCGGTG |
| | | | GGACCT |
| 180 | 190 | tRNA-Gln-CTG-3-1-CTA- | GGTTCCATGGTGTAATGGTTAG |
| | | SUP | CACTCTGGACTctaAATCCAGC |
| | | | GATCCGAGTTCAAATCTCGGTG |
| | | | GAACCT |
| 181 | 191 | tRNA-Gln-CTG-4-1-CTA- | GGTTCCATGGTGTAATGGTTAG |
| | | SUP | CACTCTGGACTctaAATCCAGC |
| | | | GATCCGAGTTCAAATCTCGGTG |
| | | | GGACCT |
| 182 | 192 | tRNA-Gln-CTG-5-1-CTA- | GGTTCCATGGTGTAATGGTGAG |
| | | SUP | CACTCTGGACTctaAATCCAGC |
| | | | GATCCGAGTTCAAATCTCGGTG |
| | | | GGACCT |
| 183 | 193 | tRNA-Gln-CTG-6-1-CTA- | GGTTCCATGGTGTAATGGCTAG |
| | | SUP | CACTCTGGACTctaAATCCAGC |
| | | | GATCCGAGTTCAAATCTCGGTG |
| | | | GGATTT |
| 184 | 194 | tRNA-Gln-CTG-7-1-CTA- | GGTTCCATGGTGTAATGGTTAG |
| | | | CACTCTGGACTctaAATCCAGC |
| | | SUP | CATACAAGTTCAAATCTCAGTG |
| | | | GAACCT |
| 185 | 195 | tRNA-Gln-TTG-1-1-CTA-SUP | GGTTCCTTGGTGTAAGATGAGC |
| | | | ACTCTGGATTctaAATCCAGCG |
| | | | ATCAGAGTTCAAATCTCGGTGG |
| | | | GACCT |
| 186 | 197 | tRNA-Gln-TTG-2-1-CTA-SUP | GGTCCCATGGTGTAATGGTTAG |
| | | | CACTCTGGACTctaAATCCAGC |
| | | | AATCTGAGTTCAAATCTCGGTG |
| | | | GGACCT |
| 187 | 198 | tRNA-Gln-TTG-3-1-CTA-SUP | GGCCCCATGGTGTAATGGTTAG |
| | | | CACTCTGGACTctaAATCCAGC |
| | | | GATCCGAGTTCAAATCTCGGTG |
| | | | GGACCT |
| 188 | 199 | tRNA-Gln-TTG-4-1-CTA-SUP | GGTCTCATGGTGTAATGGTTAG |
| | | | CACACTGGACTctaAGTCCAGC |
| | | | AATCCGAGTTCGAGTCTTGGTG |
| | | | AGACCA |
| 189 | 200 | tRNA-Gln-TTG-5-1-CTA-SUP | GGACCCATGGTGTAATGGTTAG |
| | | | CACTCTGGACTctaAATCCAGC |
| | | | AATCCAAGTTCAAATCTCGGTG |
| | | | GGACCT |
| 190 | 201 | tRNA-Gln-TTG-6-1-CTA-SUP | GTTTCCATGGTGTAATGGTTGG |
| | | | CACTCTGGACTctaAATCCAGC |
| | | | AATCCAAGTTCAAGTCTCTGTG |
| | | | GGACCT |

In this Example, all mature tRNA sequences were expressed in the context of upstream and downstream genomic flanking sequences (±200 bps) from tRNA-Gln-TTG-1-1— a highly expressed glutamine-tRNA, i.e., the tRNA sequences were expressed with a 5′ flanking sequence of SEQ ID NO: 173 and a 3′ flanking sequence of SEQ ID NO: 174. All mature tRNA sequences including upstream and downstream genomic flanking sequences were generated in a pGL4 vector backbone.

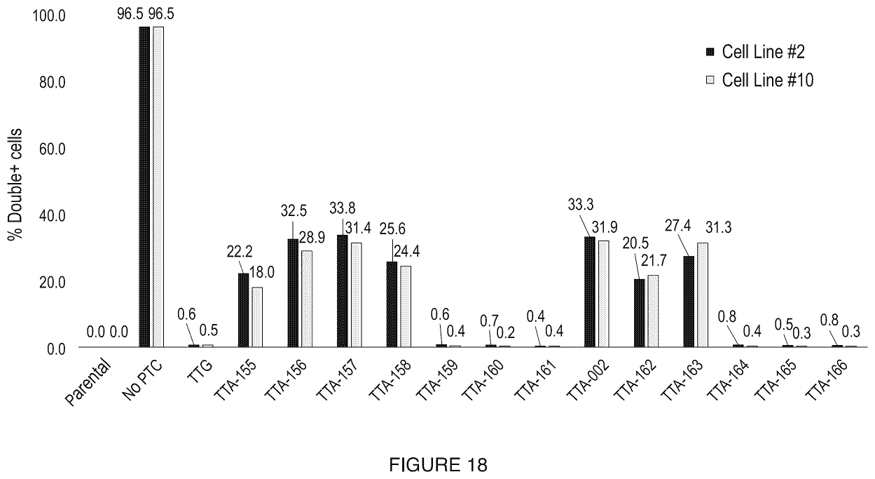

The G1n_{TTA} suppressors were tested for PTC readthrough activity by flow cytometry in two independently derived Flp-In-293 cell lines containing an integrated fluorescent readthrough reporter. Results are shown in FIG. 18. The reporters included three copies of a red fluorescent protein (tdTomato), TEV protease, a linker region containing a PTC, and three copies of a green fluorescent protein (EGFP). A schematic of the reporter construct is shown in FIG. 3. In the absence of any PTC readthrough as a result of a suppressor tRNA, translation will be terminated by the PTC within the linker region, and only tdTomato will be expressed (and therefore only red fluorescence detected). PTC readthrough activity as a result of a suppressor tRNA will allow translation to proceed through the PTC in the linker region, and for both tdTomato and EGFP to be expressed (and therefore both red and green fluorescence detected). Accordingly, readthrough was assessed with flow cytometry by quantifying the percentage of viable cells expressing both the red and green fluorescent reporters above background (double positive %). The linker was derived from a mouse Dmd^{mdx} transcript, and had the following sequence:

(SEQ ID NO: 192)
CTGCAAAGTTCTTTGAAAGAGCAAtaaAATGGCTTCAACTATCTGAGTGA
C.

A corresponding linker with a wild-type Gln codon in place of the PTC was used as a control, and had the following sequence:

(SEQ ID NO: 191)
CTGCAAAGTTCTTTGAAAGAGCAACAAAATGGCTTCAACTATCTGAGTGA
C.

An additional reporter construct was generated in a pGL4 vector backbone including EGFP with a nuclear localization signal (NLS) and containing a glutamine-to-TAA mutation (Q69X) that abolishes fluorescence in the absence of PTC readthrough. EGFP expression was driven by the CMV early enhancer/chicken β actin (CAG) promoter. The reporter construct is referred to as CAG:NLS-EGFP (Q69X-TAA) and its sequence is as follows:

(SEQ ID NO: 175)
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTACGCCCCCTATTGACG
TCAATGACGGTAAATGGCCCGCCTGGCATTATGCCCAGTACATGACCTTA
TGGGACTTTCCTACTTGGCAGTACATCTACGTATTAGTCATCGCTATTAC
CATGGTCGAGGTGAGCCCCACGTTCTGCTTCACTCTCCCCATCTCCCCCC
CCTCCCCACCCCCAATTTTGTATTTATTTATTTTTTAATTATTTTGTGCA
GCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATC
AGAGCGGCGCGCTCCGAAAGTTTCCTTTTATGGCGAGGCGGCGGCGGCGG
CGGCCCTATAAAAAGCGAAGCGCGCGGCGGGCGGGAGTCGCTGCGCGCTG
CCTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTCT
CCTCCGGGCTGTAATTAGCGCTTGGTTTAATGACGGCTTGTTTCTTTTCT
GTGGCTGCGTGAAAGCCTTGAGGGGCTCCGGGAGCGCCGGCAGGAAGGAA
ATGGGGGGGGAGGGCCTTCGTGCGTCGCCGCGCCGCCGTCCCCTTCTCCC
TCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCC
TCTGCTAACCATGTTCATGCCTTCTTCTTTTTCCTACAGCTCCTGGGCAA
CGTGCTGGTTATTGTGCTGTCTCATCATTTTGGCAAAGAATTGCGGCCCA
ACGGTACCGGATCCACCGGCCGCCACCATGGGAAGCCCAAAGAAGAAGCG
TAAGGTAATGGTGAGCAAGGGCGAGGAGCTGTTCACCGGGGTGGTGCCCA
TCCTGGTCGAGCTGGACGGCGACGTAAACGGCCACAAGTTCAGCGTGTCC
GGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCC
TGACCTACGGCGTGTAATGCTTCAGCCGCTACCCCGACCACATGAAGCAG
CACGACTTCTTCAAGTCCGCCATGCCCGAAGGCTACGTCCAGGAGCGCAC
CATCTTCTTCAAGGACGACGGCAACTACAAGACCCGCGCCGAGGTGAAGT
TCGAGGGCGACACCCTGGTGAACCGCATCGAGCTGAAGGGCATCGACTTC
AAGGAGGACGGCAACATCCTGGGGCACAAGCTGGAGTACAACTACAACAG
CCACAACGTCTATATCATGGCCGACAAGCAGAAGAACGGCATCAAGGTGA
ACTTCAAGATCCGCCACAACATCGAGGACGGCAGCGTGCAGCTCGCCGAC
CACTACCAGCAGAACACCCCCATCGGCGACGGCCCCGTGCTGCTGCCCGA
CAACCACTACCTGAGCACCCAGTCCGCCCTGAGCAAAGACCCCAACGAGA
AGCGCGATCACATGGTCCTGCTGGAGTTCGTGACCGCCGCCGGGATCACT
CTCGGCATGGACGAGCTGTACAAGGGAAGCCCCAAGAAAAAGCGGAAGGT
GTAA.

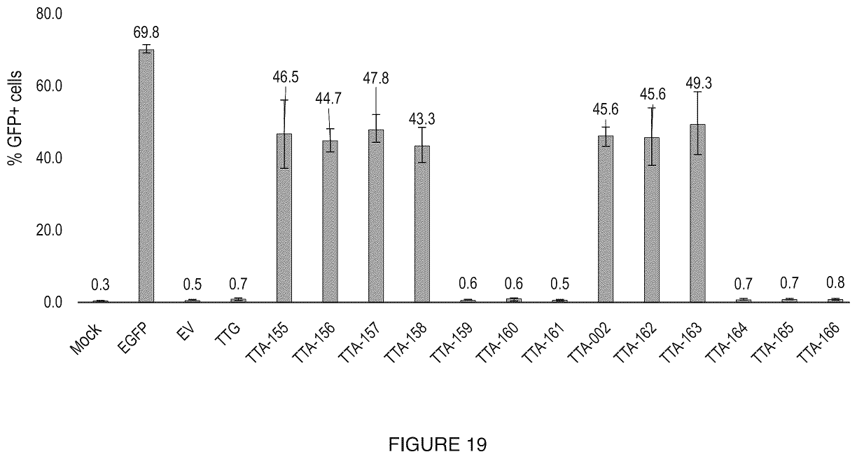

The activity of the G1n_{TTA} suppressors was assessed by flow cytometry in Neuro-2a cells transiently co-transfected with plasmids encoding a G1n_{TTA} suppressor tRNA and the CAG:NLS-EGFP (Q69X-TAA) reporter. Transfections were done using the Lipofectamine 3000 Transfection Reagent according to the manufacturer's protocol. The results are shown in FIG. 19.

An additional reporter construct was generated in a pGL4 vector backbone including EGFP with a nuclear localization signal (NLS) and containing a glutamine-to-TAG mutation (Q69X) that abolishes fluorescence in the absence of PTC readthrough. EGFP expression is driven by the CMV early enhancer/chicken β actin (CAG) promoter. The reporter construct is referred to as CAG:NLS-EGFP (Q69X-TAG) and its sequence is as follows:

(SEQ ID NO: 176)
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTACGCCCCCTATTGACG
TCAATGACGGTAAATGGCCCGCCTGGCATTATGCCCAGTACATGACCTTA
TGGGACTTTCCTACTTGGCAGTACATCTACGTATTAGTCATCGCTATTAC
CATGGTCGAGGTGAGCCCCACGTTCTGCTTCACTCTCCCCATCTCCCCCC
CCTCCCCACCCCCAATTTTGTATTTATTTATTTTTTAATTATTTTGTGCA
GCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATC
AGAGCGGCGCGCTCCGAAAGTTTCCTTTTATGGCGAGGCGGCGGCGGCGG
CGGCCCTATAAAAAGCGAAGCGCGCGGCGGGCGGGAGTCGCTGCGCGCTG
CCTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTCT
CCTCCGGGCTGTAATTAGCGCTTGGTTTAATGACGGCTTGTTTCTTTTCT
GTGGCTGCGTGAAAGCCTTGAGGGGCTCCGGGAGCGCCGGCAGGAAGGAA
ATGGGCGGGGAGGGCCTTCGTGCGTCGCCGCGCCGCCGTCCCCTTCTCCC
TCTCCAGCCTCGGGGCTGTCCGCGGGGGGACGGCTGCCTTCGGGGGGGAC
GGGGCAGGGCGGGGTTCGGCTTCTGGCGTGTGACCGGCGGCTCTAGAGCC
TCTGCTAACCATGTTCATGCCTTCTTCTTTTTCCTACAGCTCCTGGGCAA
CGTGCTGGTTATTGTGCTGTCTCATCATTTTGGCAAAGAATTGCGGCCCA
ACGGTACCGGATCCACCGGCCGCCACCATGGGAAGCCCAAAGAAGAAGCG
TAAGGTAATGGTGAGCAAGGGCGAGGAGCTGTTCACCGGGGTGGTGCCCA
TCCTGGTCGAGCTGGACGGCGACGTAAACGGCCACAAGTTCAGCGTGTCC
GGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCC
TGACCTACGGCGTGTAGTGCTTCAGCCGCTACCCCGACCACATGAAGCAG
CACGACTTCTTCAAGTCCGCCATGCCCGAAGGCTACGTCCAGGAGCGCAC
CATCTTCTTCAAGGACGACGGCAACTACAAGACCCGCGCCGAGGTGAAGT
TCGAGGGCGACACCCTGGTGAACCGCATCGAGCTGAAGGGCATCGACTTC
AAGGAGGACGGCAACATCCTGGGGCACAAGCTGGAGTACAACTACAACAG
CCACAACGTCTATATCATGGCCGACAAGCAGAAGAACGGCATCAAGGTGA
ACTTCAAGATCCGCCACAACATCGAGGACGGCAGCGTGCAGCTCGCCGAC
CACTACCAGCAGAACACCCCCATCGGCGACGGCCCCGTGCTGCTGCCCGA
CAACCACTACCTGAGCACCCAGTCCGCCCTGAGCAAAGACCCCAACGAGA
AGCGCGATCACATGGTCCTGCTGGAGTTCGTGACCGCCGCCGGGATCACT
CTCGGCATGGACGAGCTGTACAAGGGAAGCCCCAAGAAAAAGCGGAAGGT
GTAA.

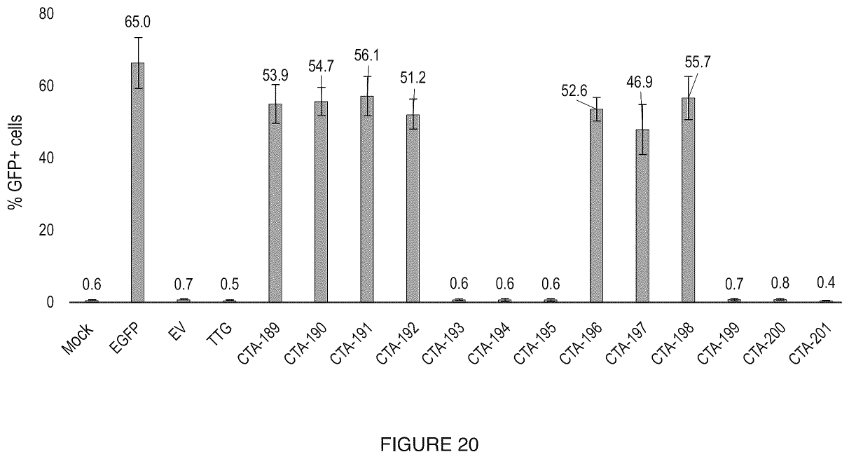

The activity of the G1n_{CTA} suppressors was assessed in Neuro-2a cells transiently co-transfected with plasmids encoding a G1n_{CTA} suppressor tRNA and the CAG:NLS-EGFP (Q69X-TAG) reporter. The results are shown in FIG. 20.

Together, these results demonstrate that the described suppressor tRNAs can facilitate expression of transcripts containing premature termination codons associated with disorders.

### Example 5

This Example describes readthrough activity of disclosed suppressor tRNAs and small molecule nonsense suppression therapies.

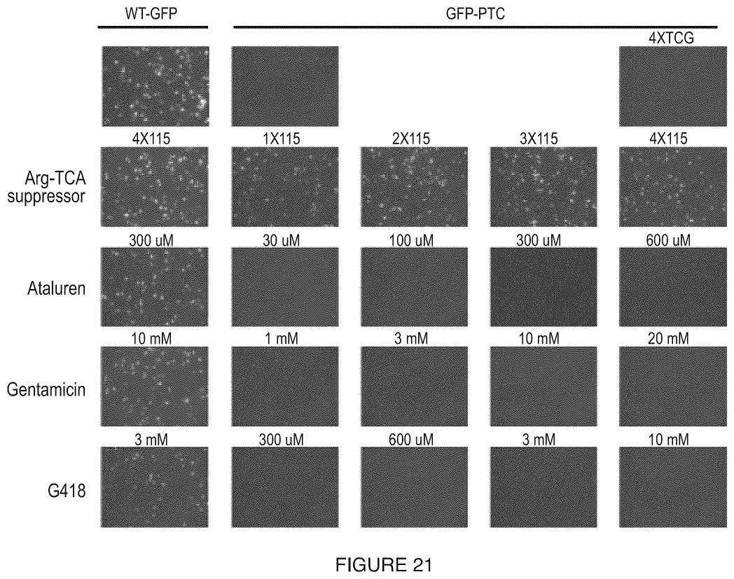

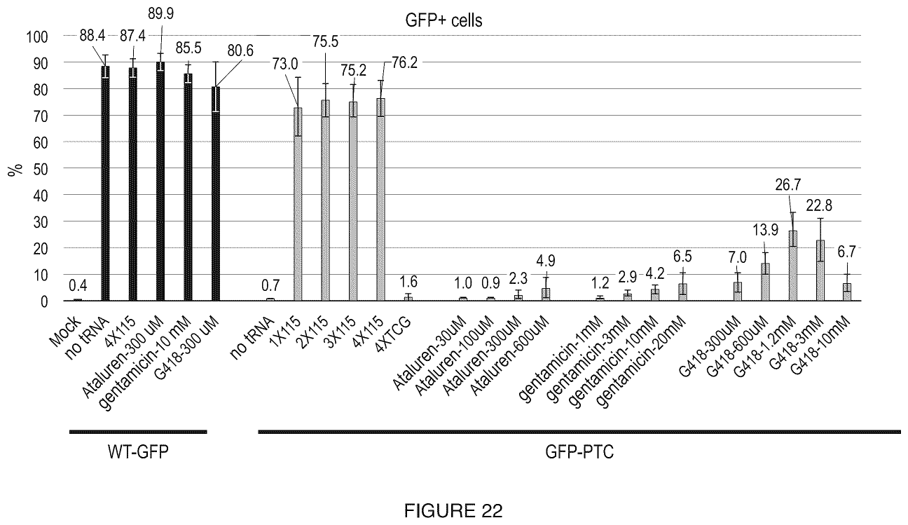

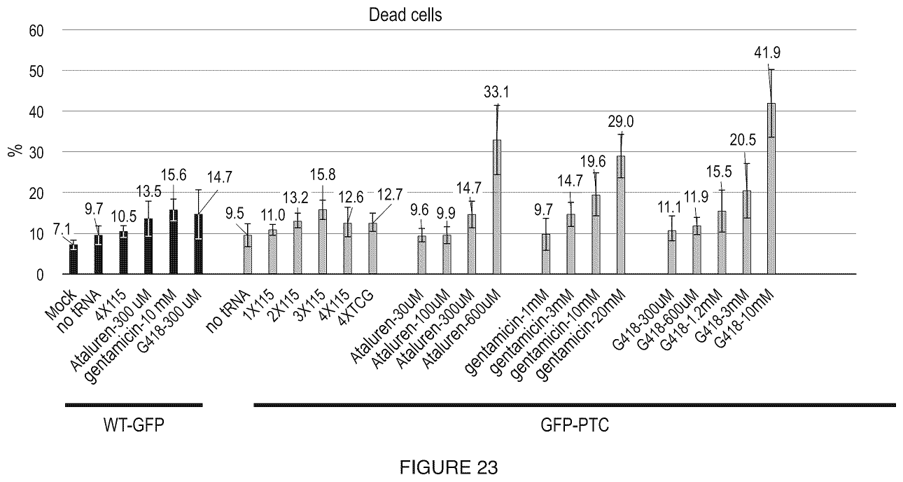

Disclosed suppressor tRNAs were tested alongside nonsense suppression drugs translarna (ataluren), gentamicin, and G418 (geneticin). PTC readthrough activity was measured in Neuro-2a cells ˜48 hours after transfection with an expression construct containing a CAG:NLS-EGFP (R96X-TGA) reporter (as described in Example 1) and either (i) including an Arg_{TCA} suppressor tRNA at the indicated copy number on the same construct, or (ii) treated with ataluren, (iii) treated with gentamicin, or (iv) treated with G418. Transfections were performed using the Lipofectamine 3000 Transfection Reagent according to the manufacturer's protocol. For all experimental conditions, cell culture medium was replaced with fresh medium ˜6 hours after transfection and the indicated drugs at the indicated concentrations were added at this point. PTC readthrough activity was measured as the percentage of EGFP positive cells. A reporter containing wildtype EGFP without a PTC was used as a control. Cell viability in cells receiving the same set of treatments was assessed by flow cytometry following staining with 7-Amino Actinomycin D (7-AAD; Thermo Fisher Scientific #006993-50), a membrane impermeant dye that is generally excluded from viable cells, which was used according to the manufacturer's protocol. The results are shown in FIGS. 21-23. Together, the results demonstrate that the Arg_{TCA} suppressor tRNA #115 (SEQ ID NO: 18) produces far greater readthrough than any of the nonsense suppression drugs. Additionally, the results show that, unlike for any of the nonsense suppression drugs, treatment with the Arg_{TCA} suppressor tRNA is not accompanied by a decrease in cell viability.

### Example 6

This Example describes aminoacylated suppressor tRNAs that facilitate read-through of a premature termination codon (PTC).

Suppressor tRNAs are generated from endogenous mouse tRNAs by converting their normal anticodons to anticodons that recognize premature termination codons (PTCs). Suppressor tRNA sequences are shown in TABLE 10.

TABLE 10

| SEQ | | |
|---|---|---|
| ID | | |
| NO | Suppressor tRNA Name | Suppressor tRNA Sequence |
| 49 | pre-tRNA-Leu-CAA->cta--1-1 | GTCAGGATGGCCGAGTGGTCTAAGGC |
| | | GCCAGACTctaGCTATGGCTTCCTCG |
| | | CTCTGAGGGTTCTGGTCTCCCCTGGA |
| | | GGCGTGGGTTCGAATCCCACTTCTGA |
| | | CA |
| 50 | pre-tRNA-Leu-CAA->cta--2-1 | GTCAGGATGGCCGAGTGGTCTAAGGC |
| | | GCCAGACTctaGCTTAGCTTCCCTGT |
| | | CTGGGGATTCTGGTCTCCGTATGGAG |
| | | GCGTGGGTTCGAATCCCACTTCTGAC |
| | | A |
| 51 | pre-tRNA-Leu-CAA->cta--3-1 | GTCAGGATGGCCGAGTGGTCTAAGGC |
| | | GCCAGACTctaGGTGACAAGCCTTAC |
| | | CTACGGGTGTTCTGGTCTCCGAATGG |
| | | AGGCGTGGGTTCGAATCCCACTTCTG |
| | | ACA |
| 52 | pre-tRNA-Leu-CAA->cta--4-1 | GTCAGGATGGCCGAGTGGTCTAAGGC |
| | | GCCAGACTctaGCGTTCGCTTCCTCT |
| | | ACTGAGGGTTCTGGTCTCCGTGTGGA |
| | | GGCGTGGGTTCGAATCCCACTTCTGA |
| | | CA |
| 53 | pre-tRNA-Leu-CAA->tca--1-1 | GTCAGGATGGCCGAGTGGTCTAAGGC |
| | | GCCAGACTtcaGCTATGGCTTCCTCG |
| | | CTCTGAGGGTTCTGGTCTCCCCTGGA |
| | | GGCGTGGGTTCGAATCCCACTTCTGA |
| | | CA |
| 54 | pre-tRNA-Leu-CAA->tca--2-1 | GTCAGGATGGCCGAGTGGTCTAAGGC |
| | | GCCAGACTtcaGCTTAGCTTCCCTGT |
| | | CTGGGGATTCTGGTCTCCGTATGGAG |
| | | GCGTGGGTTCGAATCCCACTTCTGAC |
| | | A |
| 55 | pre-tRNA-Leu-CAA->tca--3-1 | GTCAGGATGGCCGAGTGGTCTAAGGC |
| | | GCCAGACTtcaGGTGACAAGCCTTAC |
| | | CTACGGGTGTTCTGGTCTCCGAATGG |
| | | AGGCGTGGGTTCGAATCCCACTTCTG |
| | | ACA |
| 56 | pre-tRNA-Leu-CAA->tca--4-1 | GTCAGGATGGCCGAGTGGTCTAAGGC |
| | | GCCAGACTtcaGCGTTCGCTTCCTCT |
| | | ACTGAGGGTTCTGGTCTCCGTGTGGA |
| | | GGCGTGGGTTCGAATCCCACTTCTGA |
| | | CA |
| 57 | pre-tRNA-Leu-CAA->tta--1-1 | GTCAGGATGGCCGAGTGGTCTAAGGC |
| | | GCCAGACTttaGCTATGGCTTCCTCG |
| | | CTCTGAGGGTTCTGGTCTCCCCTGGA |
| | | GGCGTGGGTTCGAATCCCACTTCTGA |
| | | CA |
| 58 | pre-tRNA-Leu-CAA->tta--2-1 | GTCAGGATGGCCGAGTGGTCTAAGGC |
| | | GCCAGACTttaGCTTAGCTTCCCTGT |
| | | CTGGGGATTCTGGTCTCCGTATGGAG |
| | | GCGTGGGTTCGAATCCCACTTCTGAC |
| | | A |
| 59 | pre-tRNA-Leu-CAA->tta--3-1 | GTCAGGATGGCCGAGTGGTCTAAGGC |
| | | GCCAGACTttaGGTGACAAGCCTTAC |
| | | CTACGGGTGTTCTGGTCTCCGAATGG |
| | | AGGCGTGGGTTCGAATCCCACTTCTG |
| | | ACA |
| 60 | pre-tRNA-Leu-CAA->tta--4-1 | GTCAGGATGGCCGAGTGGTCTAAGGC |
| | | GCCAGACTttaGCGTTCGCTTCCTCT |
| | | ACTGAGGGTTCTGGTCTCCGTGTGGA |
| | | GGCGTGGGTTCGAATCCCACTTCTGA |
| | | CA |
| 61 | pre-tRNA-Tyr-GTA->cta--1-1 | CCTTCGATAGCTCAGTTGGTAGAGCG |
| | | GAGGACTctaGAGTTACTAGAATAGT |
| | | GATCCTTAGGTCGCTGGTTCGAATCC |
| | | GGCTCGAAGGA |
| 62 | pre-tRNA-Tyr-GTA->cta--2-1 | CCTTCGATAGCTCAGTTGGTAGAGCG |
| | | GAGGACTctaGTCAGTACAATATGGT |
| | | AATCCTTAGGTCGCTGGTTCGATTCC |
| | | GGCTCGAAGGA |
| 63 | pre-tRNA-Tyr-GTA->cta--3-1 | CCTTCGATAGCTCAGCTGGTAGAGCG |
| | | GAGGACTctaGGCTTGTGGCTGTGGA |
| | | CATCCTTAGGTCGCTGGTTCGATTCC |
| | | GGCTCGAAGGA |
| 64 | pre-tRNA-Tyr-GTA->cta--4-1 | CCTTCGATAGCTCAGCTGGTAGAGCG |
| | | GAGGACTctaGCTAACTCCCCGTTAG |
| | | AAGACATCCTTAGGTCGCTGGTTCGA |
| | | CTCCGGCTCGAAGGA |
| 65 | pre-tRNA-Tyr-GTA->cta--5-1 | CTTTCGATAGTTCAGTTGGTAGAGCG |
| | | GAGGACTctaGAGTATTAACGTTGGT |
| | | GATCCTTAGGTCGCTGGTTCGAGTCC |
| | | GGCTCGAAGGA |
| 66 | pre-tRNA-Tyr-GTA->tta--1-1 | CCTTCGATAGCTCAGTTGGTAGAGCG |
| | | GAGGACTttaGAGTTACTAGAATAGT |
| | | GATCCTTAGGTCGCTGGTTCGAATCC |
| | | GGCTCGAAGGA |
| 67 | pre-tRNA-Tyr-GTA->tta--2-1 | CCTTCGATAGCTCAGTTGGTAGAGCG |
| | | GAGGACTttaGTCAGTACAATATGGT |
| | | AATCCTTAGGTCGCTGGTTCGATTCC |
| | | GGCTCGAAGGA |
| 68 | pre-tRNA-Tyr-GTA->tta--3-1 | CCTTCGATAGCTCAGCTGGTAGAGCG |
| | | GAGGACTttaGGCTTGTGGCTGTGGA |
| | | CATCCTTAGGTCGCTGGTTCGATTCC |
| | | GGCTCGAAGGA |
| 69 | pre-tRNA-Tyr-GTA->tta--4-1 | CCTTCGATAGCTCAGCTGGTAGAGCG |
| | | GAGGACTttaGCTAACTCCCCGTTAG |
| | | AAGACATCCTTAGGTCGCTGGTTCGA |
| | | CTCCGGCTCGAAGGA |
| 70 | pre-tRNA-Tyr-GTA->tta--5-1 | CTTTCGATAGTTCAGTTGGTAGAGCG |
| | | GAGGACTttaGAGTATTAACGTTGGT |
| | | GATCCTTAGGTCGCTGGTTCGAGTCC |
| | | GGCTCGAAGGA |
| 71 | tRNA-Cys-GCA->tca--1-1 | GGGGGTATAGCTCAGTGGTAGAGCAT |
| | | TTGACTtcaGATCAAGAGGTCCCTGG |
| | | TTCAAATCCAGGTGCCCCCT |
| 72 | tRNA-Cys-GCA->tca--10-1 | GGGGGTATAGCTCAGGGGTAGAGCAT |
| | | TTGACTtcaGATCAAGAGGTCCCTGG |
| | | TTCAAATCCAGATGCCCCCT |
| 73 | tRNA-Cys-GCA->tca--11-1 | GGGGGTATAGCTCAGGGGTAGAGTAT |
| | | TTGGCTtcaGATCAAGAGGTCCCTGG |
| | | TTCAAATCCAGGTGCCCCCT |
| 74 | tRNA-Cys-GCA->tca--12-1 | GGGGGTATAGCTCAGGGGTAGAGCAT |
| | | TTGACTtcaGATCAAGAGGTCCTTGG |
| | | TTCAAATCCAGGTGTCCCCT |
| 75 | tRNA-Cys-GCA->tca--13-1 | GGGGGTATAGCTCAGAGGTAGAGCAT |
| | | TTGACTtcaGATCAAGAGATCTCTGG |
| | | TTCAAATCCAGGTGCCCCCT |
| 76 | tRNA-Cys-GCA->tca--14-1 | GGGGGTATAGCTCAGGGGTAGAGCAT |
| | | TTGACTtcaGATCAAGAGGTCCCTAG |
| | | TTCAAATCCAGGTGCCCCCT |
| 77 | tRNA-Cys-GCA->tca--15-1 | GGTGGTATAGCTCAGGGGTAGAGCAT |
| | | TTGACTtcaGATCAAGAGATCCCTGG |
| | | TTCGAATCCAGGTGCCCCCT |
| 78 | tRNA-Cys-GCA->tca--16-1 | GGGGGTATAACTCAGGGGTAGAGCAT |
| | | TTGACTtcaGATCAAGAGGTCCCTGG |
| | | TTCAAATCCAGGTGCCCCCT |
| 79 | tRNA-Cys-GCA->tca--17-1 | TGGGGTATAGCTCAGGGGTAGAGCAT |
| | | TTGACTtcaGATCAAGAGGTCCCTGG |
| | | TTCAAATCCAGGTGCCCCCT |
| 80 | tRNA-Cys-GCA->tca--18-1 | GGGGGTATAGCTCAGAGGAAGAGCAT |
| | | TTGACTtcaGATCAAGAGGTCCCTGA |
| | | TTCAAATCCAGGTGCCCCCT |
| 81 | tRNA-Cys-GCA->tca--19-1 | GGGGGTAAAGCTCAGGGGTAGAGCAT |
| | | TTGACTtcaGATTAAGAGGTCCCTGG |
| | | TTCAAATCCAGGTACCCCCT |
| 82 | tRNA-Cys-GCA->tca--2-1 | GGGGGTATAGCTCAGTGGTAGAGCAT |
| | | TTGACTtcaGATCAAGAGGTCCCTGG |
| | | TTCAAATCCGGGTGCCCCCT |
| 83 | tRNA-Cys-GCA->tca--21-1 | GGGGTTATAGCTCAGGTGTAGAGCAT |
| | | TTGACTtcaGATCAAGAGGTCCCTGG |
| | | TTCAAATCCAGGTGCCCCCT |
| 84 | tRNA-Cys-GCA->tca--24-1 | GGGGGTATAGCTCAGGGGTAGAGCAT |
| | | TTGACTtcaGATCACGAGGTCCCTGG |
| | | TTCAAATCGAGGTGCCCCCT |
| 85 | tRNA-Cys-GCA->tca--25-1 | GGGGGTATAGCTCAGGGGTGGAGCAT |
| | | TTGACTtcaGATCAAGGGGTCCCTGT |
| | | TTCAAATCCAGGTGCCCCCT |
| 86 | tRNA-Cys-GCA->tca--3-1 | GGGGGTATAGCTCAGTGGTAGAGCAT |
| | | TTGACTtcaGATCAAGAGGTCCCCGG |
| | | TTCAAATCCGGGTGCCCCCT |
| 87 | tRNA-Cys-GCA->tca--4-1 | GGGGGTATAGCTCAGGGGTAGAGCAT |
| | | TTGACTtcaGATCAAGAGGTCCCTGG |
| | | TTCAAATCCAGGTGCCCCCT |
| 88 | tRNA-Cys-GCA->tca--5-1 | GGGGGTATAGCTCAGGGGTAGAGCAT |
| | | TTGACTtcaGATCAAGAGGTCCCTGG |
| | | TTCAAATCCGGGTGCCCCCT |
| 89 | tRNA-Cys-GCA->tca--6-1 | GGGGGTATAGCTCAGGGGTAGAGCAT |
| | | TTGACTtcaGATCAAGAGGTCCCTGG |
| | | TTCAAATCCAGGTACCCCCT |
| 90 | tRNA-Cys-GCA->tca--7-1 | GGGGGTATAGCTCAGGGGTAGAGCAT |
| | | TTGACTtcaGATCAAGAGGTCCCCGG |
| | | TTCAAATCCGGGTGCCCCCT |
| 91 | tRNA-Cys-GCA->tca--8-1 | GGGGGCATAGCTCAGGGGTAGAGCAT |
| | | TTGACTtcaGATCAAGAGGTCCCCGG |
| | | TTCAAATCCGGGTGCTCCCT |
| 92 | tRNA-Cys-GCA->tca--9-1 | GGGGGTATAGCTCAGGGGTAGAGCAT |
| | | TTGACTtcaGATTAAGAGGTCCCTGG |
| | | TTCAAATCCAGGTGCCCCCT |
| 93 | tRNA-Glu-CTC->cta--1-1 | TCCCTGGTGGTCTAGTGGTTAGGATT |
| | | CGGCGCTctaACCGCCGCGGCCCGGG |
| | | TTCGATTCCCGGTCAGGGAA |
| 94 | tRNA-Glu-CTC->cta--2-1 | TCCCTGGTGGTCTAGTGGTTAGGATT |
| | | TGGCGCTctaACCGCCGCGGCCTGGG |
| | | TTCGATTCCCGGTCAGGGAA |
| 95 | tRNA-Glu-CTC->cta--5-1 | TCCCTGGTGGTCTAGTGGTTAGGCTT |
| | | TGGTGCTctaACCTCCATGGCCCAGG |
| | | TTTGATTCCTGGTCAGGGAA |
| 96 | tRNA-Glu-CTC->tta--1-1 | TCCCTGGTGGTCTAGTGGTTAGGATT |
| | | CGGCGCTttaACCGCCGCGGCCCGGG |
| | | TTCGATTCCCGGTCAGGGAA |
| 97 | tRNA-Glu-CTC->tta--2-1 | TCCCTGGTGGTCTAGTGGTTAGGATT |
| | | TGGCGCTttaACCGCCGCGGCCTGGG |
| | | TTCGATTCCCGGTCAGGGAA |
| 98 | tRNA-Glu-CTC->tta--5-1 | TCCCTGGTGGTCTAGTGGTTAGGCTT |
| | | TGGTGCTttaACCTCCATGGCCCAGG |
| | | TTTGATTCCTGGTCAGGGAA |
| 99 | tRNA-Glu-TTC->cta--1-1 | TCCCACATGGTCTAGCGGTTAGGATT |
| | | CCTGGTTctaACCCAGGCGGCCCGGG |
| | | TTCGACTCCCGGTGTGGGAA |
| 100 | tRNA-Glu-TTC->cta--2-1 | TCCCATATGGTCTAGCGGTTAGGATT |
| | | CCTGGTTctaACCCAGGCGGCCCGGG |
| | | TTCGACTCCCGGTATGGGAA |
| 101 | tRNA-Glu-TTC->cta--3-1 | TCCCTGGTGGTCTAGTGGCTAGGATT |
| | | CGGCGCTctaACCGCCGCGGCCCGGG |
| | | TTCGATTCCCGGTCAGGGAA |
| 102 | tRNA-Glu-TTC->tta--1-1 | TCCCACATGGTCTAGCGGTTAGGATT |
| | | CCTGGTTttaACCCAGGCGGCCCGGG |
| | | TTCGACTCCCGGTGTGGGAA |
| 103 | tRNA-Glu-TTC->tta--2-1 | TCCCATATGGTCTAGCGGTTAGGATT |
| | | CCTGGTTttaACCCAGGCGGCCCGGG |
| | | TTCGACTCCCGGTATGGGAA |
| 104 | tRNA-Glu-TTC->tta--3-1 | TCCCTGGTGGTCTAGTGGCTAGGATT |
| | | CGGCGCTttaACCGCCGCGGCCCGGG |
| | | TTCGATTCCCGGTCAGGGAA |
| 105 | tRNA-Gly-ACC->tca--1-1 | GTTTCCGTAGTGTAGTGGTTAGCGCG |
| | | TTCGCCTtcaAAAGCGAAAGGTCCCC |
| | | GGTTCGAAACCGGGCGGAAACA |
| 106 | tRNA-Gly-CCC->tca--1-1 | GCGCCGCTGGTGTAGTGGTATCATGC |
| | | AAGATTtcaATTCTTGCGACCCGGGT |
| | | TCGATTCCCGGGCGGCGCA |
| 107 | tRNA-Gly-CCC->tca--2-1 | GCATTGGTAGTTCAATGGTAGAATTC |
| | | TCGCCTtcaACGCGGGTGACCCGGGT |
| | | TCGATTCCCGGCCAATGCA |
| 108 | RNA-Gly-CCC->tca--3-1 | GCATTGGTGGTTCAATGGTAGAATTC |
| | | TCGCCTtcaACGCGGGTGACCCGGGT |
| | | TCGATTCCCGGCCAATGCA |
| 109 | tRNA-Gly-CCC->tca--4-1 | GCATTGGTGGTTCAATGGTAGAATTC |
| | | TCGCCTtcaACTCGGGTGACCCGGGT |
| | | TCGATTCCCGGCCAATGCA |
| 110 | tRNA-Gly-GCC->tca--1-1 | GCATGGGTGGTTCAGTGGTAGAATTC |
| | | TCGCCTtcaACGCGGGAGGCCCGGGT |
| | | TCGATTCCCGGCCCATGCA |
| 111 | tRNA-Gly-GCC->tca--2-1 | GCATTGGTGGTTCAGTGGTAGAATTC |
| | | TCGCCTtcaACGCGGGAGGCCCGGGT |
| | | TCGATTCCCGGCCAATGCA |
| 112 | tRNA-Gly-GCC->tca--3-1 | GCATTGGTGGTTCAGTGGTAGAATTC |
| | | TCGCCTtcaACGCGGGAGGCCCGGGT |
| | | TTGATTCCCGGCCAATGCA |
| 113 | tRNA-Gly-GCC->tca--4-1 | GCATTGGTGGTTCAGTGGTAGAATTC |
| | | TCGCCTtcaACGCGGGAGGCCCGGGT |
| | | TCGGTTCCCGGCCAATGCA |
| 114 | tRNA-Gly-TCC->tca--1-1 | GCGTTGGTGGTATAGTGGTGAGCATA |
| | | GCTGCCTtcaAAGCAGTTGACCCGGG |
| | | TTCGATTCCCGGCCAACGCA |
| 115 | tRNA-Leu-AAG->cta--1-1 | GGTAGCGTGGCCGAGCGGTCTAAGGC |
| | | GCTGGATTctaGCTCCAGTCTCTTCG |
| | | GGGGCGTGGGTTCGAATCCCACCGCT |
| | | GCCA |
| 116 | tRNA-Leu-AAG->cta--2-1 | GGTAGTGTGGCCGAGCGGTCTAAGGC |
| | | GCTGGATTctaGCTCCAGTCTCTTCG |
| | | GGGGCGTGGGTTCGAATCCCACCGCT |
| | | GCCA |
| 117 | tRNA-Leu-AAG->cta--3-1 | GGTAGTGTGGCCGAGCGGTCTAAGGC |
| | | GCTGGATTctaGCTCCAGTCTCTTCG |
| | | GGGGCGTGGGTTCGAATCCCACCACT |
| | | GCCA |
| 118 | tRNA-Leu-AAG->tca--1-1 | GGTAGCGTGGCCGAGCGGTCTAAGGC |
| | | GCTGGATTtcaGCTCCAGTCTCTTCG |
| | | GGGGCGTGGGTTCGAATCCCACCGCT |
| | | GCCA |
| 119 | tRNA-Leu-AAG->tca--2-1 | GGTAGTGTGGCCGAGCGGTCTAAGGC |
| | | GCTGGATTtcaGCTCCAGTCTCTTCG |
| | | GGGGCGTGGGTTCGAATCCCACCGCT |
| | | GCCA |
| 120 | tRNA-Leu-AAG->tca--3-1 | GGTAGTGTGGCCGAGCGGTCTAAGGC |
| | | GCTGGATTtcaGCTCCAGTCTCTTCG |
| | | GGGGCGTGGGTTCGAATCCCACCACT |
| | | GCCA |
| 121 | tRNA-Leu-AAG->tta--1-1 | GGTAGCGTGGCCGAGCGGTCTAAGGC |
| | | GCTGGATTttaGCTCCAGTCTCTTCG |
| | | GGGGCGTGGGTTCGAATCCCACCGCT |
| | | GCCA |
| 122 | tRNA-Leu-AAG->tta--2-1 | GGTAGTGTGGCCGAGCGGTCTAAGGC |
| | | GCTGGATTttaGCTCCAGTCTCTTCG |
| | | GGGGCGTGGGTTCGAATCCCACCGCT |
| | | GCCA |
| 123 | tRNA-Leu-AAG->tta--3-1 | GGTAGTGTGGCCGAGCGGTCTAAGGC |
| | | GCTGGATTttaGCTCCAGTCTCTTCG |
| | | GGGGCGTGGGTTCGAATCCCACCACT |
| | | GCCA |
| 124 | tRNA-Leu-CAA->cta--1-1 | GTCAGGATGGCCGAGTGGTCTAAGGC |
| | | GCCAGACTctaGTTCTGGTCTCCCCT |
| | | GGAGGCGTGGGTTCGAATCCCACTTC |
| | | TGACA |
| 125 | tRNA-Leu-CAA->cta--2-1 | GTCAGGATGGCCGAGTGGTCTAAGGC |
| | | GCCAGACTctaGTTCTGGTCTCCGTA |
| | | TGGAGGCGTGGGTTCGAATCCCACTT |
| | | CTGACA |
| 126 | tRNA-Leu-CAA->cta--3-1 | GTCAGGATGGCCGAGTGGTCTAAGGC |
| | | GCCAGACTctaGTTCTGGTCTCCGAA |
| | | TGGAGGCGTGGGTTCGAATCCCACTT |
| | | CTGACA |
| 127 | tRNA-Leu-CAA->cta--4-1 | GTCAGGATGGCCGAGTGGTCTAAGGC |
| | | GCCAGACTctaGTTCTGGTCTCCGTG |
| | | TGGAGGCGTGGGTTCGAATCCCACTT |
| | | CTGACA |
| 128 | tRNA-Leu-CAA->tca--1-1 | GTCAGGATGGCCGAGTGGTCTAAGGC |
| | | GCCAGACTtcaGTTCTGGTCTCCCCT |
| | | GGAGGCGTGGGTTCGAATCCCACTTC |
| | | TGACA |
| 129 | tRNA-Leu-CAA->tca--2-1 | GTCAGGATGGCCGAGTGGTCTAAGGC |
| | | GCCAGACTtcaGTTCTGGTCTCCGTA |
| | | TGGAGGCGTGGGTTCGAATCCCACTT |
| | | CTGACA |
| 130 | tRNA-Leu-CAA->tca--3-1 | GTCAGGATGGCCGAGTGGTCTAAGGC |
| | | GCCAGACTtcaGTTCTGGTCTCCGAA |
| | | TGGAGGCGTGGGTTCGAATCCCACTT |
| | | CTGACA |
| 131 | tRNA-Leu-CAA->tca--4-1 | GTCAGGATGGCCGAGTGGTCTAAGGC |
| | | GCCAGACTtcaGTTCTGGTCTCCGTG |
| | | TGGAGGCGTGGGTTCGAATCCCACTT |
| | | CTGACA |
| 132 | tRNA-Leu-CAA->tta--1-1 | GTCAGGATGGCCGAGTGGTCTAAGGC |
| | | GCCAGACTttaGTTCTGGTCTCCCCT |
| | | GGAGGCGTGGGTTCGAATCCCACTTC |
| | | TGACA |
| 133 | tRNA-Leu-CAA->tta--2-1 | GTCAGGATGGCCGAGTGGTCTAAGGC |
| | | GCCAGACTttaGTTCTGGTCTCCGTA |
| | | TGGAGGCGTGGGTTCGAATCCCACTT |
| | | CTGACA |
| 134 | tRNA-Leu-CAA->tta--3-1 | GTCAGGATGGCCGAGTGGTCTAAGGC |
| | | GCCAGACTttaGTTCTGGTCTCCGAA |
| | | TGGAGGCGTGGGTTCGAATCCCACTT |
| | | CTGACA |
| 135 | tRNA-Leu-CAA->tta--4-1 | GTCAGGATGGCCGAGTGGTCTAAGGC |
| | | GCCAGACTttaGTTCTGGTCTCCGTG |
| | | TGGAGGCGTGGGTTCGAATCCCACTT |
| | | CTGACA |
| 136 | tRNA-Leu-CAG->cta--1-1 | GTCAGGATGGCCGAGCGGTCTAAGGC |
| | | GCTGCGTTctaGTCGCAGTCTCCCCT |
| | | GGAGGCGTGGGTTCGAATCCCACTCC |
| | | TGACA |
| 137 | tRNA-Leu-CAG->cta--2-1 | GTCAGGATGGCCGAGCGGTCTAAGGC |
| | | GCTGCGTTctaGTCGCAGTCTCCCCT |
| | | GGAGGCGTGGGTTCGAATCCCACTTC |
| | | TGACA |
| 138 | tRNA-Leu-CAG->cta--3-1 | GTCAGGATGGCCGAGTGGTCTAAGGA |
| | | GCTGTGTTctaGTCGCAGTCTCCCCT |
| | | GGAGGCGTGGGTTCGAATCCCACTCC |
| | | TGACA |
| 139 | tRNA-Leu-CAG->cta--4-1 | GTCAGGATGGCCGAGCAGTCTAAGGC |
| | | ACTGCGTTctaGTCGCAGTCTCCCCT |
| | | GGAGGCGTGGATTCGAATCCCACTCC |
| | | TGACA |
| 140 | tRNA-Leu-CAG->tca--1-1 | GTCAGGATGGCCGAGCGGTCTAAGGC |
| | | GCTGCGTTtcaGTCGCAGTCTCCCCT |
| | | GGAGGCGTGGGTTCGAATCCCACTCC |
| | | TGACA |
| 141 | tRNA-Leu-CAG->tca--2-1 | GTCAGGATGGCCGAGCGGTCTAAGGC |
| | | GCTGCGTTtcaGTCGCAGTCTCCCCT |
| | | GGAGGCGTGGGTTCGAATCCCACTTC |
| | | TGACA |
| 142 | tRNA-Leu-CAG->tca--3-1 | GTCAGGATGGCCGAGTGGTCTAAGGA |
| | | GCTGTGTTtcaGTCGCAGTCTCCCCT |
| | | GGAGGCGTGGGTTCGAATCCCACTCC |
| | | TGACA |
| 143 | tRNA-Leu-CAG->tca--4-1 | GTCAGGATGGCCGAGCAGTCTAAGGC |
| | | ACTGCGTTtcaGTCGCAGTCTCCCCT |
| | | GGAGGCGTGGATTCGAATCCCACTCC |
| | | TGACA |
| 144 | tRNA-Leu-CAG->tta--1-1 | GTCAGGATGGCCGAGCGGTCTAAGGC |
| | | GCTGCGTTttaGTCGCAGTCTCCCCT |
| | | GGAGGCGTGGGTTCGAATCCCACTCC |
| | | TGACA |
| 145 | tRNA-Leu-CAG->tta--2-1 | GTCAGGATGGCCGAGCGGTCTAAGGC |
| | | GCTGCGTTttaGTCGCAGTCTCCCCT |
| | | GGAGGCGTGGGTTCGAATCCCACTTC |
| | | TGACA |
| 146 | tRNA-Leu-CAG->tta--3-1 | GTCAGGATGGCCGAGTGGTCTAAGGA |
| | | GCTGTGTTttaGTCGCAGTCTCCCCT |
| | | GGAGGCGTGGGTTCGAATCCCACTCC |
| | | TGACA |
| 147 | tRNA-Leu-CAG->tta--4-1 | GTCAGGATGGCCGAGCAGTCTAAGGC |
| | | ACTGCGTTttaGTCGCAGTCTCCCCT |
| | | GGAGGCGTGGATTCGAATCCCACTCC |
| | | TGACA |
| 148 | tRNA-Leu-TAA->cta--1-1 | ACCAGAATGGCCGAGTGGTTAAGGCG |
| | | TTGGACTctaGATCCAATGGATTTAT |
| | | ATCCGCGTGGGTTCGAACCCCACTTC |
| | | TGGTA |
| 149 | tRNA-Leu-TAA->cta--2-1 | ACCAGGATGGCCGAGTGGTTAAGGCG |
| | | TTGGACTctaGATCCAATGGACATAT |
| | | GTCTGCGTGGGTTCGAACCCCACTCC |
| | | TGGTA |
| 150 | tRNA-Leu-TAA->cta--3-1 | ACTGGGATGGCTGAGTGGTTAAGGCG |
| | | TTGGACTctaGATCCAATGGGCGGTT |
| | | GCCTGCGTGGGTTCGAACCCCACTCC |
| | | CAGTA |
| 151 | tRNA-Leu-TAA->cta--4-1 | GATGGGATGGCTGAGAGGTTAAGGCT |
| | | TTGGACTctaGATCCAATGGGCAGAT |
| | | GCCTGCGTGGGTTTGAACCCCACTCC |
| | | CAATA |
| 152 | tRNA-Leu-TAA->tca--1-1 | ACCAGAATGGCCGAGTGGTTAAGGCG |
| | | TTGGACTtcaGATCCAATGGATTTAT |
| | | ATCCGCGTGGGTTCGAACCCCACTTC |
| | | TGGTA |
| 153 | tRNA-Leu-TAA->tca--2-1 | ACCAGGATGGCCGAGTGGTTAAGGCG |
| | | TTGGACTtcaGATCCAATGGACATAT |
| | | GTCTGCGTGGGTTCGAACCCCACTCC |
| | | TGGTA |
| 154 | tRNA-Leu-TAA->tca--3-1 | ACTGGGATGGCTGAGTGGTTAAGGCG |
| | | TTGGACTtcaGATCCAATGGGCGGTT |
| | | GCCTGCGTGGGTTCGAACCCCACTCC |
| | | CAGTA |
| 155 | tRNA-Leu-TAA->tca--4-1 | GATGGGATGGCTGAGAGGTTAAGGCT |
| | | TTGGACTtcaGATCCAATGGGCAGAT |
| | | GCCTGCGTGGGTTTGAACCCCACTCC |
| | | CAATA |
| 156 | tRNA-Leu-TAA->tta--1-1 | ACCAGAATGGCCGAGTGGTTAAGGCG |
| | | TTGGACTttaGATCCAATGGATTTAT |
| | | ATCCGCGTGGGTTCGAACCCCACTTC |
| | | TGGTA |
| 157 | tRNA-Leu-TAA->tta--2-1 | ACCAGGATGGCCGAGTGGTTAAGGCG |
| | | TTGGACTttaGATCCAATGGACATAT |
| | | GTCTGCGTGGGTTCGAACCCCACTCC |
| | | TGGTA |
| 158 | tRNA-Leu-TAA->tta--3-1 | ACTGGGATGGCTGAGTGGTTAAGGCG |
| | | TTGGACTttaGATCCAATGGGCGGTT |
| | | GCCTGCGTGGGTTCGAACCCCACTCC |
| | | CAGTA |
| 159 | tRNA-Leu-TAA->tta--4-1 | GATGGGATGGCTGAGAGGTTAAGGCT |
| | | TTGGACTttaGATCCAATGGGCAGAT |
| | | GCCTGCGTGGGTTTGAACCCCACTCC |
| | | CAATA |
| 160 | tRNA-Leu-TAG->cta--1-1 | GGTAGCGTGGCCGAGCGGTCTAAGGC |
| | | GCTGGATTctaGCTCCAGTCTCTTCG |
| | | GAGGCGTGGGTTCGAATCCCACCGCT |
| | | GCCA |
| 161 | tRNA-Leu-TAG->cta--2-1 | GGTAGTGTGGCCGAGCGGTCTAAGGC |
| | | GCTGGATTctaGCTCCAGTCTCTTCG |
| | | GAGGCGTGGGTTCGAATCCCACCACT |
| | | GCCA |
| 162 | tRNA-Leu-TAG->cta--3-1 | GGTAGCGTGGCCGAGTGGTCTAAGGC |
| | | GCTGGATTctaGCTCCAGTCATTTCG |
| | | ATGGCGTGGGTTCGAATCCCACCGCT |
| | | GCCA |
| 163 | tRNA-Leu-TAG->tca--1-1 | GGTAGCGTGGCCGAGCGGTCTAAGGC |
| | | GCTGGATTtcaGCTCCAGTCTCTTCG |
| | | GAGGCGTGGGTTCGAATCCCACCGCT |
| | | GCCA |
| 164 | tRNA-Leu-TAG->tca--2-1 | GGTAGTGTGGCCGAGCGGTCTAAGGC |
| | | GCTGGATTtcaGCTCCAGTCTCTTCG |
| | | GAGGCGTGGGTTCGAATCCCACCACT |
| | | GCCA |
| 165 | tRNA-Leu-TAG->tca--3-1 | GGTAGCGTGGCCGAGTGGTCTAAGGC |
| | | GCTGGATTtcaGCTCCAGTCATTTCG |
| | | ATGGCGTGGGTTCGAATCCCACCGCT |
| | | GCCA |
| 166 | tRNA-Leu-TAG->tta--1-1 | GGTAGCGTGGCCGAGCGGTCTAAGGC |
| | | GCTGGATTttaGCTCCAGTCTCTTCG |
| | | GAGGCGTGGGTTCGAATCCCACCGCT |
| | | GCCA |
| 167 | tRNA-Leu-TAG->tta--2-1 | GGTAGTGTGGCCGAGCGGTCTAAGGC |
| | | GCTGGATTttaGCTCCAGTCTCTTCG |
| | | GAGGCGTGGGTTCGAATCCCACCACT |
| | | GCCA |
| 168 | tRNA-Leu-TAG->tta--3-1 | GGTAGCGTGGCCGAGTGGTCTAAGGC |
| | | GCTGGATTttaGCTCCAGTCATTTCG |
| | | ATGGCGTGGGTTCGAATCCCACCGCT |
| | | GCCA |
| 169 | tRNA-Lys-CTT->cta--1-1 | GCCCAGCTAGCTCAGTTGGTAGAGCG |
| | | TGGGACTctaAATCCTAGGGTCGTGG |
| | | GTTCGAACCCCACGTTGGGCG |
| 170 | tRNA-Lys-CTT->cta--12-1 | GCCCAGCTAGCTCAGTCTGTAGAGCA |
| | | TGAGACTctaAGTCTCAGGGTCATGG |
| | | GTTGGAGCCCCATGTTGTGCA |
| 171 | tRNA-Lys-CTT->cta--13-1 | GCCTAGCTAGTTCAGTCGGTAGAGCA |
| | | TGAGACTctaAATCTCAGGTTCATGA |
| | | GTTTGAGCCCCATGTTGGTTTGGCA |
| 172 | tRNA-Lys-CTT->cta--14-1 | CCCCGGCTAGCTCAGTCAGTAGAGCT |
| | | TGAGAATctaAATCTCAGGGTCGTGG |
| | | GTTGGAGCCCCACGTTGGGCG |
| 196 | tRNA-Lys-CTT->cta--2-1 | GCCCGGCTAGCTCAGTCGGTAGAGCA |
| | | TGGGACTctaAATCCCAGGGTCGTGG |
| | | GTTCGAGCCCCACGTTGGGCG |
| 197 | tRNA-Lys-CTT->cta--3-1 | GCCCGGCTAGCTCAGTCGGTAGAGCA |
| | | TGAGACTctaAATCTCAGGGTCGTGG |
| | | GTTCGAGCCCCACGTTGGGCG |
| 198 | tRNA-Lys-CTT->cta--4-1 | GCCCAGCTAGCTCAGTCTGTAGAGCA |
| | | TGAGACTctaAATCTCAGGGTCGTGA |
| | | GTTCGAGCCCCACGTTGGGTG |
| 199 | tRNA-Lys-CTT->cta--5-1 | GCCCAGATAGCTCAGTGGGTAGAGCA |
| | | TGAGACTctaAATCTCAGGGTCATGG |
| | | GTTCATGCCCCATGTTGGGTA |
| 200 | tRNA-Lys-CTT->cta--6-1 | GTCCTGCTGGCTCAGTCGGTACAGCA |
| | | TGGGACTctaAATCCCAGGGTCGTGG |
| | | GTTCGAGCTCCACGTTGGGTA |
| 201 | tRNA-Lys-CTT->cta--7-1 | GCCTGGCTAGCTCAGTCCATAGAGCA |
| | | TGGGACTctaAATCCCAGGGTCATGG |
| | | GTTCGAGCCCCATATTAGGCA |
| 202 | tRNA-Lys-CTT->cta--8-1 | GCCCAGCTAGCTTAGTTGGTAGAGCA |
| | | TGAGACTctaAATCTCAGAGTCATGG |
| | | GTTCAGGCCTCATGTTTGGCA |
| 203 | RNA-Lys-CTT->cta--9-1 | AACCTGGCTAGGTCAGTTGGTAGATC |
| | | ATGAGACTctaAATCTCAGGGTCATG |
| | | GGTTCAAGCCCCATGTTGGTTT |
| 204 | tRNA-Lys-CTT->tta--1-1 | GCCCAGCTAGCTCAGTTGGTAGAGCG |
| | | TGGGACTttaAATCCTAGGGTCGTGG |
| | | GTTCGAACCCCACGTTGGGCG |
| 205 | tRNA-Lys-CTT->tta--12-1 | GCCCAGCTAGCTCAGTCTGTAGAGCA |
| | | TGAGACTttaAGTCTCAGGGTCATGG |
| | | GTTGGAGCCCCATGTTGTGCA |
| 206 | tRNA-Lys-CTT->tta--13-1 | GCCTAGCTAGTTCAGTCGGTAGAGCA |
| | | TGAGACTttaAATCTCAGGTTCATGA |
| | | GTTTGAGCCCCATGTTGGTTTGGCA |
| 207 | tRNA-Lys-CTT->tta--14-1 | CCCCGGCTAGCTCAGTCAGTAGAGCT |
| | | TGAGAATttaAATCTCAGGGTCGTGG |
| | | GTTGGAGCCCCACGTTGGGCG |
| 208 | tRNA-Lys-CTT->tta--2-1 | GCCCGGCTAGCTCAGTCGGTAGAGCA |
| | | TGGGACTttaAATCCCAGGGTCGTGG |
| | | GTTCGAGCCCCACGTTGGGCG |
| 209 | tRNA-Lys-CTT->tta--3-1 | GCCCGGCTAGCTCAGTCGGTAGAGCA |
| | | TGAGACTttaAATCTCAGGGTCGTGG |
| | | GTTCGAGCCCCACGTTGGGCG |
| 210 | tRNA-Lys-CTT->tta--4-1 | GCCCAGCTAGCTCAGTCTGTAGAGCA |
| | | TGAGACTttaAATCTCAGGGTCGTGA |
| | | GTTCGAGCCCCACGTTGGGTG |
| 211 | tRNA-Lys-CTT->tta--5-1 | GCCCAGATAGCTCAGTGGGTAGAGCA |
| | | TGAGACTttaAATCTCAGGGTCATGG |
| | | GTTCATGCCCCATGTTGGGTA |
| 212 | tRNA-Lys-CTT->tta--6-1 | GTCCTGCTGGCTCAGTCGGTACAGCA |
| | | TGGGACTttaAATCCCAGGGTCGTGG |
| | | GTTCGAGCTCCACGTTGGGTA |
| 213 | tRNA-Lys-CTT->tta--7-1 | GCCTGGCTAGCTCAGTCCATAGAGCA |
| | | TGGGACTttaAATCCCAGGGTCATGG |
| | | GTTCGAGCCCCATATTAGGCA |
| 214 | tRNA-Lys-CTT->tta--8-1 | GCCCAGCTAGCTTAGTTGGTAGAGCA |
| | | TGAGACTttaAATCTCAGAGTCATGG |
| | | GTTCAGGCCTCATGTTTGGCA |
| 215 | tRNA-Lys-CTT->tta--9-1 | AACCTGGCTAGGTCAGTTGGTAGATC |
| | | ATGAGACTttaAATCTCAGGGTCATG |
| | | GGTTCAAGCCCCATGTTGGTTT |
| 216 | tRNA-Lys-TTT->cta--1-1 | GCCCGGATAGCTCAGTCGGTAGAGCA |
| | | TCAGACTctaAATCTGAGGGTCCAGG |
| | | GTTCAAGTCCCTGTTCGGGCG |
| 217 | tRNA-Lys-TTT->cta--2-1 | GCCTGGATAGCTCAGTCGGTAGAGCA |
| | | TCAGACTctaAATCTGAGGGTCCAGG |
| | | GTTCAAGTCCCTGTTCAGGCG |
| 218 | tRNA-Lys-TTT->cta--3-1 | GCCTGGATAGCTCAATTGGTAGAGCA |
| | | TCAGACTctaAATCTGAGGGTTCAGG |
| | | GTTCAAGTCCCTGTTCAGGCG |
| 219 | tRNA-Lys-TTT->cta--4-1 | GCCCAGCCAGCTCAGTAGGTAGAGTA |
| | | TGAGACTctaAATCTCAGGGTGGTGG |
| | | GTTCGAGCCCCATGTTGGGGG |
| 220 | tRNA-Lys-TTT->cta--5-1 | TGTGGTGTAGCTCAGTCGGTAGAGCA |
| | | TCAGACTctaAATCTGAGGGTCCAGG |
| | | GTTCAGGTCCCTGTTCGGGTGCCAAA |
| | | A |
| 221 | tRNA-Lys-TTT->tta--1-1 | GCCCGGATAGCTCAGTCGGTAGAGCA |
| | | TCAGACTttaAATCTGAGGGTCCAGG |
| | | GTTCAAGTCCCTGTTCGGGCG |
| 222 | tRNA-Lys-TTT->tta--2-1 | GCCTGGATAGCTCAGTCGGTAGAGCA |
| | | TCAGACTttaAATCTGAGGGTCCAGG |
| | | GTTCAAGTCCCTGTTCAGGCG |
| 223 | tRNA-Lys-TTT->tta--3-1 | GCCTGGATAGCTCAATTGGTAGAGCA |
| | | TCAGACTttaAATCTGAGGGTTCAGG |
| | | GTTCAAGTCCCTGTTCAGGCG |
| 224 | tRNA-Lys-TTT->tta--4-1 | GCCCAGCCAGCTCAGTAGGTAGAGTA |
| | | TGAGACTttaAATCTCAGGGTGGTGG |
| | | GTTCGAGCCCCATGTTGGGGG |
| 225 | tRNA-Lys-TTT->tta--5-1 | TGTGGTGTAGCTCAGTCGGTAGAGCA |
| | | TCAGACTttaAATCTGAGGGTCCAGG |
| | | GTTCAGGTCCCTGTTCGGGTGCCAAA |
| | | A |
| 226 | tRNA-Ser-AGA->cta--1-1 | GTAGTCGTGGCCGAGTGGTTAAGGCG |
| | | ATGGACTctaAATCCATTGGGGTTTC |
| | | CCCGCGCAGGTTCGAATCCTGCCGAC |
| | | TACG |
| 227 | tRNA-Ser-AGA->cta--2-1 | GTAGTCGTGGCCGAGTGGTTAAGGCG |
| | | ATGGACTctaAATCCATTGGGGTCTC |
| | | CCCGCGCAGGTTCGAATCCTGCCGAC |
| | | TACG |
| 228 | tRNA-Ser-AGA->cta--3-1 | GTAGTCGTGGCCAAGTGAGTAAGGCA |
| | | ATGGACTctaAATCCATTGGGGTCTC |
| | | CCAGCACAGGTTCAAATCCTGCTGAC |
| | | TATG |
| 229 | tRNA-Ser-AGA->tca--1-1 | GTAGTCGTGGCCGAGTGGTTAAGGCG |
| | | ATGGACTtcaAATCCATTGGGGTTTC |
| | | CCCGCGCAGGTTCGAATCCTGCCGAC |
| | | TACG |
| 230 | tRNA-Ser-AGA->tca--2-1 | GTAGTCGTGGCCGAGTGGTTAAGGCG |
| | | ATGGACTtcaAATCCATTGGGGTCTC |
| | | CCCGCGCAGGTTCGAATCCTGCCGAC |
| | | TACG |
| 231 | tRNA-Ser-AGA->tca--3-1 | GTAGTCGTGGCCAAGTGAGTAAGGCA |
| | | ATGGACTtcaAATCCATTGGGGTCTC |
| | | CCAGCACAGGTTCAAATCCTGCTGAC |
| | | TATG |
| 232 | tRNA-Ser-AGA->tta--1-1 | GTAGTCGTGGCCGAGTGGTTAAGGCG |
| | | ATGGACTttaAATCCATTGGGGTTTC |
| | | CCCGCGCAGGTTCGAATCCTGCCGAC |
| | | TACG |
| 233 | tRNA-Ser-AGA->tta--2-1 | GTAGTCGTGGCCGAGTGGTTAAGGCG |
| | | ATGGACTttaAATCCATTGGGGTCTC |
| | | CCCGCGCAGGTTCGAATCCTGCCGAC |
| | | TACG |
| 234 | tRNA-Ser-AGA->tta--3-1 | GTAGTCGTGGCCAAGTGAGTAAGGCA |
| | | ATGGACTttaAATCCATTGGGGTCTC |
| | | CCAGCACAGGTTCAAATCCTGCTGAC |
| | | TATG |
| 235 | tRNA-Ser-CGA->cta--1-1 | GCTGTGATGGCCGAGTGGTTAAGGCG |
| | | TTGGACTctaAATCCAATGGGGTCTC |
| | | CCCGCGCAGGTTCGAATCCTGCTCAC |
| | | AGCG |
| 236 | tRNA-Ser-CGA->cta--2-1 | GTCACGGTGGCCGAGTGGTTAAGGCG |
| | | TTGGACTctaAATCCAATGGGGTTTC |
| | | CCCGCACAGGTTCGAATCCTGTTCGT |
| | | GACG |
| 237 | tRNA-Ser-CGA->cta--3-1 | GCTGTGATGGCCGAGTGGTTAAGGCG |
| | | TTGGACTctaAATCCAATGGGTTCTT |
| | | CCCGCGCAGGTTCAAATCCTGCTCAC |
| | | AGCG |
| 238 | tRNA-Ser-CGA->tca--1-1 | GCTGTGATGGCCGAGTGGTTAAGGCG |
| | | TTGGACTtcaAATCCAATGGGGTCTC |
| | | CCCGCGCAGGTTCGAATCCTGCTCAC |
| | | AGCG |
| 239 | tRNA-Ser-CGA->tca--2-1 | GTCACGGTGGCCGAGTGGTTAAGGCG |
| | | TTGGACTtcaAATCCAATGGGGTTTC |
| | | CCCGCACAGGTTCGAATCCTGTTCGT |
| | | GACG |
| 240 | tRNA-Ser-CGA->tca--3-1 | GCTGTGATGGCCGAGTGGTTAAGGCG |
| | | TTGGACTtcaAATCCAATGGGTTCTT |
| | | CCCGCGCAGGTTCAAATCCTGCTCAC |
| | | AGCG |
| 241 | tRNA-Ser-CGA->tta--1-1 | GCTGTGATGGCCGAGTGGTTAAGGCG |
| | | TTGGACTttaAATCCAATGGGGTCTC |
| | | CCCGCGCAGGTTCGAATCCTGCTCAC |
| | | AGCG |
| 242 | tRNA-Ser-CGA->tta--2-1 | GTCACGGTGGCCGAGTGGTTAAGGCG |
| | | TTGGACTttaAATCCAATGGGGTTTC |
| | | CCCGCACAGGTTCGAATCCTGTTCGT |
| | | GACG |
| 243 | tRNA-Ser-CGA->tta--3-1 | GCTGTGATGGCCGAGTGGTTAAGGCG |
| | | TTGGACTttaAATCCAATGGGTTCTT |
| | | CCCGCGCAGGTTCAAATCCTGCTCAC |
| | | AGCG |
| 244 | tRNA-Ser-GCT->cta--1-1 | GACGAGGTGGCCGAGTGGTTAAGGCG |
| | | ATGGACTctaAATCCATTGTGCTCTG |
| | | CACGCGTGGGTTCGAATCCCACCTTC |
| | | GTCG |
| 245 | tRNA-Ser-GCT->cta--2-1 | GACGAGGTGGCCGAGTGGTTAAGGCG |
| | | ATGGACTctaAATCCATTGTGCTCTG |
| | | CACGCATGGGTTCGAATCCCATCCTC |
| | | GTCG |
| 246 | tRNA-Ser-GCT->cta--3-1 | GACGAGGTGGCCGAGTGGTTAAGGCG |
| | | ATGGACTctaAATCCATTGTGCTTTG |
| | | CACGCGTGGGTTCGAATCCCATCCTC |
| | | GTCG |
| 247 | tRNA-Ser-GCT->cta--4-1 | GACGAGGTGGCCGAGTGGTTAAGGCG |
| | | ATGGACTctaAATCCATTGTGCTCTG |
| | | CACGCGTGGGTTCGAATCCCATCCTC |
| | | GTCG |
| 248 | tRNA-Ser-GCT->cta--5-1 | GATGAGGTGGCCGAGTGGTTAAGGCG |
| | | ATGGACTctaAATCCATTGTGCTCTG |
| | | CACGCATGGGTTCGAATCCCATCCTC |
| | | ATCG |
| 249 | tRNA-Ser-GCT->tca--1-1 | GACGAGGTGGCCGAGTGGTTAAGGCG |
| | | ATGGACTtcaAATCCATTGTGCTCTG |
| | | CACGCGTGGGTTCGAATCCCACCTTC |
| | | GTCG |
| 250 | tRNA-Ser-GCT->tca--2-1 | GACGAGGTGGCCGAGTGGTTAAGGCG |
| | | ATGGACTtcaAATCCATTGTGCTCTG |
| | | CACGCATGGGTTCGAATCCCATCCTC |
| | | GTCG |
| 251 | tRNA-Ser-GCT->tca--3-1 | GACGAGGTGGCCGAGTGGTTAAGGCG |
| | | ATGGACTtcaAATCCATTGTGCTTTG |
| | | CACGCGTGGGTTCGAATCCCATCCTC |
| | | GTCG |
| 252 | tRNA-Ser-GCT->tca--4-1 | GACGAGGTGGCCGAGTGGTTAAGGCG |
| | | ATGGACTtcaAATCCATTGTGCTCTG |
| | | CACGCGTGGGTTCGAATCCCATCCTC |
| | | GTCG |
| 253 | tRNA-Ser-GCT->tca--5-1 | GATGAGGTGGCCGAGTGGTTAAGGCG |
| | | ATGGACTtcaAATCCATTGTGCTCTG |
| | | CACGCATGGGTTCGAATCCCATCCTC |
| | | ATCG |
| 254 | tRNA-Ser-GCT->tta--1-1 | GACGAGGTGGCCGAGTGGTTAAGGCG |
| | | ATGGACTttaAATCCATTGTGCTCTG |
| | | CACGCGTGGGTTCGAATCCCACCTTC |
| | | GTCG |
| 255 | tRNA-Ser-GCT->tta--2-1 | GACGAGGTGGCCGAGTGGTTAAGGCG |
| | | ATGGACTttaAATCCATTGTGCTCTG |
| | | CACGCATGGGTTCGAATCCCATCCTC |
| | | GTCG |
| 256 | tRNA-Ser-GCT->tta--3-1 | GACGAGGTGGCCGAGTGGTTAAGGCG |
| | | ATGGACTttaAATCCATTGTGCTTTG |
| | | CACGCGTGGGTTCGAATCCCATCCTC |
| | | GTCG |
| 257 | tRNA-Ser-GCT->tta--4-1 | GACGAGGTGGCCGAGTGGTTAAGGCG |
| | | ATGGACTttaAATCCATTGTGCTCTG |
| | | CACGCGTGGGTTCGAATCCCATCCTC |
| | | GTCG |
| 258 | tRNA-Ser-GCT->tta--5-1 | GATGAGGTGGCCGAGTGGTTAAGGCG |
| | | ATGGACTttaAATCCATTGTGCTCTG |
| | | CACGCATGGGTTCGAATCCCATCCTC |
| | | ATCG |
| 259 | tRNA-Ser-GGA->cta--1-1 | GCTGAAATAGCTCAGTTGGGAGAGCA |
| | | TTAGACTctaGATCTAAAGGTCCCTG |
| | | GTTTGATCCCGGGTTTCGGCA |
| 260 | tRNA-Ser-GGA->tca--1-1 | GCTGAAATAGCTCAGTTGGGAGAGCA |
| | | TTAGACTtcaGATCTAAAGGTCCCTG |
| | | GTTTGATCCCGGGTTTCGGCA |
| 261 | tRNA-Ser-GGA->tta--1-1 | GCTGAAATAGCTCAGTTGGGAGAGCA |
| | | TTAGACTttaGATCTAAAGGTCCCTG |
| | | GTTTGATCCCGGGTTTCGGCA |
| 262 | tRNA-Ser-TGA->cta--1-1 | GCAGCGATGGCCGAGTGGTTAAGGCG |
| | | TTGGACTctaAATCCAATGGGGTCTC |
| | | CCCGCGCAGGTTCGAACCCTGCTCGC |
| | | TGCG |
| 263 | tRNA-Ser-TGA->tca--1-1 | GCAGCGATGGCCGAGTGGTTAAGGCG |
| | | TTGGACTtcaAATCCAATGGGGTCTC |
| | | CCCGCGCAGGTTCGAACCCTGCTCGC |
| | | TGCG |
| 264 | tRNA-Ser-TGA->tta--1-1 | GCAGCGATGGCCGAGTGGTTAAGGCG |
| | | TTGGACTttaAATCCAATGGGGTCTC |
| | | CCCGCGCAGGTTCGAACCCTGCTCGC |
| | | TGCG |
| 265 | RNA-Trp-CCA->cta--1-1 | GACCTCGTGGCGCAATGGTAGCGCGT |
| | | CTGACTctaGATCAGAAGGTTGCGTG |
| | | TTCAAATCACGTCGGGGTCA |
| 266 | tRNA-Trp-CCA->cta--2-1 | GACCTCGTGGCGCAACGGTAGCGCGT |
| | | CTGACTctaGATCAGAAGGCTGCGTG |
| | | TTCGAATCACGTCGGGGTCA |
| 267 | tRNA-Trp-CCA->cta--3-1 | GGCCTCGTGGCGCAACGGTAGCGCGT |
| | | CTGACTctaGATCAGAAGGTTGCGTG |
| | | TTCAAATCACGTCGGGGTCA |
| 268 | tRNA-Trp-CCA->cta--4-1 | GACCTCGTGGCGCAACGGTAGCGCGT |
| | | CTGACTctaGATCAGAAGGTTGCGTG |
| | | TTCAAATCACGTCGGGGTCA |
| 269 | tRNA-Trp-CCA->cta--5-1 | GACCTCGTGGCGCAATGGTAGCGCGT |
| | | CTGACTctaGATCAGAAGGTTGCGTG |
| | | TTCAAGTCACGTCGGGGTCA |
| 270 | tRNA-Trp-CCA->cta--6-1 | GACCTCGTGGCACAATGGTAGCACGT |
| | | CTGACTctaGATCAGAAGGTTGCGTG |
| | | TTCAAATCACGTCGGGGTCA |
| 271 | tRNA-Trp-CCA->tca--1-1 | GACCTCGTGGCGCAATGGTAGCGCGT |
| | | CTGACTtcaGATCAGAAGGTTGCGTG |
| | | TTCAAATCACGTCGGGGTCA |
| 272 | tRNA-Trp-CCA->tca--2-1 | GACCTCGTGGCGCAACGGTAGCGCGT |
| | | CTGACTtcaGATCAGAAGGCTGCGTG |
| | | TTCGAATCACGTCGGGGTCA |
| 273 | tRNA-Trp-CCA->tca--3-1 | GGCCTCGTGGCGCAACGGTAGCGCGT |
| | | CTGACTtcaGATCAGAAGGTTGCGTG |
| | | TTCAAATCACGTCGGGGTCA |
| 274 | tRNA-Trp-CCA->tca--4-1 | GACCTCGTGGCGCAACGGTAGCGCGT |
| | | CTGACTtcaGATCAGAAGGTTGCGTG |
| | | TTCAAATCACGTCGGGGTCA |
| 275 | tRNA-Trp-CCA->tca--5-1 | GACCTCGTGGCGCAATGGTAGCGCGT |
| | | CTGACTtcaGATCAGAAGGTTGCGTG |
| | | TTCAAGTCACGTCGGGGTCA |
| 276 | tRNA-Trp-CCA->tca--6-1 | GACCTCGTGGCACAATGGTAGCACGT |
| | | CTGACTtcaGATCAGAAGGTTGCGTG |
| | | TTCAAATCACGTCGGGGTCA |
| 277 | tRNA-Tyr-GTA->cta--1-1 | CCTTCGATAGCTCAGTTGGTAGAGCG |
| | | GAGGACTctaGATCCTTAGGTCGCTG |
| | | GTTCGAATCCGGCTCGAAGGA |
| 278 | tRNA-Tyr-GTA->cta--2-1 | CCTTCGATAGCTCAGTTGGTAGAGCG |
| | | GAGGACTctaGATCCTTAGGTCGCTG |
| | | GTTCGATTCCGGCTCGAAGGA |
| 279 | tRNA-Tyr-GTA->cta--3-1 | CCTTCGATAGCTCAGCTGGTAGAGCG |
| | | GAGGACTctaGATCCTTAGGTCGCTG |
| | | GTTCGATTCCGGCTCGAAGGA |
| 280 | tRNA-Tyr-GTA->cta--4-1 | CCTTCGATAGCTCAGCTGGTAGAGCG |
| | | GAGGACTctaGATCCTTAGGTCGCTG |
| | | GTTCGACTCCGGCTCGAAGGA |
| 281 | tRNA-Tyr-GTA->cta--5-1 | CTTTCGATAGTTCAGTTGGTAGAGCG |
| | | GAGGACTctaGATCCTTAGGTCGCTG |
| | | GTTCGAGTCCGGCTCGAAGGA |
| 282 | tRNA-Tyr-GTA->tta--1-1 | CCTTCGATAGCTCAGTTGGTAGAGCG |
| | | GAGGACTttaGATCCTTAGGTCGCTG |
| | | GTTCGAATCCGGCTCGAAGGA |
| 283 | tRNA-Tyr-GTA->tta--2-1 | CCTTCGATAGCTCAGTTGGTAGAGCG |
| | | GAGGACTttaGATCCTTAGGTCGCTG |
| | | GTTCGATTCCGGCTCGAAGGA |
| 284 | tRNA-Tyr-GTA->tta--3-1 | CCTTCGATAGCTCAGCTGGTAGAGCG |
| | | GAGGACTttaGATCCTTAGGTCGCTG |
| | | GTTCGATTCCGGCTCGAAGGA |
| 285 | tRNA-Tyr-GTA->tta--4-1 | CCTTCGATAGCTCAGCTGGTAGAGCG |
| | | GAGGACTttaGATCCTTAGGTCGCTG |
| | | GTTCGACTCCGGCTCGAAGGA |
| 286 | tRNA-Tyr-GTA->tta--5-1 | CTTTCGATAGTTCAGTTGGTAGAGCG |
| | | GAGGACTttaGATCCTTAGGTCGCTG |
| | | GTTCGAGTCCGGCTCGAAGGA |

The suppressor tRNAs are tested for PTC readthrough activity by flow cytometry in cell lines containing dual fluorescent readthrough reporters. These reporters include three copies of a red fluorescent protein (tdTomato), TEV protease, a linker region containing a PTC, and three copies of a green fluorescent protein (EGFP). A schematic of a reporter construct is shown in FIG. 3. In the absence of any PTC readthrough as a result of a suppressor tRNA, translation will be terminated by the PTC within the linker region, and only tdTomato will be expressed (and therefore only red fluorescence detected). PTC readthrough activity as a result of a suppressor tRNA will allow translation to proceed through the PTC in the linker region, and for both tdTomato and EGFP to be expressed (and therefore both red and green fluorescence detected). Accordingly, readthrough can be assessed by quantifying the percentage of viable cells expressing both the red and green fluorescent reporters above background (double positive %).

### Example 7

This Example describes glutamine aminoacylated suppressor tRNAs that facilitate read-through of a premature termination codon (PTC).

In this Example, all mature tRNA sequences were expressed in the context of upstream and downstream genomic flanking sequences (±200 bps) from tRNA-Gln-TTG-1-1—a highly expressed glutamine-tRNA, i.e., the tRNA sequences were expressed with a 5′ flanking sequence of SEQ ID NO: 173 and a 3′ flanking sequence of SEQ ID NO: 174. All mature tRNA sequences including upstream and downstream genomic flanking sequences were generated in a pGL4 vector backbone.

The G1n_{CTA} suppressor tRNAs were tested for PTC readthrough activity by flow cytometry in Flp-In-293 cells that either contain an integrated dual fluorescent readthrough reporter or were transiently co-transfected with an expression construct containing a dual fluorescent readthrough reporter. The reporters included three copies of a red fluorescent protein (tdTomato), TEV protease, a linker region containing a PTC, and three copies of a green fluorescent protein (EGFP). A schematic of the reporter construct is shown in FIG. 3. In the absence of any PTC readthrough as a result of a suppressor tRNA, translation will be terminated by the PTC within the linker region, and only tdTomato will be expressed (and therefore only red fluorescence detected). PTC readthrough activity as a result of a suppressor tRNA allows translation to proceed through the PTC in the linker region, and for both tdTomato and EGFP to be expressed (and therefore both red and green fluorescence to be detected). Accordingly, readthrough was assessed with flow cytometry by quantifying the percentage of viable cells expressing both the red and green fluorescent reporters above background (double positive %). To screen for suppressor tRNAs with readthrough activity at PTCs relevant to Dravet syndrome, linker regions were generated containing the PTC and eight flanking codons on either side of the PTC from the SCN1A transcript of three patients with Gln(Q)-to-TAG nonsense mutations in SCN1A: patient 3 (W1397X), patient 4 (51505X), and patient 5 (Q1810X).

The linker region derived from the patient 3 SCN1A transcript is as follows, and the reporter including this linker region is referred to as the patient3-Gln-TAG (W1397X) reporter:

(SEQ ID NO: 889)
ATAGAAAGAAATGAGACTGCTCGAtagAAAAATGTG
AAAGTAAACTTTGAT.

A corresponding linker with a wild-type Trp(W) codon in place of the PTC was used as a control, and had the following sequence:

(SEQ ID NO: 890)
ATAGAAAGAAATGAGACTGCTCGATGGAAAAATGT
GAAAGTAAACTTTGAT.

The linker region derived from the patient 4 SCN1A transcript is as follows, and the reporter including this linker region is referred to as the patient4-Gln-TAG (51505X) reporter:

(SEQ ID NO: 891)
TATAATGCAATGAAAAAATTAGGAtagAAAAAACCG
CAAAAGCCTATACCT.

A corresponding linker with a wild-type Ser(S) codon in place of the PTC was used as a control, and had the following sequence:

(SEQ ID NO: 892)
TATAATGCAATGAAAAAATTAGGATCGAAAAAACCG
CAAAAGCCTATACCT.

The linker region derived from the patient 5 SCN1A transcript is as follows, and the reporter including this linker region is referred to as the patient5-Gln-TAG (Q1810X) reporter:

(SEQ ID NO: 893)
GAGAAGTTTGATCCCGATGCAACTtagTTCATGGAA
TTTGAAAAATTATCT.

A corresponding linker with a wild-type Gln codon in place of the PTC was used as a control, and had the following sequence:

(SEQ ID NO: 894)
GAGAAGTTTGATCCCGATGCAACTCAGTTCATGGAAT
TTGAAAAATTATCT.

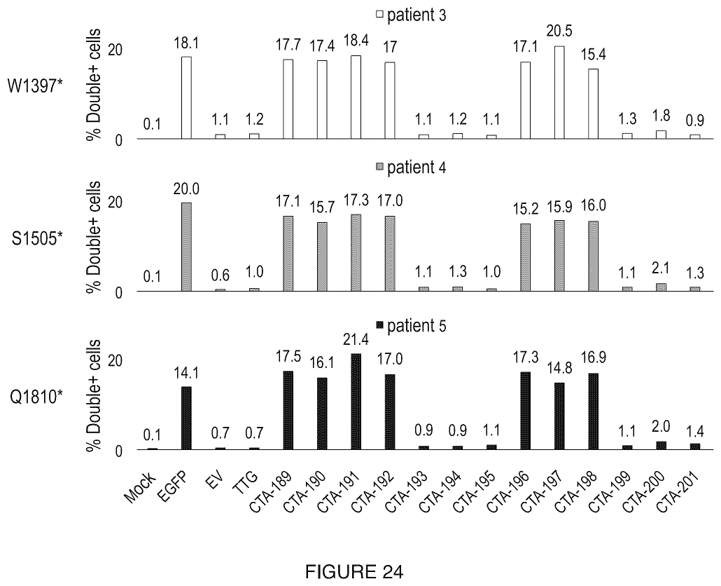

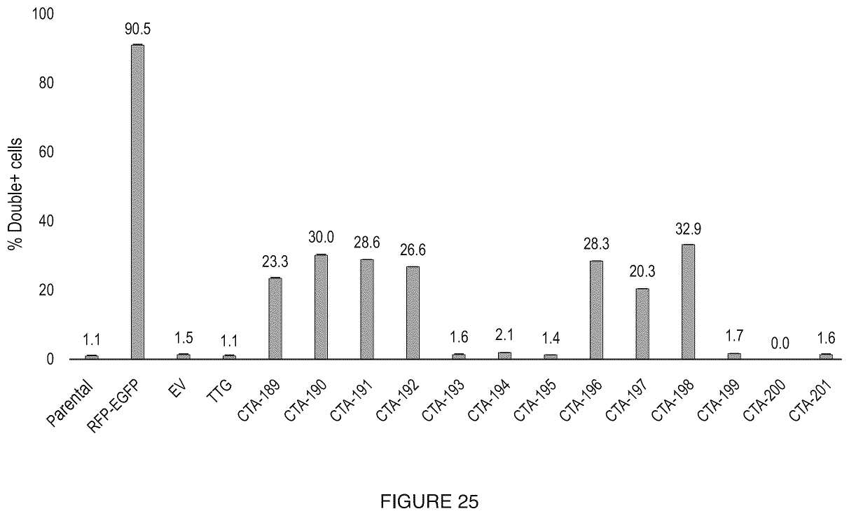

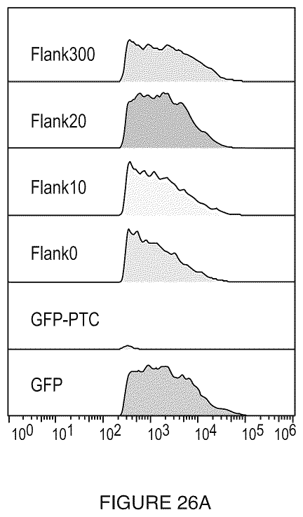

The G1n_{CTA} suppressor tRNAs (SEQ ID NOs: 178-190) were tested for PTC readthrough activity by flow cytometry in multiple assay contexts, including (i) human Flp-In-293 cells transiently co-transfected with the patient3-Gln-TAG (W1397X) reporter, the patient4-Gln-TAG (S1505X) reporter, or the patient5-Gln-TAG (Q1810X) reporter (results are shown in FIG. 24 and (ii) a human Flp-In-293 cell line cell line stably expressing the patient3-Gln-TAG (W1397X) reporter and transiently transfected with a plasmid encoding a G1n_{CTA} suppressor tRNA (results are shown in FIG. 25). Transfections were done using the Lipofectamine 3000 Transfection Reagent according to the manufacturer's protocol.

Together, these results demonstrate that the described suppressor tRNAs can facilitate expression of transcripts, e.g., SCN1A transcripts, containing premature termination codons associated with disorders, e.g., Dravet syndrome.

### Example 8

This Example describes the impact of the nucleotide sequence flanking a suppressor tRNA on the read-through of a premature termination codon (PTC) by the suppressor tRNA.

Expression vectors were generated that encoded an EGFP-R96X-TGA reporter (SEQ ID NO: 31, as described in Example 1) and a single copy of the Arg_{TCA} suppressor tRNA #115 (tRNA-Arg-TCT-2-1-TCA-SUP_no intron, SEQ ID NO: 18, as described in Example 1). The expression vectors included sequences immediately 5′ and 3′ to the tRNA coding sequence that were in each instance derived from the genomic DNA that is 5′ and 3′ to the mouse tRNA-Arg-TCG-1-1 gene but were of varying length. Details of the expression vectors are shown in TABLE 11.

TABLE 11

| | 5′ to tRNA | tRNA | 3′ to tRNA |
|---|---|---|---|
| | coding | coding | coding |
| Name | sequence | sequence | sequence |
| Flank300 | 200 nt | TCA-115 | 104 nt 3′ to |
| | 5′ to | (SEQ ID | the mouse tRNA- |
| | the mouse | NO: 18) | Arg-TCG-1-1 gene |
| | tRNA- | | (SEQ ID NO: 32) |
| | Arg- | | |
| | TCG-1-1 | | |
| | gene | | |
| | (SEQ ID | | |
| | NO: 26) | | |
| Flank20 | 20 nt 5′ | TCA-115 | 17 nt 3′ to |
| | to the | (SEQ ID | the mouse tRNA- |
| | mouse tRNA- | NO: 18) | Arg-TCG-1-1 gene |
| | Arg-TCG-1-1 | | (SEQ ID NO: 896) |
| | gene | | |
| | (SEQ ID | | |
| | NO: 895) | | |
| Flank10 | 10 nt 5′ | TCA-115 | 17 nt 3′ to |
| | to the | (SEQ ID | the mouse tRNA- |
| | mouse tRNA- | NO: 18) | Arg-TCG-1-1 gene |
| | Arg-TCG-1-1 | | (SEQ ID NO: 896) |
| | gene | | |
| | (SEQ ID | | |
| | NO: 897) | | |
| Flank0 | Random | TCA-115 | 17 nt 3′ to |
| | sequence | (SEQ ID | the mouse tRNA- |
| | | NO: 18) | Arg-TCG-1-1 gene |
| | | | (SEQ ID NO: 896) |

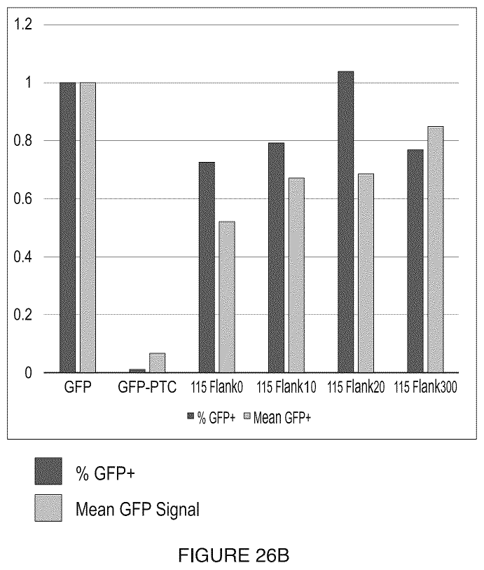

The expression vectors in TABLE 11 were tested for PTC readthrough activity by flow cytometry in Neuro-2a cells. Transfections were performed using the Lipofectamine 3000 Transfection Reagent according to the manufacturer's protocol. The results are shown in FIGS. 26A and 26B and demonstrate that, although the Arg_{TCA} suppressor tRNA #115, exhibits activity even with a random 5′ leader sequence, using a 5′ leader sequence from an endogenous tRNA gene can increase suppressor tRNA activity.

### Example 9

This Example describes the impact of the nucleotide sequence flanking a suppressor tRNA on the read-through of a premature termination codon (PTC) by the suppressor tRNA.

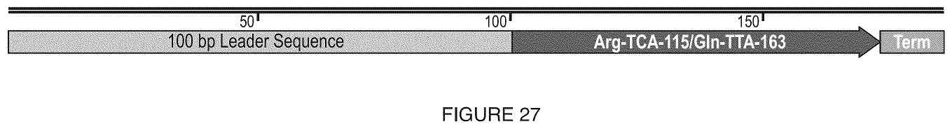

A library of expression vectors was generated that included (i) one of 20 unique 100 nt leader sequences (a sequence immediately 5′ to the tRNA coding sequence) derived from the human genomic DNA that is immediately 5′ to an endogenous tRNA gene in combination with (ii) a nucleotide sequence encoding TCA-115 (tRNA-Arg-TCT-2-1-TCA-SUP_no intron, SEQ ID NO: 18, as described in Example 1) or TTA-163 (tRNA-Gln-TTG-3-1-TTA-SUP, SEQ ID NO: 45, as described in Example 4). A schematic illustrating the design of the expression vector constructs in the library is depicted in FIG. 27. The 20 unique 100 nt leader sequences included sequences derived from the highest abundance tRNAs in human HEK293 cells and include leader sequences derived from Arg-TCT-1-1 (SEQ ID NO: 875), Tyr-GTA-5-1 (SEQ ID NO: 883), Ser-GCT-3-1 (SEQ ID NO: 878), Arg-TCG-1-1 (SEQ ID NO: 886), Arg-TCG-3-1 (SEQ ID NO: 888), Ser-TGA-1-1 (SEQ ID NO: 879), Arg-TCG-5-1 (SEQ ID NO: 871), Lys-TTT-6-1 (SEQ ID NO: 887), Asn-GTT-1-1 (SEQ ID NO: 880), Arg-CCG-2-1 (SEQ ID NO: 877), Ala-AGC-4-1 (SEQ ID NO: 874), Leu-TAA-1-1 (SEQ ID NO: 876), Ser-CGA-4-1 (SEQ ID NO: 870), Ser-TGA-4-1 (SEQ ID NO: 869), Ser-GCT-2-1 (SEQ ID NO: 872), Arg-TCT-2-1 (SEQ ID NO: 881), Thr-TGT-1-1 (SEQ ID NO: 885), Ile-AAT-4-1 (SEQ ID NO: 873), Val-CAC-2-1 (SEQ ID NO: 884), or Asn-GTT-3-1 (SEQ ID NO: 882) genes.

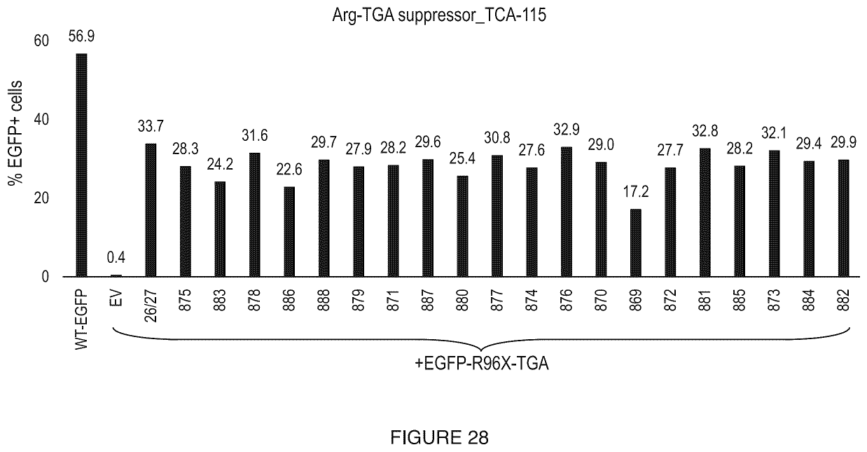

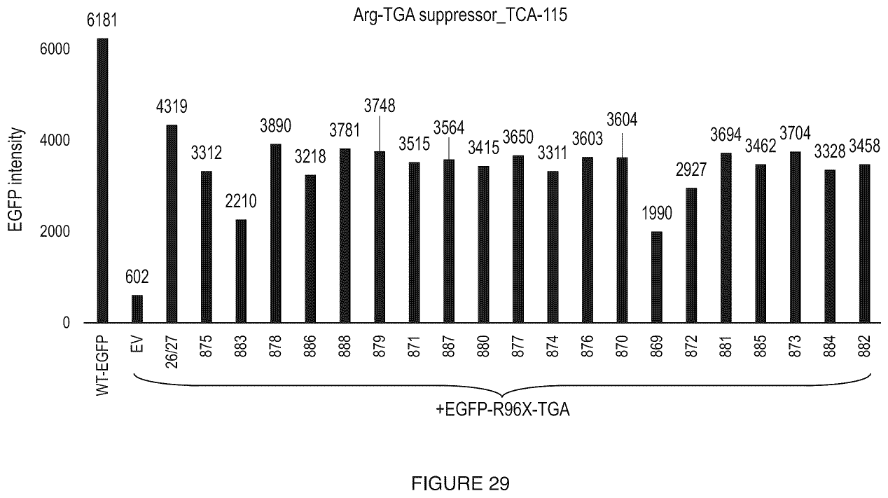

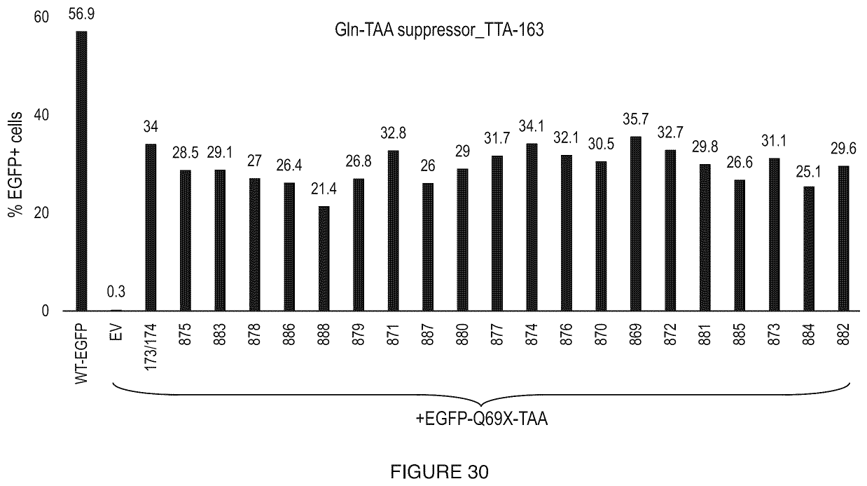

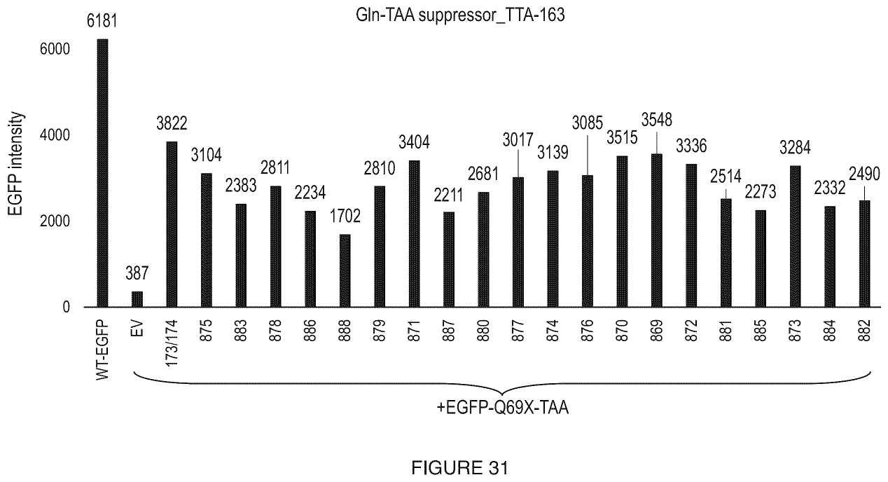

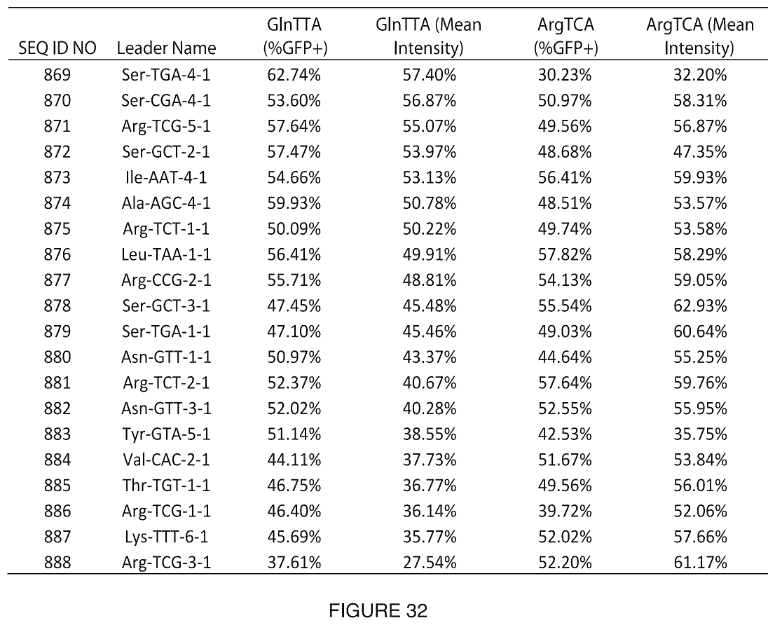

The leader sequences in combination with the Arg_{TCA} suppressor tRNA #115 (SEQ ID NO: 18) or the G1n_{TTA} suppressor tRNA #163 (SEQ ID NO: 45) were tested for PTC readthrough activity by flow cytometry in cell lines co-transfected with an EGFP-R96X-TGA reporter (SEQ ID NO: 31) for Arg_{TCA} constructs or an EGFP-Q69X-TAA reporter (SEQ ID NO: 175) for G1n_{TTA} constructs. The results are shown in FIGS. 28-32 and indicate that (i) the activity of suppressor tRNAs is influenced by the leader sequence, and (ii) suppressor tRNAs (including suppressor tRNAs of different classes) when combined with the identified leader sequences showed high readthrough activity.

### Example 10

This Example describes readthrough activity of certain suppressor tRNAs disclosed herein and small molecule nonsense suppression therapies.

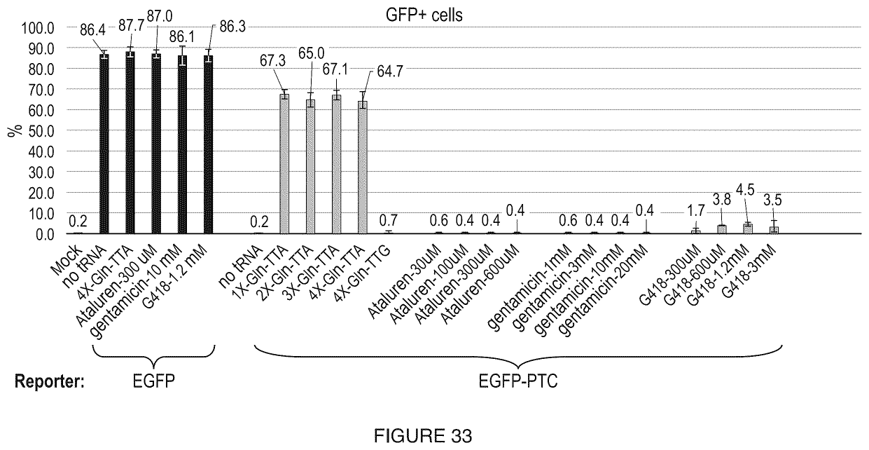

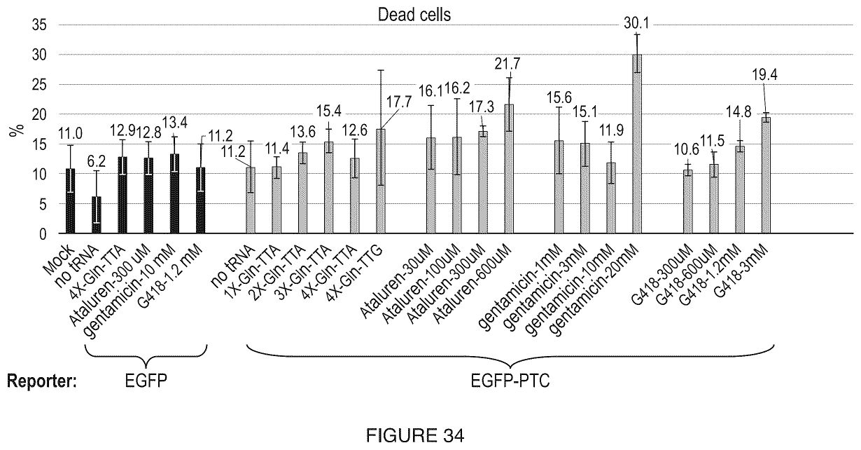

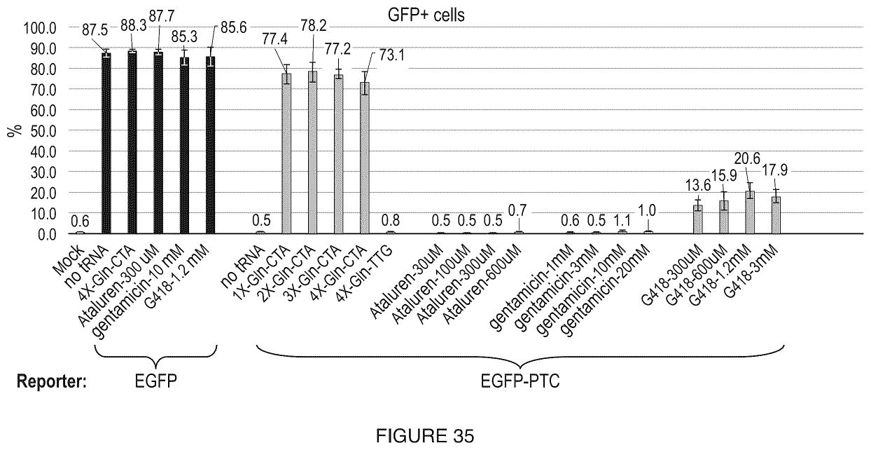

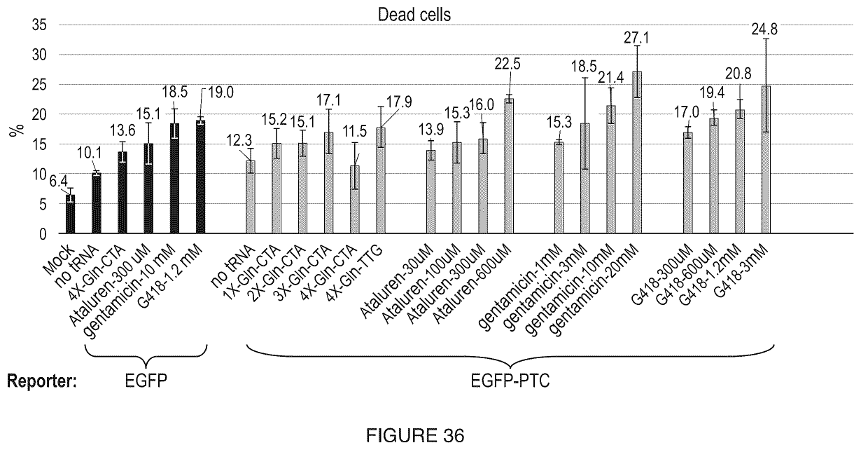

The suppressor tRNAs were tested alongside nonsense suppression drugs translarna (ataluren), gentamicin, and G418 (geneticin). PTC readthrough activity was measured in Neuro-2a cells ˜48 hours after transfection with an expression construct containing a CAG:NLS-EGFP (Q69X-TAA) reporter (as described in Example 4, SEQ ID NO: 175) and either (i) including a Gln suppressor tRNA (either #002, tRNA-Gln-TTG-1-1-TTA-SUP, SEQ ID NO: 36, or #196, tRNA-Gln-TTG-1-1-CTA-SUP, SEQ ID NO: 178, both as described in Example 4) at the indicated copy number on the same construct, or (ii) treated with ataluren, (iii) treated with gentamicin, or (iv) treated with G418. Transfections were performed using the Lipofectamine 3000 Transfection Reagent according to the manufacturer's protocol. For all experimental conditions, cell culture medium was replaced with fresh medium ˜6 hours after transfection and the indicated drugs at the indicated concentrations were added at this point. PTC readthrough activity was measured as the percentage of EGFP positive cells as measured by flow cytometry. A reporter containing wildtype EGFP without a PTC was used as a control. Cell viability in cells receiving the same set of treatments was assessed by flow cytometry following staining with 7-Amino Actinomycin D (7-AAD; Thermo Fisher Scientific #006993-50), a membrane impermeant dye that is generally excluded from viable cells, which was used according to the manufacturer's protocol. Results are shown in FIGS. 33-34 for the G1n_{TTA} suppressor tRNA #002 (SEQ ID NO: 36) and FIGS. 35-36 for the G1n_{CTA} suppressor tRNA #196 (SEQ ID NO: 178). Together, the results demonstrate that the suppressor tRNAs produce greater readthrough than any of the nonsense suppression drugs. Additionally, the results show that, unlike for any of the nonsense suppression drugs, treatment with the suppressor tRNAs is not accompanied by a decrease in cell viability.

### Example 11

This Example describes rescue of full-length SCN1a protein expression by certain suppressor tRNAs disclosed herein.

Flp-In-293 cells were (i) transfected with an expression construct containing mouse SCN1A with an Arg(R)-to-TGA PTC (R1407X) and a 3xFLAG tag peptide (DYKDHD-G-DYKDHD-I-DYKDDDDK) at the C-terminus (SEQ ID NO: 899) and (ii) either co-transfected with an expression construct containing the Arg_{TCA} suppressor tRNA #115 (tRNA-Arg-TCT-2-1-TCA-SUP_no intron, SEQ ID NO: 18), or treated with G418, gentamicin, or ataluren. An expression construct containing wild-type mouse SCN1A and a 3xFLAG tag peptide at the C-terminus (SEQ ID NO: 898) was used as a control. SEQ ID NOs: 898 and 899 are as follows:

(SEQ ID NO: 898)
ATGGAGCAAACAGTGCTTGTACCACCAGGACCTGACAGCTTCAAC
TTCTTCACCAGAGAATCCCTTGCAGCTATTGAAAGGCGCATTGCA
GAAGAGAAGGCTAAGAATCCCAAGCCAGACAAAAAAGATGATGAT
GAAAATGGCCCAAAGCCAAACAGTGACTTGGAAGCTGGGAAGAAC
CTTCCATTTATCTATGGAGACATTCCTCCAGAGATGGTGTCGGAG
CCTCTGGAGGACCTGGACCCCTACTATATCAATAAGAAGACTTTT
ATAGTATTGAATAAAGGGAAGGCCATCTTCCGGTTCAGTGCCACC
TCCGCCCTGTACATTTTAACACCCTTCAATCCTCTTAGGAAAATA
GCTATTAAGATTTTGGTACACTCATTATTCAGCATGTTAATCATG
TGCACTATTTTGACAAACTGTGTATTTATGACAATGAGTAACCCT
CCCGACTGGACAAAGAATGTGGAGTACACCTTCACAGGAATATAT
ACTTTTGAATCACTAATAAAAATTATTGCAAGGGGCTTCTGTTTA
GAAGATTTTACTTTCCTTCGCGACCCATGGAACTGGCTGGACTTC
ACTGTCATTACATTCGCATATGTGACGGAGTTTGTGGACCTGGGC
AATGTCTCAGCATTGAGAACATTCAGAGTTCTTCGAGCATTGAAA
ACTATTTCAGTCATTCCAGGCCTGAAGACCATCGTGGGGGCCCTG
ATCCAGTCGGTGAAGAAGCTGTCTGACGTCATGATACTCACTGTG
TTCTGTCTCAGTGTGTTCGCACTCATCGGGTTGCAGCTCTTCATG
GGCAACCTGAGGAATAAATGTGTACAGTGGCCTCCCACCAACGCT
TCCCTTGAGGAACATAGCATAGAGAAGAATATAACTATGGATTAC
AATGGCACACTTGTAAATGAAACCGTGTTCGAGTTTGACTGGAAA
TCATACATTCAAGACTCAAGATATCATTATTTCCTGGAGGGTGTT
TTAGATGCACTGCTGTGTGGAAATAGCTCTGATGCAGGCCAATGT
CCAGAAGGATATATGTGTGTAAAAGCTGGTAGAAACCCTAATTAT
GGTTACACAAGCTTTGATACCTTCAGTTGGGCATTTTTGTCCCTG
TTTCGACTGATGACTCAGGACTTCTGGGAAAATCTATACCAACTG
ACATTGCGTGCTGCTGGCAAAACCTACATGATATTTTTTGTGCTG
GTCATTTTCTTGGGCTCATTCTACCTGATAAACTTGATCCTGGCT
GTGGTGGCCATGGCCTATGAGGAGCAGAATCAGGCCACACTGGAG
GAGGCTGAACAGAAAGAGGCAGAATTTCAGCAGATGTTGGAGCAA
CTTAAGAAGCAGCAAGAGGCTGCACAGCAGGCAGCGGCTACAACA
GCCTCAGAACATTCCAGGGAGCCCAGTGCAGCAGGCAGGCTCTCA
GATAGCTCTTCAGAAGCCTCTAAGTTGAGTTCGAAGAGTGCTAAA
GAAAGACGAAATCGGAGGAAAAAAAGGAAACAGAAAGAGCAGTCT
GGAGGAGAAGAGAAAGATGATGATGAATTCCACAAGTCTGAGTCT
GAAGACAGCATCAGGAGGAAGGGGTTTCGCTTCTCCATAGAAGGG
AATAGACTGACATATGAAAAGAGGTACTCTTCCCCGCATCAGTCT
CTGTTAAGCATTCGTGGTTCCCTGTTCTCCCCAAGACGCAATAGC
AGAACAAGTCTTTTCAGCTTTAGAGGGCGAGCCAAGGATGTGGGG
TCTGAGAATGACTTTGCTGATGATGAACACAGCACCTTTGAGGAT
AATGAGAGCCGTAGAGACTCACTGTTCGTTCCCCGAAGACACGGA
GAGCGACGCAACAGTAACCTGAGCCAGACCAGCAGGTCCTCCCGA
ATGCTGGCGGTGTTTCCAGCCAATGGGAAGATGCACAGCACGGTG
GATTGCAATGGTGTGGTTTCCTTGGTTGGTGGACCCTCAGTTCCC
ACATCGCCAGTTGGACAGCTTCTGCCAGAGGGAACAACCACTGAA
ACTGAGATGAGAAAGAGGAGGTCGAGCTCTTTCCATGTTTCCATG
GACTTTCTAGAAGATCCTTCCCAGAGGCAAAGGGCAATGAGCATA
GCCAGCATCTTAACAAATACAGTAGAAGAACTAGAAGAATCCAGG
CAGAAATGTCCACCCTGTTGGTATAAATTTTCCAACATATTCTTA
ATTTGGGACTGTTCTCCATATTGGCTGAAAGTTAAACATATTGTC
AACCTGGTGGTGATGGACCCATTTGTTGATCTGGCCATTACCATC
TGCATTGTGTTAAATACGCTCTTCATGGCTATGGAGCACTACCCC
ATGACTGAACATTTCAACCATGTTCTTACAGTGGGAAACTTGGTC
TTCACTGGGATTTTCACAGCAGAAATGTTCCTGAAAATCATCGCA
ATGGATCCTTACTATTACTTCCAAGAAGGCTGGAATATCTTTGAT
GGTTTCATTGTGACACTCAGCCTGGTAGAACTTGGCCTTGCCAAT
GTGGAAGGATTGTCAGTTCTCCGTTCATTTCGACTGCTCCGAGTG
TTCAAGTTGGCAAAGTCTTGGCCCACACTGAATATGCTCATTAAG
ATCATTGGTAACTCGGTGGGAGCACTGGGCAACCTGACTCTGGTG
TTGGCCATCATTGTCTTTATTTTTGCCGTGGTTGGCATGCAGCTG
TTTGGAAAAAGTTACAAAGATTGTGTCTGCAAAATTGCCACTGAC
TGCAAACTCCCACGTTGGCACATGAACGACTTCTTCCACTCGTTC
CTGATCGTGTTCCGCGTGCTGTGTGGGGAGTGGATAGAGACCATG
TGGGACTGCATGGAGGTGGCAGGACAAGCTATGTGCCTTACTGTC
TTCATGATGGTCATGGTGATTGGGAACCTTGTGGTCTTGAACCTC
TTTCTGGCCTTGCTTCTGAGCTCATTTAGTGCAGACAACCTTGCA
GCCACTGATGATGACAATGAGATGAACAACCTGCAGATTGCTGTG
GACAGGATGCACAAAGGAATAGCTTATGTAAAAAGAAAAATATAT
GAATTCATTCAACAATCCTTTGTTAAGAAACAGAAGATTCTAGAT
GAAATTAAGCCACTTGATGATCTAAACAACAGAAAAGACAATTGT
ATCTCTAACCACACAACAGAAATTGGGAAAGATCTGGACTGTCTG
AAAGATGTGAATGGAACCACAAGTGGCATAGGGACGGGCAGCAGT
GTGGAGAAGTACATCATTGATGAGAGTGATTATATGTCATTCATA
AACAACCCCAGCCTCACTGTGACTGTGCCCATTGCTGTGGGAGAG
TCTGACTTTGAGAACTTAAACACAGAAGACTTTAGCAGTGAATCA
GATCTAGAAGAAAGCAAAGAGAAACTCAACGAAAGCAGTAGCTCC
TCAGAGGGAAGCACAGTAGACATTGGGGCGCCTGCAGAGGAACAG
CCTGTCATTGAACCAGAAGAAACCCTTGAGCCCGAAGCTTGCTTC
ACTGAAGGCTGTGTCCAGAGATTCAAGTGCTGTCAAATCAGCGTG
GAAGAAGGAAGAGGGAAACAGTGGTGGAACCTACGGAGGACGTGC
TTCCGAATAGTTGAACACAACTGGTTTGAGACCTTCATTGTGTTC
ATGATTCTCCTGAGTAGTGGTGCCCTGGCCTTTGAGGATATATAT
ATTGATCAGCGAAAGACGATCAAAACCATGCTGGAGTATGCTGAC
AAAGTCTTCACTTACATTTTCATCCTGGAGATGCTCCTCAAATGG
GTGGCCTATGGCTATCAAACATACTTCACCAATGCCTGGTGTTGG
CTAGACTTCTTAATTGTTGATGTTTCATTGGTCAGTTTAACAGCA
AATGCCTTGGGTTACTCTGAACTCGGGGCCATCAAATCCCTAAGG
ACACTAAGAGCTCTGAGACCCCTAAGAGCCTTATCACGATTTGAA
GGGATGAGGGTGGTTGTGAATGCCCTGTTAGGAGCAATTCCATCC
ATCATGAATGTGCTTCTGGTTTGCCTTATATTCTGGCTAATTTTC
AGCATCATGGGCGTAAATTTGTTTGCTGGCAAATTCTACCACTGT
GTTAACACCACAACTGGTGACATATTTGAGATCAGCGAAGTCAAT
AATCATTCTGATTGCCTAAAACTAATAGAAAGAAATGAGACCGCC
CGGTGGAAAAATGTGAAAGTAAACTTTGATAATGTAGGATTTGGG
TATCTTTCTTTGCTTCAAGTTGCCACATTTAAGGGCTGGATGGAT
ATCATGTATGCTGCAGTTGATTCCAGAAATGTTGAACTACAGCCT
AAGTATGAGGAAAGCCTGTACATGTATTTGTACTTCGTCATCTTC
ATCATCTTCGGGTCCTTCTTTACCCTGAACCTGTTTATTGGTGTC
ATTATCGACAATTTCAACCAGCAAAAGAAGAAGTTTGGAGGTCAA
GACATCTTTATGACAGAAGAACAGAAGAAATACTATAATGCAATG
AAGAAATTAGGATCAAAAAAGCCACAAAAGCCTATCCCTCGACCT
GGAAACAAATTTCAAGGAATGGTTTTTGACTTTGTAACCAGACAA
GTGTTTGATATCAGCATCATGATCCTCATCTGTCTGAACATGGTG
ACCATGATGGTGGAAACGGATGACCAGAGCGATTATGTGACAAGC
ATTTTGTCACGCATCAACCTGGTGTTCATCGTCCTGTTCACCGGC
GAGTGTGTGCTCAAGCTCATCTCGCTCCGCCATTATTATTTCACC
ATTGGATGGAACATTTTCGATTTTGTGGTGGTCATCCTCTCCATT
GTAGGGATGTTTCTTGCGGAGCTAATAGAAAAGTATTTTGTGTCT
CCTACCCTGTTCCGAGTCATCCGCCTGGCCAGGATTGGACGAATC
CTACGCCTGATCAAAGGTGCCAAGGGGATCCGCACGCTGCTCTTT
GCTCTGATGATGTCCCTTCCTGCGCTGTTTAACATCGGCCTCCTG
CTTTTTCTCGTCATGTTCATCTACGCCATCTTTGGGATGTCCAAC
TTTGCCTATGTTAAGAGGGAAGTTGGGATTGATGACATGTTCAAC
TTTGAGACCTTCGGCAACAGCATGATCTGCCTGTTCCAAATCACC
ACCTCTGCGGGCTGGGATGGACTGCTGGCCCCCATCCTCAACAGC
AAACCCCCTGACTGTGACCCTAATAAAGTTAACCCTGGAAGCTCG
GTGAAGGGAGACTGTGGGAACCCATCTGTGGGGATTTTCTTTTTT
GTCAGCTACATCATCATATCCTTCCTGGTTGTGGTGAACATGTAC
ATTGCTGTCATCCTGGAGAACTTCAGCGTTGCCACAGAAGAAAGT
GCAGAGCCTCTGAGTGAGGACGACTTTGAGATGTTCTACGAGGTC
TGGGAGAAGTTCGACCCTGACGCCACCCAGTTCATGGAATTTGAA
AAATTATCTCAGTTTGCAGCTGCTCTAGAACCCCCTCTCAATTTG
CCACAACCAAACAAACTTCAGCTCATTGCCATGGACCTGCCCATG
GTGAGTGGAGACCGCATCCACTGCCTGGACATCTTATTTGCTTTT
ACAAAGCGGGTGTTGGGTGAGAGTGGAGAGATGGATGCTCTTCGA
ATCCAGATGGAAGAGCGGTTCATGGCTTCCAACCCCTCCAAGGTC
TCTTATCAGCCCATCACTACTACATTAAAACGCAAACAAGAGGAG
GTGTCAGCTGTTATCATTCAGCGAGCTTATAGGCGCCACCTTTTG
AAGCGAACAGTAAAACAAGCTTCATTCACATACAATAAGAACAAA
CTCAAAGGTGGGGCTAATCTTCTTGTAAAAGAAGACATGCTCATT
GACAGAATAAACGAAAACTCTATTACGGAGAAAACTGACCTGACA
ATGTCCACAGCAGCTTGTCCGCCCTCCTACGATCGGGTGACAAAG
CCAATCGTGGAGAAACACGAGCAGGAAGGGAAGGATGAAAAAGCC
AAAGGGAAAGACTACAAAGACCATGACGGTGATTATAAAGATCAT
GACATCGATTACAAGGATGACGATGACAAGTAA;
and
(SEQ ID NO: 899)
ATGGAGCAAACAGTGCTTGTACCACCAGGACCTGACAGCTTCAAC
TTCTTCACCAGAGAATCCCTTGCAGCTATTGAAAGGCGCATTGCA
GAAGAGAAGGCTAAGAATCCCAAGCCAGACAAAAAAGATGATGAT
GAAAATGGCCCAAAGCCAAACAGTGACTTGGAAGCTGGGAAGAAC
CTTCCATTTATCTATGGAGACATTCCTCCAGAGATGGTGTCGGAG
CCTCTGGAGGACCTGGACCCCTACTATATCAATAAGAAGACTTTT
ATAGTATTGAATAAAGGGAAGGCCATCTTCCGGTTCAGTGCCACC
TCCGCCCTGTACATTTTAACACCCTTCAATCCTCTTAGGAAAATA
GCTATTAAGATTTTGGTACACTCATTATTCAGCATGTTAATCATG
TGCACTATTTTGACAAACTGTGTATTTATGACAATGAGTAACCCT
CCCGACTGGACAAAGAATGTGGAGTACACCTTCACAGGAATATAT
ACTTTTGAATCACTAATAAAAATTATTGCAAGGGGCTTCTGTTTA
GAAGATTTTACTTTCCTTCGCGACCCATGGAACTGGCTGGACTTC
ACTGTCATTACATTCGCATATGTGACGGAGTTTGTGGACCTGGGC
AATGTCTCAGCATTGAGAACATTCAGAGTTCTTCGAGCATTGAAA
ACTATTTCAGTCATTCCAGGCCTGAAGACCATCGTGGGGGCCCTG
ATCCAGTCGGTGAAGAAGCTGTCTGACGTCATGATACTCACTGTG
TTCTGTCTCAGTGTGTTCGCACTCATCGGGTTGCAGCTCTTCATG
GGCAACCTGAGGAATAAATGTGTACAGTGGCCTCCCACCAACGCT
TCCCTTGAGGAACATAGCATAGAGAAGAATATAACTATGGATTAC
AATGGCACACTTGTAAATGAAACCGTGTTCGAGTTTGACTGGAAA
TCATACATTCAAGACTCAAGATATCATTATTTCCTGGAGGGTGTT
TTAGATGCACTGCTGTGTGGAAATAGCTCTGATGCAGGCCAATGT
CCAGAAGGATATATGTGTGTAAAAGCTGGTAGAAACCCTAATTAT
GGTTACACAAGCTTTGATACCTTCAGTTGGGCATTTTTGTCCCTG
TTTCGACTGATGACTCAGGACTTCTGGGAAAATCTATACCAACTG
ACATTGCGTGCTGCTGGCAAAACCTACATGATATTTTTTGTGCTG
GTCATTTTCTTGGGCTCATTCTACCTGATAAACTTGATCCTGGCT
GTGGTGGCCATGGCCTATGAGGAGCAGAATCAGGCCACACTGGAG
GAGGCTGAACAGAAAGAGGCAGAATTTCAGCAGATGTTGGAGCAA
CTTAAGAAGCAGCAAGAGGCTGCACAGCAGGCAGCGGCTACAACA
GCCTCAGAACATTCCAGGGAGCCCAGTGCAGCAGGCAGGCTCTCA
GATAGCTCTTCAGAAGCCTCTAAGTTGAGTTCGAAGAGTGCTAAA
GAAAGACGAAATCGGAGGAAAAAAAGGAAACAGAAAGAGCAGTCT
GGAGGAGAAGAGAAAGATGATGATGAATTCCACAAGTCTGAGTCT
GAAGACAGCATCAGGAGGAAGGGGTTTCGCTTCTCCATAGAAGGG
AATAGACTGACATATGAAAAGAGGTACTCTTCCCCGCATCAGTCT
CTGTTAAGCATTCGTGGTTCCCTGTTCTCCCCAAGACGCAATAGC
AGAACAAGTCTTTTCAGCTTTAGAGGGCGAGCCAAGGATGTGGGG
TCTGAGAATGACTTTGCTGATGATGAACACAGCACCTTTGAGGAT
AATGAGAGCCGTAGAGACTCACTGTTCGTTCCCCGAAGACACGGA
GAGCGACGCAACAGTAACCTGAGCCAGACCAGCAGGTCCTCCCGA
ATGCTGGCGGTGTTTCCAGCCAATGGGAAGATGCACAGCACGGTG
GATTGCAATGGTGTGGTTTCCTTGGTTGGTGGACCCTCAGTTCCC
ACATCGCCAGTTGGACAGCTTCTGCCAGAGGGAACAACCACTGAA
ACTGAGATGAGAAAGAGGAGGTCGAGCTCTTTCCATGTTTCCATG
GACTTTCTAGAAGATCCTTCCCAGAGGCAAAGGGCAATGAGCATA
GCCAGCATCTTAACAAATACAGTAGAAGAACTAGAAGAATCCAGG
CAGAAATGTCCACCCTGTTGGTATAAATTTTCCAACATATTCTTA
ATTTGGGACTGTTCTCCATATTGGCTGAAAGTTAAACATATTGTC
AACCTGGTGGTGATGGACCCATTTGTTGATCTGGCCATTACCATC
TGCATTGTGTTAAATACGCTCTTCATGGCTATGGAGCACTACCCC
ATGACTGAACATTTCAACCATGTTCTTACAGTGGGAAACTTGGTC
TTCACTGGGATTTTCACAGCAGAAATGTTCCTGAAAATCATCGCA
ATGGATCCTTACTATTACTTCCAAGAAGGCTGGAATATCTTTGAT
GGTTTCATTGTGACACTCAGCCTGGTAGAACTTGGCCTTGCCAAT
GTGGAAGGATTGTCAGTTCTCCGTTCATTTCGACTGCTCCGAGTG
TTCAAGTTGGCAAAGTCTTGGCCCACACTGAATATGCTCATTAAG
ATCATTGGTAACTCGGTGGGAGCACTGGGCAACCTGACTCTGGTG
TTGGCCATCATTGTCTTTATTTTTGCCGTGGTTGGCATGCAGCTG
TTTGGAAAAAGTTACAAAGATTGTGTCTGCAAAATTGCCACTGAC
TGCAAACTCCCACGTTGGCACATGAACGACTTCTTCCACTCGTTC
CTGATCGTGTTCCGCGTGCTGTGTGGGGAGTGGATAGAGACCATG
TGGGACTGCATGGAGGTGGCAGGACAAGCTATGTGCCTTACTGTC
TTCATGATGGTCATGGTGATTGGGAACCTTGTGGTCTTGAACCTC
TTTCTGGCCTTGCTTCTGAGCTCATTTAGTGCAGACAACCTTGCA
GCCACTGATGATGACAATGAGATGAACAACCTGCAGATTGCTGTG
GACAGGATGCACAAAGGAATAGCTTATGTAAAAAGAAAAATATAT
GAATTCATTCAACAATCCTTTGTTAAGAAACAGAAGATTCTAGAT
GAAATTAAGCCACTTGATGATCTAAACAACAGAAAAGACAATTGT
ATCTCTAACCACACAACAGAAATTGGGAAAGATCTGGACTGTCTG
AAAGATGTGAATGGAACCACAAGTGGCATAGGGACGGGCAGCAGT
GTGGAGAAGTACATCATTGATGAGAGTGATTATATGTCATTCATA
AACAACCCCAGCCTCACTGTGACTGTGCCCATTGCTGTGGGAGAG
TCTGACTTTGAGAACTTAAACACAGAAGACTTTAGCAGTGAATCA
GATCTAGAAGAAAGCAAAGAGAAACTCAACGAAAGCAGTAGCTCC
TCAGAGGGAAGCACAGTAGACATTGGGGCGCCTGCAGAGGAACAG
CCTGTCATTGAACCAGAAGAAACCCTTGAGCCCGAAGCTTGCTTC
ACTGAAGGCTGTGTCCAGAGATTCAAGTGCTGTCAAATCAGCGTG
GAAGAAGGAAGAGGGAAACAGTGGTGGAACCTACGGAGGACGTGC
TTCCGAATAGTTGAACACAACTGGTTTGAGACCTTCATTGTGTTC
ATGATTCTCCTGAGTAGTGGTGCCCTGGCCTTTGAGGATATATAT
ATTGATCAGCGAAAGACGATCAAAACCATGCTGGAGTATGCTGAC
AAAGTCTTCACTTACATTTTCATCCTGGAGATGCTCCTCAAATGG
GTGGCCTATGGCTATCAAACATACTTCACCAATGCCTGGTGTTGG
CTAGACTTCTTAATTGTTGATGTTTCATTGGTCAGTTTAACAGCA
AATGCCTTGGGTTACTCTGAACTCGGGGCCATCAAATCCCTAAGG
ACACTAAGAGCTCTGAGACCCCTAAGAGCCTTATCACGATTTGAA
GGGATGAGGGTGGTTGTGAATGCCCTGTTAGGAGCAATTCCATCC
ATCATGAATGTGCTTCTGGTTTGCCTTATATTCTGGCTAATTTTC
AGCATCATGGGCGTAAATTTGTTTGCTGGCAAATTCTACCACTGT
GTTAACACCACAACTGGTGACATATTTGAGATCAGCGAAGTCAAT
AATCATTCTGATTGCCTAAAACTAATAGAAAGAAATGAGACCGCC
TGATGGAAAAATGTGAAAGTAAACTTTGATAATGTAGGATTTGGG
TATCTTTCTTTGCTTCAAGTTGCCACATTTAAGGGCTGGATGGAT
ATCATGTATGCTGCAGTTGATTCCAGAAATGTTGAACTACAGCCT
AAGTATGAGGAAAGCCTGTACATGTATTTGTACTTCGTCATCTTC
ATCATCTTCGGGTCCTTCTTTACCCTGAACCTGTTTATTGGTGTC
ATTATCGACAATTTCAACCAGCAAAAGAAGAAGTTTGGAGGTCAA
GACATCTTTATGACAGAAGAACAGAAGAAATACTATAATGCAATG
AAGAAATTAGGATCAAAAAAGCCACAAAAGCCTATCCCTCGACCT
GGAAACAAATTTCAAGGAATGGTTTTTGACTTTGTAACCAGACAA
GTGTTTGATATCAGCATCATGATCCTCATCTGTCTGAACATGGTG
ACCATGATGGTGGAAACGGATGACCAGAGCGATTATGTGACAAGC
ATTTTGTCACGCATCAACCTGGTGTTCATCGTCCTGTTCACCGGC
GAGTGTGTGCTCAAGCTCATCTCGCTCCGCCATTATTATTTCACC
ATTGGATGGAACATTTTCGATTTTGTGGTGGTCATCCTCTCCATT
GTAGGGATGTTTCTTGCGGAGCTAATAGAAAAGTATTTTGTGTCT
CCTACCCTGTTCCGAGTCATCCGCCTGGCCAGGATTGGACGAATC
CTACGCCTGATCAAAGGTGCCAAGGGGATCCGCACGCTGCTCTTT
GCTCTGATGATGTCCCTTCCTGCGCTGTTTAACATCGGCCTCCTG
CTTTTTCTCGTCATGTTCATCTACGCCATCTTTGGGATGTCCAAC
TTTGCCTATGTTAAGAGGGAAGTTGGGATTGATGACATGTTCAAC
TTTGAGACCTTCGGCAACAGCATGATCTGCCTGTTCCAAATCACC
ACCTCTGCGGGCTGGGATGGACTGCTGGCCCCCATCCTCAACAGC
AAACCCCCTGACTGTGACCCTAATAAAGTTAACCCTGGAAGCTCG
GTGAAGGGAGACTGTGGGAACCCATCTGTGGGGATTTTCTTTTTT
GTCAGCTACATCATCATATCCTTCCTGGTTGTGGTGAACATGTAC
ATTGCTGTCATCCTGGAGAACTTCAGCGTTGCCACAGAAGAAAGT
GCAGAGCCTCTGAGTGAGGACGACTTTGAGATGTTCTACGAGGTC
TGGGAGAAGTTCGACCCTGACGCCACCCAGTTCATGGAATTTGAA
AAATTATCTCAGTTTGCAGCTGCTCTAGAACCCCCTCTCAATTTG
CCACAACCAAACAAACTTCAGCTCATTGCCATGGACCTGCCCATG
GTGAGTGGAGACCGCATCCACTGCCTGGACATCTTATTTGCTTTT
ACAAAGCGGGTGTTGGGTGAGAGTGGAGAGATGGATGCTCTTCGA
ATCCAGATGGAAGAGCGGTTCATGGCTTCCAACCCCTCCAAGGTC
TCTTATCAGCCCATCACTACTACATTAAAACGCAAACAAGAGGAG
GTGTCAGCTGTTATCATTCAGCGAGCTTATAGGCGCCACCTTTTG
AAGCGAACAGTAAAACAAGCTTCATTCACATACAATAAGAACAAA
CTCAAAGGTGGGGCTAATCTTCTTGTAAAAGAAGACATGCTCATT
GACAGAATAAACGAAAACTCTATTACGGAGAAAACTGACCTGACA
ATGTCCACAGCAGCTTGTCCGCCCTCCTACGATCGGGTGACAAAG
CCAATCGTGGAGAAACACGAGCAGGAAGGGAAGGATGAAAAAGCC
AAAGGGAAAGACTACAAAGACCATGACGGTGATTATAAAGATCAT
GACATCGATTACAAGGATGACGATGACAAGTAA.

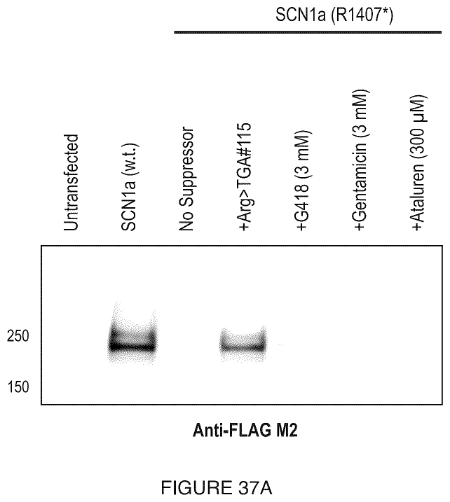

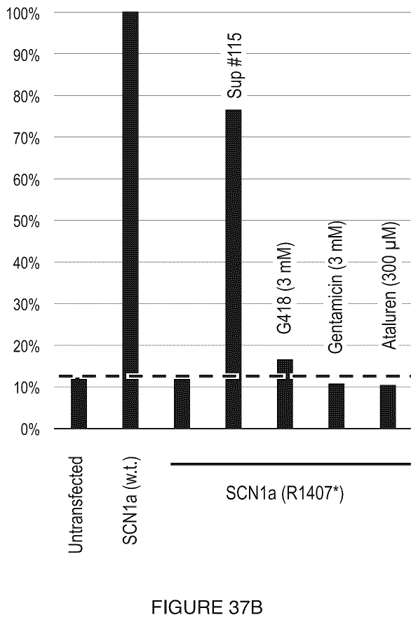

SCN1A was detected by Western blot as follows. Protein was isolated at 24 hours post transfection in RIPA Lysis and Extraction Buffer (Thermo Fisher Scientific #89900) containing Halt Protease Inhibitor Cocktail (Thermo Fisher Scientific #87786) according to the manufacturer's protocol. Protein concentrations were determined using the Pierce BCA Protein Assay Kit (Thermo Fisher Scientific #23225). 30 μg of protein was separated on either NuPAGE 4-12% Bis-Tris (Thermo Fisher Scientific #NP0322BOX) or NuPAGE 3-8%, Tris-Acetate (Thermo Fisher Scientific #EA0375BOX) protein gels at 150V for 1.5 hours and transferred to PVDF membranes at 30V overnight followed by 250 mA for 30 min at 4° C. Blots were blocked in SuperBlock T20 Blocking Buffer (Thermo Fisher Scientific #37536) at room temperature for 1 hour, incubated with the primary anti-FLAG M2 antibody (Sigma, F1804-200UG, 1:1000) in TBST overnight at 4° C., washed 3 times with TBST, and incubated with the Goat anti-Mouse IgG (H+L) Secondary Antibody, HRP secondary antibody (Thermo Fisher Scientific #31431, 1:30,000) at room temperature for 1 hour. The blots were developed by applying the SuperSignal West Femto Maximum Sensitivity Substrate (Thermo Fisher Scientific #34094) and signals were detected by the iBright Imaging system. The results are shown in FIG. 37 and demonstrate that the Arg_{TCA} suppressor tRNA #115, but not the small molecules drugs, was able to rescue full-length SCN1A protein expression.

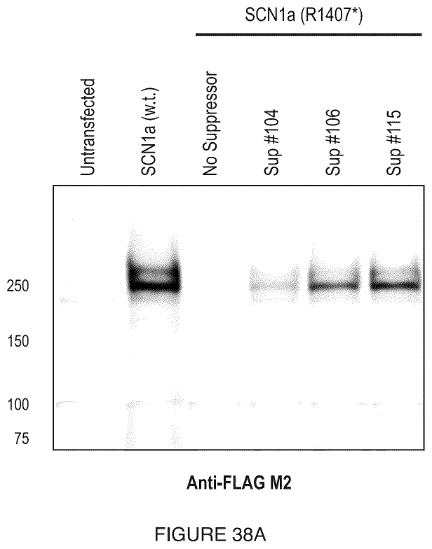

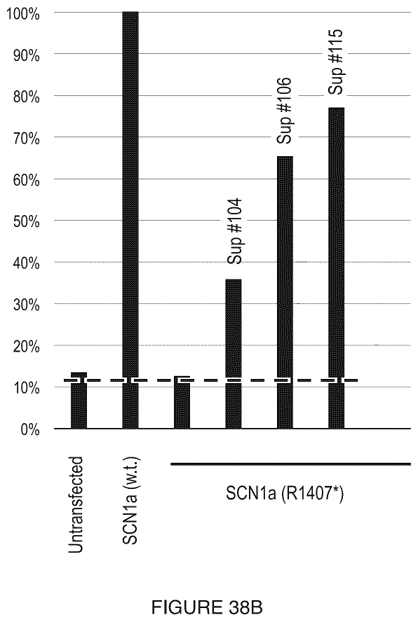

Flp-In-293 cells were also co-transfected with (i) an expression construct containing mouse SCN1A with an Arg(R)-to-TGA PTC (R1407X) and a 3xFLAG tag peptide at the C-terminus (SEQ ID NO: 899) and (ii) an expression construct containing the Arg_{TCA} suppressor tRNA #104 (tRNA-Arg-CCG-3-1-TCA-SUP, SEQ ID NO: 6), the Arg_{TCA} suppressor tRNA #106 (tRNA-Arg-CCT-2-1-TCA-SUP, SEQ ID NO: 8), or the Arg_{TCA} suppressor tRNA #115 (tRNA-Arg-TCT-2-1-TCA-SUP_no intron, SEQ ID NO: 18). SCN1A expression was measured by Western blot using an anti-FLAG antibody as described above in this Example. The results are shown in FIG. 38 and demonstrate that each suppressor tRNA tested rescued full-length SCN1A protein expression.

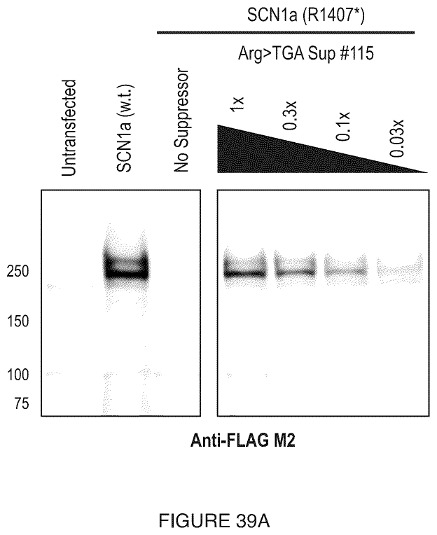

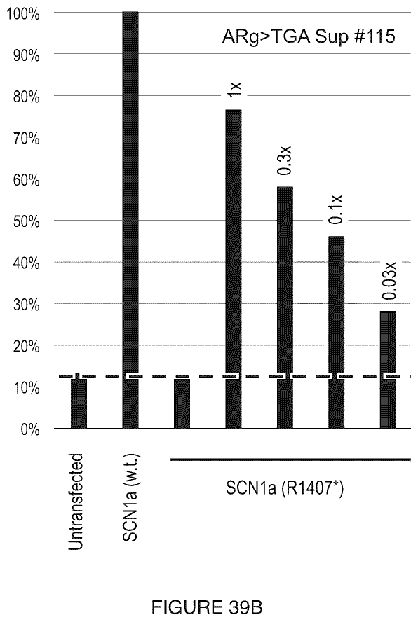

Flp-In-293 cells were also co-transfected with (i) an expression construct containing mouse SCN1A with an Arg(R)-to-TGA PTC (R1407X) and a 3xFLAG tag peptide at the C-terminus (SEQ ID NO: 899) and (ii) an expression construct containing the Arg_{TCA} suppressor tRNA #115 (tRNA-Arg-TCT-2-1-TCA-SUP_no intron, SEQ ID NO: 18) at a range of doses (13 ng per well, 40 ng per well, 113 ng per well, or 400 ng per well; 6-well cell culture plate). SCN1A expression was measured by Western blot using an anti-FLAG antibody as described above in this Example. The results are shown in FIG. 39 and demonstrate that tRNA 115 was able to rescue full-length SCN1A protein expression over a broad dose range.

### Example 12

This Example describes readthrough activity of disclosed suppressor tRNAs delivered by adeno-associated virus (AAV) vectors.

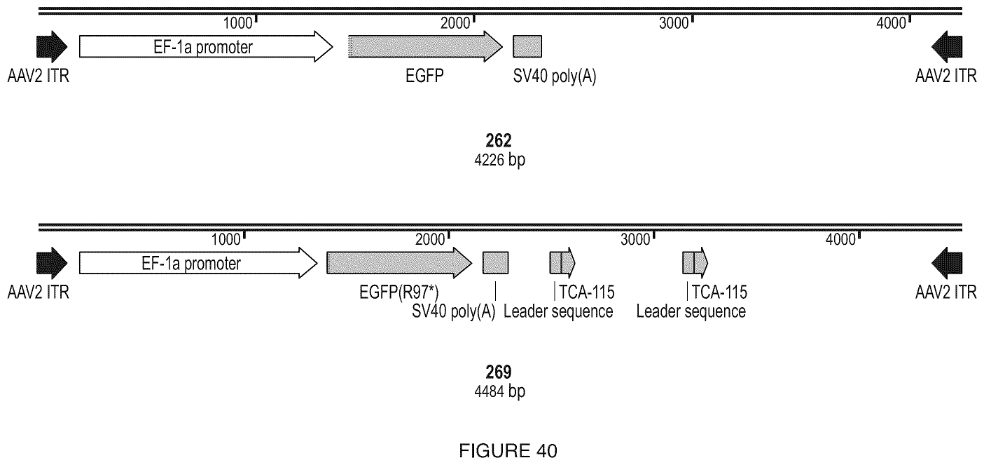

Constructs that were packaged into AAV-PHP.eB capsids are depicted in FIG. 40. Construct 262 contains wild-type EGFP driven by an EF1a promoter. Construct 269 contains EGFP-R96X-TGA driven by an EF1a promoter (SEQ ID NO: 177) and two copies of the Arg_{TCA} suppressor tRNA #115 (tRNA-Arg-TCT-2-1-TCA-SUP_no intron, SEQ ID NO: 18, as described in Example 1) in the context of 55 bps upstream flanking genomic DNA from tRNA-Tyr-GTA-5-1 (SEQ ID NO: 900). Both constructs contain 5′ and 3′ ITR sequences from AAV2, which provide cis-acting elements for AAV replication and packaging. AAV-PHP.eB containing construct 262 and construct 269 was produced by Vigene Biosciences.

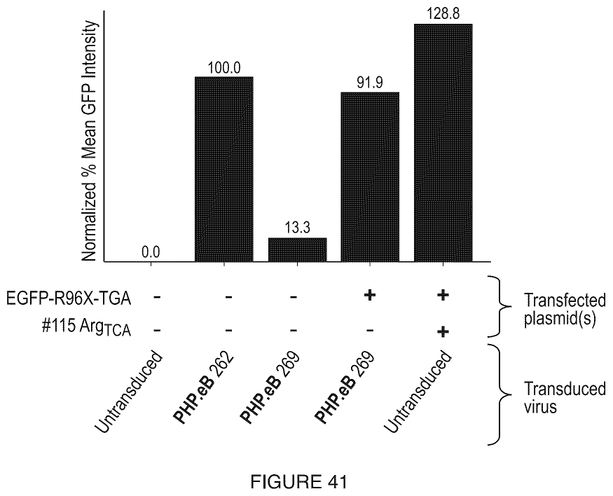

Prior to AAV transduction, 293 cells (Agilent #240073) were pre-transfected with an expression construct containing the LY6A gene (CCDS ID 27540.1) driven by the CMV early enhancer/chicken β actin (CAG) promoter, which is required for robust transduction by AAV-PHP.eB. For pre-transfection, cells were transiently transfected with the LY6A expression construct using lipofectamine 3000 Transfection Reagent according to the manufacturer's protocol. At ˜24 hours post-transfection the media was exchanged for fresh media and the cells were allowed to recover for another 24 hours prior to viral transduction. The cells were then transduced at an MOI of 1E5 vg/cell. Results are depicted in FIG. 41. AAV delivered Arg_{TCA} suppressor tRNA #115 resulted in ˜13.2% PTC readthrough based on GFP intensity. The suppressor tRNA shows comparable readthrough activity when delivered by AAV or transient transfection.

### Example 13

This Example describes ribosome profiling experiments that demonstrate that disclosed suppressor tRNAs do not cause a significant amount of off-target native stop codon read-through.

In order to determine whether suppressor tRNAs cause readthrough of native stop codons, ribosome profiling was used to quantify the number of ribosomes found in the 3′ UTR of mRNAs from (i) cells transfected with an expression construct containing a suppressor tRNA relative to (ii) cells transfected with an expression construct that lacks a suppressor tRNA. Ribosomes typically terminate translation upon encountering a stop codon, so if suppressor tRNAs cause increased readthrough of native stop codons, this will be indicated by an increase in the density of ribosomes found in the 3′ UTR of mRNAs in cells expressing a suppressor tRNA, especially in the 3′ UTR mRNAs containing a native stop codon that is recognized by the expressed suppressor tRNA.

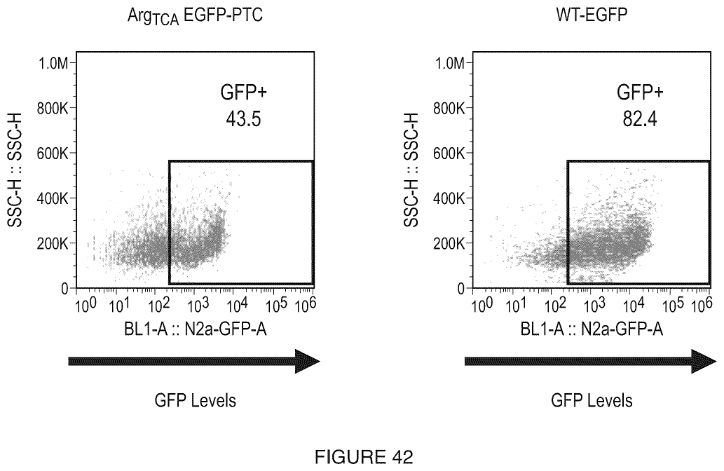

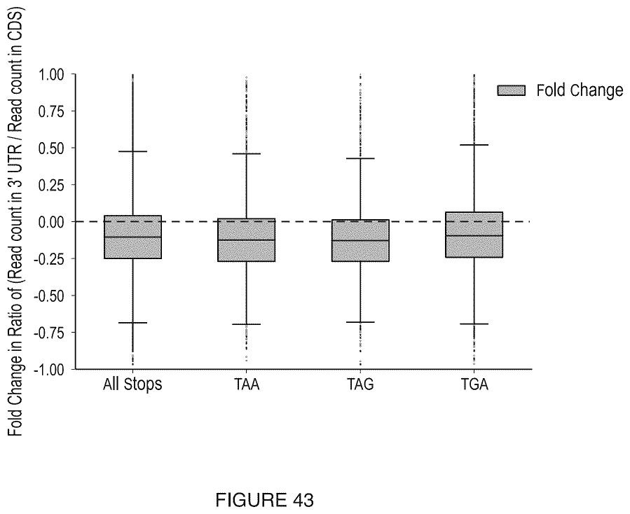

Neuro-2a cells were transfected with either (i) an expression construct containing an EGFP-R96X-TGA reporter (SEQ ID NO: 177) and the Arg_{TCA} suppressor tRNA #001 (SEQ ID NO: 11) on the same construct or (ii) an expression construct lacking a suppressor tRNA and containing a wild-type version of the EGFP reporter. Expression of EGFP is depicted in FIG. 42. At ˜48 hours post transfection, cells were subjected to ribosome footprint profiling as follows. Cells were lysed in the lysis buffer (10 mM Tris-HCl pH7.5, 5 mM MgCl2, 100 mM KCl, 1% Triton X-100, 1 Mm DTT, 50 μg/mL Emetine (Sigma #324693), and 500 U/mL RNAsin (Promega #N2615)) and cell lysates were sheared ten times with a 25-gauge needle followed by centrifugation at 20,000 g for 10 minutes at 4° C. The supernatants were digested with micrococcal nuclease (MNase; 120 units/OD A260 lysates; New England Biolabs #M02475) at room temperature for 30 minutes prior to adding 5 uL SuperAse-IN (Thermo Fisher Scientific #AM2694) to stop the reaction. MNase-treated extracts were loaded to a 15-45% sucrose gradient and separated by density in a SW 41Ti swinging-bucket rotor (Beckman Coulter #331362) at 41,000 rpm at 4° C. for 2:26 hours. After fractionation and collection of fractions containing monosomes, ribosome protected mRNA fragments were precipitated from the sucrose overnight at ˜20° C. with 1.25 mL of 95% ethanol. mRNA fragments were re-suspended with 10 mM Tris-HCl, pH 8.0, and separated on a 15% denaturing polyacrylamide gel (TBE-urea gel; Thermo Fisher Scientific #EC68852BOX). RNA fragments with sizes ranging from 26 to 34 nt were excised from the gel and isolated to generate the ribosome-protected fragment library. After 3′ linker ligation, rRNA depletion with the Ribo-Zero reagents in the TruSeq Stranded Total RNA Library Prep Gold Kit (Illumina #20020598), reverse transcription, circularization, and PCR amplification with indexing primers, the PCR products were separated on an 8% nondenaturing polyacrylamide gel (Thermo Fisher Scientific #EC62152BOX). The barcoded cDNA libraries were extracted from the gel and sequenced by the NextSeq 550 Sequencing System with the single read run type. Following sequencing, raw reads were trimmed of adapters using Trimmomatic then depleted for non-coding RNA by aligning against Ensembl's mouse mm10 ncRNA reference using bowtie2. Remaining reads were aligned against UCSC's mm10 mouse reference assembly, again using bowtie2. Multi-mapping reads were discarded. The resulting final set of aligned reads was quantified using the RiboProfiling package in R and custom Python scripting. Python was used to generate plots examining 3′ UTR occupancy and fold change for each gene with 20 or more uniquely mapping reads, and the distributions for genes with each native stop codon were compared using the 2 sample Kolmogorov-Smirnov test. The results are depicted in FIG. 43.

Together, these results demonstrate that suppressor tRNAs can facilitate expression of transcripts, e.g., EGFP-R96X-TGA, containing premature termination codons while not causing a significant amount of off-target native stop codon read-through in the expressing cells.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein

What is claimed is:

1. A tRNA comprising a nucleotide sequence set forth in Table 2.

2. The tRNA of claim 1, wherein the tRNA comprises a nucleotide sequence selected from SEQ ID NOs: 19-21, 37, 39, 40, 44, 179, 181, 182, and 186.

3. The tRNA of claim 1 or 2, wherein the tRNA comprises a naturally occurring nucleotide modification.

4. The tRNA of any one of claims 1-3, wherein the tRNA comprises one or more nucleotide modifications selected from 5-methyl uridine, 5-carbamoylmethyluridine, 5-carbamoyl-methyl-2-O-methyluridine, 5-methoxy-carbonylmethyluridine, 5-methoxycarbonylmethyl-2-thiouridine, pseudouridine, dihydrouridine, 1-methyladenosine, and inosine.

5. An expression vector comprising a nucleotide sequence encoding the tRNA of any one of claims 1-4.

6. The expression vector of claim 5, wherein the expression vector comprises 1, 2, 3, 4, or more than 4 copy numbers of the nucleotide sequence encoding the tRNA.

7. The expression vector of claim 5 or 6, wherein the expression vector further comprises a nucleotide sequence set forth in Table 4.

8. The expression vector of claim 7, wherein the expression vector comprises a nucleotide sequence selected from SEQ ID NOs: 869-888.

9. An expression vector comprising a nucleotide sequence encoding a tRNA set forth in Table 3, wherein the expression vector comprises 1, 2, 3, 4, or more than 4 copy numbers of the nucleotide sequence encoding the tRNA.

10. An expression vector comprising a nucleotide sequence encoding a tRNA set forth in Table 3, wherein the expression vector further comprises a nucleotide sequence set forth in Table 4.

11. The expression vector of claim 9 or 10, wherein the tRNA comprises a nucleotide sequence selected from SEQ ID NOs: 6-9, 11, 16-18, 22, 35, 36, 38, 45, 178, 180, and 187.

12. The expression vector of claim 10 or 11, wherein the expression vector comprises a nucleotide sequence selected from SEQ ID NOs: 869-888.

13. The expression vector of any one of claims 5-12, wherein the expression vector is a viral vector.

14. The expression vector of claim 13, wherein the viral vector is a DNA virus vector.

15. The expression vector of claim 13 or 14, wherein the viral vector is an adeno-associated virus (AAV) vector.

16. A pharmaceutical composition comprising the tRNA of any one of claims 1-4 or the expression vector of any one of claims 5-15 and a pharmaceutically acceptable excipient.

17. The pharmaceutical composition of claim 16, wherein the tRNA or expression vector is not conjugated to or associated with another moiety.

18. The pharmaceutical composition of claim 17, wherein the tRNA or expression vector is not conjugated to or associated with a carrier particle.

19. The pharmaceutical composition of claim 8, wherein the carrier particle is an aminolipid particle.

20. The pharmaceutical composition of any one of claims 15-19, wherein the composition does not comprise a nanoparticle.

21. The pharmaceutical composition of any one of claims 15-20, wherein the composition does not comprise an aminolipid delivery compound.

22. A method of expressing in a mammalian cell a functional gene product encoded by a gene containing a premature termination codon, the method comprising introducing into the cell an effective amount of the tRNA of any one of claims 1-4 or the expression vector of any one of claims 5-15, thereby permitting an amino acid to be incorporated into the gene product at a position that would otherwise result in a truncated gene product caused by the premature termination codon.

23. The method of claim 22, wherein the cell contains less truncated gene product than a cell without the tRNA.

24. The method of claim 22 or 23, wherein the cell contains a greater amount of functional gene product than a cell without the tRNA.

25. The method of any one of claims 22-24, wherein the gene is a gene set forth in Table 5 or Table 6.

26. The method of claim 25, wherein the gene is a gene set forth in Table 5.

27. A method of expressing in a mammalian cell a functional gene product encoded by a gene containing a premature termination codon, the method comprising introducing into the cell an effective amount of a tRNA set forth in Table 3, or an expression vector comprising a nucleotide sequence encoding the tRNA, thereby permitting an amino acid to be incorporated into the gene product at a position that would otherwise result in a truncated gene product caused by the premature termination codon, wherein the gene is a gene set forth in Table 5.

28. The method of claim 27, wherein the tRNA comprises a nucleotide sequence selected from SEQ ID NOs: 6-9, 11, 16-18, 22, 35, 36, 38, 45, 178, 180, and 187.

29. The method of any one of claims 22-28, wherein the gene is a SCN1A gene.

30. A method of increasing in a cell voltage-gated sodium channel activity encoded by a SCN1A gene containing a premature termination codon, the method comprising introducing into the cell an effective amount of the tRNA of any one of claims 1-4 or the expression vector of any one of claims 5-15, thereby permitting an amino acid to be incorporated into the SCN1A gene product at a position that would otherwise result in a truncated SCN1A gene product caused by the premature termination codon.

31. A method of increasing in a cell voltage-gated sodium channel activity encoded by a SCN1A gene containing a premature termination codon, the method comprising introducing into the cell an effective amount of a tRNA set forth in Table 3, or an expression vector comprising a nucleotide sequence encoding the tRNA, thereby permitting an amino acid to be incorporated into the SCN1A gene product at a position that would otherwise result in a truncated SCN1A gene product caused by the premature termination codon.

32. The method of claim 31, wherein the tRNA comprises a nucleotide sequence selected from SEQ ID NOs: 6-9, 11, 16-18, 22, 35, 36, 38, 45, 178, 180, and 187.

33. The method of any one of claims 29-32, wherein the SCN1A gene product produced with the tRNA is a functional SCN1A gene product.

34. The method of claim 33, wherein the functional SCN1A gene product has greater activity than the truncated SCN1A gene product.

35. The method of claim 33 or 34, wherein the functional SCN1A gene product is the Nav1.1 protein.

36. The method of any one of claims 33-35, wherein the functional SCN1A gene product comprises any one of SEQ ID NOs: 863-868.

37. The method of claim 36, wherein the functional SCN1A gene product comprises SEQ ID NO: 863 or SEQ ID NO: 864.

38. The method of any one of claims 22-37, wherein the cell is a human cell.

39. The method of any one of claims 22-38, wherein the tRNA becomes aminoacylated in the cell.

40. A method of treating a premature termination codon-mediated disorder in a subject in need thereof wherein the subject has a gene with a premature termination codon, the method comprising administering to the subject an effective amount of the tRNA of any one of claims 1-4 or the expression vector of any one of claims 5-15, thereby to treat the disorder in the subject.

41. The method of claim 40, wherein the disorder is a disorder set forth in Table 5 or Table 6.

42. The method of claim 41, wherein the disorder is a disorder set forth in Table 5.

43. A method of treating a premature termination codon-mediated disorder in a subject in need thereof wherein the subject has a gene with a premature termination codon, the method comprising administering to the subject an effective amount of a tRNA set forth in Table 3, or an expression vector comprising a nucleotide sequence encoding the tRNA, thereby to treat the disorder in the subject, wherein the disorder is a disorder set forth in Table 5.

44. The method of claim 43, wherein the tRNA comprises a nucleotide sequence selected from SEQ ID NOs: 6-9, 11, 16-18, 22, 35, 36, 38, 45, 178, 180, and 187.

45. The method of any one of claims 40-44, wherein the disorder is Dravet syndrome.

46. The method of claim 45, wherein the method further comprises administering stiripentol, cannabidiol, a ketogenic diet, clobazam, topiramate, fenfluramine, or valproic acid to the subject.

47. The method of claim 45 or 46, wherein the gene is SCN1A.

48. The method of any one of claims 40-47, wherein the subject is a human.

49. The method of any one of claim 29-39 or 47, wherein the premature termination codon in the SCN1A gene is caused by a mutation selected from c.664C>T, c.1129C>T, c.1492A>T, c.1624C>T, c.1738C>T, c.1837C>T, c.2134C>T, c.2593C>T, c.3637C>T, c.3733C>T, c.3985C>T, c.4573C>T, c.5656C>T, and c.5734C>T.

50. The method of claim 49, wherein the premature termination codon in the SCN1A gene is caused by a mutation selected from c.1738C>T and c.3985C>T.


SEQUENCE LISTING (902 sequences)

SEQ ID NO: 1 - DNA, 73 bp, Artificial Sequence
  Description of Artificial Sequence Synthetic oligonucleotide
gggccagtggcgcaatggataacgcgtctgacttcagatcagaagattccaggttcgactcctggctggctcg

SEQ ID NO: 2 - DNA, 73 bp, Artificial Sequence
  Description of Artificial Sequence Synthetic oligonucleotide
gggccagtggcgcaatggataacgcgtctgacttcagatcagaagattgtaggttcgactcctacctggctcg

SEQ ID NO: 3 - DNA, 73 bp, Artificial Sequence
  Description of Artificial Sequence Synthetic oligonucleotide
gggccagtggcgcaatggataacgcgtctgacttcagatcagaagattctaggttcgactcctggctggctcg

SEQ ID NO: 4 - DNA, 73 bp, Artificial Sequence
  Description of Artificial Sequence Synthetic oligonucleotide
ggccgcgtggcctaatggataaggcgtctgatttcagatcagaagattgagggttcgagtcccttcgtggtcg

SEQ ID NO: 5 - DNA, 73 bp, Artificial Sequence
  Description of Artificial Sequence Synthetic oligonucleotide
ggccgcgtggcctaatggataaggcgtctgatttcagatcagaagattgggggttcgagtcccttcgtggtcg

SEQ ID NO: 6 - DNA, 73 bp, Artificial Sequence
  Description of Artificial Sequence Synthetic oligonucleotide
gacccagtggcctaatggataaggcatcagccttcagagctggggattgtgggttcgagtcccatctgggtcg

SEQ ID NO: 7 - DNA, 73 bp, Artificial Sequence
  Description of Artificial Sequence Synthetic oligonucleotide
gccccagtggcctaatggataaggcactggccttcaaagccagggattgtgggttcgagtcccacctggggta

SEQ ID NO: 8 - DNA, 73 bp, Artificial Sequence
  Description of Artificial Sequence Synthetic oligonucleotide
gccccagtggcctaatggataaggcactggccttcaaagccagggattgtgggttcgagtcccacctggggtg

SEQ ID NO: 9 - DNA, 73 bp, Artificial Sequence
  Description of Artificial Sequence Synthetic oligonucleotide
gccccggtggcctaatggataaggcattggccttcaaagccagggattgtgggttcgagtcccacccggggta

SEQ ID NO: 10 - DNA, 73 bp, Artificial Sequence
  Description of Artificial Sequence Synthetic oligonucleotide
gccccagtggcctaatggataaggcattggccttcaaagccagggattgtgggttcgagtcccatctggggtg

SEQ ID NO: 11 - DNA, 73 bp, Artificial Sequence
  Description of Artificial Sequence Synthetic oligonucleotide
ggccgcgtggcctaatggataaggcgtctgacttcagatcagaagattgcaggttcgagtcctgccgcggtcg

SEQ ID NO: 12 - DNA, 73 bp, Artificial Sequence
  Description of Artificial Sequence Synthetic oligonucleotide
gaccgcgtggcctaatggataaggcgtctgacttcagatcagaagattgagggttcgagtcccttcgtggtcg

SEQ ID NO: 13 - DNA, 73 bp, Artificial Sequence
  Description of Artificial Sequence Synthetic oligonucleotide
gaccacgtggcctaatggataaggcgtctgacttcagatcagaagattgagggttcgaatcccttcgtggttg

SEQ ID NO: 14 - DNA, 73 bp, Artificial Sequence
  Description of Artificial Sequence Synthetic oligonucleotide
gaccacgtggcctaacggataaggcgtctgacttcagatcagaagattgagggttcgaatcccttcgtggtta

SEQ ID NO: 15 - DNA, 87 bp, Artificial Sequence
  Description of Artificial Sequence Synthetic oligonucleotide
ggctctgtggcgcaatggatagcgcattggacttcaagtgacgagaaagcgattcaaaggttgtgggttcgaatcccaccagagtcg

SEQ ID NO: 16 - DNA, 73 bp, Artificial Sequence
  Description of Artificial Sequence Synthetic oligonucleotide
ggctctgtggcgcaatggatagcgcattggacttcaaattcaaaggttgtgggttcgaatcccaccagagtcg

SEQ ID NO: 17 - DNA, 85 bp, Artificial Sequence
  Description of Artificial Sequence Synthetic oligonucleotide
ggctccgtggcgcaatggatagcgcattggacttcaagaggctgaaggcattcaaaggttccgggttcgagtcccggcggagtcg

SEQ ID NO: 18 - DNA, 73 bp, Artificial Sequence
  Description of Artificial Sequence Synthetic oligonucleotide
ggctccgtggcgcaatggatagcgcattggacttcaaattcaaaggttccgggttcgagtcccggcggagtcg

SEQ ID NO: 19 - DNA, 86 bp, Artificial Sequence
  Description of Artificial Sequence Synthetic oligonucleotide
ggctctgtggcgcaatggatagcgcattggacttcaagcatgattgagagattcaaaggttgcgggttcgagtcccgccagagtcg

SEQ ID NO: 20 - DNA, 73 bp, Artificial Sequence
  Description of Artificial Sequence Synthetic oligonucleotide
ggctctgtggcgcaatggatagcgcattggacttcaaattcaaaggttgcgggttcgagtcccgccagagtcg

SEQ ID NO: 21 - DNA, 86 bp, Artificial Sequence
  Description of Artificial Sequence Synthetic oligonucleotide
ggctctgtggcgcaatggatagcgcattggacttcaagacaaatggaggcattcaaaggttgtgggttcgagtcccaccagagtcg

SEQ ID NO: 22 - DNA, 73 bp, Artificial Sequence
  Description of Artificial Sequence Synthetic oligonucleotide
ggctctgtggcgcaatggatagcgcattggacttcaaattcaaaggttgtgggttcgagtcccaccagagtcg

SEQ ID NO: 23 - DNA, 74 bp, Artificial Sequence
  Description of Artificial Sequence Synthetic oligonucleotide
gtctctgtggcgcaatggacgagcgcgctggacttcaaatccagaggttctgggttcgagtcccggcagagatg

SEQ ID NO: 24 - DNA, 86 bp, Artificial Sequence
  Description of Artificial Sequence Synthetic oligonucleotide
ggctctgtggagcaatggatagcacattggacttcaagcatgaccgagagattcaaaggttgcgggttcgagtcccaccagagttg

SEQ ID NO: 25 - DNA, 73 bp, Artificial Sequence
  Description of Artificial Sequence Synthetic oligonucleotide
ggctctgtggagcaatggatagcacattggacttcaaattcaaaggttgcgggttcgagtcccaccagagttg

SEQ ID NO: 26 - DNA, 200 bp, Artificial Sequence
  Description of Artificial Sequence Synthetic polynucleotide
ctacccagaggcaggcgggagactcccccgagcgtccaataagagcgccgccaatggagccgcccgcccgcgggggtgcagagggacttccgggtgaggtcctccgctacttccctccccacggaaaagatagaccagtctgacgcgagcctgaaggcggctacacgctttaagctaagtaaaggcaccttctcgctggc

SEQ ID NO: 27 - DNA, 200 bp, Artificial Sequence
  Description of Artificial Sequence Synthetic polynucleotide
acttgtatgttgtttttatctgtcagtttgttaatcccaagattccctttggaaataaagcgaaattgaccgtagtggttatgaccaacttctagtctaaacttaattcttggaactcaaggatctgagcaaacaactgtcagggtgacacattgcttaaacggtgacagcggtcgagagccttgtcccggatggagagt

SEQ ID NO: 28 - DNA, 51 bp, Artificial Sequence
  Description of Artificial Sequence Synthetic oligonucleotide
ctgagacctctaagagccttatcttgatttgaagggatgagggtggttgtg

SEQ ID NO: 29 - DNA, 51 bp, Artificial Sequence
  Description of Artificial Sequence Synthetic oligonucleotide
acaagccttttcagctttagagggtgagcaaaggatgtgggatctgagaac

SEQ ID NO: 30 - DNA, 51 bp, Artificial Sequence
  Description of Artificial Sequence Synthetic oligonucleotide
ctaatagaaagaaatgagaccgcctgatggaaaaatgtgaaagtaaacttt

SEQ ID NO: 31 - DNA, 1854 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagtgaaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 32 - DNA, 104 bp, Artificial Sequence
  Description of Artificial Sequence Synthetic polynucleotide
acttgtatgttgtttttatctgtcagtttgttaatcccaagattccctttggaaataaagcgaaattgaccgtagtggttatgaccaacttctagtctaaactt

SEQ ID NO: 33 - DNA, 269 bp, Artificial Sequence
  Description of Artificial Sequence Synthetic polynucleotide
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgggcggaggaaggcaccttctcgctggc

SEQ ID NO: 34 - DNA, 46 bp, Artificial Sequence
  Description of Artificial Sequence Synthetic oligonucleotide
acttgtatgttgtttttatctgtcagtttgttaatcccaagattcc

SEQ ID NO: 35 - DNA, 74 bp, Artificial Sequence
  Description of Artificial Sequence Synthetic oligonucleotide
gtctctgtggcgcaatggacgagcgcgctggacttcaaatccagaggttccgggttcgagtcccggcagagatg

SEQ ID NO: 36 - DNA, 72 bp, Artificial Sequence
  Description of Artificial Sequence Synthetic oligonucleotide
ggtcccatggtgtaatggttagcactctggactttaaatccagcgatccgagttcaaatctcggtgggacct

SEQ ID NO: 37 - DNA, 72 bp, Artificial Sequence
  Description of Artificial Sequence Synthetic oligonucleotide
ggttccatggtgtaatggttagcactctggactttaaatccagcgacccgagttcaaatctcggtgggacct

SEQ ID NO: 38 - DNA, 72 bp, Artificial Sequence
  Description of Artificial Sequence Synthetic oligonucleotide
ggttccatggtgtaatggttagcactctggactttaaatccagcgatccgagttcaaatctcggtggaacct

SEQ ID NO: 39 - DNA, 72 bp, Artificial Sequence
  Description of Artificial Sequence Synthetic oligonucleotide
ggttccatggtgtaatggttagcactctggactttaaatccagcgatccgagttcaaatctcggtgggacct

SEQ ID NO: 40 - DNA, 72 bp, Artificial Sequence
  Description of Artificial Sequence Synthetic oligonucleotide
ggttccatggtgtaatggtgagcactctggactttaaatccagcgatccgagttcaaatctcggtgggacct

SEQ ID NO: 41 - DNA, 72 bp, Artificial Sequence
  Description of Artificial Sequence Synthetic oligonucleotide
ggttccatggtgtaatggctagcactctggactttaaatccagcgatccgagttcaaatctcggtgggattt

SEQ ID NO: 42 - DNA, 72 bp, Artificial Sequence
  Description of Artificial Sequence Synthetic oligonucleotide
ggttccatggtgtaatggttagcactctggactttaaatccagccatacaagttcaaatctcagtggaacct

SEQ ID NO: 43 - DNA, 71 bp, Artificial Sequence
  Description of Artificial Sequence Synthetic oligonucleotide
ggttccttggtgtaagatgagcactctggattttaaatccagcgatcagagttcaaatctcggtgggacct

SEQ ID NO: 44 - DNA, 72 bp, Artificial Sequence
  Description of Artificial Sequence Synthetic oligonucleotide
ggtcccatggtgtaatggttagcactctggactttaaatccagcaatctgagttcaaatctcggtgggacct

SEQ ID NO: 45 - DNA, 72 bp, Artificial Sequence
  Description of Artificial Sequence Synthetic oligonucleotide
ggccccatggtgtaatggttagcactctggactttaaatccagcgatccgagttcaaatctcggtgggacct

SEQ ID NO: 46 - DNA, 72 bp, Artificial Sequence
  Description of Artificial Sequence Synthetic oligonucleotide
ggtctcatggtgtaatggttagcacactggactttaagtccagcaatccgagttcgagtcttggtgagacca

SEQ ID NO: 47 - DNA, 72 bp, Artificial Sequence
  Description of Artificial Sequence Synthetic oligonucleotide
ggacccatggtgtaatggttagcactctggactttaaatccagcaatccaagttcaaatctcggtgggacct

SEQ ID NO: 48 - DNA, 72 bp, Artificial Sequence
  Description of Artificial Sequence Synthetic oligonucleotide
gtttccatggtgtaatggttggcactctggactttaaatccagcaatccaagttcaagtctctgtgggacct

SEQ ID NO: 49 - DNA, 106 bp, Artificial Sequence
  Description of Artificial Sequence Synthetic polynucleotide
gtcaggatggccgagtggtctaaggcgccagactctagctatggcttcctcgctctgagggttctggtctcccctggaggcgtgggttcgaatcccacttctgaca

SEQ ID NO: 50 - DNA, 105 bp, Artificial Sequence
  Description of Artificial Sequence Synthetic polynucleotide
gtcaggatggccgagtggtctaaggcgccagactctagcttagcttccctgtctggggattctggtctccgtatggaggcgtgggttcgaatcccacttctgaca

SEQ ID NO: 51 - DNA, 107 bp, Artificial Sequence
  Description of Artificial Sequence Synthetic polynucleotide
gtcaggatggccgagtggtctaaggcgccagactctaggtgacaagccttacctacgggtgttctggtctccgaatggaggcgtgggttcgaatcccacttctgaca

SEQ ID NO: 52 - DNA, 106 bp, Artificial Sequence
  Description of Artificial Sequence Synthetic polynucleotide
gtcaggatggccgagtggtctaaggcgccagactctagcgttcgcttcctctactgagggttctggtctccgtgtggaggcgtgggttcgaatcccacttctgaca

SEQ ID NO: 53 - DNA, 106 bp, Artificial Sequence
  Description of Artificial Sequence Synthetic polynucleotide
gtcaggatggccgagtggtctaaggcgccagacttcagctatggcttcctcgctctgagggttctggtctcccctggaggcgtgggttcgaatcccacttctgaca

SEQ ID NO: 54 - DNA, 105 bp, Artificial Sequence
  Description of Artificial Sequence Synthetic polynucleotide
gtcaggatggccgagtggtctaaggcgccagacttcagcttagcttccctgtctggggattctggtctccgtatggaggcgtgggttcgaatcccacttctgaca

SEQ ID NO: 55 - DNA, 107 bp, Artificial Sequence
  Description of Artificial Sequence Synthetic polynucleotide
gtcaggatggccgagtggtctaaggcgccagacttcaggtgacaagccttacctacgggtgttctggtctccgaatggaggcgtgggttcgaatcccacttctgaca

SEQ ID NO: 56 - DNA, 106 bp, Artificial Sequence
  Description of Artificial Sequence Synthetic polynucleotide
gtcaggatggccgagtggtctaaggcgccagacttcagcgttcgcttcctctactgagggttctggtctccgtgtggaggcgtgggttcgaatcccacttctgaca

SEQ ID NO: 57 - DNA, 106 bp, Artificial Sequence
  Description of Artificial Sequence Synthetic polynucleotide
gtcaggatggccgagtggtctaaggcgccagactttagctatggcttcctcgctctgagggttctggtctcccctggaggcgtgggttcgaatcccacttctgaca

SEQ ID NO: 58 - DNA, 105 bp, Artificial Sequence
  Description of Artificial Sequence Synthetic polynucleotide
gtcaggatggccgagtggtctaaggcgccagactttagcttagcttccctgtctggggattctggtctccgtatggaggcgtgggttcgaatcccacttctgaca

SEQ ID NO: 59 - DNA, 107 bp, Artificial Sequence
  Description of Artificial Sequence Synthetic polynucleotide
gtcaggatggccgagtggtctaaggcgccagactttaggtgacaagccttacctacgggtgttctggtctccgaatggaggcgtgggttcgaatcccacttctgaca

SEQ ID NO: 60 - DNA, 106 bp, Artificial Sequence
  Description of Artificial Sequence Synthetic polynucleotide
gtcaggatggccgagtggtctaaggcgccagactttagcgttcgcttcctctactgagggttctggtctccgtgtggaggcgtgggttcgaatcccacttctgaca

SEQ ID NO: 61 - DNA, 89 bp, Artificial Sequence
  Description of Artificial Sequence Synthetic oligonucleotide
ccttcgatagctcagttggtagagcggaggactctagagttactagaatagtgatccttaggtcgctggttcgaatccggctcgaagga

SEQ ID NO: 62 - DNA, 89 bp, Artificial Sequence
  Description of Artificial Sequence Synthetic oligonucleotide
ccttcgatagctcagttggtagagcggaggactctagtcagtacaatatggtaatccttaggtcgctggttcgattccggctcgaagga

SEQ ID NO: 63 - DNA, 89 bp, Artificial Sequence
  Description of Artificial Sequence Synthetic oligonucleotide
ccttcgatagctcagctggtagagcggaggactctaggcttgtggctgtggacatccttaggtcgctggttcgattccggctcgaagga

SEQ ID NO: 64 - DNA, 93 bp, Artificial Sequence
  Description of Artificial Sequence Synthetic oligonucleotide
ccttcgatagctcagctggtagagcggaggactctagctaactccccgttagaagacatccttaggtcgctggttcgactccggctcgaagga

SEQ ID NO: 65 - DNA, 89 bp, Artificial Sequence
  Description of Artificial Sequence Synthetic oligonucleotide
ctttcgatagttcagttggtagagcggaggactctagagtattaacgttggtgatccttaggtcgctggttcgagtccggctcgaagga

SEQ ID NO: 66 - DNA, 89 bp, Artificial Sequence
  Description of Artificial Sequence Synthetic oligonucleotide
ccttcgatagctcagttggtagagcggaggactttagagttactagaatagtgatccttaggtcgctggttcgaatccggctcgaagga

SEQ ID NO: 67 - DNA, 89 bp, Artificial Sequence
  Description of Artificial Sequence Synthetic oligonucleotide
ccttcgatagctcagttggtagagcggaggactttagtcagtacaatatggtaatccttaggtcgctggttcgattccggctcgaagga

SEQ ID NO: 68 - DNA, 89 bp, Artificial Sequence
  Description of Artificial Sequence Synthetic oligonucleotide
ccttcgatagctcagctggtagagcggaggactttaggcttgtggctgtggacatccttaggtcgctggttcgattccggctcgaagga

SEQ ID NO: 69 - DNA, 93 bp, Artificial Sequence
  Description of Artificial Sequence Synthetic oligonucleotide
ccttcgatagctcagctggtagagcggaggactttagctaactccccgttagaagacatccttaggtcgctggttcgactccggctcgaagga

SEQ ID NO: 70 - DNA, 89 bp, Artificial Sequence
  Description of Artificial Sequence Synthetic oligonucleotide
ctttcgatagttcagttggtagagcggaggactttagagtattaacgttggtgatccttaggtcgctggttcgagtccggctcgaagga

SEQ ID NO: 71 - DNA, 72 bp, Artificial Sequence
  Description of Artificial Sequence Synthetic oligonucleotide
gggggtatagctcagtggtagagcatttgacttcagatcaagaggtccctggttcaaatccaggtgccccct

SEQ ID NO: 72 - DNA, 72 bp, Artificial Sequence
  Description of Artificial Sequence Synthetic oligonucleotide
gggggtatagctcaggggtagagcatttgacttcagatcaagaggtccctggttcaaatccagatgccccct

SEQ ID NO: 73 - DNA, 72 bp, Artificial Sequence
  Description of Artificial Sequence Synthetic oligonucleotide
gggggtatagctcaggggtagagtatttggcttcagatcaagaggtccctggttcaaatccaggtgccccct

SEQ ID NO: 74 - DNA, 72 bp, Artificial Sequence
  Description of Artificial Sequence Synthetic oligonucleotide
gggggtatagctcaggggtagagcatttgacttcagatcaagaggtccttggttcaaatccaggtgtcccct

SEQ ID NO: 75 - DNA, 72 bp, Artificial Sequence
  Description of Artificial Sequence Synthetic oligonucleotide
gggggtatagctcagaggtagagcatttgacttcagatcaagagatctctggttcaaatccaggtgccccct

SEQ ID NO: 76 - DNA, 72 bp, Artificial Sequence
  Description of Artificial Sequence Synthetic oligonucleotide
gggggtatagctcaggggtagagcatttgacttcagatcaagaggtccctagttcaaatccaggtgccccct

SEQ ID NO: 77 - DNA, 72 bp, Artificial Sequence
  Description of Artificial Sequence Synthetic oligonucleotide
ggtggtatagctcaggggtagagcatttgacttcagatcaagagatccctggttcgaatccaggtgccccct

SEQ ID NO: 78 - DNA, 72 bp, Artificial Sequence
  Description of Artificial Sequence Synthetic oligonucleotide
gggggtataactcaggggtagagcatttgacttcagatcaagaggtccctggttcaaatccaggtgccccct

SEQ ID NO: 79 - DNA, 72 bp, Artificial Sequence
  Description of Artificial Sequence Synthetic oligonucleotide
tggggtatagctcaggggtagagcatttgacttcagatcaagaggtccctggttcaaatccaggtgccccct

SEQ ID NO: 80 - DNA, 72 bp, Artificial Sequence
  Description of Artificial Sequence Synthetic oligonucleotide
gggggtatagctcagaggaagagcatttgacttcagatcaagaggtccctgattcaaatccaggtgccccct

SEQ ID NO: 81 - DNA, 72 bp, Artificial Sequence
  Description of Artificial Sequence Synthetic oligonucleotide
gggggtaaagctcaggggtagagcatttgacttcagattaagaggtccctggttcaaatccaggtaccccct

SEQ ID NO: 82 - DNA, 72 bp, Artificial Sequence
  Description of Artificial Sequence Synthetic oligonucleotide
gggggtatagctcagtggtagagcatttgacttcagatcaagaggtccctggttcaaatccgggtgccccct

SEQ ID NO: 83 - DNA, 72 bp, Artificial Sequence
  Description of Artificial Sequence Synthetic oligonucleotide
ggggttatagctcaggtgtagagcatttgacttcagatcaagaggtccctggttcaaatccaggtgccccct

SEQ ID NO: 84 - DNA, 72 bp, Artificial Sequence
  Description of Artificial Sequence Synthetic oligonucleotide
gggggtatagctcaggggtagagcatttgacttcagatcacgaggtccctggttcaaatcgaggtgccccct

SEQ ID NO: 85 - DNA, 72 bp, Artificial Sequence
  Description of Artificial Sequence Synthetic oligonucleotide
gggggtatagctcaggggtggagcatttgacttcagatcaaggggtccctgtttcaaatccaggtgccccct

SEQ ID NO: 86 - DNA, 72 bp, Artificial Sequence
  Description of Artificial Sequence Synthetic oligonucleotide
gggggtatagctcagtggtagagcatttgacttcagatcaagaggtccccggttcaaatccgggtgccccct

SEQ ID NO: 87 - DNA, 72 bp, Artificial Sequence
  Description of Artificial Sequence Synthetic oligonucleotide
gggggtatagctcaggggtagagcatttgacttcagatcaagaggtccctggttcaaatccaggtgccccct

SEQ ID NO: 88 - DNA, 72 bp, Artificial Sequence
  Description of Artificial Sequence Synthetic oligonucleotide
gggggtatagctcaggggtagagcatttgacttcagatcaagaggtccctggttcaaatccgggtgccccct

SEQ ID NO: 89 - DNA, 72 bp, Artificial Sequence
  Description of Artificial Sequence Synthetic oligonucleotide
gggggtatagctcaggggtagagcatttgacttcagatcaagaggtccctggttcaaatccaggtaccccct

SEQ ID NO: 90 - DNA, 72 bp, Artificial Sequence
  Description of Artificial Sequence Synthetic oligonucleotide
gggggtatagctcaggggtagagcatttgacttcagatcaagaggtccccggttcaaatccgggtgccccct

SEQ ID NO: 91 - DNA, 72 bp, Artificial Sequence
  Description of Artificial Sequence Synthetic oligonucleotide
gggggcatagctcaggggtagagcatttgacttcagatcaagaggtccccggttcaaatccgggtgctccct

SEQ ID NO: 92 - DNA, 72 bp, Artificial Sequence
  Description of Artificial Sequence Synthetic oligonucleotide
gggggtatagctcaggggtagagcatttgacttcagattaagaggtccctggttcaaatccaggtgccccct

SEQ ID NO: 93 - DNA, 72 bp, Artificial Sequence
  Description of Artificial Sequence Synthetic oligonucleotide
tccctggtggtctagtggttaggattcggcgctctaaccgccgcggcccgggttcgattcccggtcagggaa

SEQ ID NO: 94 - DNA, 72 bp, Artificial Sequence
  Description of Artificial Sequence Synthetic oligonucleotide
tccctggtggtctagtggttaggatttggcgctctaaccgccgcggcctgggttcgattcccggtcagggaa

SEQ ID NO: 95 - DNA, 72 bp, Artificial Sequence
  Description of Artificial Sequence Synthetic oligonucleotide
tccctggtggtctagtggttaggctttggtgctctaacctccatggcccaggtttgattcctggtcagggaa

SEQ ID NO: 96 - DNA, 72 bp, Artificial Sequence
  Description of Artificial Sequence Synthetic oligonucleotide
tccctggtggtctagtggttaggattcggcgctttaaccgccgcggcccgggttcgattcccggtcagggaa

SEQ ID NO: 97 - DNA, 72 bp, Artificial Sequence
  Description of Artificial Sequence Synthetic oligonucleotide
tccctggtggtctagtggttaggatttggcgctttaaccgccgcggcctgggttcgattcccggtcagggaa

SEQ ID NO: 98 - DNA, 72 bp, Artificial Sequence
  Description of Artificial Sequence Synthetic oligonucleotide
tccctggtggtctagtggttaggctttggtgctttaacctccatggcccaggtttgattcctggtcagggaa

SEQ ID NO: 99 - DNA, 72 bp, Artificial Sequence
  Description of Artificial Sequence Synthetic oligonucleotide
tcccacatggtctagcggttaggattcctggttctaacccaggcggcccgggttcgactcccggtgtgggaa

SEQ ID NO: 100 - DNA, 72 bp, Artificial Sequence
  Description of Artificial Sequence Synthetic oligonucleotide
tcccatatggtctagcggttaggattcctggttctaacccaggcggcccgggttcgactcccggtatgggaa

SEQ ID NO: 101 - DNA, 72 bp, Artificial Sequence
  Description of Artificial Sequence Synthetic oligonucleotide
tccctggtggtctagtggctaggattcggcgctctaaccgccgcggcccgggttcgattcccggtcagggaa

SEQ ID NO: 102 - DNA, 72 bp, Artificial Sequence
  Description of Artificial Sequence Synthetic oligonucleotide
tcccacatggtctagcggttaggattcctggttttaacccaggcggcccgggttcgactcccggtgtgggaa

SEQ ID NO: 103 - DNA, 72 bp, Artificial Sequence
  Description of Artificial Sequence Synthetic oligonucleotide
tcccatatggtctagcggttaggattcctggttttaacccaggcggcccgggttcgactcccggtatgggaa

SEQ ID NO: 104 - DNA, 72 bp, Artificial Sequence
  Description of Artificial Sequence Synthetic oligonucleotide
tccctggtggtctagtggctaggattcggcgctttaaccgccgcggcccgggttcgattcccggtcagggaa

SEQ ID NO: 105 - DNA, 74 bp, Artificial Sequence
  Description of Artificial Sequence Synthetic oligonucleotide
gtttccgtagtgtagtggttagcgcgttcgccttcaaaagcgaaaggtccccggttcgaaaccgggcggaaaca

SEQ ID NO: 106 - DNA, 71 bp, Artificial Sequence
  Description of Artificial Sequence Synthetic oligonucleotide
gcgccgctggtgtagtggtatcatgcaagatttcaattcttgcgacccgggttcgattcccgggcggcgca

SEQ ID NO: 107 - DNA, 71 bp, Artificial Sequence
  Description of Artificial Sequence Synthetic oligonucleotide
gcattggtagttcaatggtagaattctcgccttcaacgcgggtgacccgggttcgattcccggccaatgca

SEQ ID NO: 108 - DNA, 71 bp, Artificial Sequence
  Description of Artificial Sequence Synthetic oligonucleotide
gcattggtggttcaatggtagaattctcgccttcaacgcgggtgacccgggttcgattcccggccaatgca

SEQ ID NO: 109 - DNA, 71 bp, Artificial Sequence
  Description of Artificial Sequence Synthetic oligonucleotide
gcattggtggttcaatggtagaattctcgccttcaactcgggtgacccgggttcgattcccggccaatgca

SEQ ID NO: 110 - DNA, 71 bp, Artificial Sequence
  Description of Artificial Sequence Synthetic oligonucleotide
gcatgggtggttcagtggtagaattctcgccttcaacgcgggaggcccgggttcgattcccggcccatgca

SEQ ID NO: 111 - DNA, 71 bp, Artificial Sequence
  Description of Artificial Sequence Synthetic oligonucleotide
gcattggtggttcagtggtagaattctcgccttcaacgcgggaggcccgggttcgattcccggccaatgca

SEQ ID NO: 112 - DNA, 71 bp, Artificial Sequence
  Description of Artificial Sequence Synthetic oligonucleotide
gcattggtggttcagtggtagaattctcgccttcaacgcgggaggcccgggtttgattcccggccaatgca

SEQ ID NO: 113 - DNA, 71 bp, Artificial Sequence
  Description of Artificial Sequence Synthetic oligonucleotide
gcattggtggttcagtggtagaattctcgccttcaacgcgggaggcccgggttcggttcccggccaatgca

SEQ ID NO: 114 - DNA, 72 bp, Artificial Sequence
  Description of Artificial Sequence Synthetic oligonucleotide
gcgttggtggtatagtggtgagcatagctgccttcaaagcagttgacccgggttcgattcccggccaacgca

SEQ ID NO: 115 - DNA, 82 bp, Artificial Sequence
  Description of Artificial Sequence Synthetic oligonucleotide
ggtagcgtggccgagcggtctaaggcgctggattctagctccagtctcttcgggggcgtgggttcgaatcccaccgctgcca

SEQ ID NO: 116 - DNA, 82 bp, Artificial Sequence
  Description of Artificial Sequence Synthetic oligonucleotide
ggtagtgtggccgagcggtctaaggcgctggattctagctccagtctcttcgggggcgtgggttcgaatcccaccgctgcca

SEQ ID NO: 117 - DNA, 82 bp, Artificial Sequence
  Description of Artificial Sequence Synthetic oligonucleotide
ggtagtgtggccgagcggtctaaggcgctggattctagctccagtctcttcgggggcgtgggttcgaatcccaccactgcca

SEQ ID NO: 118 - DNA, 82 bp, Artificial Sequence
  Description of Artificial Sequence Synthetic oligonucleotide
ggtagcgtggccgagcggtctaaggcgctggatttcagctccagtctcttcgggggcgtgggttcgaatcccaccgctgcca

SEQ ID NO: 119 - DNA, 82 bp, Artificial Sequence
  Description of Artificial Sequence Synthetic oligonucleotide
ggtagtgtggccgagcggtctaaggcgctggatttcagctccagtctcttcgggggcgtgggttcgaatcccaccgctgcca

SEQ ID NO: 120 - DNA, 82 bp, Artificial Sequence
  Description of Artificial Sequence Synthetic oligonucleotide
ggtagtgtggccgagcggtctaaggcgctggatttcagctccagtctcttcgggggcgtgggttcgaatcccaccactgcca

SEQ ID NO: 121 - DNA, 82 bp, Artificial Sequence
  Description of Artificial Sequence Synthetic oligonucleotide
ggtagcgtggccgagcggtctaaggcgctggattttagctccagtctcttcgggggcgtgggttcgaatcccaccgctgcca

SEQ ID NO: 122 - DNA, 82 bp, Artificial Sequence
  Description of Artificial Sequence Synthetic oligonucleotide
ggtagtgtggccgagcggtctaaggcgctggattttagctccagtctcttcgggggcgtgggttcgaatcccaccgctgcca

SEQ ID NO: 123 - DNA, 82 bp, Artificial Sequence
  Description of Artificial Sequence Synthetic oligonucleotide
ggtagtgtggccgagcggtctaaggcgctggattttagctccagtctcttcgggggcgtgggttcgaatcccaccactgcca

SEQ ID NO: 124 - DNA, 83 bp, Artificial Sequence
  Description of Artificial Sequence Synthetic oligonucleotide
gtcaggatggccgagtggtctaaggcgccagactctagttctggtctcccctggaggcgtgggttcgaatcccacttctgaca

SEQ ID NO: 125 - DNA, 84 bp, Artificial Sequence
  Description of Artificial Sequence Synthetic oligonucleotide
gtcaggatggccgagtggtctaaggcgccagactctagttctggtctccgtatggaggcgtgggttcgaatcccacttctgaca

SEQ ID NO: 126 - DNA, 84 bp, Artificial Sequence
  Description of Artificial Sequence Synthetic oligonucleotide
gtcaggatggccgagtggtctaaggcgccagactctagttctggtctccgaatggaggcgtgggttcgaatcccacttctgaca

SEQ ID NO: 127 - DNA, 84 bp, Artificial Sequence
  Description of Artificial Sequence Synthetic oligonucleotide
gtcaggatggccgagtggtctaaggcgccagactctagttctggtctccgtgtggaggcgtgggttcgaatcccacttctgaca

SEQ ID NO: 128 - DNA, 83 bp, Artificial Sequence
  Description of Artificial Sequence Synthetic oligonucleotide
gtcaggatggccgagtggtctaaggcgccagacttcagttctggtctcccctggaggcgtgggttcgaatcccacttctgaca

SEQ ID NO: 129 - DNA, 84 bp, Artificial Sequence
  Description of Artificial Sequence Synthetic oligonucleotide
gtcaggatggccgagtggtctaaggcgccagacttcagttctggtctccgtatggaggcgtgggttcgaatcccacttctgaca

SEQ ID NO: 130 - DNA, 84 bp, Artificial Sequence
  Description of Artificial Sequence Synthetic oligonucleotide
gtcaggatggccgagtggtctaaggcgccagacttcagttctggtctccgaatggaggcgtgggttcgaatcccacttctgaca

SEQ ID NO: 131 - DNA, 84 bp, Artificial Sequence
  Description of Artificial Sequence Synthetic oligonucleotide
gtcaggatggccgagtggtctaaggcgccagacttcagttctggtctccgtgtggaggcgtgggttcgaatcccacttctgaca

SEQ ID NO: 132 - DNA, 83 bp, Artificial Sequence
  Description of Artificial Sequence Synthetic oligonucleotide
gtcaggatggccgagtggtctaaggcgccagactttagttctggtctcccctggaggcgtgggttcgaatcccacttctgaca

SEQ ID NO: 133 - DNA, 84 bp, Artificial Sequence
  Description of Artificial Sequence Synthetic oligonucleotide
gtcaggatggccgagtggtctaaggcgccagactttagttctggtctccgtatggaggcgtgggttcgaatcccacttctgaca

SEQ ID NO: 134 - DNA, 84 bp, Artificial Sequence
  Description of Artificial Sequence Synthetic oligonucleotide
gtcaggatggccgagtggtctaaggcgccagactttagttctggtctccgaatggaggcgtgggttcgaatcccacttctgaca

SEQ ID NO: 135 - DNA, 84 bp, Artificial Sequence
  Description of Artificial Sequence Synthetic oligonucleotide
gtcaggatggccgagtggtctaaggcgccagactttagttctggtctccgtgtggaggcgtgggttcgaatcccacttctgaca

SEQ ID NO: 136 - DNA, 83 bp, Artificial Sequence
  Description of Artificial Sequence Synthetic oligonucleotide
gtcaggatggccgagcggtctaaggcgctgcgttctagtcgcagtctcccctggaggcgtgggttcgaatcccactcctgaca

SEQ ID NO: 137 - DNA, 83 bp, Artificial Sequence
  Description of Artificial Sequence Synthetic oligonucleotide
gtcaggatggccgagcggtctaaggcgctgcgttctagtcgcagtctcccctggaggcgtgggttcgaatcccacttctgaca

SEQ ID NO: 138 - DNA, 83 bp, Artificial Sequence
  Description of Artificial Sequence Synthetic oligonucleotide
gtcaggatggccgagtggtctaaggagctgtgttctagtcgcagtctcccctggaggcgtgggttcgaatcccactcctgaca

SEQ ID NO: 139 - DNA, 83 bp, Artificial Sequence
  Description of Artificial Sequence Synthetic oligonucleotide
gtcaggatggccgagcagtctaaggcactgcgttctagtcgcagtctcccctggaggcgtggattcgaatcccactcctgaca

SEQ ID NO: 140 - DNA, 83 bp, Artificial Sequence
  Description of Artificial Sequence Synthetic oligonucleotide
gtcaggatggccgagcggtctaaggcgctgcgtttcagtcgcagtctcccctggaggcgtgggttcgaatcccactcctgaca

SEQ ID NO: 141 - DNA, 83 bp, Artificial Sequence
  Description of Artificial Sequence Synthetic oligonucleotide
gtcaggatggccgagcggtctaaggcgctgcgtttcagtcgcagtctcccctggaggcgtgggttcgaatcccacttctgaca

SEQ ID NO: 142 - DNA, 83 bp, Artificial Sequence
  Description of Artificial Sequence Synthetic oligonucleotide
gtcaggatggccgagtggtctaaggagctgtgtttcagtcgcagtctcccctggaggcgtgggttcgaatcccactcctgaca

SEQ ID NO: 143 - DNA, 83 bp, Artificial Sequence
  Description of Artificial Sequence Synthetic oligonucleotide
gtcaggatggccgagcagtctaaggcactgcgtttcagtcgcagtctcccctggaggcgtggattcgaatcccactcctgaca

SEQ ID NO: 144 - DNA, 83 bp, Artificial Sequence
  Description of Artificial Sequence Synthetic oligonucleotide
gtcaggatggccgagcggtctaaggcgctgcgttttagtcgcagtctcccctggaggcgtgggttcgaatcccactcctgaca

SEQ ID NO: 145 - DNA, 83 bp, Artificial Sequence
  Description of Artificial Sequence Synthetic oligonucleotide
gtcaggatggccgagcggtctaaggcgctgcgttttagtcgcagtctcccctggaggcgtgggttcgaatcccacttctgaca

SEQ ID NO: 146 - DNA, 83 bp, Artificial Sequence
  Description of Artificial Sequence Synthetic oligonucleotide
gtcaggatggccgagtggtctaaggagctgtgttttagtcgcagtctcccctggaggcgtgggttcgaatcccactcctgaca

SEQ ID NO: 147 - DNA, 83 bp, Artificial Sequence
  Description of Artificial Sequence Synthetic oligonucleotide
gtcaggatggccgagcagtctaaggcactgcgttttagtcgcagtctcccctggaggcgtggattcgaatcccactcctgaca

SEQ ID NO: 148 - DNA, 83 bp, Artificial Sequence
  Description of Artificial Sequence Synthetic oligonucleotide
accagaatggccgagtggttaaggcgttggactctagatccaatggatttatatccgcgtgggttcgaaccccacttctggta

SEQ ID NO: 149 - DNA, 83 bp, Artificial Sequence
  Description of Artificial Sequence Synthetic oligonucleotide
accaggatggccgagtggttaaggcgttggactctagatccaatggacatatgtctgcgtgggttcgaaccccactcctggta

SEQ ID NO: 150 - DNA, 83 bp, Artificial Sequence
  Description of Artificial Sequence Synthetic oligonucleotide
actgggatggctgagtggttaaggcgttggactctagatccaatgggcggttgcctgcgtgggttcgaaccccactcccagta

SEQ ID NO: 151 - DNA, 83 bp, Artificial Sequence
  Description of Artificial Sequence Synthetic oligonucleotide
gatgggatggctgagaggttaaggctttggactctagatccaatgggcagatgcctgcgtgggtttgaaccccactcccaata

SEQ ID NO: 152 - DNA, 83 bp, Artificial Sequence
  Description of Artificial Sequence Synthetic oligonucleotide
accagaatggccgagtggttaaggcgttggacttcagatccaatggatttatatccgcgtgggttcgaaccccacttctggta

SEQ ID NO: 153 - DNA, 83 bp, Artificial Sequence
  Description of Artificial Sequence Synthetic oligonucleotide
accaggatggccgagtggttaaggcgttggacttcagatccaatggacatatgtctgcgtgggttcgaaccccactcctggta

SEQ ID NO: 154 - DNA, 83 bp, Artificial Sequence
  Description of Artificial Sequence Synthetic oligonucleotide
actgggatggctgagtggttaaggcgttggacttcagatccaatgggcggttgcctgcgtgggttcgaaccccactcccagta

SEQ ID NO: 155 - DNA, 83 bp, Artificial Sequence
  Description of Artificial Sequence Synthetic oligonucleotide
gatgggatggctgagaggttaaggctttggacttcagatccaatgggcagatgcctgcgtgggtttgaaccccactcccaata

SEQ ID NO: 156 - DNA, 83 bp, Artificial Sequence
  Description of Artificial Sequence Synthetic oligonucleotide
accagaatggccgagtggttaaggcgttggactttagatccaatggatttatatccgcgtgggttcgaaccccacttctggta

SEQ ID NO: 157 - DNA, 83 bp, Artificial Sequence
  Description of Artificial Sequence Synthetic oligonucleotide
accaggatggccgagtggttaaggcgttggactttagatccaatggacatatgtctgcgtgggttcgaaccccactcctggta

SEQ ID NO: 158 - DNA, 83 bp, Artificial Sequence
  Description of Artificial Sequence Synthetic oligonucleotide
actgggatggctgagtggttaaggcgttggactttagatccaatgggcggttgcctgcgtgggttcgaaccccactcccagta

SEQ ID NO: 159 - DNA, 83 bp, Artificial Sequence
  Description of Artificial Sequence Synthetic oligonucleotide
gatgggatggctgagaggttaaggctttggactttagatccaatgggcagatgcctgcgtgggtttgaaccccactcccaata

SEQ ID NO: 160 - DNA, 82 bp, Artificial Sequence
  Description of Artificial Sequence Synthetic oligonucleotide
ggtagcgtggccgagcggtctaaggcgctggattctagctccagtctcttcggaggcgtgggttcgaatcccaccgctgcca

SEQ ID NO: 161 - DNA, 82 bp, Artificial Sequence
  Description of Artificial Sequence Synthetic oligonucleotide
ggtagtgtggccgagcggtctaaggcgctggattctagctccagtctcttcggaggcgtgggttcgaatcccaccactgcca

SEQ ID NO: 162 - DNA, 82 bp, Artificial Sequence
  Description of Artificial Sequence Synthetic oligonucleotide
ggtagcgtggccgagtggtctaaggcgctggattctagctccagtcatttcgatggcgtgggttcgaatcccaccgctgcca

SEQ ID NO: 163 - DNA, 82 bp, Artificial Sequence
  Description of Artificial Sequence Synthetic oligonucleotide
ggtagcgtggccgagcggtctaaggcgctggatttcagctccagtctcttcggaggcgtgggttcgaatcccaccgctgcca

SEQ ID NO: 164 - DNA, 82 bp, Artificial Sequence
  Description of Artificial Sequence Synthetic oligonucleotide
ggtagtgtggccgagcggtctaaggcgctggatttcagctccagtctcttcggaggcgtgggttcgaatcccaccactgcca

SEQ ID NO: 165 - DNA, 82 bp, Artificial Sequence
  Description of Artificial Sequence Synthetic oligonucleotide
ggtagcgtggccgagtggtctaaggcgctggatttcagctccagtcatttcgatggcgtgggttcgaatcccaccgctgcca

SEQ ID NO: 166 - DNA, 82 bp, Artificial Sequence
  Description of Artificial Sequence Synthetic oligonucleotide
ggtagcgtggccgagcggtctaaggcgctggattttagctccagtctcttcggaggcgtgggttcgaatcccaccgctgcca

SEQ ID NO: 167 - DNA, 82 bp, Artificial Sequence
  Description of Artificial Sequence Synthetic oligonucleotide
ggtagtgtggccgagcggtctaaggcgctggattttagctccagtctcttcggaggcgtgggttcgaatcccaccactgcca

SEQ ID NO: 168 - DNA, 82 bp, Artificial Sequence
  Description of Artificial Sequence Synthetic oligonucleotide
ggtagcgtggccgagtggtctaaggcgctggattttagctccagtcatttcgatggcgtgggttcgaatcccaccgctgcca

SEQ ID NO: 169 - DNA, 73 bp, Artificial Sequence
  Description of Artificial Sequence Synthetic oligonucleotide
gcccagctagctcagttggtagagcgtgggactctaaatcctagggtcgtgggttcgaaccccacgttgggcg

SEQ ID NO: 170 - DNA, 73 bp, Artificial Sequence
  Description of Artificial Sequence Synthetic oligonucleotide
gcccagctagctcagtctgtagagcatgagactctaagtctcagggtcatgggttggagccccatgttgtgca

SEQ ID NO: 171 - DNA, 77 bp, Artificial Sequence
  Description of Artificial Sequence Synthetic oligonucleotide
gcctagctagttcagtcggtagagcatgagactctaaatctcaggttcatgagtttgagccccatgttggtttggca

SEQ ID NO: 172 - DNA, 73 bp, Artificial Sequence
  Description of Artificial Sequence Synthetic oligonucleotide
ccccggctagctcagtcagtagagcttgagaatctaaatctcagggtcgtgggttggagccccacgttgggcg

SEQ ID NO: 173 - DNA, 200 bp, Artificial Sequence
  Description of Artificial Sequence Synthetic polynucleotide
gatcaccggaagaggtgacagacacctcggggcccatgaacgtttggaattcgtaaggacatgagaatctcggtggttccgtgtctgcccgccatcgcggccaccggccacgggcccaagccaagtgtagcgaagcttagaaaaggttgcccaacgtcatgtggcttgagaaggctgccgggcgccttaagccgccagca

SEQ ID NO: 174 - DNA, 200 bp, Artificial Sequence
  Description of Artificial Sequence Synthetic polynucleotide
cactgaacctttttttggccttagaatccctgttttggggcctgcaggaagtagcaaccaacccgagcctccgcagggaatgcactgacctgtagaatggacgttcagcttccctccctgtgtctcaacacgattacatttcaggaacagcctgggctgggaggcactgcgcacgcgcgccgagtcgggcggaaaaataa

SEQ ID NO: 175 - DNA, 1854 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgtaatgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 176 - DNA, 1854 bp, Artificial Sequence
  Description of Artificial Sequence Synthetic polynucleotide
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcggcccaacggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgtagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 177 - DNA, 1981 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggtaccggatccaccggccgccaccatgggaagcccaaagaagaagcgtaaggtaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagtgaaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagccccaagaaaaagcggaaggtgtaa

SEQ ID NO: 178 - DNA, 72 bp, Artificial Sequence
  Description of Artificial Sequence Synthetic oligonucleotide
ggtcccatggtgtaatggttagcactctggactctaaatccagcgatccgagttcaaatctcggtgggacct

SEQ ID NO: 179 - DNA, 72 bp, Artificial Sequence
  Description of Artificial Sequence Synthetic oligonucleotide
ggttccatggtgtaatggttagcactctggactctaaatccagcgacccgagttcaaatctcggtgggacct

SEQ ID NO: 180 - DNA, 72 bp, Artificial Sequence
  Description of Artificial Sequence Synthetic oligonucleotide
ggttccatggtgtaatggttagcactctggactctaaatccagcgatccgagttcaaatctcggtggaacct

SEQ ID NO: 181 - DNA, 72 bp, Artificial Sequence
  Description of Artificial Sequence Synthetic oligonucleotide
ggttccatggtgtaatggttagcactctggactctaaatccagcgatccgagttcaaatctcggtgggacct

SEQ ID NO: 182 - DNA, 72 bp, Artificial Sequence
  Description of Artificial Sequence Synthetic oligonucleotide
ggttccatggtgtaatggtgagcactctggactctaaatccagcgatccgagttcaaatctcggtgggacct

SEQ ID NO: 183 - DNA, 72 bp, Artificial Sequence
  Description of Artificial Sequence Synthetic oligonucleotide
ggttccatggtgtaatggctagcactctggactctaaatccagcgatccgagttcaaatctcggtgggattt

SEQ ID NO: 184 - DNA, 72 bp, Artificial Sequence
  Description of Artificial Sequence Synthetic oligonucleotide
ggttccatggtgtaatggttagcactctggactctaaatccagccatacaagttcaaatctcagtggaacct

SEQ ID NO: 185 - DNA, 71 bp, Artificial Sequence
  Description of Artificial Sequence Synthetic oligonucleotide
ggttccttggtgtaagatgagcactctggattctaaatccagcgatcagagttcaaatctcggtgggacct

SEQ ID NO: 186 - DNA, 72 bp, Artificial Sequence
  Description of Artificial Sequence Synthetic oligonucleotide
ggtcccatggtgtaatggttagcactctggactctaaatccagcaatctgagttcaaatctcggtgggacct

SEQ ID NO: 187 - DNA, 72 bp, Artificial Sequence
  Description of Artificial Sequence Synthetic oligonucleotide
ggccccatggtgtaatggttagcactctggactctaaatccagcgatccgagttcaaatctcggtgggacct

SEQ ID NO: 188 - DNA, 72 bp, Artificial Sequence
  Description of Artificial Sequence Synthetic oligonucleotide
ggtctcatggtgtaatggttagcacactggactctaagtccagcaatccgagttcgagtcttggtgagacca

SEQ ID NO: 189 - DNA, 72 bp, Artificial Sequence
  Description of Artificial Sequence Synthetic oligonucleotide
ggacccatggtgtaatggttagcactctggactctaaatccagcaatccaagttcaaatctcggtgggacct

SEQ ID NO: 190 - DNA, 72 bp, Artificial Sequence
  Description of Artificial Sequence Synthetic oligonucleotide
gtttccatggtgtaatggttggcactctggactctaaatccagcaatccaagttcaagtctctgtgggacct

SEQ ID NO: 191 - DNA, 51 bp, Artificial Sequence
  Description of Artificial Sequence Synthetic oligonucleotide
ctgcaaagttctttgaaagagcaacaaaatggcttcaactatctgagtgac

SEQ ID NO: 192 - DNA, 51 bp, Artificial Sequence
  Description of Artificial Sequence Synthetic oligonucleotide
ctgcaaagttctttgaaagagcaataaaatggcttcaactatctgagtgac

SEQ ID NO: 193 - DNA, 51 bp, Artificial Sequence
  Description of Artificial Sequence Synthetic oligonucleotide
ctgagacctctaagagccttatctcgatttgaagggatgagggtggttgtg

SEQ ID NO: 194 - DNA, 51 bp, Artificial Sequence
  Description of Artificial Sequence Synthetic oligonucleotide
acaagccttttcagctttagagggcgagcaaaggatgtgggatctgagaac

SEQ ID NO: 195 - DNA, 51 bp, Artificial Sequence
  Description of Artificial Sequence Synthetic oligonucleotide
ctaatagaaagaaatgagaccgcccggtggaaaaatgtgaaagtaaacttt

SEQ ID NO: 196 - DNA, 73 bp, Artificial Sequence
  Description of Artificial Sequence Synthetic oligonucleotide
gcccggctagctcagtcggtagagcatgggactctaaatcccagggtcgtgggttcgagccccacgttgggcg

SEQ ID NO: 197 - DNA, 73 bp, Artificial Sequence
  Description of Artificial Sequence Synthetic oligonucleotide
gcccggctagctcagtcggtagagcatgagactctaaatctcagggtcgtgggttcgagccccacgttgggcg

SEQ ID NO: 198 - DNA, 73 bp, Artificial Sequence
  Description of Artificial Sequence Synthetic oligonucleotide
gcccagctagctcagtctgtagagcatgagactctaaatctcagggtcgtgagttcgagccccacgttgggtg

SEQ ID NO: 199 - DNA, 73 bp, Artificial Sequence
  Description of Artificial Sequence Synthetic oligonucleotide
gcccagatagctcagtgggtagagcatgagactctaaatctcagggtcatgggttcatgccccatgttgggta

SEQ ID NO: 200 - DNA, 73 bp, Artificial Sequence
  Description of Artificial Sequence Synthetic oligonucleotide
gtcctgctggctcagtcggtacagcatgggactctaaatcccagggtcgtgggttcgagctccacgttgggta

SEQ ID NO: 201 - DNA, 73 bp, Artificial Sequence
  Description of Artificial Sequence Synthetic oligonucleotide
gcctggctagctcagtccatagagcatgggactctaaatcccagggtcatgggttcgagccccatattaggca

SEQ ID NO: 202 - DNA, 73 bp, Artificial Sequence
  Description of Artificial Sequence Synthetic oligonucleotide
gcccagctagcttagttggtagagcatgagactctaaatctcagagtcatgggttcaggcctcatgtttggca

SEQ ID NO: 203 - DNA, 74 bp, Artificial Sequence
  Description of Artificial Sequence Synthetic oligonucleotide
aacctggctaggtcagttggtagatcatgagactctaaatctcagggtcatgggttcaagccccatgttggttt

SEQ ID NO: 204 - DNA, 73 bp, Artificial Sequence
  Description of Artificial Sequence Synthetic oligonucleotide
gcccagctagctcagttggtagagcgtgggactttaaatcctagggtcgtgggttcgaaccccacgttgggcg

SEQ ID NO: 205 - DNA, 73 bp, Artificial Sequence
  Description of Artificial Sequence Synthetic oligonucleotide
gcccagctagctcagtctgtagagcatgagactttaagtctcagggtcatgggttggagccccatgttgtgca

SEQ ID NO: 206 - DNA, 77 bp, Artificial Sequence
  Description of Artificial Sequence Synthetic oligonucleotide
gcctagctagttcagtcggtagagcatgagactttaaatctcaggttcatgagtttgagccccatgttggtttggca

SEQ ID NO: 207 - DNA, 73 bp, Artificial Sequence
  Description of Artificial Sequence Synthetic oligonucleotide
ccccggctagctcagtcagtagagcttgagaatttaaatctcagggtcgtgggttggagccccacgttgggcg

SEQ ID NO: 208 - DNA, 73 bp, Artificial Sequence
  Description of Artificial Sequence Synthetic oligonucleotide
gcccggctagctcagtcggtagagcatgggactttaaatcccagggtcgtgggttcgagccccacgttgggcg

SEQ ID NO: 209 - DNA, 73 bp, Artificial Sequence
  Description of Artificial Sequence Synthetic oligonucleotide
gcccggctagctcagtcggtagagcatgagactttaaatctcagggtcgtgggttcgagccccacgttgggcg

SEQ ID NO: 210 - DNA, 73 bp, Artificial Sequence
  Description of Artificial Sequence Synthetic oligonucleotide
gcccagctagctcagtctgtagagcatgagactttaaatctcagggtcgtgagttcgagccccacgttgggtg

SEQ ID NO: 211 - DNA, 73 bp, Artificial Sequence
  Description of Artificial Sequence Synthetic oligonucleotide
gcccagatagctcagtgggtagagcatgagactttaaatctcagggtcatgggttcatgccccatgttgggta

SEQ ID NO: 212 - DNA, 73 bp, Artificial Sequence
  Description of Artificial Sequence Synthetic oligonucleotide
gtcctgctggctcagtcggtacagcatgggactttaaatcccagggtcgtgggttcgagctccacgttgggta

SEQ ID NO: 213 - DNA, 73 bp, Artificial Sequence
  Description of Artificial Sequence Synthetic oligonucleotide
gcctggctagctcagtccatagagcatgggactttaaatcccagggtcatgggttcgagccccatattaggca

SEQ ID NO: 214 - DNA, 73 bp, Artificial Sequence
  Description of Artificial Sequence Synthetic oligonucleotide
gcccagctagcttagttggtagagcatgagactttaaatctcagagtcatgggttcaggcctcatgtttggca

SEQ ID NO: 215 - DNA, 74 bp, Artificial Sequence
  Description of Artificial Sequence Synthetic oligonucleotide
aacctggctaggtcagttggtagatcatgagactttaaatctcagggtcatgggttcaagccccatgttggttt

SEQ ID NO: 216 - DNA, 73 bp, Artificial Sequence
  Description of Artificial Sequence Synthetic oligonucleotide
gcccggatagctcagtcggtagagcatcagactctaaatctgagggtccagggttcaagtccctgttcgggcg

SEQ ID NO: 217 - DNA, 73 bp, Artificial Sequence
  Description of Artificial Sequence Synthetic oligonucleotide
gcctggatagctcagtcggtagagcatcagactctaaatctgagggtccagggttcaagtccctgttcaggcg

SEQ ID NO: 218 - DNA, 73 bp, Artificial Sequence
  Description of Artificial Sequence Synthetic oligonucleotide
gcctggatagctcaattggtagagcatcagactctaaatctgagggttcagggttcaagtccctgttcaggcg

SEQ ID NO: 219 - DNA, 73 bp, Artificial Sequence
  Description of Artificial Sequence Synthetic oligonucleotide
gcccagccagctcagtaggtagagtatgagactctaaatctcagggtggtgggttcgagccccatgttggggg

SEQ ID NO: 220 - DNA, 79 bp, Artificial Sequence
  Description of Artificial Sequence Synthetic oligonucleotide
tgtggtgtagctcagtcggtagagcatcagactctaaatctgagggtccagggttcaggtccctgttcgggtgccaaaa

SEQ ID NO: 221 - DNA, 73 bp, Artificial Sequence
  Description of Artificial Sequence Synthetic oligonucleotide
gcccggatagctcagtcggtagagcatcagactttaaatctgagggtccagggttcaagtccctgttcgggcg

SEQ ID NO: 222 - DNA, 73 bp, Artificial Sequence
  Description of Artificial Sequence Synthetic oligonucleotide
gcctggatagctcagtcggtagagcatcagactttaaatctgagggtccagggttcaagtccctgttcaggcg

SEQ ID NO: 223 - DNA, 73 bp, Artificial Sequence
  Description of Artificial Sequence Synthetic oligonucleotide
gcctggatagctcaattggtagagcatcagactttaaatctgagggttcagggttcaagtccctgttcaggcg

SEQ ID NO: 224 - DNA, 73 bp, Artificial Sequence
  Description of Artificial Sequence Synthetic oligonucleotide
gcccagccagctcagtaggtagagtatgagactttaaatctcagggtggtgggttcgagccccatgttggggg

SEQ ID NO: 225 - DNA, 79 bp, Artificial Sequence
  Description of Artificial Sequence Synthetic oligonucleotide
tgtggtgtagctcagtcggtagagcatcagactttaaatctgagggtccagggttcaggtccctgttcgggtgccaaaa

SEQ ID NO: 226 - DNA, 82 bp, Artificial Sequence
  Description of Artificial Sequence Synthetic oligonucleotide
gtagtcgtggccgagtggttaaggcgatggactctaaatccattggggtttccccgcgcaggttcgaatcctgccgactacg

SEQ ID NO: 227 - DNA, 82 bp, Artificial Sequence
  Description of Artificial Sequence Synthetic oligonucleotide
gtagtcgtggccgagtggttaaggcgatggactctaaatccattggggtctccccgcgcaggttcgaatcctgccgactacg

SEQ ID NO: 228 - DNA, 82 bp, Artificial Sequence
  Description of Artificial Sequence Synthetic oligonucleotide
gtagtcgtggccaagtgagtaaggcaatggactctaaatccattggggtctcccagcacaggttcaaatcctgctgactatg

SEQ ID NO: 229 - DNA, 82 bp, Artificial Sequence
  Description of Artificial Sequence Synthetic oligonucleotide
gtagtcgtggccgagtggttaaggcgatggacttcaaatccattggggtttccccgcgcaggttcgaatcctgccgactacg

SEQ ID NO: 230 - DNA, 82 bp, Artificial Sequence
  Description of Artificial Sequence Synthetic oligonucleotide
gtagtcgtggccgagtggttaaggcgatggacttcaaatccattggggtctccccgcgcaggttcgaatcctgccgactacg

SEQ ID NO: 231 - DNA, 82 bp, Artificial Sequence
  Description of Artificial Sequence Synthetic oligonucleotide
gtagtcgtggccaagtgagtaaggcaatggacttcaaatccattggggtctcccagcacaggttcaaatcctgctgactatg

SEQ ID NO: 232 - DNA, 82 bp, Artificial Sequence
  Description of Artificial Sequence Synthetic oligonucleotide
gtagtcgtggccgagtggttaaggcgatggactttaaatccattggggtttccccgcgcaggttcgaatcctgccgactacg

SEQ ID NO: 233 - DNA, 82 bp, Artificial Sequence
  Description of Artificial Sequence Synthetic oligonucleotide
gtagtcgtggccgagtggttaaggcgatggactttaaatccattggggtctccccgcgcaggttcgaatcctgccgactacg

SEQ ID NO: 234 - DNA, 82 bp, Artificial Sequence
  Description of Artificial Sequence Synthetic oligonucleotide
gtagtcgtggccaagtgagtaaggcaatggactttaaatccattggggtctcccagcacaggttcaaatcctgctgactatg

SEQ ID NO: 235 - DNA, 82 bp, Artificial Sequence
  Description of Artificial Sequence Synthetic oligonucleotide
gctgtgatggccgagtggttaaggcgttggactctaaatccaatggggtctccccgcgcaggttcgaatcctgctcacagcg

SEQ ID NO: 236 - DNA, 82 bp, Artificial Sequence
  Description of Artificial Sequence Synthetic oligonucleotide
gtcacggtggccgagtggttaaggcgttggactctaaatccaatggggtttccccgcacaggttcgaatcctgttcgtgacg

SEQ ID NO: 237 - DNA, 82 bp, Artificial Sequence
  Description of Artificial Sequence Synthetic oligonucleotide
gctgtgatggccgagtggttaaggcgttggactctaaatccaatgggttcttcccgcgcaggttcaaatcctgctcacagcg

SEQ ID NO: 238 - DNA, 82 bp, Artificial Sequence
  Description of Artificial Sequence Synthetic oligonucleotide
gctgtgatggccgagtggttaaggcgttggacttcaaatccaatggggtctccccgcgcaggttcgaatcctgctcacagcg

SEQ ID NO: 239 - DNA, 82 bp, Artificial Sequence
  Description of Artificial Sequence Synthetic oligonucleotide
gtcacggtggccgagtggttaaggcgttggacttcaaatccaatggggtttccccgcacaggttcgaatcctgttcgtgacg

SEQ ID NO: 240 - DNA, 82 bp, Artificial Sequence
  Description of Artificial Sequence Synthetic oligonucleotide
gctgtgatggccgagtggttaaggcgttggacttcaaatccaatgggttcttcccgcgcaggttcaaatcctgctcacagcg

SEQ ID NO: 241 - DNA, 82 bp, Artificial Sequence
  Description of Artificial Sequence Synthetic oligonucleotide
gctgtgatggccgagtggttaaggcgttggactttaaatccaatggggtctccccgcgcaggttcgaatcctgctcacagcg

SEQ ID NO: 242 - DNA, 82 bp, Artificial Sequence
  Description of Artificial Sequence Synthetic oligonucleotide
gtcacggtggccgagtggttaaggcgttggactttaaatccaatggggtttccccgcacaggttcgaatcctgttcgtgacg

SEQ ID NO: 243 - DNA, 82 bp, Artificial Sequence
  Description of Artificial Sequence Synthetic oligonucleotide
gctgtgatggccgagtggttaaggcgttggactttaaatccaatgggttcttcccgcgcaggttcaaatcctgctcacagcg

SEQ ID NO: 244 - DNA, 82 bp, Artificial Sequence
  Description of Artificial Sequence Synthetic oligonucleotide
gacgaggtggccgagtggttaaggcgatggactctaaatccattgtgctctgcacgcgtgggttcgaatcccaccttcgtcg

SEQ ID NO: 245 - DNA, 82 bp, Artificial Sequence
  Description of Artificial Sequence Synthetic oligonucleotide
gacgaggtggccgagtggttaaggcgatggactctaaatccattgtgctctgcacgcatgggttcgaatcccatcctcgtcg

SEQ ID NO: 246 - DNA, 82 bp, Artificial Sequence
  Description of Artificial Sequence Synthetic oligonucleotide
gacgaggtggccgagtggttaaggcgatggactctaaatccattgtgctttgcacgcgtgggttcgaatcccatcctcgtcg

SEQ ID NO: 247 - DNA, 82 bp, Artificial Sequence
  Description of Artificial Sequence Synthetic oligonucleotide
gacgaggtggccgagtggttaaggcgatggactctaaatccattgtgctctgcacgcgtgggttcgaatcccatcctcgtcg

SEQ ID NO: 248 - DNA, 82 bp, Artificial Sequence
  Description of Artificial Sequence Synthetic oligonucleotide
gatgaggtggccgagtggttaaggcgatggactctaaatccattgtgctctgcacgcatgggttcgaatcccatcctcatcg

SEQ ID NO: 249 - DNA, 82 bp, Artificial Sequence
  Description of Artificial Sequence Synthetic oligonucleotide
gacgaggtggccgagtggttaaggcgatggacttcaaatccattgtgctctgcacgcgtgggttcgaatcccaccttcgtcg

SEQ ID NO: 250 - DNA, 82 bp, Artificial Sequence
  Description of Artificial Sequence Synthetic oligonucleotide
gacgaggtggccgagtggttaaggcgatggacttcaaatccattgtgctctgcacgcatgggttcgaatcccatcctcgtcg

SEQ ID NO: 251 - DNA, 82 bp, Artificial Sequence
  Description of Artificial Sequence Synthetic oligonucleotide
gacgaggtggccgagtggttaaggcgatggacttcaaatccattgtgctttgcacgcgtgggttcgaatcccatcctcgtcg

SEQ ID NO: 252 - DNA, 82 bp, Artificial Sequence
  Description of Artificial Sequence Synthetic oligonucleotide
gacgaggtggccgagtggttaaggcgatggacttcaaatccattgtgctctgcacgcgtgggttcgaatcccatcctcgtcg

SEQ ID NO: 253 - DNA, 82 bp, Artificial Sequence
  Description of Artificial Sequence Synthetic oligonucleotide
gatgaggtggccgagtggttaaggcgatggacttcaaatccattgtgctctgcacgcatgggttcgaatcccatcctcatcg

SEQ ID NO: 254 - DNA, 82 bp, Artificial Sequence
  Description of Artificial Sequence Synthetic oligonucleotide
gacgaggtggccgagtggttaaggcgatggactttaaatccattgtgctctgcacgcgtgggttcgaatcccaccttcgtcg

SEQ ID NO: 255 - DNA, 82 bp, Artificial Sequence
  Description of Artificial Sequence Synthetic oligonucleotide
gacgaggtggccgagtggttaaggcgatggactttaaatccattgtgctctgcacgcatgggttcgaatcccatcctcgtcg

SEQ ID NO: 256 - DNA, 82 bp, Artificial Sequence
  Description of Artificial Sequence Synthetic oligonucleotide
gacgaggtggccgagtggttaaggcgatggactttaaatccattgtgctttgcacgcgtgggttcgaatcccatcctcgtcg

SEQ ID NO: 257 - DNA, 82 bp, Artificial Sequence
  Description of Artificial Sequence Synthetic oligonucleotide
gacgaggtggccgagtggttaaggcgatggactttaaatccattgtgctctgcacgcgtgggttcgaatcccatcctcgtcg

SEQ ID NO: 258 - DNA, 82 bp, Artificial Sequence
  Description of Artificial Sequence Synthetic oligonucleotide
gatgaggtggccgagtggttaaggcgatggactttaaatccattgtgctctgcacgcatgggttcgaatcccatcctcatcg

SEQ ID NO: 259 - DNA, 73 bp, Artificial Sequence
  Description of Artificial Sequence Synthetic oligonucleotide
gctgaaatagctcagttgggagagcattagactctagatctaaaggtccctggtttgatcccgggtttcggca

SEQ ID NO: 260 - DNA, 73 bp, Artificial Sequence
  Description of Artificial Sequence Synthetic oligonucleotide
gctgaaatagctcagttgggagagcattagacttcagatctaaaggtccctggtttgatcccgggtttcggca

SEQ ID NO: 261 - DNA, 73 bp, Artificial Sequence
  Description of Artificial Sequence Synthetic oligonucleotide
gctgaaatagctcagttgggagagcattagactttagatctaaaggtccctggtttgatcccgggtttcggca

SEQ ID NO: 262 - DNA, 82 bp, Artificial Sequence
  Description of Artificial Sequence Synthetic oligonucleotide
gcagcgatggccgagtggttaaggcgttggactctaaatccaatggggtctccccgcgcaggttcgaaccctgctcgctgcg

SEQ ID NO: 263 - DNA, 82 bp, Artificial Sequence
  Description of Artificial Sequence Synthetic oligonucleotide
gcagcgatggccgagtggttaaggcgttggacttcaaatccaatggggtctccccgcgcaggttcgaaccctgctcgctgcg

SEQ ID NO: 264 - DNA, 82 bp, Artificial Sequence
  Description of Artificial Sequence Synthetic oligonucleotide
gcagcgatggccgagtggttaaggcgttggactttaaatccaatggggtctccccgcgcaggttcgaaccctgctcgctgcg

SEQ ID NO: 265 - DNA, 72 bp, Artificial Sequence
  Description of Artificial Sequence Synthetic oligonucleotide
gacctcgtggcgcaatggtagcgcgtctgactctagatcagaaggttgcgtgttcaaatcacgtcggggtca

SEQ ID NO: 266 - DNA, 72 bp, Artificial Sequence
  Description of Artificial Sequence Synthetic oligonucleotide
gacctcgtggcgcaacggtagcgcgtctgactctagatcagaaggctgcgtgttcgaatcacgtcggggtca

SEQ ID NO: 267 - DNA, 72 bp, Artificial Sequence
  Description of Artificial Sequence Synthetic oligonucleotide
ggcctcgtggcgcaacggtagcgcgtctgactctagatcagaaggttgcgtgttcaaatcacgtcggggtca

SEQ ID NO: 268 - DNA, 72 bp, Artificial Sequence
  Description of Artificial Sequence Synthetic oligonucleotide
gacctcgtggcgcaacggtagcgcgtctgactctagatcagaaggttgcgtgttcaaatcacgtcggggtca

SEQ ID NO: 269 - DNA, 72 bp, Artificial Sequence
  Description of Artificial Sequence Synthetic oligonucleotide
gacctcgtggcgcaatggtagcgcgtctgactctagatcagaaggttgcgtgttcaagtcacgtcggggtca

SEQ ID NO: 270 - DNA, 72 bp, Artificial Sequence
  Description of Artificial Sequence Synthetic oligonucleotide
gacctcgtggcacaatggtagcacgtctgactctagatcagaaggttgcgtgttcaaatcacgtcggggtca

SEQ ID NO: 271 - DNA, 72 bp, Artificial Sequence
  Description of Artificial Sequence Synthetic oligonucleotide
gacctcgtggcgcaatggtagcgcgtctgacttcagatcagaaggttgcgtgttcaaatcacgtcggggtca

SEQ ID NO: 272 - DNA, 72 bp, Artificial Sequence
  Description of Artificial Sequence Synthetic oligonucleotide
gacctcgtggcgcaacggtagcgcgtctgacttcagatcagaaggctgcgtgttcgaatcacgtcggggtca

SEQ ID NO: 273 - DNA, 72 bp, Artificial Sequence
  Description of Artificial Sequence Synthetic oligonucleotide
ggcctcgtggcgcaacggtagcgcgtctgacttcagatcagaaggttgcgtgttcaaatcacgtcggggtca

SEQ ID NO: 274 - DNA, 72 bp, Artificial Sequence
  Description of Artificial Sequence Synthetic oligonucleotide
gacctcgtggcgcaacggtagcgcgtctgacttcagatcagaaggttgcgtgttcaaatcacgtcggggtca

SEQ ID NO: 275 - DNA, 72 bp, Artificial Sequence
  Description of Artificial Sequence Synthetic oligonucleotide
gacctcgtggcgcaatggtagcgcgtctgacttcagatcagaaggttgcgtgttcaagtcacgtcggggtca

SEQ ID NO: 276 - DNA, 72 bp, Artificial Sequence
  Description of Artificial Sequence Synthetic oligonucleotide
gacctcgtggcacaatggtagcacgtctgacttcagatcagaaggttgcgtgttcaaatcacgtcggggtca

SEQ ID NO: 277 - DNA, 73 bp, Artificial Sequence
  Description of Artificial Sequence Synthetic oligonucleotide
ccttcgatagctcagttggtagagcggaggactctagatccttaggtcgctggttcgaatccggctcgaagga

SEQ ID NO: 278 - DNA, 73 bp, Artificial Sequence
  Description of Artificial Sequence Synthetic oligonucleotide
ccttcgatagctcagttggtagagcggaggactctagatccttaggtcgctggttcgattccggctcgaagga

SEQ ID NO: 279 - DNA, 73 bp, Artificial Sequence
  Description of Artificial Sequence Synthetic oligonucleotide
ccttcgatagctcagctggtagagcggaggactctagatccttaggtcgctggttcgattccggctcgaagga

SEQ ID NO: 280 - DNA, 73 bp, Artificial Sequence
  Description of Artificial Sequence Synthetic oligonucleotide
ccttcgatagctcagctggtagagcggaggactctagatccttaggtcgctggttcgactccggctcgaagga

SEQ ID NO: 281 - DNA, 73 bp, Artificial Sequence
  Description of Artificial Sequence Synthetic oligonucleotide
ctttcgatagttcagttggtagagcggaggactctagatccttaggtcgctggttcgagtccggctcgaagga

SEQ ID NO: 282 - DNA, 73 bp, Artificial Sequence
  Description of Artificial Sequence Synthetic oligonucleotide
ccttcgatagctcagttggtagagcggaggactttagatccttaggtcgctggttcgaatccggctcgaagga

SEQ ID NO: 283 - DNA, 73 bp, Artificial Sequence
  Description of Artificial Sequence Synthetic oligonucleotide
ccttcgatagctcagttggtagagcggaggactttagatccttaggtcgctggttcgattccggctcgaagga

SEQ ID NO: 284 - DNA, 73 bp, Artificial Sequence
  Description of Artificial Sequence Synthetic oligonucleotide
ccttcgatagctcagctggtagagcggaggactttagatccttaggtcgctggttcgattccggctcgaagga

SEQ ID NO: 285 - DNA, 73 bp, Artificial Sequence
  Description of Artificial Sequence Synthetic oligonucleotide
ccttcgatagctcagctggtagagcggaggactttagatccttaggtcgctggttcgactccggctcgaagga

SEQ ID NO: 286 - DNA, 73 bp, Artificial Sequence
  Description of Artificial Sequence Synthetic oligonucleotide
ctttcgatagttcagttggtagagcggaggactttagatccttaggtcgctggttcgagtccggctcgaagga

SEQ ID NO: 287 - DNA, 72 bp, Artificial Sequence
  Description of Artificial Sequence Synthetic oligonucleotide
ggcctcgtggcgcaacggtagcgcgtctgacttcagatcagaaggttgcgggttcaaatcccgtcggggtca

SEQ ID NO: 288 - DNA, 72 bp, Artificial Sequence
  Description of Artificial Sequence Synthetic oligonucleotide
ggcctcgtggcgcaacggtagcgcgtctgacttcagatcagaaggttacgggttcaaatcccgtcggggtca

SEQ ID NO: 289 - DNA, 72 bp, Artificial Sequence
  Description of Artificial Sequence Synthetic oligonucleotide
ggcctcgtggcgcaacggtagcgcgtctgacttcagatcagaaggttccgggttcaaatcccggcggggtca

SEQ ID NO: 290 - DNA, 74 bp, Artificial Sequence
  Description of Artificial Sequence Synthetic oligonucleotide
cgtcggctctgtggcgcaatggatagcgcattggacttcaaattcaaaggttgtgggttcgagtcccagagtcg

SEQ ID NO: 291 - DNA, 77 bp, Artificial Sequence
  Description of Artificial Sequence Synthetic oligonucleotide
cgtcgccccagtggcctaatggataaggcactggccttcaaagccagggattgtgggttcgagtcccacctggggtg

SEQ ID NO: 292 - DNA, 77 bp, Artificial Sequence
  Description of Artificial Sequence Synthetic oligonucleotide
cgtcggctccgtggcgcaatggatagcgcattggacttcaaattcaaaggttccgggttcgagtcccggcggagtcg

SEQ ID NO: 293 - DNA, 77 bp, Artificial Sequence
  Description of Artificial Sequence Synthetic oligonucleotide
cgtcgccccagtggcctaatggataaggcattggccttcaaagccagggattgtgggttcgagtcccatctggggtg

SEQ ID NO: 294 - DNA, 77 bp, Artificial Sequence
  Description of Artificial Sequence Synthetic oligonucleotide
cgtcggctctgtggcgcaatggatagcgcattggacttcaaattcaaaggttgtgggttcgaatcccaccagagtcg

SEQ ID NO: 295 - DNA, 95 bp, Artificial Sequence
  Description of Artificial Sequence Synthetic oligonucleotide
cgtcggctctgtggcgcaatggatagcgcattggacttcaagctgagcctagtgtggtcattcaaaggttgtgggttcgagtcccaccagagtcg

SEQ ID NO: 296 - DNA, 77 bp, Artificial Sequence
  Description of Artificial Sequence Synthetic oligonucleotide
cgtcgccccggtggcctaatggataaggcattggccttcaaagccagggattgtgggttcgagtcccacccggggta

SEQ ID NO: 297 - DNA, 89 bp, Artificial Sequence
  Description of Artificial Sequence Synthetic oligonucleotide
cgtcggctccgtggcgcaatggatagcgcattggacttcaagaggctgaaggcattcaaaggttccgggttcgagtcccggcggagtcg

SEQ ID NO: 298 - DNA, 92 bp, Artificial Sequence
  Description of Artificial Sequence Synthetic oligonucleotide
cgtcggctctgtggcgcaatggatagcgcattggacttcaagtgacgaatagagcaattcaaaggttgtgggttcgaatcccaccagagtcg

SEQ ID NO: 299 - DNA, 77 bp, Artificial Sequence
  Description of Artificial Sequence Synthetic oligonucleotide
cgtcggccgcgtggcctaatggataaggcgtctgacttcagatcagaagattgcaggttcgagtcctgccgcggtcg

SEQ ID NO: 300 - DNA, 77 bp, Artificial Sequence
  Description of Artificial Sequence Synthetic oligonucleotide
cgtcgaccgcgtggcctaatggataaggcgtctgacttcagatcagaagattgagggttcgagtcccttcgtggtcg

SEQ ID NO: 301 - DNA, 90 bp, Artificial Sequence
  Description of Artificial Sequence Synthetic oligonucleotide
cgtcggctctgtggcgcaatggatagcgcattggacttcaagatagttagagaaattcaaaggttgtgggttcgagtcccaccagagtcg

SEQ ID NO: 302 - DNA, 76 bp, Artificial Sequence
  Description of Artificial Sequence Synthetic oligonucleotide
cgtcggttccatggtgtaatggtgagcactctggactctaaatccagcgatccgagttcgagtctcggtggaacct

SEQ ID NO: 303 - DNA, 76 bp, Artificial Sequence
  Description of Artificial Sequence Synthetic oligonucleotide
cgtcggccccatggtgtaatggttagcactctggactctaaatccagcgatccgagttcaaatctcggtgggacct

SEQ ID NO: 304 - DNA, 76 bp, Artificial Sequence
  Description of Artificial Sequence Synthetic oligonucleotide
cgtcggtcccatggtgtaatggttagcactctggactctaaatccagcaatccgagttcgaatctcggtgggacct

SEQ ID NO: 305 - DNA, 76 bp, Artificial Sequence
  Description of Artificial Sequence Synthetic oligonucleotide
cgtcggtcccatggtgtaatggttagcactctggactctaaatccagcgatccgagttcaaatctcggtgggacct

SEQ ID NO: 306 - DNA, 76 bp, Artificial Sequence
  Description of Artificial Sequence Synthetic oligonucleotide
cgtcggccccatggtgtaatggtcagcactctggactctaaatccagcgatccgagttcaaatctcggtgggaccc

SEQ ID NO: 307 - DNA, 76 bp, Artificial Sequence
  Description of Artificial Sequence Synthetic oligonucleotide
cgtcggttccatggtgtaatggtaagcactctggactctaaatccagcgatccgagttcgagtctcggtggaacct

SEQ ID NO: 308 - DNA, 76 bp, Artificial Sequence
  Description of Artificial Sequence Synthetic oligonucleotide
cgtcggttccatggtgtaatggttagcactctggactctaaatccggtaatccgagttcaaatctcggtggaacct

SEQ ID NO: 309 - DNA, 76 bp, Artificial Sequence
  Description of Artificial Sequence Synthetic oligonucleotide
cgtcggttccatggtgtaatggttagcactctggactctaaatccagcgatccgagttcaagtctcggtggaacct

SEQ ID NO: 310 - DNA, 76 bp, Artificial Sequence
  Description of Artificial Sequence Synthetic oligonucleotide
cgtcggttccatggtgtaatggtaagcactctggactttaaatccagcgatccgagttcgagtctcggtggaacct

SEQ ID NO: 311 - DNA, 76 bp, Artificial Sequence
  Description of Artificial Sequence Synthetic oligonucleotide
cgtcggccccatggtgtaatggttagcactctggactttaaatccagcgatccgagttcaaatctcggtgggacct

SEQ ID NO: 312 - DNA, 76 bp, Artificial Sequence
  Description of Artificial Sequence Synthetic oligonucleotide
cgtcggttccatggtgtaatggtgagcactctggactttaaatccagcgatccgagttcgagtctcggtggaacct

SEQ ID NO: 313 - DNA, 76 bp, Artificial Sequence
  Description of Artificial Sequence Synthetic oligonucleotide
cgtcggttccatggtgtaatggttagcactctggactttaaatccagcgatccgagttcaaatctcggtggaacct

SEQ ID NO: 314 - DNA, 76 bp, Artificial Sequence
  Description of Artificial Sequence Synthetic oligonucleotide
cgtcggtcccatggtgtaatggttagcactctggactttaaatccagcgatccgagttcaaatctcggtgggacct

SEQ ID NO: 315 - DNA, 76 bp, Artificial Sequence
  Description of Artificial Sequence Synthetic oligonucleotide
cgtcggtcccatggtgtaatggttagcactctggactttaaatccagcaatccgagttcgaatctcggtgggacct

SEQ ID NO: 316 - DNA, 76 bp, Artificial Sequence
  Description of Artificial Sequence Synthetic oligonucleotide
cgtcggttccatggtgtaatggttagcactctggactttaaatccggtaatccgagttcaaatctcggtggaacct

SEQ ID NO: 317 - DNA, 76 bp, Artificial Sequence
  Description of Artificial Sequence Synthetic oligonucleotide
cgtcggccccatggtgtaatggtcagcactctggactttaaatccagcgatccgagttcaaatctcggtgggaccc

SEQ ID NO: 318 - DNA, 76 bp, Artificial Sequence
  Description of Artificial Sequence Synthetic oligonucleotide
cgtcggttccatggtgtaatggttagcactctggactttaaatccagcgatccgagttcaagtctcggtggaacct

SEQ ID NO: 319 - DNA, 76 bp, Artificial Sequence
  Description of Artificial Sequence Synthetic oligonucleotide
cgtcgacctcgtggcgcaatggtagcgcgtctgactctagatcagaaggttgcgtgttcaagtcacgtcggggtca

SEQ ID NO: 320 - DNA, 76 bp, Artificial Sequence
  Description of Artificial Sequence Synthetic oligonucleotide
cgtcgacctcgtggcgcaacggtagcgcgtctgactctagatcagaaggttgcgtgttcaaatcacgtcggggtca

SEQ ID NO: 321 - DNA, 76 bp, Artificial Sequence
  Description of Artificial Sequence Synthetic oligonucleotide
cgtcggcctcgtggcgcaacggtagcgcgtctgactctagatcagaaggttgcgtgttcaaatcacgtcggggtca

SEQ ID NO: 322 - DNA, 76 bp, Artificial Sequence
  Description of Artificial Sequence Synthetic oligonucleotide
cgtcgacctcgtggcgcaacggtagcgcgtctgactctagatcagaaggctgcgtgttcgaatcacgtcggggtca

SEQ ID NO: 323 - DNA, 76 bp, Artificial Sequence
  Description of Artificial Sequence Synthetic oligonucleotide
cgtcgacctcgtggcgcaacggcagcgcgtctgactctagatcagaaggttgcgtgttcaaatcacgtcggggtca

SEQ ID NO: 324 - DNA, 76 bp, Artificial Sequence
  Description of Artificial Sequence Synthetic oligonucleotide
cgtctcccacatggtctagcggttaggattcctggttctaacccaggcggcccgggttcgactcccggtgtgggaa

SEQ ID NO: 325 - DNA, 76 bp, Artificial Sequence
  Description of Artificial Sequence Synthetic oligonucleotide
cgtctcccatatggtctagcggttaggattcctggttctaacccaggtggcccgggttcgactcccggtatgggaa

SEQ ID NO: 326 - DNA, 76 bp, Artificial Sequence
  Description of Artificial Sequence Synthetic oligonucleotide
cgtctccctggtggtctagtggctaggattcggcgctctaaccgccgcggcccgggttcgattcccggtcagggaa

SEQ ID NO: 327 - DNA, 76 bp, Artificial Sequence
  Description of Artificial Sequence Synthetic oligonucleotide
cgtctccctggtggtctagtggttaggattcggcgctctaaccgccgcggcccgggttcgattcccggtcagggaa

SEQ ID NO: 328 - DNA, 73 bp, Artificial Sequence
  Description of Artificial Sequence Synthetic oligonucleotide
cgtctccctggtctagtggctaggattcggcgctctaaccgccgcggcccgggttcgattcccggccagggaa

SEQ ID NO: 329 - DNA, 76 bp, Artificial Sequence; identical to SEQ ID NO: 324
  Description of Artificial Sequence Synthetic oligonucleotide

SEQ ID NO: 330 - DNA, 76 bp, Artificial Sequence; identical to SEQ ID NO: 325
  Description of Artificial Sequence Synthetic oligonucleotide

SEQ ID NO: 331 - DNA, 76 bp, Artificial Sequence; identical to SEQ ID NO: 327
  Description of Artificial Sequence Synthetic oligonucleotide

SEQ ID NO: 332 - DNA, 76 bp, Artificial Sequence
  Description of Artificial Sequence Synthetic oligonucleotide
cgtctccctggtggtctagtggttaggattcggcgctctaaccgccgcggcccgggttcgattcccggtcaggaaa

SEQ ID NO: 333 - DNA, 76 bp, Artificial Sequence
  Description of Artificial Sequence Synthetic oligonucleotide
cgtctccctggtggtctagtggctaggattcggcgctctaaccgccgcggcccgggttcgattcccggccagggaa

SEQ ID NO: 334 - DNA, 72 bp, Artificial Sequence; identical to SEQ ID NO: 273
  Description of Artificial Sequence Synthetic oligonucleotide

SEQ ID NO: 335 - DNA, 72 bp, Artificial Sequence; identical to SEQ ID NO: 275
  Description of Artificial Sequence Synthetic oligonucleotide

SEQ ID NO: 336 - DNA, 72 bp, Artificial Sequence; identical to SEQ ID NO: 274
  Description of Artificial Sequence Synthetic oligonucleotide

SEQ ID NO: 337 - DNA, 72 bp, Artificial Sequence; identical to SEQ ID NO: 272
  Description of Artificial Sequence Synthetic oligonucleotide

SEQ ID NO: 338 - DNA, 72 bp, Artificial Sequence
  Description of Artificial Sequence Synthetic oligonucleotide
gacctcgtggcgcaacggcagcgcgtctgacttcagatcagaaggttgcgtgttcaaatcacgtcggggtca

SEQ ID NO: 339 - DNA, 72 bp, Artificial Sequence
  Description of Artificial Sequence Synthetic oligonucleotide
gcgttggtggtatagtggttagcatagctgccttcaaagcagttgacccgggttcgattcccggccaacgca

SEQ ID NO: 340 - DNA, 72 bp, Artificial Sequence; identical to SEQ ID NO: 114
  Description of Artificial Sequence Synthetic oligonucleotide

SEQ ID NO: 341 - DNA, 72 bp, Artificial Sequence
  Description of Artificial Sequence Synthetic oligonucleotide
gcgttggtggtatagtggtaagcatagctgccttcaaagcagttgacccgggttcgattcccggccaacgca

SEQ ID NO: 342 - DNA, 72 bp, Artificial Sequence; identical to SEQ ID NO: 273
  Description of Artificial Sequence Synthetic oligonucleotide

SEQ ID NO: 343 - DNA, 72 bp, Artificial Sequence; identical to SEQ ID NO: 275
  Description of Artificial Sequence Synthetic oligonucleotide

SEQ ID NO: 344 - DNA, 72 bp, Artificial Sequence; identical to SEQ ID NO: 274
  Description of Artificial Sequence Synthetic oligonucleotide

SEQ ID NO: 345 - DNA, 72 bp, Artificial Sequence; identical to SEQ ID NO: 272
  Description of Artificial Sequence Synthetic oligonucleotide

SEQ ID NO: 346 - DNA, 72 bp, Artificial Sequence; identical to SEQ ID NO: 338
  Description of Artificial Sequence Synthetic oligonucleotide

SEQ ID NO: 347 - DNA, 72 bp, Artificial Sequence
  Description of Artificial Sequence Synthetic oligonucleotide
ggcctcatggtgcaacagtagtgtgtctgacttcagatcagaaggttgtatgttcaaatcacgtaggggtca

SEQ ID NO: 348 - DNA, 72 bp, Artificial Sequence; identical to SEQ ID NO: 267
  Description of Artificial Sequence Synthetic oligonucleotide

SEQ ID NO: 349 - DNA, 72 bp, Artificial Sequence; identical to SEQ ID NO: 269
  Description of Artificial Sequence Synthetic oligonucleotide

SEQ ID NO: 350 - DNA, 72 bp, Artificial Sequence; identical to SEQ ID NO: 268
  Description of Artificial Sequence Synthetic oligonucleotide

SEQ ID NO: 351 - DNA, 72 bp, Artificial Sequence; identical to SEQ ID NO: 266
  Description of Artificial Sequence Synthetic oligonucleotide

SEQ ID NO: 352 - DNA, 72 bp, Artificial Sequence
  Description of Artificial Sequence Synthetic oligonucleotide
gacctcgtggcgcaacggcagcgcgtctgactctagatcagaaggttgcgtgttcaaatcacgtcggggtca

SEQ ID NO: 353 - DNA, 72 bp, Artificial Sequence
  Description of Artificial Sequence Synthetic oligonucleotide
ggcctcatggtgcaacagtagtgtgtctgactctagatcagaaggttgtatgttcaaatcacgtaggggtca

SEQ ID NO: 354 - DNA, 71 bp, Artificial Sequence
  Description of Artificial Sequence Synthetic oligonucleotide
gcattggtggttcagtggtagaattctcgccttcaacgcgggagacccgggttcaattcccggccaatgca

SEQ ID NO: 355 - DNA, 71 bp, Artificial Sequence; identical to SEQ ID NO: 106
  Description of Artificial Sequence Synthetic oligonucleotide

SEQ ID NO: 356 - DNA, 71 bp, Artificial Sequence
  Description of Artificial Sequence Synthetic oligonucleotide
gcattggtggttcaatggtagaattctcgccttcaacgcaggagacccaggttcgattcctggccaatgca

SEQ ID NO: 357 - DNA, 71 bp, Artificial Sequence
  Description of Artificial Sequence Synthetic oligonucleotide
gcgttggtggtttagtggtagaattctcgccttcaatgcgggagacccgggttcaattcccggccactgca

SEQ ID NO: 358 - DNA, 71 bp, Artificial Sequence
  Description of Artificial Sequence Synthetic oligonucleotide
gccttggtggtgcagtggtagaattctcgccttcaacgtgggagacccgggttcaattcccggccaatgca

SEQ ID NO: 359 - DNA, 61 bp, Artificial Sequence
  Description of Artificial Sequence Synthetic oligonucleotide
ggtggttcagtggtagaattctcgccttcaacgcgggagacccgggtttaattcccggtca

SEQ ID NO: 360 - DNA, 61 bp, Artificial Sequence
  Description of Artificial Sequence Synthetic oligonucleotide
gtggtctagtggttaggattcagcgcttcaaccgccgcagcccgggttcgattcccggtca

SEQ ID NO: 361 - DNA, 71 bp, Artificial Sequence
  Description of Artificial Sequence Synthetic oligonucleotide
gcgtcagtggtttagtggtggaattcctgccttcaatgcacgagatccgtgttcaactcctggttggtgca

SEQ ID NO: 362 - DNA, 72 bp, Artificial Sequence
  Description of Artificial Sequence Synthetic oligonucleotide
gcgtcagtggttttagtggtggaattcctgccttcaatgcacgagatccgtgttcaactcctggttggtgca

SEQ ID NO: 363 - DNA, 71 bp, Artificial Sequence
  Description of Artificial Sequence Synthetic oligonucleotide
gcgttggcagttcagtggtagaattctcgccttcaacccgggagacctggattccatttccggcaaatgca

SEQ ID NO: 364 - DNA, 71 bp, Artificial Sequence; identical to SEQ ID NO: 110
  Description of Artificial Sequence Synthetic oligonucleotide

SEQ ID NO: 365 - DNA, 71 bp, Artificial Sequence; identical to SEQ ID NO: 111
  Description of Artificial Sequence Synthetic oligonucleotide

SEQ ID NO: 366 - DNA, 71 bp, Artificial Sequence
  Description of Artificial Sequence Synthetic oligonucleotide
gcattggtggttcagtggtagaattctcgccttcaacgcgggaggcccgggtttgattcccggccagtgca

SEQ ID NO: 367 - DNA, 71 bp, Artificial Sequence
  Description of Artificial Sequence Synthetic oligonucleotide
gcataggtggttcagtggtagaattcttgccttcaacgcaggaggcccaggtttgattcctggcccatgca

SEQ ID NO: 368 - DNA, 71 bp, Artificial Sequence
  Description of Artificial Sequence Synthetic oligonucleotide
gcattggtggttcagtggtagaattctcgccttcaatgcgggcggccgggcttcgattcctggccaatgca

SEQ ID NO: 369 - DNA, 71 bp, Artificial Sequence
  Description of Artificial Sequence Synthetic oligonucleotide
gcatgggtgattcagtggtagaattttcaccttcaatgcaggaggtccaggttcatttcctggcctatgca

SEQ ID NO: 370 - DNA, 72 bp, Artificial Sequence; identical to SEQ ID NO: 339
  Description of Artificial Sequence Synthetic oligonucleotide

SEQ ID NO: 371 - DNA, 72 bp, Artificial Sequence; identical to SEQ ID NO: 114
  Description of Artificial Sequence Synthetic oligonucleotide

SEQ ID NO: 372 - DNA, 72 bp, Artificial Sequence; identical to SEQ ID NO: 341
  Description of Artificial Sequence Synthetic oligonucleotide

SEQ ID NO: 373 - DNA, 72 bp, Artificial Sequence
  Description of Artificial Sequence Synthetic oligonucleotide
gcgttggtggtatagtggtgagcatagttgccttcaaagcagttgacccgggctcgattcccgcccaacgca

SEQ ID NO: 374 - DNA, 72 bp, Artificial Sequence
  Description of Artificial Sequence Synthetic oligonucleotide
gcgttggtggtatagtggtgagcatagttgccttcaaagcagttgacccgggctcgattcccggccaacgca

SEQ ID NO: 375 - DNA, 73 bp, Artificial Sequence; identical to SEQ ID NO: 1
  Description of Artificial Sequence Synthetic oligonucleotide

SEQ ID NO: 376 - DNA, 73 bp, Artificial Sequence; identical to SEQ ID NO: 3
  Description of Artificial Sequence Synthetic oligonucleotide

SEQ ID NO: 377 - DNA, 73 bp, Artificial Sequence; identical to SEQ ID NO: 4
  Description of Artificial Sequence Synthetic oligonucleotide

SEQ ID NO: 378 - DNA, 73 bp, Artificial Sequence; identical to SEQ ID NO: 6
  Description of Artificial Sequence Synthetic oligonucleotide

SEQ ID NO: 379 - DNA, 73 bp, Artificial Sequence; identical to SEQ ID NO: 7
  Description of Artificial Sequence Synthetic oligonucleotide

SEQ ID NO: 380 - DNA, 73 bp, Artificial Sequence; identical to SEQ ID NO: 8
  Description of Artificial Sequence Synthetic oligonucleotide

SEQ ID NO: 381 - DNA, 73 bp, Artificial Sequence; identical to SEQ ID NO: 9
  Description of Artificial Sequence Synthetic oligonucleotide

SEQ ID NO: 382 - DNA, 73 bp, Artificial Sequence; identical to SEQ ID NO: 10
  Description of Artificial Sequence Synthetic oligonucleotide

SEQ ID NO: 383 - DNA, 73 bp, Artificial Sequence
  Description of Artificial Sequence Synthetic oligonucleotide
gccccagtggcctgatggataaggtactggccttcaaagccagggattgtgggttcgagttccacctggggta

SEQ ID NO: 384 - DNA, 73 bp, Artificial Sequence; identical to SEQ ID NO: 11
  Description of Artificial Sequence Synthetic oligonucleotide

SEQ ID NO: 385 - DNA, 73 bp, Artificial Sequence
  Description of Artificial Sequence Synthetic oligonucleotide
gaccacgtggcctaatggataaggcgtctgacttcagatcagaagattgagggttcgaatccctccgtggtta

SEQ ID NO: 386 - DNA, 73 bp, Artificial Sequence; identical to SEQ ID NO: 12
  Description of Artificial Sequence Synthetic oligonucleotide

SEQ ID NO: 387 - DNA, 73 bp, Artificial Sequence
  Description of Artificial Sequence Synthetic oligonucleotide
gaccacgtggcctaatggataaggcgtctgacttcagatcagaagattgagggttcgaatcccttcgtggtta

SEQ ID NO: 388 - DNA, 73 bp, Artificial Sequence; identical to SEQ ID NO: 13
  Description of Artificial Sequence Synthetic oligonucleotide

SEQ ID NO: 389 - DNA, 73 bp, Artificial Sequence
  Description of Artificial Sequence Synthetic oligonucleotide
ggccgtgtggcctaatggataaggcgtctgacttcagatcaaaagattgcaggtttgagttctgccacggtcg

SEQ ID NO: 390 - DNA, 85 bp, Artificial Sequence; identical to SEQ ID NO: 17
  Description of Artificial Sequence Synthetic oligonucleotide

SEQ ID NO: 391 - DNA, 73 bp, Artificial Sequence; identical to SEQ ID NO: 18
  Description of Artificial Sequence Synthetic oligonucleotide

SEQ ID NO: 392 - DNA, 88 bp, Artificial Sequence
  Description of Artificial Sequence Synthetic oligonucleotide
ggctctgtggcgcaatggatagcgcattggacttcaagtgacgaatagagcaattcaaaggttgtgggttcgaatcccaccagagtcg

SEQ ID NO: 393 - DNA, 73 bp, Artificial Sequence; identical to SEQ ID NO: 16
  Description of Artificial Sequence Synthetic oligonucleotide

SEQ ID NO: 394 - DNA, 91 bp, Artificial Sequence
  Description of Artificial Sequence Synthetic oligonucleotide
ggctctgtggcgcaatggatagcgcattggacttcaagctgagcctagtgtggtcattcaaaggttgtgggttcgagtcccaccagagtcg

SEQ ID NO: 395 - DNA, 73 bp, Artificial Sequence; identical to SEQ ID NO: 22
  Description of Artificial Sequence Synthetic oligonucleotide

SEQ ID NO: 396 - DNA, 86 bp, Artificial Sequence
  Description of Artificial Sequence Synthetic oligonucleotide
ggctctgtggcgcaatggatagcgcattggacttcaagatagttagagaaattcaaaggttgtgggttcgagtcccaccagagtcg

SEQ ID NO: 397 - DNA, 74 bp, Artificial Sequence; identical to SEQ ID NO: 35
  Description of Artificial Sequence Synthetic oligonucleotide

SEQ ID NO: 398 - DNA, 87 bp, Artificial Sequence
  Description of Artificial Sequence Synthetic oligonucleotide
ggctctgtggcgcaatggatagcgcattggacttcaagcctaaatcaagagattcaaaggttgcgggttcgagtccctccagagtcg

SEQ ID NO: 399 - DNA, 73 bp, Artificial Sequence
  Description of Artificial Sequence Synthetic oligonucleotide
ggctctgtggcgcaatggatagcgcattggacttcaaattcaaaggttgcgggttcgagtccctccagagtcg

SEQ ID NO: 400 - DNA, 69 bp, Artificial Sequence
  Description of Artificial Sequence Synthetic oligonucleotide
ggcagcatagcagagtggttcaggttacaggttcaagatgtaaactgagttcaaatcccagttctgcca

SEQ ID NO: 401 - DNA, 57 bp, Artificial Sequence
  Description of Artificial Sequence Synthetic oligonucleotide
tggtgtaataggtagcacagagaattctagattctcaggggtaggttcaattcctat

SEQ ID NO: 402 - DNA, 72 bp, Artificial Sequence
  Description of Artificial Sequence Synthetic oligonucleotide
taggacatggtgtgataggtagcatggagaattctagattctcaggggtaggttcaattcctacagttctag

SEQ ID NO: 403 - DNA, 72 bp, Artificial Sequence
  Description of Artificial Sequence Synthetic oligonucleotide
taggacgtggtgtgataggtagcatggggaattctagattctcaggggtgggttcaattcctatagttctag

SEQ ID NO: 404 - DNA, 72 bp, Artificial Sequence
  Description of Artificial Sequence Synthetic oligonucleotide
taggacgtggtgtagtaggtagcatggagaatgctaaattctcaggggtaggttcaattcctatagttctag

SEQ ID NO: 405 - DNA, 72 bp, Artificial Sequence
  Description of Artificial Sequence Synthetic oligonucleotide
taggacatggtgtaataggtagaatggagaattctaaattctcaggggtaggttcaattcctatagttctag

SEQ ID NO: 406 - DNA, 72 bp, Artificial Sequence
  Description of Artificial Sequence Synthetic oligonucleotide
taggatgtggtgtattaggtagcacagagaattctagattctcaggggtaggttcgattcctataattctac

SEQ ID NO: 407 - DNA, 72 bp, Artificial Sequence
  Description of Artificial Sequence Synthetic oligonucleotide
taggacttggtgtaatgggtagcacagagaattctagattctcaggggtgggttcaattcctttcgtcctag

SEQ ID NO: 408 - DNA, 75 bp, Artificial Sequence
  Description of Artificial Sequence Synthetic oligonucleotide
tctaggatgtggtgtgataggtagcatggagaattctagattctcaggggtaggttcaattcctatattctagaa

SEQ ID NO: 409 - DNA, 72 bp, Artificial Sequence
  Description of Artificial Sequence Synthetic oligonucleotide
taggacgtggtgtgataggtagcatggagaattctagattctcagggatgggttcaattcctatagtcctag

SEQ ID NO: 410 - DNA, 72 bp, Artificial Sequence
  Description of Artificial Sequence Synthetic oligonucleotide
taggacgtggtgtgataggtagcacggagaattctagattctcagggatgggttcaattcctgtagttctag

SEQ ID NO: 411 - DNA, 72 bp, Artificial Sequence; identical to SEQ ID NO: 180
  Description of Artificial Sequence Synthetic oligonucleotide

SEQ ID NO: 412 - DNA, 72 bp, Artificial Sequence
  Description of Artificial Sequence Synthetic oligonucleotide
ggttccatggtgtaatggtgaccactttggactctaaatacagtgatcagagttcaagtctcactggaacct

SEQ ID NO: 413 - DNA, 72 bp, Artificial Sequence
  Description of Artificial Sequence Synthetic oligonucleotide
ggttccatggtgtaatggtgagggctttggactctaactacagtgatcagagttcaagtctcagtgggacct

SEQ ID NO: 414 - DNA, 73 bp, Artificial Sequence
  Description of Artificial Sequence Synthetic oligonucleotide
ggttccatggtgtaatggtaagcaccctggactctaaatccagcaaccagagttccagtctcagcgtggacct

SEQ ID NO: 415 - DNA, 72 bp, Artificial Sequence
  Description of Artificial Sequence Synthetic oligonucleotide
ggtagtgtagtctactggttaaacgcttgggctctaacattaacgtcctgggttcaaatcccagctttgtca

SEQ ID NO: 416 - DNA, 72 bp, Artificial Sequence
  Description of Artificial Sequence Synthetic oligonucleotide
ggttccatggtgtaatggttagcactctggactctaaatccagcgatccgagttcaagtctcggtggaacct

SEQ ID NO: 417 - DNA, 72 bp, Artificial Sequence
  Description of Artificial Sequence Synthetic oligonucleotide
ggttccatggtgtaatggtgagcactctggactctaaatccagcgatccgagttcgagtctcggtggaacct

SEQ ID NO: 418 - DNA, 72 bp, Artificial Sequence
  Description of Artificial Sequence Synthetic oligonucleotide
ggttccatggtgtaatggtaagcactctggactctaaatccagcgatccgagttcgagtctcggtggaacct

SEQ ID NO: 419 - DNA, 72 bp, Artificial Sequence
  Description of Artificial Sequence Synthetic oligonucleotide
ggttccatggtgtaatggttagcactctggactctaaatccggtaatccgagttcaaatctcggtggaacct

SEQ ID NO: 420 - DNA, 72 bp, Artificial Sequence
  Description of Artificial Sequence Synthetic oligonucleotide
ggccccatggtgtaatggtcagcactctggactctaaatccagcgatccgagttcaaatctcggtgggaccc

SEQ ID NO: 421 - DNA, 72 bp, Artificial Sequence
  Description of Artificial Sequence Synthetic oligonucleotide
ggttccatggtgtaatggtaagcactctggactctaaatccagccatctgagttcgagtctctgtggaacct

SEQ ID NO: 422 - DNA, 72 bp, Artificial Sequence
  Description of Artificial Sequence Synthetic oligonucleotide
ggttccatggtgtaatggtgagcactttggactctaaatacagtgatcagagttcaagtctcactgggacct

SEQ ID NO: 423 - DNA, 72 bp, Artificial Sequence
  Description of Artificial Sequence Synthetic oligonucleotide
ggttccatgggttaatggtgagcaccctggactctaaatcaagcgatccgagttcaaatctcggtggtacct

SEQ ID NO: 424 - DNA, 68 bp, Artificial Sequence
  Description of Artificial Sequence Synthetic oligonucleotide
gtttccatggtgtaatggtgagcactctggactctaaatccagaaatacattcaaagaattaagaaca

SEQ ID NO: 425 - DNA, 72 bp, Artificial Sequence; identical to SEQ ID NO: 178
  Description of Artificial Sequence Synthetic oligonucleotide

SEQ ID NO: 426 - DNA, 72 bp, Artificial Sequence
  Description of Artificial Sequence Synthetic oligonucleotide
ggtcccatggtgtaatggttagcactctggactctaaatccagcaatccgagttcgaatctcggtgggacct

SEQ ID NO: 427 - DNA, 72 bp, Artificial Sequence; identical to SEQ ID NO: 187
  Description of Artificial Sequence Synthetic oligonucleotide

SEQ ID NO: 428 - DNA, 72 bp, Artificial Sequence
  Description of Artificial Sequence Synthetic oligonucleotide
ggtcccatggtgtaatggttagcactctgggctctaaatccagcaatccgagttcgaatcttggtgggacct

SEQ ID NO: 429 - DNA, 73 bp, Artificial Sequence
  Description of Artificial Sequence Synthetic oligonucleotide
ggctgtgtacctcagtgggcaagggtatggactctaaagccagactatttgggttcaaatcccagcttggcct

SEQ ID NO: 430 - DNA, 73 bp, Artificial Sequence
  Description of Artificial Sequence Synthetic oligonucleotide
gaccatgtggcctaagggaaaagacatctcactctaggtcagaagattgagggttcaagtcctttcatggtca

SEQ ID NO: 431 - DNA, 74 bp, Artificial Sequence
  Description of Artificial Sequence Synthetic oligonucleotide
ggtacagtgttaaaggggagaaaaattgctgactctaaatacagtagacctaggtttgaatcctggctttacca

SEQ ID NO: 432 - DNA, 57 bp, Artificial Sequence
  Description of Artificial Sequence Synthetic oligonucleotide
tggtgtaataggtagcacagagaattttagattctcaggggtaggttcaattcctat

SEQ ID NO: 433 - DNA, 72 bp, Artificial Sequence
  Description of Artificial Sequence Synthetic oligonucleotide
taggacatggtgtgataggtagcatggagaattttagattctcaggggtaggttcaattcctacagttctag

SEQ ID NO: 434 - DNA, 72 bp, Artificial Sequence
  Description of Artificial Sequence Synthetic oligonucleotide
taggacgtggtgtgataggtagcatggggaattttagattctcaggggtgggttcaattcctatagttctag

SEQ ID NO: 435 - DNA, 72 bp, Artificial Sequence
  Description of Artificial Sequence Synthetic oligonucleotide
taggacgtggtgtagtaggtagcatggagaatgttaaattctcaggggtaggttcaattcctatagttctag

SEQ ID NO: 436 - DNA, 72 bp, Artificial Sequence
  Description of Artificial Sequence Synthetic oligonucleotide
taggacatggtgtaataggtagaatggagaattttaaattctcaggggtaggttcaattcctatagttctag

SEQ ID NO: 437 - DNA, 72 bp, Artificial Sequence
  Description of Artificial Sequence Synthetic oligonucleotide
taggatgtggtgtattaggtagcacagagaattttagattctcaggggtaggttcgattcctataattctac

SEQ ID NO: 438 - DNA, 72 bp, Artificial Sequence
  Description of Artificial Sequence Synthetic oligonucleotide
taggacttggtgtaatgggtagcacagagaattttagattctcaggggtgggttcaattcctttcgtcctag

SEQ ID NO: 439 - DNA, 75 bp, Artificial Sequence
  Description of Artificial Sequence Synthetic oligonucleotide
tctaggatgtggtgtgataggtagcatggagaattttagattctcaggggtaggttcaattcctatattctagaa

SEQ ID NO: 440 - DNA, 72 bp, Artificial Sequence
  Description of Artificial Sequence Synthetic oligonucleotide
taggacgtggtgtgataggtagcatggagaattttagattctcagggatgggttcaattcctatagtcctag

SEQ ID NO: 441 - DNA, 72 bp, Artificial Sequence
  Description of Artificial Sequence Synthetic oligonucleotide
taggacgtggtgtgataggtagcacggagaattttagattctcagggatgggttcaattcctgtagttctag

SEQ ID NO: 442 - DNA, 72 bp, Artificial Sequence; identical to SEQ ID NO: 38
  Description of Artificial Sequence Synthetic oligonucleotide

SEQ ID NO: 443 - DNA, 72 bp, Artificial Sequence
  Description of Artificial Sequence Synthetic oligonucleotide
ggttccatggtgtaatggtgaccactttggactttaaatacagtgatcagagttcaagtctcactggaacct

SEQ ID NO: 444 - DNA, 72 bp, Artificial Sequence
  Description of Artificial Sequence Synthetic oligonucleotide
ggttccatggtgtaatggtgagggctttggactttaactacagtgatcagagttcaagtctcagtgggacct

SEQ ID NO: 445 - DNA, 73 bp, Artificial Sequence
  Description of Artificial Sequence Synthetic oligonucleotide
ggttccatggtgtaatggtaagcaccctggactttaaatccagcaaccagagttccagtctcagcgtggacct

SEQ ID NO: 446 - DNA, 72 bp, Artificial Sequence
  Description of Artificial Sequence Synthetic oligonucleotide
ggtagtgtagtctactggttaaacgcttgggctttaacattaacgtcctgggttcaaatcccagctttgtca

SEQ ID NO: 447 - DNA, 72 bp, Artificial Sequence
  Description of Artificial Sequence Synthetic oligonucleotide
ggttccatggtgtaatggttagcactctggactttaaatccagcgatccgagttcaagtctcggtggaacct

SEQ ID NO: 448 - DNA, 72 bp, Artificial Sequence
  Description of Artificial Sequence Synthetic oligonucleotide
ggttccatggtgtaatggtgagcactctggactttaaatccagcgatccgagttcgagtctcggtggaacct

SEQ ID NO: 449 - DNA, 72 bp, Artificial Sequence
  Description of Artificial Sequence Synthetic oligonucleotide
ggttccatggtgtaatggtaagcactctggactttaaatccagcgatccgagttcgagtctcggtggaacct

SEQ ID NO: 450 - DNA, 72 bp, Artificial Sequence
  Description of Artificial Sequence Synthetic oligonucleotide
ggttccatggtgtaatggttagcactctggactttaaatccggtaatccgagttcaaatctcggtggaacct

SEQ ID NO: 451 - DNA, 72 bp, Artificial Sequence
  Description of Artificial Sequence Synthetic oligonucleotide
ggccccatggtgtaatggtcagcactctggactttaaatccagcgatccgagttcaaatctcggtgggaccc

SEQ ID NO: 452 - DNA, 72 bp, Artificial Sequence
  Description of Artificial Sequence Synthetic oligonucleotide
ggttccatggtgtaatggtaagcactctggactttaaatccagccatctgagttcgagtctctgtggaacct

SEQ ID NO: 453 - DNA, 72 bp, Artificial Sequence
  Description of Artificial Sequence Synthetic oligonucleotide
ggttccatggtgtaatggtgagcactttggactttaaatacagtgatcagagttcaagtctcactgggacct

SEQ ID NO: 454 - DNA, 72 bp, Artificial Sequence
  Description of Artificial Sequence Synthetic oligonucleotide
ggttccatgggttaatggtgagcaccctggactttaaatcaagcgatccgagttcaaatctcggtggtacct

SEQ ID NO: 455 - DNA, 68 bp, Artificial Sequence
  Description of Artificial Sequence Synthetic oligonucleotide
gtttccatggtgtaatggtgagcactctggactttaaatccagaaatacattcaaagaattaagaaca

SEQ ID NO: 456 - DNA, 72 bp, Artificial Sequence; identical to SEQ ID NO: 36
  Description of Artificial Sequence Synthetic oligonucleotide

SEQ ID NO: 457 - DNA, 72 bp, Artificial Sequence
  Description of Artificial Sequence Synthetic oligonucleotide
ggtcccatggtgtaatggttagcactctggactttaaatccagcaatccgagttcgaatctcggtgggacct

SEQ ID NO: 458 - DNA, 72 bp, Artificial Sequence; identical to SEQ ID NO: 45
  Description of Artificial Sequence Synthetic oligonucleotide

SEQ ID NO: 459 - DNA, 72 bp, Artificial Sequence
  Description of Artificial Sequence Synthetic oligonucleotide
ggtcccatggtgtaatggttagcactctgggctttaaatccagcaatccgagttcgaatcttggtgggacct

SEQ ID NO: 460 - DNA, 73 bp, Artificial Sequence
  Description of Artificial Sequence Synthetic oligonucleotide
ggctgtgtacctcagtgggcaagggtatggactttaaagccagactatttgggttcaaatcccagcttggcct

SEQ ID NO: 461 - DNA, 73 bp, Artificial Sequence
  Description of Artificial Sequence Synthetic oligonucleotide
gaccatgtggcctaagggaaaagacatctcactttaggtcagaagattgagggttcaagtcctttcatggtca

SEQ ID NO: 462 - DNA, 74 bp, Artificial Sequence
  Description of Artificial Sequence Synthetic oligonucleotide
ggtacagtgttaaaggggagaaaaattgctgactttaaatacagtagacctaggtttgaatcctggctttacca

SEQ ID NO: 463 - DNA, 72 bp, Artificial Sequence; identical to SEQ ID NO: 96
  Description of Artificial Sequence Synthetic oligonucleotide

SEQ ID NO: 464 - DNA, 72 bp, Artificial Sequence
  Description of Artificial Sequence Synthetic oligonucleotide
tccctggtggtctagtggttaggattcggcgctttaaccgccgcggcccgggttcgattcccggtcaggaaa

SEQ ID NO: 465 - DNA, 72 bp, Artificial Sequence
  Description of Artificial Sequence Synthetic oligonucleotide
cccctggtggtctagtgcttaggattcggtgctttaaccgctgctgcctgcgttcgattcccggtcagggaa

SEQ ID NO: 466 - DNA, 71 bp, Artificial Sequence
  Description of Artificial Sequence Synthetic oligonucleotide
tccttgatgtctagtggttaggatttggtgctttaactgcagcagcctgggttcatttctcagtcagggaa

SEQ ID NO: 467 - DNA, 72 bp, Artificial Sequence
  Description of Artificial Sequence Synthetic oligonucleotide
tcccatatggtctagcggttaggattcctggttttaacccaggtggcccgggttcgactcccggtatgggaa

SEQ ID NO: 468 - DNA, 73 bp, Artificial Sequence
  Description of Artificial Sequence Synthetic oligonucleotide
tccgtggtggtctagtggctaggattcggcgctttaaccgcctgcagctcgagttcgattcctggtcagggaa

SEQ ID NO: 469 - DNA, 69 bp, Artificial Sequence
  Description of Artificial Sequence Synthetic oligonucleotide
ccctgtggtctagtggctaagactttgtgctttaattgctgcatcctaggttcaattcccagtcaggga

SEQ ID NO: 470 - DNA, 72 bp, Artificial Sequence; identical to SEQ ID NO: 102
  Description of Artificial Sequence Synthetic oligonucleotide

SEQ ID NO: 471 - DNA, 72 bp, Artificial Sequence
  Description of Artificial Sequence Synthetic oligonucleotide
tccctggtggtctagtggctaggattcggcgctttaaccgccgcggcccgggttcgattcccggccagggaa

SEQ ID NO: 472 - DNA, 72 bp, Artificial Sequence; identical to SEQ ID NO: 104
  Description of Artificial Sequence Synthetic oligonucleotide

SEQ ID NO: 473 - DNA, 72 bp, Artificial Sequence
  Description of Artificial Sequence Synthetic oligonucleotide
gcgttggtggtgtagtggtgagcacagctgcctttaaagcagttaacgcgggttcgattcccgggtaacgaa

SEQ ID NO: 474 - DNA, 72 bp, Artificial Sequence
  Description of Artificial Sequence Synthetic oligonucleotide
tccttggtggtctagtggctaggattcggtgctttaacctgtgcggcccgggttcaattcccgatgaaggaa

SEQ ID NO: 475 - DNA, 72 bp, Artificial Sequence
  Description of Artificial Sequence Synthetic oligonucleotide
tgtctggtggtcaagtggctaggatttggcgctttaactgccgcggcccgcgttcgattcccggtcagggaa

SEQ ID NO: 476 - DNA, 73 bp, Artificial Sequence
  Description of Artificial Sequence Synthetic oligonucleotide
tccctggtggtctagtggctaggattcggcgctttaaccgcctgcagctcgagttcgattcctggtcagggaa

SEQ ID NO: 477 - DNA, 71 bp, Artificial Sequence
  Description of Artificial Sequence Synthetic oligonucleotide
gcaatggtggttcagtggtagaattctcgcctttaacacaggagacccgggttcaattcctgacccatgta

SEQ ID NO: 478 - DNA, 72 bp, Artificial Sequence; identical to SEQ ID NO: 93
  Description of Artificial Sequence Synthetic oligonucleotide

SEQ ID NO: 479 - DNA, 72 bp, Artificial Sequence
  Description of Artificial Sequence Synthetic oligonucleotide
tccctggtggtctagtggttaggattcggcgctctaaccgccgcggcccgggttcgattcccggtcaggaaa

SEQ ID NO: 480 - DNA, 72 bp, Artificial Sequence
  Description of Artificial Sequence Synthetic oligonucleotide
cccctggtggtctagtgcttaggattcggtgctctaaccgctgctgcctgcgttcgattcccggtcagggaa

SEQ ID NO: 481 - DNA, 71 bp, Artificial Sequence
  Description of Artificial Sequence Synthetic oligonucleotide
tccttgatgtctagtggttaggatttggtgctctaactgcagcagcctgggttcatttctcagtcagggaa

SEQ ID NO: 482 - DNA, 72 bp, Artificial Sequence
  Description of Artificial Sequence Synthetic oligonucleotide
tcccatatggtctagcggttaggattcctggttctaacccaggtggcccgggttcgactcccggtatgggaa

SEQ ID NO: 483 - DNA, 73 bp, Artificial Sequence
  Description of Artificial Sequence Synthetic oligonucleotide
tccgtggtggtctagtggctaggattcggcgctctaaccgcctgcagctcgagttcgattcctggtcagggaa

SEQ ID NO: 484 - DNA, 69 bp, Artificial Sequence
  Description of Artificial Sequence Synthetic oligonucleotide
ccctgtggtctagtggctaagactttgtgctctaattgctgcatcctaggttcaattcccagtcaggga

SEQ ID NO: 485 - DNA, 72 bp, Artificial Sequence; identical to SEQ ID NO: 99
  Description of Artificial Sequence Synthetic oligonucleotide

SEQ ID NO: 486 - DNA, 72 bp, Artificial Sequence
  Description of Artificial Sequence Synthetic oligonucleotide
tccctggtggtctagtggctaggattcggcgctctaaccgccgcggcccgggttcgattcccggccagggaa

SEQ ID NO: 487 - DNA, 72 bp, Artificial Sequence; identical to SEQ ID NO: 101
  Description of Artificial Sequence Synthetic oligonucleotide

SEQ ID NO: 488 - DNA, 72 bp, Artificial Sequence
  Description of Artificial Sequence Synthetic oligonucleotide
gcgttggtggtgtagtggtgagcacagctgcctctaaagcagttaacgcgggttcgattcccgggtaacgaa

SEQ ID NO: 489 - DNA, 72 bp, Artificial Sequence
  Description of Artificial Sequence Synthetic oligonucleotide
tccttggtggtctagtggctaggattcggtgctctaacctgtgcggcccgggttcaattcccgatgaaggaa

SEQ ID NO: 490 - DNA, 72 bp, Artificial Sequence
  Description of Artificial Sequence Synthetic oligonucleotide
tgtctggtggtcaagtggctaggatttggcgctctaactgccgcggcccgcgttcgattcccggtcagggaa

SEQ ID NO: 491 - DNA, 73 bp, Artificial Sequence
  Description of Artificial Sequence Synthetic oligonucleotide
tccctggtggtctagtggctaggattcggcgctctaaccgcctgcagctcgagttcgattcctggtcagggaa

SEQ ID NO: 492 - DNA, 71 bp, Artificial Sequence
  Description of Artificial Sequence Synthetic oligonucleotide
gcaatggtggttcagtggtagaattctcgcctctaacacaggagacccgggttcaattcctgacccatgta

SEQ ID NO: 493 - DNA, 93 bp, Artificial Sequence
  Description of Artificial Sequence Synthetic oligonucleotide
ccttcaatagttcagctggtagagcagaggactttagctacttcctcagtaggagacgtccttaggttgctggttcgattccagcttgaagga

SEQ ID NO: 494 - DNA, 73 bp, Artificial Sequence
  Description of Artificial Sequence Synthetic oligonucleotide
ccttcaatagttcagctggtagagcagaggactttaggtccttaggttgctggttcgattccagcttgaagga

SEQ ID NO: 495 - DNA, 64 bp, Artificial Sequence
  Description of Artificial Sequence Synthetic oligonucleotide
ggtaaaatggctgagtaagctttagactttaaatctaaagagagattgagctctctttttacca

SEQ ID NO: 496 - DNA, 66 bp, Artificial Sequence
  Description of Artificial Sequence Synthetic oligonucleotide
ggtaaaatgactgagtaagcattagactttaaatctaaagacagaggtcaagacctctttttacca

SEQ ID NO: 497 - DNA, 66 bp, Artificial Sequence
  Description of Artificial Sequence Synthetic oligonucleotide
ggtaaaatggctgagtaagcattagactttaaatctaaagacagaggtcaaggcctctttttacca

SEQ ID NO: 498 - DNA, 64 bp, Artificial Sequence
  Description of Artificial Sequence Synthetic oligonucleotide
ggtaaaatggctgagtaagcattagactttaaatctaaagacagaggtcaaggcctttttacca

SEQ ID NO: 499 - DNA, 91 bp, Artificial Sequence
  Description of Artificial Sequence Synthetic oligonucleotide
ccttcgatagctcagttggtagagcggaggactttagttggctgtgtccttagacatccttaggtcgctggttcgaatccggctcgaagga

SEQ ID NO: 500 - DNA, 73 bp, Artificial Sequence; identical to SEQ ID NO: 282
  Description of Artificial Sequence Synthetic oligonucleotide

SEQ ID NO: 501 - DNA, 73 bp, Artificial Sequence
  Description of Artificial Sequence Synthetic oligonucleotide
gggggtatagctcagggctagagctttttgactttagagcaagaggtccctggttcaaatccaggttctccct

SEQ ID NO: 502 - DNA, 61 bp, Artificial Sequence
  Description of Artificial Sequence Synthetic oligonucleotide
tatagctcagtggtagagcatttaactttagatcaagaggtccctggatcaactctgggtg

SEQ ID NO: 503 - DNA, 73 bp, Artificial Sequence
  Description of Artificial Sequence Synthetic oligonucleotide
gtcagtgttgcacaacggttaagtgaagaggctttaaacccagactggatgggttcaattcccatctctgccg

SEQ ID NO: 504 - DNA, 89 bp, Artificial Sequence
  Description of Artificial Sequence Synthetic oligonucleotide
ccttcgatagctcagttggtagagcggaggactttagtggatagggcgtggcaatccttaggtcgctggttcgattccggctcgaagga

SEQ ID NO: 505 - DNA, 73 bp, Artificial Sequence; identical to SEQ ID NO: 283
  Description of Artificial Sequence Synthetic oligonucleotide

SEQ ID NO: 506 - DNA, 89 bp, Artificial Sequence
  Description of Artificial Sequence Synthetic oligonucleotide
ccttcgatagctcagttggtagagcggaggactttaggctcattaagcaaggtatccttaggtcgctggttcgaatccggctcggagga

SEQ ID NO: 507 - DNA, 73 bp, Artificial Sequence
  Description of Artificial Sequence Synthetic oligonucleotide
ccttcgatagctcagttggtagagcggaggactttagatccttaggtcgctggttcgaatccggctcggagga

SEQ ID NO: 508 - DNA, 94 bp, Artificial Sequence
  Description of Artificial Sequence Synthetic oligonucleotide
ccttcgatagctcagctggtagagcggaggactttagattgtatagacatttgcggacatccttaggtcgctggttcgattccagctcgaagga

SEQ ID NO: 509 - DNA, 73 bp, Artificial Sequence
  Description of Artificial Sequence Synthetic oligonucleotide
ccttcgatagctcagctggtagagcggaggactttagatccttaggtcgctggttcgattccagctcgaagga

SEQ ID NO: 510 - DNA, 93 bp, Artificial Sequence
  Description of Artificial Sequence Synthetic oligonucleotide
ccttcgatagctcagctggtagagcggaggactttagctacttcctcagcaggagacatccttaggtcgctggttcgattccggctcgaagga

SEQ ID NO: 511 - DNA, 73 bp, Artificial Sequence; identical to SEQ ID NO: 284
  Description of Artificial Sequence Synthetic oligonucleotide

SEQ ID NO: 512 - DNA, 89 bp, Artificial Sequence
  Description of Artificial Sequence Synthetic oligonucleotide
ccttcgatagctcagctggtagagcggaggactttaggcgcgcgcccgtggccatccttaggtcgctggttcgattccggctcgaagga

SEQ ID NO: 513 - DNA, 73 bp, Artificial Sequence; identical to SEQ ID NO: 284
  Description of Artificial Sequence Synthetic oligonucleotide

SEQ ID NO: 514 - DNA, 94 bp, Artificial Sequence
  Description of Artificial Sequence Synthetic oligonucleotide
ccttcgatagctcagctggtagagcggaggactttagcctgtagaaacatttgtggacatccttaggtcgctggttcgattccggctcgaagga

SEQ ID NO: 515 - DNA, 73 bp, Artificial Sequence; identical to SEQ ID NO: 284
  Description of Artificial Sequence Synthetic oligonucleotide

SEQ ID NO: 516 - DNA, 94 bp, Artificial Sequence
  Description of Artificial Sequence Synthetic oligonucleotide
ccttcgatagctcagctggtagagcggaggactttagattgtacagacatttgcggacatccttaggtcgctggttcgattccggctcgaagga

SEQ ID NO: 517 - DNA, 73 bp, Artificial Sequence; identical to SEQ ID NO: 284
  Description of Artificial Sequence Synthetic oligonucleotide

SEQ ID NO: 518 - DNA, 89 bp, Artificial Sequence
  Description of Artificial Sequence Synthetic oligonucleotide
ccttcgatagctcagctggtagagcggaggactttagtacttaatgtgtggtcatccttaggtcgctggttcgattccggctcgaagga

SEQ ID NO: 519 - DNA, 73 bp, Artificial Sequence; identical to SEQ ID NO: 284
  Description of Artificial Sequence Synthetic oligonucleotide

SEQ ID NO: 520 - DNA, 89 bp, Artificial Sequence
  Description of Artificial Sequence Synthetic oligonucleotide
ccttcgatagctcagctggtagagcggaggactttaggggtttgaatgtggtcatccttaggtcgctggttcgaatccggctcggagga

SEQ ID NO: 521 - DNA, 73 bp, Artificial Sequence
  Description of Artificial Sequence Synthetic oligonucleotide
ccttcgatagctcagctggtagagcggaggactttagatccttaggtcgctggttcgaatccggctcggagga

SEQ ID NO: 522 - DNA, 94 bp, Artificial Sequence
  Description of Artificial Sequence Synthetic oligonucleotide
ccttcgatagctcagctggtagagcggaggactttagactgcggaaacgtttgtggacatccttaggtcgctggttcaattccggctcgaagga

SEQ ID NO: 523 - DNA, 73 bp, Artificial Sequence
  Description of Artificial Sequence Synthetic oligonucleotide
ccttcgatagctcagctggtagagcggaggactttagatccttaggtcgctggttcaattccggctcgaagga

SEQ ID NO: 524 - DNA, 90 bp, Artificial Sequence
  Description of Artificial Sequence Synthetic oligonucleotide
ctttcgatagctcagttggtagagcggaggactttaggttcattaaactaaggcatccttaggtcgctggttcgaatccggctcgaagga

SEQ ID NO: 525 - DNA, 73 bp, Artificial Sequence
  Description of Artificial Sequence Synthetic oligonucleotide
ctttcgatagctcagttggtagagcggaggactttagatccttaggtcgctggttcgaatccggctcgaagga

SEQ ID NO: 526 - DNA, 88 bp, Artificial Sequence
  Description of Artificial Sequence Synthetic oligonucleotide
tcttcaatagctcagctggtagagcggaggactttaggtgcacgcccgtggccattcttaggtgctggtttgattccgacttggagag

SEQ ID NO: 527 - DNA, 72 bp, Artificial Sequence
  Description of Artificial Sequence Synthetic oligonucleotide
tcttcaatagctcagctggtagagcggaggactttagattcttaggtgctggtttgattccgacttggagag

SEQ ID NO: 528 - DNA, 66 bp, Artificial Sequence
  Description of Artificial Sequence Synthetic oligonucleotide
ggtaaaatggctgagtgaagcattggactttaaatctaaagacaggggttaagcctctttttacca

SEQ ID NO: 529 - DNA, 66 bp, Artificial Sequence
  Description of Artificial Sequence Synthetic oligonucleotide
ggtaaaatggctgagcaagcattggactttaaatctaaagacagatgttgagccatctttttagca

SEQ ID NO: 530 - DNA, 66 bp, Artificial Sequence
  Description of Artificial Sequence Synthetic oligonucleotide
ggtaaaatggctgagtgaagcattggactttaaatctaaagacaggggctaagcctctttttacca

SEQ ID NO: 531 - DNA, 66 bp, Artificial Sequence
  Description of Artificial Sequence Synthetic oligonucleotide
ggtaaaatggctgagcaagcattagactttaaatctaaagacagaggttaaggcctctttttacca

SEQ ID NO: 532 - DNA, 66 bp, Artificial Sequence
  Description of Artificial Sequence Synthetic oligonucleotide
ggtaaaatggctgagtaagcattagactttaaatctaaagacagaggtcaaggcctcttttttcct

SEQ ID NO: 533 - DNA, 67 bp, Artificial Sequence
  Description of Artificial Sequence Synthetic oligonucleotide
ggtaaaatggctgagcaagcattagactttaaatctgaaaacagaggtcaaaggtctctttttacca

SEQ ID NO: 534 - DNA, 66 bp, Artificial Sequence; identical to SEQ ID NO: 497
  Description of Artificial Sequence Synthetic oligonucleotide

SEQ ID NO: 535 - DNA, 66 bp, Artificial Sequence
  Description of Artificial Sequence Synthetic oligonucleotide
ggtaaaatgactgaataagccttagactttaaatctgaagacagaggtcaaggcctctttttacca

SEQ ID NO: 536 - DNA, 66 bp, Artificial Sequence
  Description of Artificial Sequence Synthetic oligonucleotide
ggtaaaatggctgagtaagcattggactttaaatctaaagacagaggtcaagacctctttttacca

SEQ ID NO: 537 - DNA, 66 bp, Artificial Sequence
  Description of Artificial Sequence Synthetic oligonucleotide
ggtaaaatggctgagtaaagcattagactttaaatctaaggacagaggctaaacctctttttacca

SEQ ID NO: 538 - DNA, 93 bp, Artificial Sequence
  Description of Artificial Sequence Synthetic oligonucleotide
ccttcaatagttcagctggtagagcagaggactctagctacttcctcagtaggagacgtccttaggttgctggttcgattccagcttgaagga

SEQ ID NO: 539 - DNA, 73 bp, Artificial Sequence
  Description of Artificial Sequence Synthetic oligonucleotide
ccttcaatagttcagctggtagagcagaggactctaggtccttaggttgctggttcgattccagcttgaagga

SEQ ID NO: 540 - DNA, 64 bp, Artificial Sequence
  Description of Artificial Sequence Synthetic oligonucleotide
ggtaaaatggctgagtaagctttagactctaaatctaaagagagattgagctctctttttacca

SEQ ID NO: 541 - DNA, 66 bp, Artificial Sequence
  Description of Artificial Sequence Synthetic oligonucleotide
ggtaaaatgactgagtaagcattagactctaaatctaaagacagaggtcaagacctctttttacca

SEQ ID NO: 542 - DNA, 66 bp, Artificial Sequence
  Description of Artificial Sequence Synthetic oligonucleotide
ggtaaaatggctgagtaagcattagactctaaatctaaagacagaggtcaaggcctctttttacca

SEQ ID NO: 543 - DNA, 64 bp, Artificial Sequence
  Description of Artificial Sequence Synthetic oligonucleotide
ggtaaaatggctgagtaagcattagactctaaatctaaagacagaggtcaaggcctttttacca

SEQ ID NO: 544 - DNA, 91 bp, Artificial Sequence
  Description of Artificial Sequence Synthetic oligonucleotide
ccttcgatagctcagttggtagagcggaggactctagttggctgtgtccttagacatccttaggtcgctggttcgaatccggctcgaagga

SEQ ID NO: 545 - DNA, 73 bp, Artificial Sequence; identical to SEQ ID NO: 277
  Description of Artificial Sequence Synthetic oligonucleotide

SEQ ID NO: 546 - DNA, 73 bp, Artificial Sequence
  Description of Artificial Sequence Synthetic oligonucleotide
gggggtatagctcagggctagagctttttgactctagagcaagaggtccctggttcaaatccaggttctccct

SEQ ID NO: 547 - DNA, 61 bp, Artificial Sequence
  Description of Artificial Sequence Synthetic oligonucleotide
tatagctcagtggtagagcatttaactctagatcaagaggtccctggatcaactctgggtg

SEQ ID NO: 548 - DNA, 73 bp, Artificial Sequence
  Description of Artificial Sequence Synthetic oligonucleotide
gtcagtgttgcacaacggttaagtgaagaggctctaaacccagactggatgggttcaattcccatctctgccg

SEQ ID NO: 549 - DNA, 89 bp, Artificial Sequence
  Description of Artificial Sequence Synthetic oligonucleotide
ccttcgatagctcagttggtagagcggaggactctagtggatagggcgtggcaatccttaggtcgctggttcgattccggctcgaagga

SEQ ID NO: 550 - DNA, 73 bp, Artificial Sequence; identical to SEQ ID NO: 278
  Description of Artificial Sequence Synthetic oligonucleotide

SEQ ID NO: 551 - DNA, 89 bp, Artificial Sequence
  Description of Artificial Sequence Synthetic oligonucleotide
ccttcgatagctcagttggtagagcggaggactctaggctcattaagcaaggtatccttaggtcgctggttcgaatccggctcggagga

SEQ ID NO: 552 - DNA, 73 bp, Artificial Sequence
  Description of Artificial Sequence Synthetic oligonucleotide
ccttcgatagctcagttggtagagcggaggactctagatccttaggtcgctggttcgaatccggctcggagga

SEQ ID NO: 553 - DNA, 94 bp, Artificial Sequence
  Description of Artificial Sequence Synthetic oligonucleotide
ccttcgatagctcagctggtagagcggaggactctagattgtatagacatttgcggacatccttaggtcgctggttcgattccagctcgaagga

SEQ ID NO: 554 - DNA, 73 bp, Artificial Sequence
  Description of Artificial Sequence Synthetic oligonucleotide
ccttcgatagctcagctggtagagcggaggactctagatccttaggtcgctggttcgattccagctcgaagga

SEQ ID NO: 555 - DNA, 93 bp, Artificial Sequence
  Description of Artificial Sequence Synthetic oligonucleotide
ccttcgatagctcagctggtagagcggaggactctagctacttcctcagcaggagacatccttaggtcgctggttcgattccggctcgaagga

SEQ ID NO: 556 - DNA, 73 bp, Artificial Sequence; identical to SEQ ID NO: 279
  Description of Artificial Sequence Synthetic oligonucleotide

SEQ ID NO: 557 - DNA, 89 bp, Artificial Sequence
  Description of Artificial Sequence Synthetic oligonucleotide
ccttcgatagctcagctggtagagcggaggactctaggcgcgcgcccgtggccatccttaggtcgctggttcgattccggctcgaagga

SEQ ID NO: 558 - DNA, 73 bp, Artificial Sequence; identical to SEQ ID NO: 279
  Description of Artificial Sequence Synthetic oligonucleotide

SEQ ID NO: 559 - DNA, 94 bp, Artificial Sequence
  Description of Artificial Sequence Synthetic oligonucleotide
ccttcgatagctcagctggtagagcggaggactctagcctgtagaaacatttgtggacatccttaggtcgctggttcgattccggctcgaagga

SEQ ID NO: 560 - DNA, 73 bp, Artificial Sequence; identical to SEQ ID NO: 279
  Description of Artificial Sequence Synthetic oligonucleotide

SEQ ID NO: 561 - DNA, 94 bp, Artificial Sequence
  Description of Artificial Sequence Synthetic oligonucleotide
ccttcgatagctcagctggtagagcggaggactctagattgtacagacatttgcggacatccttaggtcgctggttcgattccggctcgaagga

SEQ ID NO: 562 - DNA, 73 bp, Artificial Sequence; identical to SEQ ID NO: 279
  Description of Artificial Sequence Synthetic oligonucleotide

SEQ ID NO: 563 - DNA, 89 bp, Artificial Sequence
  Description of Artificial Sequence Synthetic oligonucleotide
ccttcgatagctcagctggtagagcggaggactctagtacttaatgtgtggtcatccttaggtcgctggttcgattccggctcgaagga

SEQ ID NO: 564 - DNA, 73 bp, Artificial Sequence; identical to SEQ ID NO: 279
  Description of Artificial Sequence Synthetic oligonucleotide

SEQ ID NO: 565 - DNA, 89 bp, Artificial Sequence
  Description of Artificial Sequence Synthetic oligonucleotide
ccttcgatagctcagctggtagagcggaggactctaggggtttgaatgtggtcatccttaggtcgctggttcgaatccggctcggagga

SEQ ID NO: 566 - DNA, 73 bp, Artificial Sequence
  Description of Artificial Sequence Synthetic oligonucleotide
ccttcgatagctcagctggtagagcggaggactctagatccttaggtcgctggttcgaatccggctcggagga

SEQ ID NO: 567 - DNA, 94 bp, Artificial Sequence
  Description of Artificial Sequence Synthetic oligonucleotide
ccttcgatagctcagctggtagagcggaggactctagactgcggaaacgtttgtggacatccttaggtcgctggttcaattccggctcgaagga

SEQ ID NO: 568 - DNA, 73 bp, Artificial Sequence
  Description of Artificial Sequence Synthetic oligonucleotide
ccttcgatagctcagctggtagagcggaggactctagatccttaggtcgctggttcaattccggctcgaagga

SEQ ID NO: 569 - DNA, 90 bp, Artificial Sequence
  Description of Artificial Sequence Synthetic oligonucleotide
ctttcgatagctcagttggtagagcggaggactctaggttcattaaactaaggcatccttaggtcgctggttcgaatccggctcgaagga

SEQ ID NO: 570 - DNA, 73 bp, Artificial Sequence
  Description of Artificial Sequence Synthetic oligonucleotide
ctttcgatagctcagttggtagagcggaggactctagatccttaggtcgctggttcgaatccggctcgaagga

SEQ ID NO: 571 - DNA, 88 bp, Artificial Sequence
  Description of Artificial Sequence Synthetic oligonucleotide
tcttcaatagctcagctggtagagcggaggactctaggtgcacgcccgtggccattcttaggtgctggtttgattccgacttggagag

SEQ ID NO: 572 - DNA, 72 bp, Artificial Sequence
  Description of Artificial Sequence Synthetic oligonucleotide
tcttcaatagctcagctggtagagcggaggactctagattcttaggtgctggtttgattccgacttggagag

SEQ ID NO: 573 - DNA, 66 bp, Artificial Sequence
  Description of Artificial Sequence Synthetic oligonucleotide
ggtaaaatggctgagtgaagcattggactctaaatctaaagacaggggttaagcctctttttacca

SEQ ID NO: 574 - DNA, 66 bp, Artificial Sequence
  Description of Artificial Sequence Synthetic oligonucleotide
ggtaaaatggctgagcaagcattggactctaaatctaaagacagatgttgagccatctttttagca

SEQ ID NO: 575 - DNA, 66 bp, Artificial Sequence
  Description of Artificial Sequence Synthetic oligonucleotide
ggtaaaatggctgagtgaagcattggactctaaatctaaagacaggggctaagcctctttttacca

SEQ ID NO: 576 - DNA, 66 bp, Artificial Sequence
  Description of Artificial Sequence Synthetic oligonucleotide
ggtaaaatggctgagcaagcattagactctaaatctaaagacagaggttaaggcctctttttacca

SEQ ID NO: 577 - DNA, 66 bp, Artificial Sequence
  Description of Artificial Sequence Synthetic oligonucleotide
ggtaaaatggctgagtaagcattagactctaaatctaaagacagaggtcaaggcctcttttttcct

SEQ ID NO: 578 - DNA, 67 bp, Artificial Sequence
  Description of Artificial Sequence Synthetic oligonucleotide
ggtaaaatggctgagcaagcattagactctaaatctgaaaacagaggtcaaaggtctctttttacca

SEQ ID NO: 579 - DNA, 66 bp, Artificial Sequence; identical to SEQ ID NO: 542
  Description of Artificial Sequence Synthetic oligonucleotide

SEQ ID NO: 580 - DNA, 66 bp, Artificial Sequence
  Description of Artificial Sequence Synthetic oligonucleotide
ggtaaaatgactgaataagccttagactctaaatctgaagacagaggtcaaggcctctttttacca

SEQ ID NO: 581 - DNA, 66 bp, Artificial Sequence
  Description of Artificial Sequence Synthetic oligonucleotide
ggtaaaatggctgagtaagcattggactctaaatctaaagacagaggtcaagacctctttttacca

SEQ ID NO: 582 - DNA, 66 bp, Artificial Sequence
  Description of Artificial Sequence Synthetic oligonucleotide
ggtaaaatggctgagtaaagcattagactctaaatctaaggacagaggctaaacctctttttacca

SEQ ID NO: 583 - DNA, 75 bp, Artificial Sequence
  Description of Artificial Sequence Synthetic oligonucleotide
gttaagatggcagagcctggtaattgcattaaacttaaaattttataatcagaggttcaactcctcttcttaaca

SEQ ID NO: 584 - DNA, 75 bp, Artificial Sequence
  Description of Artificial Sequence Synthetic oligonucleotide
gttaagatggcagagcccggcaattgcattagacttaaaactttataatcagaggttcaactcctctcattaaca

SEQ ID NO: 585 - DNA, 82 bp, Artificial Sequence
  Description of Artificial Sequence Synthetic oligonucleotide
ggtagcgtggccgagcggtctaaggcgctggattttagctccagtctcttcgggggcgtgggttcaaatcccaccgctgcca

SEQ ID NO: 586 - DNA, 82 bp, Artificial Sequence
  Description of Artificial Sequence Synthetic oligonucleotide
ggtagcgtggccgagtggtctaagacgctggattttagctccagtctcttcgggggcgtgggtttgaatcccaccgctgcca

SEQ ID NO: 587 - DNA, 73 bp, Artificial Sequence
  Description of Artificial Sequence Synthetic oligonucleotide
gggccagtggctcaatggataatgcgtctgactttaaatcagaagattccagccttgactcctggctggctca

SEQ ID NO: 588 - DNA, 82 bp, Artificial Sequence
  Description of Artificial Sequence Synthetic oligonucleotide
ggtagggtggccgagcggtctaaggcactgtattttaactccagtctcttcagaggcatgggtttgaatcccactgctgcca

SEQ ID NO: 589 - DNA, 65 bp, Artificial Sequence
  Description of Artificial Sequence Synthetic oligonucleotide
gccgagcggtctaaggctccggattttagcgccggtgtcttcggaggcatgggttcgaattccac

SEQ ID NO: 590 - DNA, 106 bp, Artificial Sequence
  Description of Artificial Sequence Synthetic polynucleotide
gtcaggatggccgagtggtctaaggcgccagactttagctaagcttcctccgcggtggggattctggtctccaatggaggcgtgggttcgaatcccacttctgaca

SEQ ID NO: 591 - DNA, 83 bp, Artificial Sequence
  Description of Artificial Sequence Synthetic oligonucleotide
gtcaggatggccgagtggtctaaggcgccagactttagttctggtctccaatggaggcgtgggttcgaatcccacttctgaca

SEQ ID NO: 592 - DNA, 105 bp, Artificial Sequence
  Description of Artificial Sequence Synthetic polynucleotide
gtcaggatggccgagtggtctaaggcgccagactttagcttggcttcctcgtgttgaggattctggtctccaatggaggcgtgggttcgaatcccacttctgaca

SEQ ID NO: 593 - DNA, 83 bp, Artificial Sequence; identical to SEQ ID NO: 591
  Description of Artificial Sequence Synthetic oligonucleotide

SEQ ID NO: 594 - DNA, 108 bp, Artificial Sequence
  Description of Artificial Sequence Synthetic polynucleotide
gtcaggatggccgagtggtctaaggcgccagactttagcttactgcttcctgtgttcgggtcttctggtctccgtatggaggcgtgggttcgaatcccacttctgaca

SEQ ID NO: 595 - DNA, 84 bp, Artificial Sequence; identical to SEQ ID NO: 133
  Description of Artificial Sequence Synthetic oligonucleotide

SEQ ID NO: 596 - DNA, 107 bp, Artificial Sequence
  Description of Artificial Sequence Synthetic polynucleotide
gtcaggatggccgagtggtctaaggcgccagactttagttgctacttcccaggtttggggcttctggtctccgcatggaggcgtgggttcgaatcccacttctgaca

SEQ ID NO: 597 - DNA, 84 bp, Artificial Sequence
  Description of Artificial Sequence Synthetic oligonucleotide
gtcaggatggccgagtggtctaaggcgccagactttagttctggtctccgcatggaggcgtgggttcgaatcccacttctgaca

SEQ ID NO: 598 - DNA, 106 bp, Artificial Sequence
  Description of Artificial Sequence Synthetic polynucleotide
gtcaggatggccgagtggtctaaggcgccagactttaggtaagcaccttgcctgcgggctttctggtctccggatggaggcgtgggttcgaatcccacttctgaca

SEQ ID NO: 599 - DNA, 85 bp, Artificial Sequence
  Description of Artificial Sequence Synthetic oligonucleotide
gtcaggatggccgagtggtctaaggcgccagactttagtttctggtctccggatggaggcgtgggttcgaatcccacttctgaca

SEQ ID NO: 600 - DNA, 74 bp, Artificial Sequence
  Description of Artificial Sequence Synthetic oligonucleotide
gcctccttagtgcagtaggtagcgcatcagtctttaaatctgaatggtcctgagttcaagcctcagagggggca

SEQ ID NO: 601 - DNA, 84 bp, Artificial Sequence
  Description of Artificial Sequence Synthetic oligonucleotide
gtcaggatggccgagcagtcttaaggcgctgcgttttaatcgcaccctccgctggaggcgtgggttcgaatcccacttttgaca

SEQ ID NO: 602 - DNA, 60 bp, Artificial Sequence
  Description of Artificial Sequence Synthetic oligonucleotide
ggttccatggtgtaatggtgagcactctggactttaaatccagaagtagtgctggaacaa

SEQ ID NO: 603 - DNA, 83 bp, Artificial Sequence
  Description of Artificial Sequence Synthetic oligonucleotide
gtcagggtggctgagcagtctgaggggctgcgttttagtcgcagtctgccctggaggcgtgggttcgaatcccactcctgaaa

SEQ ID NO: 604 - DNA, 83 bp, Artificial Sequence
  Description of Artificial Sequence Synthetic oligonucleotide
accaggatggccgagtggttaaggcgttggactttagatccaatggacatatgtccgcgtgggttcgaaccccactcctggta

SEQ ID NO: 605 - DNA, 83 bp, Artificial Sequence
  Description of Artificial Sequence Synthetic oligonucleotide
accgggatggccgagtggttaaggcgttggactttagatccaatgggctggtgcccgcgtgggttcgaaccccactctcggta

SEQ ID NO: 606 - DNA, 83 bp, Artificial Sequence
  Description of Artificial Sequence Synthetic oligonucleotide
accagaatggccgagtggttaaggcgttggactttagatccaatggattcatatccgcgtgggttcgaaccccacttctggta

SEQ ID NO: 607 - DNA, 83 bp, Artificial Sequence
  Description of Artificial Sequence Synthetic oligonucleotide
accgggatggctgagtggttaaggcgttggactttagatccaatggacaggtgtccgcgtgggttcgagccccactcccggta

SEQ ID NO: 608 - DNA, 83 bp, Artificial Sequence
  Description of Artificial Sequence Synthetic oligonucleotide
actcatttggctgagtggttaaggcattggactttagatccaatggagtagtggctgtgtgggtttaaaccccactactggta

SEQ ID NO: 609 - DNA, 69 bp, Artificial Sequence
  Description of Artificial Sequence Synthetic oligonucleotide
gagaaagtcatcgtagttacgaagttggctttaacccagttttgggaggttcaattccttcctttctct

SEQ ID NO: 610 - DNA, 84 bp, Artificial Sequence
  Description of Artificial Sequence Synthetic oligonucleotide
accaggatggccaagtagttaaaggcactggactttagagccaatggacatatgtctgtgtgggtttgaaccccactcctggtg

SEQ ID NO: 611 - DNA, 82 bp, Artificial Sequence; identical to SEQ ID NO: 166
  Description of Artificial Sequence Synthetic oligonucleotide

SEQ ID NO: 612 - DNA, 82 bp, Artificial Sequence; identical to SEQ ID NO: 123
  Description of Artificial Sequence Synthetic oligonucleotide

SEQ ID NO: 613 - DNA, 82 bp, Artificial Sequence; identical to SEQ ID NO: 168
  Description of Artificial Sequence Synthetic oligonucleotide

SEQ ID NO: 614 - DNA, 82 bp, Artificial Sequence
  Description of Artificial Sequence Synthetic oligonucleotide
ggtagtgtggttgaatggtctaaggcactgaattttagctccagtctctttggggacgtgggtttaaatcccactgctgcaa

SEQ ID NO: 615 - DNA, 75 bp, Artificial Sequence
  Description of Artificial Sequence Synthetic oligonucleotide
gttaagatggcagagcctggtaattgcactaaacttaaaattttataatcagaggttcaactcctcttcttaaca

SEQ ID NO: 616 - DNA, 75 bp, Artificial Sequence
  Description of Artificial Sequence Synthetic oligonucleotide
gttaagatggcagagcccggcaattgcactagacttaaaactttataatcagaggttcaactcctctcattaaca

SEQ ID NO: 617 - DNA, 82 bp, Artificial Sequence
  Description of Artificial Sequence Synthetic oligonucleotide
ggtagcgtggccgagcggtctaaggcgctggattctagctccagtctcttcgggggcgtgggttcaaatcccaccgctgcca

SEQ ID NO: 618 - DNA, 82 bp, Artificial Sequence
  Description of Artificial Sequence Synthetic oligonucleotide
ggtagcgtggccgagtggtctaagacgctggattctagctccagtctcttcgggggcgtgggtttgaatcccaccgctgcca

SEQ ID NO: 619 - DNA, 73 bp, Artificial Sequence
  Description of Artificial Sequence Synthetic oligonucleotide
gggccagtggctcaatggataatgcgtctgactctaaatcagaagattccagccttgactcctggctggctca

SEQ ID NO: 620 - DNA, 82 bp, Artificial Sequence
  Description of Artificial Sequence Synthetic oligonucleotide
ggtagggtggccgagcggtctaaggcactgtattctaactccagtctcttcagaggcatgggtttgaatcccactgctgcca

SEQ ID NO: 621 - DNA, 65 bp, Artificial Sequence
  Description of Artificial Sequence Synthetic oligonucleotide
gccgagcggtctaaggctccggattctagcgccggtgtcttcggaggcatgggttcgaattccac

SEQ ID NO: 622 - DNA, 106 bp, Artificial Sequence
  Description of Artificial Sequence Synthetic polynucleotide
gtcaggatggccgagtggtctaaggcgccagactctagctaagcttcctccgcggtggggattctggtctccaatggaggcgtgggttcgaatcccacttctgaca

SEQ ID NO: 623 - DNA, 83 bp, Artificial Sequence
  Description of Artificial Sequence Synthetic oligonucleotide
gtcaggatggccgagtggtctaaggcgccagactctagttctggtctccaatggaggcgtgggttcgaatcccacttctgaca

SEQ ID NO: 624 - DNA, 105 bp, Artificial Sequence
  Description of Artificial Sequence Synthetic polynucleotide
gtcaggatggccgagtggtctaaggcgccagactctagcttggcttcctcgtgttgaggattctggtctccaatggaggcgtgggttcgaatcccacttctgaca

SEQ ID NO: 625 - DNA, 83 bp, Artificial Sequence; identical to SEQ ID NO: 623
  Description of Artificial Sequence Synthetic oligonucleotide

SEQ ID NO: 626 - DNA, 108 bp, Artificial Sequence
  Description of Artificial Sequence Synthetic polynucleotide
gtcaggatggccgagtggtctaaggcgccagactctagcttactgcttcctgtgttcgggtcttctggtctccgtatggaggcgtgggttcgaatcccacttctgaca

SEQ ID NO: 627 - DNA, 84 bp, Artificial Sequence; identical to SEQ ID NO: 125
  Description of Artificial Sequence Synthetic oligonucleotide

SEQ ID NO: 628 - DNA, 107 bp, Artificial Sequence
  Description of Artificial Sequence Synthetic polynucleotide
gtcaggatggccgagtggtctaaggcgccagactctagttgctacttcccaggtttggggcttctggtctccgcatggaggcgtgggttcgaatcccacttctgaca

SEQ ID NO: 629 - DNA, 84 bp, Artificial Sequence
  Description of Artificial Sequence Synthetic oligonucleotide
gtcaggatggccgagtggtctaaggcgccagactctagttctggtctccgcatggaggcgtgggttcgaatcccacttctgaca

SEQ ID NO: 630 - DNA, 106 bp, Artificial Sequence
  Description of Artificial Sequence Synthetic polynucleotide
gtcaggatggccgagtggtctaaggcgccagactctaggtaagcaccttgcctgcgggctttctggtctccggatggaggcgtgggttcgaatcccacttctgaca

SEQ ID NO: 631 - DNA, 85 bp, Artificial Sequence
  Description of Artificial Sequence Synthetic oligonucleotide
gtcaggatggccgagtggtctaaggcgccagactctagtttctggtctccggatggaggcgtgggttcgaatcccacttctgaca

SEQ ID NO: 632 - DNA, 74 bp, Artificial Sequence
  Description of Artificial Sequence Synthetic oligonucleotide
gcctccttagtgcagtaggtagcgcatcagtctctaaatctgaatggtcctgagttcaagcctcagagggggca

SEQ ID NO: 633 - DNA, 84 bp, Artificial Sequence
  Description of Artificial Sequence Synthetic oligonucleotide
gtcaggatggccgagcagtcttaaggcgctgcgttctaatcgcaccctccgctggaggcgtgggttcgaatcccacttttgaca

SEQ ID NO: 634 - DNA, 60 bp, Artificial Sequence
  Description of Artificial Sequence Synthetic oligonucleotide
ggttccatggtgtaatggtgagcactctggactctaaatccagaagtagtgctggaacaa

SEQ ID NO: 635 - DNA, 83 bp, Artificial Sequence
  Description of Artificial Sequence Synthetic oligonucleotide
gtcagggtggctgagcagtctgaggggctgcgttctagtcgcagtctgccctggaggcgtgggttcgaatcccactcctgaaa

SEQ ID NO: 636 - DNA, 83 bp, Artificial Sequence
  Description of Artificial Sequence Synthetic oligonucleotide
accaggatggccgagtggttaaggcgttggactctagatccaatggacatatgtccgcgtgggttcgaaccccactcctggta

SEQ ID NO: 637 - DNA, 83 bp, Artificial Sequence
  Description of Artificial Sequence Synthetic oligonucleotide
accgggatggccgagtggttaaggcgttggactctagatccaatgggctggtgcccgcgtgggttcgaaccccactctcggta

SEQ ID NO: 638 - DNA, 83 bp, Artificial Sequence
  Description of Artificial Sequence Synthetic oligonucleotide
accagaatggccgagtggttaaggcgttggactctagatccaatggattcatatccgcgtgggttcgaaccccacttctggta

SEQ ID NO: 639 - DNA, 83 bp, Artificial Sequence
  Description of Artificial Sequence Synthetic oligonucleotide
accgggatggctgagtggttaaggcgttggactctagatccaatggacaggtgtccgcgtgggttcgagccccactcccggta

SEQ ID NO: 640 - DNA, 83 bp, Artificial Sequence
  Description of Artificial Sequence Synthetic oligonucleotide
actcatttggctgagtggttaaggcattggactctagatccaatggagtagtggctgtgtgggtttaaaccccactactggta

SEQ ID NO: 641 - DNA, 69 bp, Artificial Sequence
  Description of Artificial Sequence Synthetic oligonucleotide
gagaaagtcatcgtagttacgaagttggctctaacccagttttgggaggttcaattccttcctttctct

SEQ ID NO: 642 - DNA, 84 bp, Artificial Sequence
  Description of Artificial Sequence Synthetic oligonucleotide
accaggatggccaagtagttaaaggcactggactctagagccaatggacatatgtctgtgtgggtttgaaccccactcctggtg

SEQ ID NO: 643 - DNA, 82 bp, Artificial Sequence; identical to SEQ ID NO: 160
  Description of Artificial Sequence Synthetic oligonucleotide

SEQ ID NO: 644 - DNA, 82 bp, Artificial Sequence; identical to SEQ ID NO: 117
  Description of Artificial Sequence Synthetic oligonucleotide

SEQ ID NO: 645 - DNA, 82 bp, Artificial Sequence; identical to SEQ ID NO: 162
  Description of Artificial Sequence Synthetic oligonucleotide

SEQ ID NO: 646 - DNA, 82 bp, Artificial Sequence
  Description of Artificial Sequence Synthetic oligonucleotide
ggtagtgtggttgaatggtctaaggcactgaattctagctccagtctctttggggacgtgggtttaaatcccactgctgcaa

SEQ ID NO: 647 - DNA, 75 bp, Artificial Sequence
  Description of Artificial Sequence Synthetic oligonucleotide
gttaagatggcagagcctggtaattgcatcaaacttaaaattttataatcagaggttcaactcctcttcttaaca

SEQ ID NO: 648 - DNA, 75 bp, Artificial Sequence
  Description of Artificial Sequence Synthetic oligonucleotide
gttaagatggcagagcccggcaattgcatcagacttaaaactttataatcagaggttcaactcctctcattaaca

SEQ ID NO: 649 - DNA, 82 bp, Artificial Sequence
  Description of Artificial Sequence Synthetic oligonucleotide
ggtagcgtggccgagcggtctaaggcgctggatttcagctccagtctcttcgggggcgtgggttcaaatcccaccgctgcca

SEQ ID NO: 650 - DNA, 82 bp, Artificial Sequence
  Description of Artificial Sequence Synthetic oligonucleotide
ggtagcgtggccgagtggtctaagacgctggatttcagctccagtctcttcgggggcgtgggtttgaatcccaccgctgcca

SEQ ID NO: 651 - DNA, 73 bp, Artificial Sequence
  Description of Artificial Sequence Synthetic oligonucleotide
gggccagtggctcaatggataatgcgtctgacttcaaatcagaagattccagccttgactcctggctggctca

SEQ ID NO: 652 - DNA, 82 bp, Artificial Sequence
  Description of Artificial Sequence Synthetic oligonucleotide
ggtagggtggccgagcggtctaaggcactgtatttcaactccagtctcttcagaggcatgggtttgaatcccactgctgcca

SEQ ID NO: 653 - DNA, 65 bp, Artificial Sequence
  Description of Artificial Sequence Synthetic oligonucleotide
gccgagcggtctaaggctccggatttcagcgccggtgtcttcggaggcatgggttcgaattccac

SEQ ID NO: 654 - DNA, 106 bp, Artificial Sequence
  Description of Artificial Sequence Synthetic polynucleotide
gtcaggatggccgagtggtctaaggcgccagacttcagctaagcttcctccgcggtggggattctggtctccaatggaggcgtgggttcgaatcccacttctgaca

SEQ ID NO: 655 - DNA, 83 bp, Artificial Sequence
  Description of Artificial Sequence Synthetic oligonucleotide
gtcaggatggccgagtggtctaaggcgccagacttcagttctggtctccaatggaggcgtgggttcgaatcccacttctgaca

SEQ ID NO: 656 - DNA, 105 bp, Artificial Sequence
  Description of Artificial Sequence Synthetic polynucleotide
gtcaggatggccgagtggtctaaggcgccagacttcagcttggcttcctcgtgttgaggattctggtctccaatggaggcgtgggttcgaatcccacttctgaca

SEQ ID NO: 657 - DNA, 83 bp, Artificial Sequence; identical to SEQ ID NO: 655
  Description of Artificial Sequence Synthetic oligonucleotide

SEQ ID NO: 658 - DNA, 108 bp, Artificial Sequence
  Description of Artificial Sequence Synthetic polynucleotide
gtcaggatggccgagtggtctaaggcgccagacttcagcttactgcttcctgtgttcgggtcttctggtctccgtatggaggcgtgggttcgaatcccacttctgaca

SEQ ID NO: 659 - DNA, 84 bp, Artificial Sequence; identical to SEQ ID NO: 129
  Description of Artificial Sequence Synthetic oligonucleotide

SEQ ID NO: 660 - DNA, 107 bp, Artificial Sequence
  Description of Artificial Sequence Synthetic polynucleotide
gtcaggatggccgagtggtctaaggcgccagacttcagttgctacttcccaggtttggggcttctggtctccgcatggaggcgtgggttcgaatcccacttctgaca

SEQ ID NO: 661 - DNA, 84 bp, Artificial Sequence
  Description of Artificial Sequence Synthetic oligonucleotide
gtcaggatggccgagtggtctaaggcgccagacttcagttctggtctccgcatggaggcgtgggttcgaatcccacttctgaca

SEQ ID NO: 662 - DNA, 106 bp, Artificial Sequence
  Description of Artificial Sequence Synthetic polynucleotide
gtcaggatggccgagtggtctaaggcgccagacttcaggtaagcaccttgcctgcgggctttctggtctccggatggaggcgtgggttcgaatcccacttctgaca

SEQ ID NO: 663 - DNA, 85 bp, Artificial Sequence
  Description of Artificial Sequence Synthetic oligonucleotide
gtcaggatggccgagtggtctaaggcgccagacttcagtttctggtctccggatggaggcgtgggttcgaatcccacttctgaca

SEQ ID NO: 664 - DNA, 74 bp, Artificial Sequence
  Description of Artificial Sequence Synthetic oligonucleotide
gcctccttagtgcagtaggtagcgcatcagtcttcaaatctgaatggtcctgagttcaagcctcagagggggca

SEQ ID NO: 665 - DNA, 84 bp, Artificial Sequence
  Description of Artificial Sequence Synthetic oligonucleotide
gtcaggatggccgagcagtcttaaggcgctgcgtttcaatcgcaccctccgctggaggcgtgggttcgaatcccacttttgaca

SEQ ID NO: 666 - DNA, 60 bp, Artificial Sequence
  Description of Artificial Sequence Synthetic oligonucleotide
ggttccatggtgtaatggtgagcactctggacttcaaatccagaagtagtgctggaacaa

SEQ ID NO: 667 - DNA, 83 bp, Artificial Sequence
  Description of Artificial Sequence Synthetic oligonucleotide
gtcagggtggctgagcagtctgaggggctgcgtttcagtcgcagtctgccctggaggcgtgggttcgaatcccactcctgaaa

SEQ ID NO: 668 - DNA, 83 bp, Artificial Sequence
  Description of Artificial Sequence Synthetic oligonucleotide
accaggatggccgagtggttaaggcgttggacttcagatccaatggacatatgtccgcgtgggttcgaaccccactcctggta

SEQ ID NO: 669 - DNA, 83 bp, Artificial Sequence
  Description of Artificial Sequence Synthetic oligonucleotide
accgggatggccgagtggttaaggcgttggacttcagatccaatgggctggtgcccgcgtgggttcgaaccccactctcggta

SEQ ID NO: 670 - DNA, 83 bp, Artificial Sequence
  Description of Artificial Sequence Synthetic oligonucleotide
accagaatggccgagtggttaaggcgttggacttcagatccaatggattcatatccgcgtgggttcgaaccccacttctggta

SEQ ID NO: 671 - DNA, 83 bp, Artificial Sequence
  Description of Artificial Sequence Synthetic oligonucleotide
accgggatggctgagtggttaaggcgttggacttcagatccaatggacaggtgtccgcgtgggttcgagccccactcccggta

SEQ ID NO: 672 - DNA, 83 bp, Artificial Sequence
  Description of Artificial Sequence Synthetic oligonucleotide
actcatttggctgagtggttaaggcattggacttcagatccaatggagtagtggctgtgtgggtttaaaccccactactggta

SEQ ID NO: 673 - DNA, 69 bp, Artificial Sequence
  Description of Artificial Sequence Synthetic oligonucleotide
gagaaagtcatcgtagttacgaagttggcttcaacccagttttgggaggttcaattccttcctttctct

SEQ ID NO: 674 - DNA, 84 bp, Artificial Sequence
  Description of Artificial Sequence Synthetic oligonucleotide
accaggatggccaagtagttaaaggcactggacttcagagccaatggacatatgtctgtgtgggtttgaaccccactcctggtg

SEQ ID NO: 675 - DNA, 82 bp, Artificial Sequence; identical to SEQ ID NO: 163
  Description of Artificial Sequence Synthetic oligonucleotide

SEQ ID NO: 676 - DNA, 82 bp, Artificial Sequence; identical to SEQ ID NO: 120
  Description of Artificial Sequence Synthetic oligonucleotide

SEQ ID NO: 677 - DNA, 82 bp, Artificial Sequence; identical to SEQ ID NO: 165
  Description of Artificial Sequence Synthetic oligonucleotide

SEQ ID NO: 678 - DNA, 82 bp, Artificial Sequence
  Description of Artificial Sequence Synthetic oligonucleotide
ggtagtgtggttgaatggtctaaggcactgaatttcagctccagtctctttggggacgtgggtttaaatcccactgctgcaa

SEQ ID NO: 679 - DNA, 70 bp, Artificial Sequence
  Description of Artificial Sequence Synthetic oligonucleotide
gagaaggtcacagaggttatgggattggctttaaaccagtctgtggggggttcgattccctcctttttca

SEQ ID NO: 680 - DNA, 70 bp, Artificial Sequence
  Description of Artificial Sequence Synthetic oligonucleotide
gagaaggtcatagaggttatgggattggctttaaaccagtctctggggggttcgattccctcctttttca

SEQ ID NO: 681 - DNA, 69 bp, Artificial Sequence
  Description of Artificial Sequence Synthetic oligonucleotide
gaaaaagtcataggggttatgaggctggctttaaaccagccttaggaggttcaattccttccttttttg

SEQ ID NO: 682 - DNA, 74 bp, Artificial Sequence
  Description of Artificial Sequence Synthetic oligonucleotide
ggccggttagctcagttggttagagcgtgctgctttaaatgccagggtcgaggtttcgatccccgtacgggcct

SEQ ID NO: 683 - DNA, 82 bp, Artificial Sequence; identical to SEQ ID NO: 232
  Description of Artificial Sequence Synthetic oligonucleotide

SEQ ID NO: 684 - DNA, 82 bp, Artificial Sequence
  Description of Artificial Sequence Synthetic oligonucleotide
gtagtcgtggccgagtggttaaggcgatggactttaaatccattggggtttccccacgcaggttcgaatcctgccgactacg

SEQ ID NO: 685 - DNA, 82 bp, Artificial Sequence
  Description of Artificial Sequence Synthetic oligonucleotide
gtagtcgtggccgagtggttaaggtgatggactttaaacccattggggtctccccgcgcaggttcgaatcctgccgactacg

SEQ ID NO: 686 - DNA, 72 bp, Artificial Sequence
  Description of Artificial Sequence Synthetic oligonucleotide
gggtgtatggctcaggggtagagaatttgactttagatcaagaggtccctggttcaaatccaggtgccccct

SEQ ID NO: 687 - DNA, 78 bp, Artificial Sequence
  Description of Artificial Sequence Synthetic oligonucleotide
agttgtagctgagtggttaaggcaacgagctttaaattcgttggtttctctctgtgcaggtttgaatcctgctaatta

SEQ ID NO: 688 - DNA, 70 bp, Artificial Sequence
  Description of Artificial Sequence Synthetic oligonucleotide
caagaaattcatagaggttatgggattggctttaaaccagtttcaggaggttcgattccttcctttttgg

SEQ ID NO: 689 - DNA, 82 bp, Artificial Sequence; identical to SEQ ID NO: 241
  Description of Artificial Sequence Synthetic oligonucleotide

SEQ ID NO: 690 - DNA, 82 bp, Artificial Sequence
  Description of Artificial Sequence Synthetic oligonucleotide
gctgtgatggccgagtggttaaggcgttggactttaaatccaatggggtctccccgcgcaggttcaaatcctgctcacagcg

SEQ ID NO: 691 - DNA, 82 bp, Artificial Sequence
  Description of Artificial Sequence Synthetic oligonucleotide
gctgtgatggccgagtggttaaggtgttggactttaaatccaatgggggttccccgcgcaggttcaaatcctgctcacagcg

SEQ ID NO: 692 - DNA, 82 bp, Artificial Sequence; identical to SEQ ID NO: 242
  Description of Artificial Sequence Synthetic oligonucleotide

SEQ ID NO: 693 - DNA, 82 bp, Artificial Sequence
  Description of Artificial Sequence Synthetic oligonucleotide
gacgaggtggccgagtggttaaggcgatggactttaaatccattgtgctctgcacgcgtgggttcgaatcccaccctcgtcg

SEQ ID NO: 694 - DNA, 82 bp, Artificial Sequence; identical to SEQ ID NO: 254
  Description of Artificial Sequence Synthetic oligonucleotide

SEQ ID NO: 695 - DNA, 74 bp, Artificial Sequence
  Description of Artificial Sequence Synthetic oligonucleotide
ggccggttagctcagttggttagagcgtgctttaactaatgccagggtcgaggtttcgatccccgtacgggcct

SEQ ID NO: 696 - DNA, 82 bp, Artificial Sequence
  Description of Artificial Sequence Synthetic oligonucleotide
gacgaggtggccgagtggttaaggcgatggactttaaatccattgtgctctgcacacgtgggttcgaatcccatcctcgtcg

SEQ ID NO: 697 - DNA, 78 bp, Artificial Sequence
  Description of Artificial Sequence Synthetic oligonucleotide
gaggcctggccgagtggttaaggcgatggactttaaatccattgtgctctgcacgcgtgggttcgaatcccatcctcg

SEQ ID NO: 698 - DNA, 82 bp, Artificial Sequence; identical to SEQ ID NO: 264
  Description of Artificial Sequence Synthetic oligonucleotide

SEQ ID NO: 699 - DNA, 82 bp, Artificial Sequence; identical to SEQ ID NO: 232
  Description of Artificial Sequence Synthetic oligonucleotide

SEQ ID NO: 700 - DNA, 82 bp, Artificial Sequence; identical to SEQ ID NO: 233
  Description of Artificial Sequence Synthetic oligonucleotide

SEQ ID NO: 701 - DNA, 82 bp, Artificial Sequence
  Description of Artificial Sequence Synthetic oligonucleotide
gtagtcgtggccgagtggttaaggcgatggactttaaatccattggggtttccccgcgcaggttcgaatcctgtcggctacg

SEQ ID NO: 702 - DNA, 70 bp, Artificial Sequence
  Description of Artificial Sequence Synthetic oligonucleotide
gagaaggtcacagaggttatgggattggctctaaaccagtctgtggggggttcgattccctcctttttca

SEQ ID NO: 703 - DNA, 70 bp, Artificial Sequence
  Description of Artificial Sequence Synthetic oligonucleotide
gagaaggtcatagaggttatgggattggctctaaaccagtctctggggggttcgattccctcctttttca

SEQ ID NO: 704 - DNA, 69 bp, Artificial Sequence
  Description of Artificial Sequence Synthetic oligonucleotide
gaaaaagtcataggggttatgaggctggctctaaaccagccttaggaggttcaattccttccttttttg

SEQ ID NO: 705 - DNA, 74 bp, Artificial Sequence
  Description of Artificial Sequence Synthetic oligonucleotide
ggccggttagctcagttggttagagcgtgctgctctaaatgccagggtcgaggtttcgatccccgtacgggcct

SEQ ID NO: 706 - DNA, 82 bp, Artificial Sequence; identical to SEQ ID NO: 226
  Description of Artificial Sequence Synthetic oligonucleotide

SEQ ID NO: 707 - DNA, 82 bp, Artificial Sequence
  Description of Artificial Sequence Synthetic oligonucleotide
gtagtcgtggccgagtggttaaggcgatggactctaaatccattggggtttccccacgcaggttcgaatcctgccgactacg

SEQ ID NO: 708 - DNA, 82 bp, Artificial Sequence
  Description of Artificial Sequence Synthetic oligonucleotide
gtagtcgtggccgagtggttaaggtgatggactctaaacccattggggtctccccgcgcaggttcgaatcctgccgactacg

SEQ ID NO: 709 - DNA, 72 bp, Artificial Sequence
  Description of Artificial Sequence Synthetic oligonucleotide
gggtgtatggctcaggggtagagaatttgactctagatcaagaggtccctggttcaaatccaggtgccccct

SEQ ID NO: 710 - DNA, 78 bp, Artificial Sequence
  Description of Artificial Sequence Synthetic oligonucleotide
agttgtagctgagtggttaaggcaacgagctctaaattcgttggtttctctctgtgcaggtttgaatcctgctaatta

SEQ ID NO: 711 - DNA, 70 bp, Artificial Sequence
  Description of Artificial Sequence Synthetic oligonucleotide
caagaaattcatagaggttatgggattggctctaaaccagtttcaggaggttcgattccttcctttttgg

SEQ ID NO: 712 - DNA, 82 bp, Artificial Sequence; identical to SEQ ID NO: 235
  Description of Artificial Sequence Synthetic oligonucleotide

SEQ ID NO: 713 - DNA, 82 bp, Artificial Sequence
  Description of Artificial Sequence Synthetic oligonucleotide
gctgtgatggccgagtggttaaggcgttggactctaaatccaatggggtctccccgcgcaggttcaaatcctgctcacagcg

SEQ ID NO: 714 - DNA, 82 bp, Artificial Sequence
  Description of Artificial Sequence Synthetic oligonucleotide
gctgtgatggccgagtggttaaggtgttggactctaaatccaatgggggttccccgcgcaggttcaaatcctgctcacagcg

SEQ ID NO: 715 - DNA, 82 bp, Artificial Sequence; identical to SEQ ID NO: 236
  Description of Artificial Sequence Synthetic oligonucleotide

SEQ ID NO: 716 - DNA, 82 bp, Artificial Sequence
  Description of Artificial Sequence Synthetic oligonucleotide
gacgaggtggccgagtggttaaggcgatggactctaaatccattgtgctctgcacgcgtgggttcgaatcccaccctcgtcg

SEQ ID NO: 717 - DNA, 82 bp, Artificial Sequence; identical to SEQ ID NO: 244
  Description of Artificial Sequence Synthetic oligonucleotide

SEQ ID NO: 718 - DNA, 74 bp, Artificial Sequence
  Description of Artificial Sequence Synthetic oligonucleotide
ggccggttagctcagttggttagagcgtgctctaactaatgccagggtcgaggtttcgatccccgtacgggcct

SEQ ID NO: 719 - DNA, 82 bp, Artificial Sequence
  Description of Artificial Sequence Synthetic oligonucleotide
gacgaggtggccgagtggttaaggcgatggactctaaatccattgtgctctgcacacgtgggttcgaatcccatcctcgtcg

SEQ ID NO: 720 - DNA, 78 bp, Artificial Sequence
  Description of Artificial Sequence Synthetic oligonucleotide
gaggcctggccgagtggttaaggcgatggactctaaatccattgtgctctgcacgcgtgggttcgaatcccatcctcg

SEQ ID NO: 721 - DNA, 82 bp, Artificial Sequence; identical to SEQ ID NO: 262
  Description of Artificial Sequence Synthetic oligonucleotide

SEQ ID NO: 722 - DNA, 82 bp, Artificial Sequence; identical to SEQ ID NO: 226
  Description of Artificial Sequence Synthetic oligonucleotide

SEQ ID NO: 723 - DNA, 82 bp, Artificial Sequence; identical to SEQ ID NO: 227
  Description of Artificial Sequence Synthetic oligonucleotide

SEQ ID NO: 724 - DNA, 82 bp, Artificial Sequence
  Description of Artificial Sequence Synthetic oligonucleotide
gtagtcgtggccgagtggttaaggcgatggactctaaatccattggggtttccccgcgcaggttcgaatcctgtcggctacg

SEQ ID NO: 725 - DNA, 70 bp, Artificial Sequence
  Description of Artificial Sequence Synthetic oligonucleotide
gagaaggtcacagaggttatgggattggcttcaaaccagtctgtggggggttcgattccctcctttttca

SEQ ID NO: 726 - DNA, 70 bp, Artificial Sequence
  Description of Artificial Sequence Synthetic oligonucleotide
gagaaggtcatagaggttatgggattggcttcaaaccagtctctggggggttcgattccctcctttttca

SEQ ID NO: 727 - DNA, 69 bp, Artificial Sequence
  Description of Artificial Sequence Synthetic oligonucleotide
gaaaaagtcataggggttatgaggctggcttcaaaccagccttaggaggttcaattccttccttttttg

SEQ ID NO: 728 - DNA, 74 bp, Artificial Sequence
  Description of Artificial Sequence Synthetic oligonucleotide
ggccggttagctcagttggttagagcgtgctgcttcaaatgccagggtcgaggtttcgatccccgtacgggcct

SEQ ID NO: 729 - DNA, 82 bp, Artificial Sequence; identical to SEQ ID NO: 229
  Description of Artificial Sequence Synthetic oligonucleotide

SEQ ID NO: 730 - DNA, 82 bp, Artificial Sequence
  Description of Artificial Sequence Synthetic oligonucleotide
gtagtcgtggccgagtggttaaggcgatggacttcaaatccattggggtttccccacgcaggttcgaatcctgccgactacg

SEQ ID NO: 731 - DNA, 82 bp, Artificial Sequence
  Description of Artificial Sequence Synthetic oligonucleotide
gtagtcgtggccgagtggttaaggtgatggacttcaaacccattggggtctccccgcgcaggttcgaatcctgccgactacg

SEQ ID NO: 732 - DNA, 72 bp, Artificial Sequence
  Description of Artificial Sequence Synthetic oligonucleotide
gggtgtatggctcaggggtagagaatttgacttcagatcaagaggtccctggttcaaatccaggtgccccct

SEQ ID NO: 733 - DNA, 78 bp, Artificial Sequence
  Description of Artificial Sequence Synthetic oligonucleotide
agttgtagctgagtggttaaggcaacgagcttcaaattcgttggtttctctctgtgcaggtttgaatcctgctaatta

SEQ ID NO: 734 - DNA, 70 bp, Artificial Sequence
  Description of Artificial Sequence Synthetic oligonucleotide
caagaaattcatagaggttatgggattggcttcaaaccagtttcaggaggttcgattccttcctttttgg

SEQ ID NO: 735 - DNA, 82 bp, Artificial Sequence; identical to SEQ ID NO: 238
  Description of Artificial Sequence Synthetic oligonucleotide

SEQ ID NO: 736 - DNA, 82 bp, Artificial Sequence
  Description of Artificial Sequence Synthetic oligonucleotide
gctgtgatggccgagtggttaaggcgttggacttcaaatccaatggggtctccccgcgcaggttcaaatcctgctcacagcg

SEQ ID NO: 737 - DNA, 82 bp, Artificial Sequence
  Description of Artificial Sequence Synthetic oligonucleotide
gctgtgatggccgagtggttaaggtgttggacttcaaatccaatgggggttccccgcgcaggttcaaatcctgctcacagcg

SEQ ID NO: 738 - DNA, 82 bp, Artificial Sequence; identical to SEQ ID NO: 239
  Description of Artificial Sequence Synthetic oligonucleotide

SEQ ID NO: 739 - DNA, 82 bp, Artificial Sequence
  Description of Artificial Sequence Synthetic oligonucleotide
gacgaggtggccgagtggttaaggcgatggacttcaaatccattgtgctctgcacgcgtgggttcgaatcccaccctcgtcg

SEQ ID NO: 740 - DNA, 82 bp, Artificial Sequence; identical to SEQ ID NO: 249
  Description of Artificial Sequence Synthetic oligonucleotide

SEQ ID NO: 741 - DNA, 74 bp, Artificial Sequence
  Description of Artificial Sequence Synthetic oligonucleotide
ggccggttagctcagttggttagagcgtgcttcaactaatgccagggtcgaggtttcgatccccgtacgggcct

SEQ ID NO: 742 - DNA, 82 bp, Artificial Sequence
  Description of Artificial Sequence Synthetic oligonucleotide
gacgaggtggccgagtggttaaggcgatggacttcaaatccattgtgctctgcacacgtgggttcgaatcccatcctcgtcg

SEQ ID NO: 743 - DNA, 78 bp, Artificial Sequence
  Description of Artificial Sequence Synthetic oligonucleotide
gaggcctggccgagtggttaaggcgatggacttcaaatccattgtgctctgcacgcgtgggttcgaatcccatcctcg

SEQ ID NO: 744 - DNA, 82 bp, Artificial Sequence; identical to SEQ ID NO: 263
  Description of Artificial Sequence Synthetic oligonucleotide

SEQ ID NO: 745 - DNA, 82 bp, Artificial Sequence; identical to SEQ ID NO: 229
  Description of Artificial Sequence Synthetic oligonucleotide

SEQ ID NO: 746 - DNA, 82 bp, Artificial Sequence; identical to SEQ ID NO: 230
  Description of Artificial Sequence Synthetic oligonucleotide

SEQ ID NO: 747 - DNA, 82 bp, Artificial Sequence
  Description of Artificial Sequence Synthetic oligonucleotide
gtagtcgtggccgagtggttaaggcgatggacttcaaatccattggggtttccccgcgcaggttcgaatcctgtcggctacg

SEQ ID NO: 748 - DNA, 73 bp, Artificial Sequence
  Description of Artificial Sequence Synthetic oligonucleotide
gcccagctagctcagtcggtagagcataagactttaaatctcagggttgtggattcgtgccccatgctgggtg

SEQ ID NO: 749 - DNA, 71 bp, Artificial Sequence
  Description of Artificial Sequence Synthetic oligonucleotide
ctgcagctagctcagtcggtagagcatgagactttaaatctcagggtcatgggttcgtgccccatgttggg

SEQ ID NO: 750 - DNA, 70 bp, Artificial Sequence
  Description of Artificial Sequence Synthetic oligonucleotide
ccagcatgtctcagtcggtatagtgtgagactttaaatctcagggtcgtgggttcaagccccacattggg

SEQ ID NO: 751 - DNA, 73 bp, Artificial Sequence
  Description of Artificial Sequence Synthetic oligonucleotide
gtctagctagatcagttggtagagcataagactttaaatctcagggtcatgggtttgagccctacgttgggcg

SEQ ID NO: 752 - DNA, 73 bp, Artificial Sequence
  Description of Artificial Sequence Synthetic oligonucleotide
gcccagctagctcagccggtagagcacaagactttaaatctcagggtcgtgggtttgagccctgtgttgagca

SEQ ID NO: 753 - DNA, 69 bp, Artificial Sequence
  Description of Artificial Sequence Synthetic oligonucleotide
ccgaatagcttagttgatgaagcgtgagactttaaatctcagggtagtgggttcaagccccacattgga

SEQ ID NO: 754 - DNA, 74 bp, Artificial Sequence
  Description of Artificial Sequence Synthetic oligonucleotide
gcctggctacctcagttggtagagcatgggactttaaatcccagagtcagtgggttcaagcctcacattgagtg

SEQ ID NO: 755 - DNA, 73 bp, Artificial Sequence
  Description of Artificial Sequence Synthetic oligonucleotide
gcccggctagctcagtcggtagagcatgagaccttaaatctcagggtcgtgggttcgagccccacgttgggcg

SEQ ID NO: 756 - DNA, 73 bp, Artificial Sequence
  Description of Artificial Sequence Synthetic oligonucleotide
gcccggctagctcagtcggtagagcatgggactttaaatctcagggtcgtgggttcgagccccacgttgggcg

SEQ ID NO: 757 - DNA, 73 bp, Artificial Sequence
  Description of Artificial Sequence Synthetic oligonucleotide
gcccggctagctcagtcgatagagcatgagactttaaatctcagggtcgtgggttcgagccgcacgttgggcg

SEQ ID NO: 758 - DNA, 73 bp, Artificial Sequence
  Description of Artificial Sequence Synthetic oligonucleotide
gcccagctagctcagtcggtagagcatgagactttaaatctcagggtcatgggtttgagccccacgtttggtg

SEQ ID NO: 759 - DNA, 73 bp, Artificial Sequence
  Description of Artificial Sequence Synthetic oligonucleotide
gcctggctagctcagtcggcaaagcatgagactttaaatctcagggtcgtgggctcgagctccatgttgggcg

SEQ ID NO: 760 - DNA, 73 bp, Artificial Sequence
  Description of Artificial Sequence Synthetic oligonucleotide
gcccgactacctcagtcggtggagcatgggactttacatcccagggttgtgggttcgagccccacattgggca

SEQ ID NO: 761 - DNA, 73 bp, Artificial Sequence
  Description of Artificial Sequence Synthetic oligonucleotide
ccccggctggctcagtcagtagatcatgagactttaaatctcagggtcgtgggttcacgccccacactgggcg

SEQ ID NO: 762 - DNA, 65 bp, Artificial Sequence
  Description of Artificial Sequence Synthetic oligonucleotide
gcgctagtcagtagagcatgagactttaaatctcagggtcgtgggttcgagccccacatcgggcg

SEQ ID NO: 763 - DNA, 73 bp, Artificial Sequence
  Description of Artificial Sequence Synthetic oligonucleotide
gcctggatagctcagttggtagagcatcagactttaaatctgagggtccagggttcaagtccctgttcaggca

SEQ ID NO: 764 - DNA, 73 bp, Artificial Sequence
  Description of Artificial Sequence Synthetic oligonucleotide
gccaggatagttcaggtggtagagcatcagactttaaacctgagggttcagggttcaagtctctgtttgggcg

SEQ ID NO: 765 - DNA, 73 bp, Artificial Sequence
  Description of Artificial Sequence Synthetic oligonucleotide
acccagatagctcagtcagtagagcatcagactttaaatctgagggtccaaggttcatgtccctttttgggtg

SEQ ID NO: 766 - DNA, 73 bp, Artificial Sequence
  Description of Artificial Sequence Synthetic oligonucleotide
acctgggtagcttagttggtagagcattggactttaaatttgagggcccaggtttcaagtccctgtttgggtg

SEQ ID NO: 767 - DNA, 74 bp, Artificial Sequence
  Description of Artificial Sequence Synthetic oligonucleotide
gcctgggtagctcagtcggtagagctatcagactttaagcctgaggattcagggttcaatcccttgctggggcg

SEQ ID NO: 768 - DNA, 62 bp, Artificial Sequence
  Description of Artificial Sequence Synthetic oligonucleotide
gatagctcagttgatagagcatcagactttaaatctgagggtccagggttcatgtccctgtt

SEQ ID NO: 769 - DNA, 73 bp, Artificial Sequence
  Description of Artificial Sequence Synthetic oligonucleotide
gttggggtaactcagttggtagagtagcagactttacatctgagggtccagggtttaagtccatgtccaggca

SEQ ID NO: 770 - DNA, 73 bp, Artificial Sequence
  Description of Artificial Sequence Synthetic oligonucleotide
gcctggatagctcagttggtagagcatcagactttaaatctgagggtccagggttcaagtccctgttcaggcg

SEQ ID NO: 771 - DNA, 73 bp, Artificial Sequence; identical to SEQ ID NO: 222
  Description of Artificial Sequence Synthetic oligonucleotide

SEQ ID NO: 772 - DNA, 73 bp, Artificial Sequence
  Description of Artificial Sequence Synthetic oligonucleotide
gcccggatagctcagtcggtagagcatcagactttaaatctgagggtccggggttcaagtccctgttcgggcg

SEQ ID NO: 773 - DNA, 73 bp, Artificial Sequence
  Description of Artificial Sequence Synthetic oligonucleotide
gcctgggtagctcagtcggtagagcatcagactttaaatctgagggtccagggttcaagtccctgtccaggcg

SEQ ID NO: 774 - DNA, 73 bp, Artificial Sequence
  Description of Artificial Sequence Synthetic oligonucleotide
gcctggatagctcagttggtagaacatcagactttaaatctgacggtgcagggttcaagtccctgttcaggcg

SEQ ID NO: 775 - DNA, 73 bp, Artificial Sequence
  Description of Artificial Sequence Synthetic oligonucleotide
gcccggagagctcagtgggtagagcatcagactttaaatctgagggtccagggttcaagtcctcgttcgggca

SEQ ID NO: 776 - DNA, 73 bp, Artificial Sequence
  Description of Artificial Sequence Synthetic oligonucleotide
acctgggtagctcagtaggtagaacatcagactttaaatctgagggtctagggttcaagtccctgtccaggcg

SEQ ID NO: 777 - DNA, 76 bp, Artificial Sequence
  Description of Artificial Sequence Synthetic oligonucleotide
gcctggatagctccttcggtagagcatcatcagactttaaatgtgagggtccagggttcaagttcctgtttgggcg

SEQ ID NO: 778 - DNA, 73 bp, Artificial Sequence
  Description of Artificial Sequence Synthetic oligonucleotide
gcccagctagctcagtcggtagagcataagactctaaatctcagggttgtggattcgtgccccatgctgggtg

SEQ ID NO: 779 - DNA, 71 bp, Artificial Sequence
  Description of Artificial Sequence Synthetic oligonucleotide
ctgcagctagctcagtcggtagagcatgagactctaaatctcagggtcatgggttcgtgccccatgttggg

SEQ ID NO: 780 - DNA, 70 bp, Artificial Sequence
  Description of Artificial Sequence Synthetic oligonucleotide
ccagcatgtctcagtcggtatagtgtgagactctaaatctcagggtcgtgggttcaagccccacattggg

SEQ ID NO: 781 - DNA, 73 bp, Artificial Sequence
  Description of Artificial Sequence Synthetic oligonucleotide
gtctagctagatcagttggtagagcataagactctaaatctcagggtcatgggtttgagccctacgttgggcg

SEQ ID NO: 782 - DNA, 73 bp, Artificial Sequence
  Description of Artificial Sequence Synthetic oligonucleotide
gcccagctagctcagccggtagagcacaagactctaaatctcagggtcgtgggtttgagccctgtgttgagca

SEQ ID NO: 783 - DNA, 69 bp, Artificial Sequence
  Description of Artificial Sequence Synthetic oligonucleotide
ccgaatagcttagttgatgaagcgtgagactctaaatctcagggtagtgggttcaagccccacattgga

SEQ ID NO: 784 - DNA, 74 bp, Artificial Sequence
  Description of Artificial Sequence Synthetic oligonucleotide
gcctggctacctcagttggtagagcatgggactctaaatcccagagtcagtgggttcaagcctcacattgagtg

SEQ ID NO: 785 - DNA, 73 bp, Artificial Sequence
  Description of Artificial Sequence Synthetic oligonucleotide
gcccggctagctcagtcggtagagcatgagaccctaaatctcagggtcgtgggttcgagccccacgttgggcg

SEQ ID NO: 786 - DNA, 73 bp, Artificial Sequence
  Description of Artificial Sequence Synthetic oligonucleotide
gcccggctagctcagtcggtagagcatgggactctaaatctcagggtcgtgggttcgagccccacgttgggcg

SEQ ID NO: 787 - DNA, 73 bp, Artificial Sequence
  Description of Artificial Sequence Synthetic oligonucleotide
gcccggctagctcagtcgatagagcatgagactctaaatctcagggtcgtgggttcgagccgcacgttgggcg

SEQ ID NO: 788 - DNA, 73 bp, Artificial Sequence
  Description of Artificial Sequence Synthetic oligonucleotide
gcccagctagctcagtcggtagagcatgagactctaaatctcagggtcatgggtttgagccccacgtttggtg

SEQ ID NO: 789 - DNA, 73 bp, Artificial Sequence
  Description of Artificial Sequence Synthetic oligonucleotide
gcctggctagctcagtcggcaaagcatgagactctaaatctcagggtcgtgggctcgagctccatgttgggcg

SEQ ID NO: 790 - DNA, 73 bp, Artificial Sequence
  Description of Artificial Sequence Synthetic oligonucleotide
gcccgactacctcagtcggtggagcatgggactctacatcccagggttgtgggttcgagccccacattgggca

SEQ ID NO: 791 - DNA, 73 bp, Artificial Sequence
  Description of Artificial Sequence Synthetic oligonucleotide
ccccggctggctcagtcagtagatcatgagactctaaatctcagggtcgtgggttcacgccccacactgggcg

SEQ ID NO: 792 - DNA, 65 bp, Artificial Sequence
  Description of Artificial Sequence Synthetic oligonucleotide
gcgctagtcagtagagcatgagactctaaatctcagggtcgtgggttcgagccccacatcgggcg

SEQ ID NO: 793 - DNA, 73 bp, Artificial Sequence
  Description of Artificial Sequence Synthetic oligonucleotide
gcctggatagctcagttggtagagcatcagactctaaatctgagggtccagggttcaagtccctgttcaggca

SEQ ID NO: 794 - DNA, 73 bp, Artificial Sequence
  Description of Artificial Sequence Synthetic oligonucleotide
gccaggatagttcaggtggtagagcatcagactctaaacctgagggttcagggttcaagtctctgtttgggcg

SEQ ID NO: 795 - DNA, 73 bp, Artificial Sequence
  Description of Artificial Sequence Synthetic oligonucleotide
acccagatagctcagtcagtagagcatcagactctaaatctgagggtccaaggttcatgtccctttttgggtg

SEQ ID NO: 796 - DNA, 73 bp, Artificial Sequence
  Description of Artificial Sequence Synthetic oligonucleotide
acctgggtagcttagttggtagagcattggactctaaatttgagggcccaggtttcaagtccctgtttgggtg

SEQ ID NO: 797 - DNA, 74 bp, Artificial Sequence
  Description of Artificial Sequence Synthetic oligonucleotide
gcctgggtagctcagtcggtagagctatcagactctaagcctgaggattcagggttcaatcccttgctggggcg

SEQ ID NO: 798 - DNA, 62 bp, Artificial Sequence
  Description of Artificial Sequence Synthetic oligonucleotide
gatagctcagttgatagagcatcagactctaaatctgagggtccagggttcatgtccctgtt

SEQ ID NO: 799 - DNA, 73 bp, Artificial Sequence
  Description of Artificial Sequence Synthetic oligonucleotide
gttggggtaactcagttggtagagtagcagactctacatctgagggtccagggtttaagtccatgtccaggca

SEQ ID NO: 800 - DNA, 73 bp, Artificial Sequence
  Description of Artificial Sequence Synthetic oligonucleotide
gcctggatagctcagttggtagagcatcagactctaaatctgagggtccagggttcaagtccctgttcaggcg

SEQ ID NO: 801 - DNA, 73 bp, Artificial Sequence; identical to SEQ ID NO: 217
  Description of Artificial Sequence Synthetic oligonucleotide

SEQ ID NO: 802 - DNA, 73 bp, Artificial Sequence
  Description of Artificial Sequence Synthetic oligonucleotide
gcccggatagctcagtcggtagagcatcagactctaaatctgagggtccggggttcaagtccctgttcgggcg

SEQ ID NO: 803 - DNA, 73 bp, Artificial Sequence
  Description of Artificial Sequence Synthetic oligonucleotide
gcctgggtagctcagtcggtagagcatcagactctaaatctgagggtccagggttcaagtccctgtccaggcg

SEQ ID NO: 804 - DNA, 73 bp, Artificial Sequence
  Description of Artificial Sequence Synthetic oligonucleotide
gcctggatagctcagttggtagaacatcagactctaaatctgacggtgcagggttcaagtccctgttcaggcg

SEQ ID NO: 805 - DNA, 73 bp, Artificial Sequence
  Description of Artificial Sequence Synthetic oligonucleotide
gcccggagagctcagtgggtagagcatcagactctaaatctgagggtccagggttcaagtcctcgttcgggca

SEQ ID NO: 806 - DNA, 73 bp, Artificial Sequence
  Description of Artificial Sequence Synthetic oligonucleotide
acctgggtagctcagtaggtagaacatcagactctaaatctgagggtctagggttcaagtccctgtccaggcg

SEQ ID NO: 807 - DNA, 76 bp, Artificial Sequence
  Description of Artificial Sequence Synthetic oligonucleotide
gcctggatagctccttcggtagagcatcatcagactctaaatgtgagggtccagggttcaagttcctgtttgggcg

SEQ ID NO: 808 - DNA, 75 bp, Artificial Sequence
  Description of Artificial Sequence Synthetic oligonucleotide
ggcagaatggtgcagcggttcagcacccaggctcttcagccagctgttgcctgggctcaaatcccagctctgcca

SEQ ID NO: 809 - DNA, 176 bp, Artificial Sequence
  Description of Artificial Sequence Synthetic polynucleotide
ggctgtatagctcagtggtagagcatttgacttcagaatcctatactcaggggaaggagaactgggggtttctcagtgggtcaaaggacttgtagtggtaaatcaaaagcaactctataagctatgtaacaaactttaaagtcatatgtagctgggttcaaatcctgtttctgcca

SEQ ID NO: 810 - DNA, 79 bp, Artificial Sequence
  Description of Artificial Sequence Synthetic oligonucleotide
ggctgtatagctcagtggtagagcatttgacttcagctttaaagtcatatgtagctgggttcaaatcctgtttctgcca

SEQ ID NO: 811 - DNA, 72 bp, Artificial Sequence
  Description of Artificial Sequence Synthetic oligonucleotide
gggggcatagctcagtggtagagcatttgacttcagatcaagaggtccctggttcaaatccaggtgccccct

SEQ ID NO: 812 - DNA, 72 bp, Artificial Sequence
  Description of Artificial Sequence Synthetic oligonucleotide
gggggtatagctcaggggtagagcatttgacttcagatcaagaggtccctggttcaaatccaggtgcccccc

SEQ ID NO: 813 - DNA, 72 bp, Artificial Sequence
  Description of Artificial Sequence Synthetic oligonucleotide
gggggtatagcttagcggtagagcatttgacttcagatcaagaggtccccggttcaaatccgggtgccccct

SEQ ID NO: 814 - DNA, 72 bp, Artificial Sequence
  Description of Artificial Sequence Synthetic oligonucleotide
gggggtatagcttaggggtagagcatttgacttcagatcaaaaggtccctggttcaaatccaggtgcccctt

SEQ ID NO: 815 - DNA, 72 bp, Artificial Sequence
  Description of Artificial Sequence Synthetic oligonucleotide
gggggtatagctcaggggtagagcatttgacttcagatcaagaggtccccagttcaaatctgggtgccccct

SEQ ID NO: 816 - DNA, 72 bp, Artificial Sequence
  Description of Artificial Sequence Synthetic oligonucleotide
gggggtatagctcaggggtagagcatttgacttcagatcaagaagtccccggttcaaatccgggtgccccct

SEQ ID NO: 817 - DNA, 72 bp, Artificial Sequence
  Description of Artificial Sequence Synthetic oligonucleotide
gggggtatagctcaggggtagagcatttgacttcagatcaagaggtctctggttcaaatccaggtgccccct

SEQ ID NO: 818 - DNA, 72 bp, Artificial Sequence
  Description of Artificial Sequence Synthetic oligonucleotide
gggggtatagctcaggggtagagcacttgacttcagatcaagaagtccttggttcaaatccaggtgccccct

SEQ ID NO: 819 - DNA, 72 bp, Artificial Sequence
  Description of Artificial Sequence Synthetic oligonucleotide
ggggatatagctcaggggtagagcatttgacttcagatcaagaggtccccggttcaaatccgggtgcccccc

SEQ ID NO: 820 - DNA, 72 bp, Artificial Sequence
  Description of Artificial Sequence Synthetic oligonucleotide
gggggtatagttcaggggtagagcatttgacttcagatcaagaggtccctggttcaaatccaggtgccccct

SEQ ID NO: 821 - DNA, 72 bp, Artificial Sequence
  Description of Artificial Sequence Synthetic oligonucleotide
gggggtatagctcaggggtagagcatttgacttcaaatcaagaggtccctgattcaaatccaggtgccccct

SEQ ID NO: 822 - DNA, 72 bp, Artificial Sequence
  Description of Artificial Sequence Synthetic oligonucleotide
gggcgtatagctcaggggtagagcatttgacttcagatcaagaggtccccagttcaaatctgggtgccccct

SEQ ID NO: 823 - DNA, 72 bp, Artificial Sequence
  Description of Artificial Sequence Synthetic oligonucleotide
gggggtatagctcacaggtagagcatttgacttcagatcaagaggtccccggttcaaatctgggtgccccct

SEQ ID NO: 824 - DNA, 70 bp, Artificial Sequence
  Description of Artificial Sequence Synthetic oligonucleotide
gggcgtatagctcaggggtagagcatttgacttcagatcaagaggtccccagttcaaatctgggtgccca

SEQ ID NO: 825 - DNA, 72 bp, Artificial Sequence
  Description of Artificial Sequence Synthetic oligonucleotide
gggggtatagctcacaggtagagcatttgacttcagatcaagaggtccccggttcaaatccggttactccct

SEQ ID NO: 826 - DNA, 72 bp, Artificial Sequence
  Description of Artificial Sequence Synthetic oligonucleotide
gggggtagggctcagggatagagcatttgacttcagatcaagaggtccccggttcgaatctaggtgccccct

SEQ ID NO: 827 - DNA, 66 bp, Artificial Sequence
  Description of Artificial Sequence Synthetic oligonucleotide
ggtatatctcagggggcagagcatttgacttcagatcaagaggtccccggttgaaatccgggtgct

SEQ ID NO: 828 - DNA, 72 bp, Artificial Sequence
  Description of Artificial Sequence Synthetic oligonucleotide
gggggtatagctcaggggtagagcacttgacttcagatcaagaggtccctggttcaaatccaggtgccccct

SEQ ID NO: 829 - DNA, 72 bp, Artificial Sequence; identical to SEQ ID NO: 82
  Description of Artificial Sequence Synthetic oligonucleotide

SEQ ID NO: 830 - DNA, 73 bp, Artificial Sequence
  Description of Artificial Sequence Synthetic oligonucleotide
gggggtatagctcagtgggtagagcatttgacttcagatcaagaggtccccggttcaaatccgggtgccccct

SEQ ID NO: 831 - DNA, 72 bp, Artificial Sequence
  Description of Artificial Sequence Synthetic oligonucleotide
gggggtgtagctcagtggtagagcatttgacttcagatcaagaggtccctggttcaaatccaggtgccccct

SEQ ID NO: 832 - DNA, 72 bp, Artificial Sequence; identical to SEQ ID NO: 90
  Description of Artificial Sequence Synthetic oligonucleotide

SEQ ID NO: 833 - DNA, 72 bp, Artificial Sequence; identical to SEQ ID NO: 87
  Description of Artificial Sequence Synthetic oligonucleotide

SEQ ID NO: 834 - DNA, 72 bp, Artificial Sequence; identical to SEQ ID NO: 271
  Description of Artificial Sequence Synthetic oligonucleotide

SEQ ID NO: 835 - DNA, 72 bp, Artificial Sequence; identical to SEQ ID NO: 276
  Description of Artificial Sequence Synthetic oligonucleotide

SEQ ID NO: 836 - DNA, 106 bp, Artificial Sequence
  Description of Artificial Sequence Synthetic polynucleotide
gaagcggtggctcaatggtagagctttcgacttcaattaaatcttggaaattccacggaataagattgcaatcgaagggttgcaggttcaattcctgtccgtttca

SEQ ID NO: 837 - DNA, 72 bp, Artificial Sequence
  Description of Artificial Sequence Synthetic oligonucleotide
gaagcggtggctcaatggtagagctttcgacttcaaatcgaagggttgcaggttcaattcctgtccgtttca

SEQ ID NO: 838 - DNA, 72 bp, Artificial Sequence
  Description of Artificial Sequence Synthetic oligonucleotide
ggcctcatggtgcaacagtagtgtgtctgacttcagatcagaaggttgtatgttcaaatcacataggggtca

SEQ ID NO: 839 - DNA, 72 bp, Artificial Sequence
  Description of Artificial Sequence Synthetic oligonucleotide
gacctcgtggtgaaatggtagcatgtttgacttcaaatcaggaggttgtgtgttcaagtcacatcagggtca

SEQ ID NO: 840 - DNA, 72 bp, Artificial Sequence
  Description of Artificial Sequence Synthetic oligonucleotide
gaccttgtggcgcaatggtagcatgtttgacttcaaatcaggaggttgtgtgttcaagtcacatcagggtca

SEQ ID NO: 841 - DNA, 72 bp, Artificial Sequence
  Description of Artificial Sequence Synthetic oligonucleotide
gacctcgtggcgcaacggtagcgcgtctgacttcagatcagaaggctgcgtgttcgaatcacgccggggtca

SEQ ID NO: 842 - DNA, 72 bp, Artificial Sequence
  Description of Artificial Sequence Synthetic oligonucleotide
gaccttgtggctcaatggtagcgcatctgacttcagatcaggaggttgcacgttcaaatcatgccggggtca

SEQ ID NO: 843 - DNA, 72 bp, Artificial Sequence
  Description of Artificial Sequence Synthetic oligonucleotide
gaccttgtggcgcaacggtagcgcgtctgacttcagatcagaaggttgcgtgttcaaatcacgtcggggtca

SEQ ID NO: 844 - DNA, 72 bp, Artificial Sequence
  Description of Artificial Sequence Synthetic oligonucleotide
gacctcgtggcgcaacggtagcgcgtctgacttcagatcagaaggttgcgtattcaaatcacgtcggggtca

SEQ ID NO: 845 - DNA, 72 bp, Artificial Sequence
  Description of Artificial Sequence Synthetic oligonucleotide
gacctcgtggcgcaacggcagcgcgtctgacttcacattagaaggttgcgtgttcaaatcacgtcggggtca

SEQ ID NO: 846 - DNA, 72 bp, Artificial Sequence
  Description of Artificial Sequence Synthetic oligonucleotide
gacctcatggcgcaacggtagcgcgtctgacttcagatcagaaggttgcgtgttcaaatcacatcggggtca

SEQ ID NO: 847 - DNA, 72 bp, Artificial Sequence
  Description of Artificial Sequence Synthetic oligonucleotide
gacctcgtggtgcaacggtagcgcgtatgatttcagatcagaaggttgcgtgttcaaatcacgtcggggtca

SEQ ID NO: 848 - DNA, 72 bp, Artificial Sequence
  Description of Artificial Sequence Synthetic oligonucleotide
gacctcgtagcgcaacggtagcgcgtctgacttcagatcagaaggttgcgtgttcaaatcacgtcggggtca

SEQ ID NO: 849 - DNA, 76 bp, Artificial Sequence
  Description of Artificial Sequence Synthetic oligonucleotide
aggggtatagctcaattggcagagcgtcggtcttcaaaaccgaaggttgtaggttcgattcctactgcccctgcca

SEQ ID NO: 850 - DNA, 72 bp, Artificial Sequence
  Description of Artificial Sequence Synthetic oligonucleotide
gacctcatggcgcaacggtagcgcgtctgacttcagatcagaaggttgcgtgttcaaatcacgtcggggtca

SEQ ID NO: 851 - DNA, 72 bp, Artificial Sequence
  Description of Artificial Sequence Synthetic oligonucleotide
gacctcgtggcgcaacggtagcgcgtctaacttcagatcagaaggttgcgtgttcaaatcacgtcggggtca

SEQ ID NO: 852 - DNA, 73 bp, Artificial Sequence
  Description of Artificial Sequence Synthetic oligonucleotide
acgggagtagctcagttggtagagcaccggtcttcaaaaccgggtgtcgggagttcgagcctctcctcccgtg

SEQ ID NO: 853 - DNA, 72 bp, Artificial Sequence
  Description of Artificial Sequence Synthetic oligonucleotide
gacctcgtggcgcaacggtagcgcgtctgacttcagatcagaaggttgcatgttcaaatcacgtcggggtca

SEQ ID NO: 854 - DNA, 72 bp, Artificial Sequence
  Description of Artificial Sequence Synthetic oligonucleotide
gactccgtggcgcaacggtagcgcgtccgacttcagatcggaaggttgcgtgttcaaatcacgtcggggtca

SEQ ID NO: 855 - DNA, 72 bp, Artificial Sequence
  Description of Artificial Sequence Synthetic oligonucleotide
gactccgtggcgcaacggtagcgcgtctgacttcagatcagaaggttgcgtgttcaaatcacgtcggggtca

SEQ ID NO: 856 - DNA, 72 bp, Artificial Sequence
  Description of Artificial Sequence Synthetic oligonucleotide
ggcctcgtggcgcaacggtagcgcgtctgactccagatcagaaggttgcgtgttcaaatcacgtcggggtca

SEQ ID NO: 857 - DNA, 72 bp, Artificial Sequence
  Description of Artificial Sequence Synthetic oligonucleotide
ggcctcgtggcgcaacggtagcacgtctgactccagatcagaaggttgcgtgttcaaatcacgtcggggtca

SEQ ID NO: 858 - DNA, 73 bp, Artificial Sequence
  Description of Artificial Sequence Synthetic oligonucleotide
cggcctcgtggcgcaacggtagcacgtctgacttcagatcagaaggttgcgtgttcaaatcacgtcggggtca

SEQ ID NO: 859 - DNA, 72 bp, Artificial Sequence
  Description of Artificial Sequence Synthetic oligonucleotide
ggcctcgtcgcgcaacggtagcgcgtctgactccagatcagaaggttgcgtgttcaaatcacgtcggggtca

SEQ ID NO: 860 - DNA, 72 bp, Artificial Sequence
  Description of Artificial Sequence Synthetic oligonucleotide
ggcctcgtcgcgcaacggtagcgcgtctgacttcagatcagaaggttgcgtgttcaaatcacgtcggggtca

SEQ ID NO: 861 - DNA, 72 bp, Artificial Sequence
  Description of Artificial Sequence Synthetic oligonucleotide
ggcctcgtcgcgcaacggtagcacgtctgactccagatcagaaggttgcgtgttcaaatcacgtcggggtca

SEQ ID NO: 862 - DNA, 72 bp, Artificial Sequence
  Description of Artificial Sequence Synthetic oligonucleotide
ggcctcgtcgcgcaacggtagcacgtctgacttcagatcagaaggttgcgtgttcaaatcacgtcggggtca

SEQ ID NO: 863 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 864 - protein, 1998 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 865 - protein, 1981 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 866 - protein, 1997 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 867 - protein, 1980 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 868 - protein, 1195 aa, Homo sapiens
MFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 869 - DNA, 100 bp, Artificial Sequence
  Description of Artificial Sequence Synthetic polynucleotide
ccaaaacatcttttactgtagtatctacttaccatactacccaagaatggcacactgctcacatcttcaaaagcttaaaccaagagcactacacaggtgc

SEQ ID NO: 870 - DNA, 100 bp, Artificial Sequence
  Description of Artificial Sequence Synthetic polynucleotide
tgtgtgtcggggccggtaccctgcttccggttcccgcacgcattcccggattgcagtgcggaccccttctgtaagcgcgcgataaagcgcggttttggaa

SEQ ID NO: 871 - DNA, 100 bp, Artificial Sequence
  Description of Artificial Sequence Synthetic polynucleotide
tcatgtcatataagtagaaccatacaatatatatataaaatccaggttaatagccaatcttacaacatttctcatattttttgcagttgctaagccatgg

SEQ ID NO: 872 - DNA, 100 bp, Artificial Sequence
  Description of Artificial Sequence Synthetic polynucleotide
acattacaatacatatcaacatatcaccataattaaattgcaagtcttcgtcaaaagcaagccttaaaggagtatcccaaaaacacattttccccagaag

SEQ ID NO: 873 - DNA, 100 bp, Artificial Sequence
  Description of Artificial Sequence Synthetic polynucleotide
agacctttagagcgtggttaaacccatatgttgggatttatgctgcttttatggtagcaataccctatattaagatttgaagtagacccggaaagttagt

SEQ ID NO: 874 - DNA, 100 bp, Artificial Sequence
  Description of Artificial Sequence Synthetic polynucleotide
gttcatgaaagaataaataaatgtttaaaaaaaaaaaaaactgaggtaaatttctatattctttcataaaagcagtttaaagacgaacgtttttcgaggt

SEQ ID NO: 875 - DNA, 100 bp, Artificial Sequence
  Description of Artificial Sequence Synthetic polynucleotide
gctgggtctcggtgacactgacgacgggaggcgcggtcggaagagcgcggggccgtcgcctctggcttaacatagcagatgcgctgagactccaacaggt

SEQ ID NO: 876 - DNA, 100 bp, Artificial Sequence
  Description of Artificial Sequence Synthetic polynucleotide
cagtggcggcgaaaactctctgcgttctggagggagggtgcgggcaggaggaggtagaggatgccttgtaagcggagcaaaaacaaggttcaacgtctgc

SEQ ID NO: 877 - DNA, 100 bp, Artificial Sequence
  Description of Artificial Sequence Synthetic polynucleotide
caaatcacttgcctctcggcgcgagaccgcgatgcgcgggggcgggagcgtgatgatggcatcgcgtaaggagagggtgtgagaagccggatcctgtggt

SEQ ID NO: 878 - DNA, 100 bp, Artificial Sequence
  Description of Artificial Sequence Synthetic polynucleotide
ccctgtgtccgaagaggtctgcgttgcgacttacgtggtagtgcttggaaggtgcggagtagatgagagataagtgaatgtggacaaacctgtcacgtag

SEQ ID NO: 879 - DNA, 100 bp, Artificial Sequence
  Description of Artificial Sequence Synthetic polynucleotide
gagcggagctcagagggtgcgcgctccgccctttcgcgggcctggcatgagcgcagtggttgttacactaaagtgtctccgcctgtcgaatattctcgtg

SEQ ID NO: 880 - DNA, 100 bp, Artificial Sequence
  Description of Artificial Sequence Synthetic polynucleotide
gtgtcactggtttcaaatcaacctcaatttttttggagacgtgagtgctgagcattttttcttcagtgaagtgacttggcagccaaaatcgccaacgccc

SEQ ID NO: 881 - DNA, 100 bp, Artificial Sequence
  Description of Artificial Sequence Synthetic polynucleotide
tcctggcatgtcccgcccaagtcccttagccccgctccccaaccctgccccattcccactctagtacccgtaagctacaagacgccgccgttcgtcgggt

SEQ ID NO: 882 - DNA, 100 bp, Artificial Sequence
  Description of Artificial Sequence Synthetic polynucleotide
tgctcagtcgtcctgccgggcgggccctgaggttgcaagggacggaggaagtttcgtgcgtgcgcccttcctatagcgcccagtagaactgacagtacct

SEQ ID NO: 883 - DNA, 100 bp, Artificial Sequence
  Description of Artificial Sequence Synthetic polynucleotide
tcctcggattacgcatgctcagtgcaatcttcggttgcctggactagcgctccggtttttctgtgctgaacctcaggggacgccgacacacgtacacgtc

SEQ ID NO: 884 - DNA, 100 bp, Artificial Sequence
  Description of Artificial Sequence Synthetic polynucleotide
gataatttcctgaaagaaaagatcaattcgatgttaccaaatctgggatatccagaaaaattttcttcttctcctaggagaaaaactatcaaatgtcagg

SEQ ID NO: 885 - DNA, 100 bp, Artificial Sequence
  Description of Artificial Sequence Synthetic polynucleotide
tctctcacggcaaactgttgcagactgtagagacgctatgccaagaatcttttacttaaaagcaggaatagattcaataggcaacttcactgcacatgta

SEQ ID NO: 886 - DNA, 100 bp, Artificial Sequence
  Description of Artificial Sequence Synthetic polynucleotide
caacctccccttctcaaggagcaggtggattggtcccgagctagctggtgggcggaggtgacgtttttataagttgctcaagagacggtaacaaccgacg

SEQ ID NO: 887 - DNA, 100 bp, Artificial Sequence
  Description of Artificial Sequence Synthetic polynucleotide
gtggaacttccactgaattactcttttcgcatgtaagatcactgaaccgtgataatcattgatcctatttgtagaactgtatgaaacagttccctaagga

SEQ ID NO: 888 - DNA, 100 bp, Artificial Sequence
  Description of Artificial Sequence Synthetic polynucleotide
tcgctcaacaggcggccagggtgcgagcagtgaagctgcggcacgccggagcgtttaatggccatcaaattggcctctctaggaggtagctgcagccgga

SEQ ID NO: 889 - DNA, 51 bp, Artificial Sequence
  Description of Artificial Sequence Synthetic oligonucleotide
atagaaagaaatgagactgctcgatagaaaaatgtgaaagtaaactttgat

SEQ ID NO: 890 - DNA, 51 bp, Artificial Sequence
  Description of Artificial Sequence Synthetic oligonucleotide
atagaaagaaatgagactgctcgacagaaaaatgtgaaagtaaactttgat

SEQ ID NO: 891 - DNA, 51 bp, Artificial Sequence
  Description of Artificial Sequence Synthetic oligonucleotide
tataatgcaatgaaaaaattaggatagaaaaaaccgcaaaagcctatacct

SEQ ID NO: 892 - DNA, 51 bp, Artificial Sequence
  Description of Artificial Sequence Synthetic oligonucleotide
tataatgcaatgaaaaaattaggacagaaaaaaccgcaaaagcctatacct

SEQ ID NO: 893 - DNA, 51 bp, Artificial Sequence
  Description of Artificial Sequence Synthetic oligonucleotide
gagaagtttgatcccgatgcaacttagttcatggaatttgaaaaattatct

SEQ ID NO: 894 - DNA, 51 bp, Artificial Sequence
  Description of Artificial Sequence Synthetic oligonucleotide
gagaagtttgatcccgatgcaactcagttcatggaatttgaaaaattatct

SEQ ID NO: 895 - DNA, 20 bp, Artificial Sequence
  Description of Artificial Sequence Synthetic oligonucleotide
aaaggcaccttctcgctggc

SEQ ID NO: 896 - DNA, 17 bp, Artificial Sequence
  Description of Artificial Sequence Synthetic oligonucleotide
acttgtatgttgttttt

SEQ ID NO: 897 - DNA, 10 bp, Artificial Sequence
  Description of Artificial Sequence Synthetic oligonucleotide
tctcgctggc

SEQ ID NO: 898 - DNA, 6063 bp, Artificial Sequence
  Description of Artificial Sequence Synthetic polynucleotide
atggagcaaacagtgcttgtaccaccaggacctgacagcttcaacttcttcaccagagaatcccttgcagctattgaaaggcgcattgcagaagagaaggctaagaatcccaagccagacaaaaaagatgatgatgaaaatggcccaaagccaaacagtgacttggaagctgggaagaaccttccatttatctatggagacattcctccagagatggtgtcggagcctctggaggacctggacccctactatatcaataagaagacttttatagtattgaataaagggaaggccatcttccggttcagtgccacctccgccctgtacattttaacacccttcaatcctcttaggaaaatagctattaagattttggtacactcattattcagcatgttaatcatgtgcactattttgacaaactgtgtatttatgacaatgagtaaccctcccgactggacaaagaatgtggagtacaccttcacaggaatatatacttttgaatcactaataaaaattattgcaaggggcttctgtttagaagattttactttccttcgcgacccatggaactggctggacttcactgtcattacattcgcatatgtgacggagtttgtggacctgggcaatgtctcagcattgagaacattcagagttcttcgagcattgaaaactatttcagtcattccaggcctgaagaccatcgtgggggccctgatccagtcggtgaagaagctgtctgacgtcatgatactcactgtgttctgtctcagtgtgttcgcactcatcgggttgcagctcttcatgggcaacctgaggaataaatgtgtacagtggcctcccaccaacgcttcccttgaggaacatagcatagagaagaatataactatggattacaatggcacacttgtaaatgaaaccgtgttcgagtttgactggaaatcatacattcaagactcaagatatcattatttcctggagggtgttttagatgcactgctgtgtggaaatagctctgatgcaggccaatgtccagaaggatatatgtgtgtaaaagctggtagaaaccctaattatggttacacaagctttgataccttcagttgggcatttttgtccctgtttcgactgatgactcaggacttctgggaaaatctataccaactgacattgcgtgctgctggcaaaacctacatgatattttttgtgctggtcattttcttgggctcattctacctgataaacttgatcctggctgtggtggccatggcctatgaggagcagaatcaggccacactggaggaggctgaacagaaagaggcagaatttcagcagatgttggagcaacttaagaagcagcaagaggctgcacagcaggcagcggctacaacagcctcagaacattccagggagcccagtgcagcaggcaggctctcagatagctcttcagaagcctctaagttgagttcgaagagtgctaaagaaagacgaaatcggaggaaaaaaaggaaacagaaagagcagtctggaggagaagagaaagatgatgatgaattccacaagtctgagtctgaagacagcatcaggaggaaggggtttcgcttctccatagaagggaatagactgacatatgaaaagaggtactcttccccgcatcagtctctgttaagcattcgtggttccctgttctccccaagacgcaatagcagaacaagtcttttcagctttagagggcgagccaaggatgtggggtctgagaatgactttgctgatgatgaacacagcacctttgaggataatgagagccgtagagactcactgttcgttccccgaagacacggagagcgacgcaacagtaacctgagccagaccagcaggtcctcccgaatgctggcggtgtttccagccaatgggaagatgcacagcacggtggattgcaatggtgtggtttccttggttggtggaccctcagttcccacatcgccagttggacagcttctgccagagggaacaaccactgaaactgagatgagaaagaggaggtcgagctctttccatgtttccatggactttctagaagatccttcccagaggcaaagggcaatgagcatagccagcatcttaacaaatacagtagaagaactagaagaatccaggcagaaatgtccaccctgttggtataaattttccaacatattcttaatttgggactgttctccatattggctgaaagttaaacatattgtcaacctggtggtgatggacccatttgttgatctggccattaccatctgcattgtgttaaatacgctcttcatggctatggagcactaccccatgactgaacatttcaaccatgttcttacagtgggaaacttggtcttcactgggattttcacagcagaaatgttcctgaaaatcatcgcaatggatccttactattacttccaagaaggctggaatatctttgatggtttcattgtgacactcagcctggtagaacttggccttgccaatgtggaaggattgtcagttctccgttcatttcgactgctccgagtgttcaagttggcaaagtcttggcccacactgaatatgctcattaagatcattggtaactcggtgggagcactgggcaacctgactctggtgttggccatcattgtctttatttttgccgtggttggcatgcagctgtttggaaaaagttacaaagattgtgtctgcaaaattgccactgactgcaaactcccacgttggcacatgaacgacttcttccactcgttcctgatcgtgttccgcgtgctgtgtggggagtggatagagaccatgtgggactgcatggaggtggcaggacaagctatgtgccttactgtcttcatgatggtcatggtgattgggaaccttgtggtcttgaacctctttctggccttgcttctgagctcatttagtgcagacaaccttgcagccactgatgatgacaatgagatgaacaacctgcagattgctgtggacaggatgcacaaaggaatagcttatgtaaaaagaaaaatatatgaattcattcaacaatcctttgttaagaaacagaagattctagatgaaattaagccacttgatgatctaaacaacagaaaagacaattgtatctctaaccacacaacagaaattgggaaagatctggactgtctgaaagatgtgaatggaaccacaagtggcatagggacgggcagcagtgtggagaagtacatcattgatgagagtgattatatgtcattcataaacaaccccagcctcactgtgactgtgcccattgctgtgggagagtctgactttgagaacttaaacacagaagactttagcagtgaatcagatctagaagaaagcaaagagaaactcaacgaaagcagtagctcctcagagggaagcacagtagacattggggcgcctgcagaggaacagcctgtcattgaaccagaagaaacccttgagcccgaagcttgcttcactgaaggctgtgtccagagattcaagtgctgtcaaatcagcgtggaagaaggaagagggaaacagtggtggaacctacggaggacgtgcttccgaatagttgaacacaactggtttgagaccttcattgtgttcatgattctcctgagtagtggtgccctggcctttgaggatatatatattgatcagcgaaagacgatcaaaaccatgctggagtatgctgacaaagtcttcacttacattttcatcctggagatgctcctcaaatgggtggcctatggctatcaaacatacttcaccaatgcctggtgttggctagacttcttaattgttgatgtttcattggtcagtttaacagcaaatgccttgggttactctgaactcggggccatcaaatccctaaggacactaagagctctgagacccctaagagccttatcacgatttgaagggatgagggtggttgtgaatgccctgttaggagcaattccatccatcatgaatgtgcttctggtttgccttatattctggctaattttcagcatcatgggcgtaaatttgtttgctggcaaattctaccactgtgttaacaccacaactggtgacatatttgagatcagcgaagtcaataatcattctgattgcctaaaactaatagaaagaaatgagaccgcccggtggaaaaatgtgaaagtaaactttgataatgtaggatttgggtatctttctttgcttcaagttgccacatttaagggctggatggatatcatgtatgctgcagttgattccagaaatgttgaactacagcctaagtatgaggaaagcctgtacatgtatttgtacttcgtcatcttcatcatcttcgggtccttctttaccctgaacctgtttattggtgtcattatcgacaatttcaaccagcaaaagaagaagtttggaggtcaagacatctttatgacagaagaacagaagaaatactataatgcaatgaagaaattaggatcaaaaaagccacaaaagcctatccctcgacctggaaacaaatttcaaggaatggtttttgactttgtaaccagacaagtgtttgatatcagcatcatgatcctcatctgtctgaacatggtgaccatgatggtggaaacggatgaccagagcgattatgtgacaagcattttgtcacgcatcaacctggtgttcatcgtcctgttcaccggcgagtgtgtgctcaagctcatctcgctccgccattattatttcaccattggatggaacattttcgattttgtggtggtcatcctctccattgtagggatgtttcttgcggagctaatagaaaagtattttgtgtctcctaccctgttccgagtcatccgcctggccaggattggacgaatcctacgcctgatcaaaggtgccaaggggatccgcacgctgctctttgctctgatgatgtcccttcctgcgctgtttaacatcggcctcctgctttttctcgtcatgttcatctacgccatctttgggatgtccaactttgcctatgttaagagggaagttgggattgatgacatgttcaactttgagaccttcggcaacagcatgatctgcctgttccaaatcaccacctctgcgggctgggatggactgctggcccccatcctcaacagcaaaccccctgactgtgaccctaataaagttaaccctggaagctcggtgaagggagactgtgggaacccatctgtggggattttcttttttgtcagctacatcatcatatccttcctggttgtggtgaacatgtacattgctgtcatcctggagaacttcagcgttgccacagaagaaagtgcagagcctctgagtgaggacgactttgagatgttctacgaggtctgggagaagttcgaccctgacgccacccagttcatggaatttgaaaaattatctcagtttgcagctgctctagaaccccctctcaatttgccacaaccaaacaaacttcagctcattgccatggacctgcccatggtgagtggagaccgcatccactgcctggacatcttatttgcttttacaaagcgggtgttgggtgagagtggagagatggatgctcttcgaatccagatggaagagcggttcatggcttccaacccctccaaggtctcttatcagcccatcactactacattaaaacgcaaacaagaggaggtgtcagctgttatcattcagcgagcttataggcgccaccttttgaagcgaacagtaaaacaagcttcattcacatacaataagaacaaactcaaaggtggggctaatcttcttgtaaaagaagacatgctcattgacagaataaacgaaaactctattacggagaaaactgacctgacaatgtccacagcagcttgtccgccctcctacgatcgggtgacaaagccaatcgtggagaaacacgagcaggaagggaaggatgaaaaagccaaagggaaagactacaaagaccatgacggtgattataaagatcatgacatcgattacaaggatgacgatgacaagtaa

SEQ ID NO: 899 - DNA, 6063 bp, Artificial Sequence
  Description of Artificial Sequence Synthetic polynucleotide
atggagcaaacagtgcttgtaccaccaggacctgacagcttcaacttcttcaccagagaatcccttgcagctattgaaaggcgcattgcagaagagaaggctaagaatcccaagccagacaaaaaagatgatgatgaaaatggcccaaagccaaacagtgacttggaagctgggaagaaccttccatttatctatggagacattcctccagagatggtgtcggagcctctggaggacctggacccctactatatcaataagaagacttttatagtattgaataaagggaaggccatcttccggttcagtgccacctccgccctgtacattttaacacccttcaatcctcttaggaaaatagctattaagattttggtacactcattattcagcatgttaatcatgtgcactattttgacaaactgtgtatttatgacaatgagtaaccctcccgactggacaaagaatgtggagtacaccttcacaggaatatatacttttgaatcactaataaaaattattgcaaggggcttctgtttagaagattttactttccttcgcgacccatggaactggctggacttcactgtcattacattcgcatatgtgacggagtttgtggacctgggcaatgtctcagcattgagaacattcagagttcttcgagcattgaaaactatttcagtcattccaggcctgaagaccatcgtgggggccctgatccagtcggtgaagaagctgtctgacgtcatgatactcactgtgttctgtctcagtgtgttcgcactcatcgggttgcagctcttcatgggcaacctgaggaataaatgtgtacagtggcctcccaccaacgcttcccttgaggaacatagcatagagaagaatataactatggattacaatggcacacttgtaaatgaaaccgtgttcgagtttgactggaaatcatacattcaagactcaagatatcattatttcctggagggtgttttagatgcactgctgtgtggaaatagctctgatgcaggccaatgtccagaaggatatatgtgtgtaaaagctggtagaaaccctaattatggttacacaagctttgataccttcagttgggcatttttgtccctgtttcgactgatgactcaggacttctgggaaaatctataccaactgacattgcgtgctgctggcaaaacctacatgatattttttgtgctggtcattttcttgggctcattctacctgataaacttgatcctggctgtggtggccatggcctatgaggagcagaatcaggccacactggaggaggctgaacagaaagaggcagaatttcagcagatgttggagcaacttaagaagcagcaagaggctgcacagcaggcagcggctacaacagcctcagaacattccagggagcccagtgcagcaggcaggctctcagatagctcttcagaagcctctaagttgagttcgaagagtgctaaagaaagacgaaatcggaggaaaaaaaggaaacagaaagagcagtctggaggagaagagaaagatgatgatgaattccacaagtctgagtctgaagacagcatcaggaggaaggggtttcgcttctccatagaagggaatagactgacatatgaaaagaggtactcttccccgcatcagtctctgttaagcattcgtggttccctgttctccccaagacgcaatagcagaacaagtcttttcagctttagagggcgagccaaggatgtggggtctgagaatgactttgctgatgatgaacacagcacctttgaggataatgagagccgtagagactcactgttcgttccccgaagacacggagagcgacgcaacagtaacctgagccagaccagcaggtcctcccgaatgctggcggtgtttccagccaatgggaagatgcacagcacggtggattgcaatggtgtggtttccttggttggtggaccctcagttcccacatcgccagttggacagcttctgccagagggaacaaccactgaaactgagatgagaaagaggaggtcgagctctttccatgtttccatggactttctagaagatccttcccagaggcaaagggcaatgagcatagccagcatcttaacaaatacagtagaagaactagaagaatccaggcagaaatgtccaccctgttggtataaattttccaacatattcttaatttgggactgttctccatattggctgaaagttaaacatattgtcaacctggtggtgatggacccatttgttgatctggccattaccatctgcattgtgttaaatacgctcttcatggctatggagcactaccccatgactgaacatttcaaccatgttcttacagtgggaaacttggtcttcactgggattttcacagcagaaatgttcctgaaaatcatcgcaatggatccttactattacttccaagaaggctggaatatctttgatggtttcattgtgacactcagcctggtagaacttggccttgccaatgtggaaggattgtcagttctccgttcatttcgactgctccgagtgttcaagttggcaaagtcttggcccacactgaatatgctcattaagatcattggtaactcggtgggagcactgggcaacctgactctggtgttggccatcattgtctttatttttgccgtggttggcatgcagctgtttggaaaaagttacaaagattgtgtctgcaaaattgccactgactgcaaactcccacgttggcacatgaacgacttcttccactcgttcctgatcgtgttccgcgtgctgtgtggggagtggatagagaccatgtgggactgcatggaggtggcaggacaagctatgtgccttactgtcttcatgatggtcatggtgattgggaaccttgtggtcttgaacctctttctggccttgcttctgagctcatttagtgcagacaaccttgcagccactgatgatgacaatgagatgaacaacctgcagattgctgtggacaggatgcacaaaggaatagcttatgtaaaaagaaaaatatatgaattcattcaacaatcctttgttaagaaacagaagattctagatgaaattaagccacttgatgatctaaacaacagaaaagacaattgtatctctaaccacacaacagaaattgggaaagatctggactgtctgaaagatgtgaatggaaccacaagtggcatagggacgggcagcagtgtggagaagtacatcattgatgagagtgattatatgtcattcataaacaaccccagcctcactgtgactgtgcccattgctgtgggagagtctgactttgagaacttaaacacagaagactttagcagtgaatcagatctagaagaaagcaaagagaaactcaacgaaagcagtagctcctcagagggaagcacagtagacattggggcgcctgcagaggaacagcctgtcattgaaccagaagaaacccttgagcccgaagcttgcttcactgaaggctgtgtccagagattcaagtgctgtcaaatcagcgtggaagaaggaagagggaaacagtggtggaacctacggaggacgtgcttccgaatagttgaacacaactggtttgagaccttcattgtgttcatgattctcctgagtagtggtgccctggcctttgaggatatatatattgatcagcgaaagacgatcaaaaccatgctggagtatgctgacaaagtcttcacttacattttcatcctggagatgctcctcaaatgggtggcctatggctatcaaacatacttcaccaatgcctggtgttggctagacttcttaattgttgatgtttcattggtcagtttaacagcaaatgccttgggttactctgaactcggggccatcaaatccctaaggacactaagagctctgagacccctaagagccttatcacgatttgaagggatgagggtggttgtgaatgccctgttaggagcaattccatccatcatgaatgtgcttctggtttgccttatattctggctaattttcagcatcatgggcgtaaatttgtttgctggcaaattctaccactgtgttaacaccacaactggtgacatatttgagatcagcgaagtcaataatcattctgattgcctaaaactaatagaaagaaatgagaccgcctgatggaaaaatgtgaaagtaaactttgataatgtaggatttgggtatctttctttgcttcaagttgccacatttaagggctggatggatatcatgtatgctgcagttgattccagaaatgttgaactacagcctaagtatgaggaaagcctgtacatgtatttgtacttcgtcatcttcatcatcttcgggtccttctttaccctgaacctgtttattggtgtcattatcgacaatttcaaccagcaaaagaagaagtttggaggtcaagacatctttatgacagaagaacagaagaaatactataatgcaatgaagaaattaggatcaaaaaagccacaaaagcctatccctcgacctggaaacaaatttcaaggaatggtttttgactttgtaaccagacaagtgtttgatatcagcatcatgatcctcatctgtctgaacatggtgaccatgatggtggaaacggatgaccagagcgattatgtgacaagcattttgtcacgcatcaacctggtgttcatcgtcctgttcaccggcgagtgtgtgctcaagctcatctcgctccgccattattatttcaccattggatggaacattttcgattttgtggtggtcatcctctccattgtagggatgtttcttgcggagctaatagaaaagtattttgtgtctcctaccctgttccgagtcatccgcctggccaggattggacgaatcctacgcctgatcaaaggtgccaaggggatccgcacgctgctctttgctctgatgatgtcccttcctgcgctgtttaacatcggcctcctgctttttctcgtcatgttcatctacgccatctttgggatgtccaactttgcctatgttaagagggaagttgggattgatgacatgttcaactttgagaccttcggcaacagcatgatctgcctgttccaaatcaccacctctgcgggctgggatggactgctggcccccatcctcaacagcaaaccccctgactgtgaccctaataaagttaaccctggaagctcggtgaagggagactgtgggaacccatctgtggggattttcttttttgtcagctacatcatcatatccttcctggttgtggtgaacatgtacattgctgtcatcctggagaacttcagcgttgccacagaagaaagtgcagagcctctgagtgaggacgactttgagatgttctacgaggtctgggagaagttcgaccctgacgccacccagttcatggaatttgaaaaattatctcagtttgcagctgctctagaaccccctctcaatttgccacaaccaaacaaacttcagctcattgccatggacctgcccatggtgagtggagaccgcatccactgcctggacatcttatttgcttttacaaagcgggtgttgggtgagagtggagagatggatgctcttcgaatccagatggaagagcggttcatggcttccaacccctccaaggtctcttatcagcccatcactactacattaaaacgcaaacaagaggaggtgtcagctgttatcattcagcgagcttataggcgccaccttttgaagcgaacagtaaaacaagcttcattcacatacaataagaacaaactcaaaggtggggctaatcttcttgtaaaagaagacatgctcattgacagaataaacgaaaactctattacggagaaaactgacctgacaatgtccacagcagcttgtccgccctcctacgatcgggtgacaaagccaatcgtggagaaacacgagcaggaagggaaggatgaaaaagccaaagggaaagactacaaagaccatgacggtgattataaagatcatgacatcgattacaaggatgacgatgacaagtaa

SEQ ID NO: 900 - DNA, 55 bp, Artificial Sequence
  Description of Artificial Sequence Synthetic oligonucleotide
agcgctccggtttttctgtgctgaacctcaggggacgccgacacacgtacacgtc

SEQ ID NO: 901 - protein, 22 aa, Artificial Sequence
  Description of Artificial Sequence Synthetic peptide
DYKDHDGDYKDHDIDYKDDDDK

SEQ ID NO: 902 - protein, 7 aa, Tobacco etch virus
  MOD_RES (7)..(7): Gly or Ser
ENLYFQX